(19) United States
(12) Patent Application Publication
(10) Pub. No.: US 2025/0137015 A1
(43) Pub. Date: May 1, 2025

(54) YEAST PLATFORM FOR RENEWABLE INDUSTRIAL TERPENE PRODUCTION

(71) Applicants: Zhen Wang, Getzville, NY (US); Minakshi Mukherjee, Stillwater, OK (US)

(72) Inventors: Zhen Q. Wang, Getzville, NY (US); Minakshi Mukherjee, Stillwater, OK (US)

(73) Assignees: Zhen Wang, Getzville, NY (US); Minakshi Mukherjee, Stillwater, OK (US)

(21) Appl. No.: 18/929,487

(22) Filed: Oct. 28, 2024

Related U.S. Application Data
(60) Provisional application No. 63/593,799, filed on Oct. 27, 2023.

(51) Int. Cl. C12P 5/00; C12N 15/81

(52) U.S. Cl. CPC C12P 5/007; C12N 15/81; C12N 2800/102

(57) ABSTRACT

The disclosure relates to compositions, methods of making terpenes, methods of making cells, methods of culturing cells, and kits for making terpenes.

### CROSS REFERENCE TO RELATED APPLICATIONS

This application claims the benefit of U.S. provisional application No. 63/593,799, which was filed Oct. 27, 2023, is entitled “A Yeast Platform for Renewable Industrial Terpene Production,” which is incorporated herein by reference in its entirety.

### SEQUENCE LISTING

The Sequence Listing filed herewith has the filename ZHE-001-US—Sequence Listing.xml, was created on Oct. 28, 2024, has a file size of 174,239 bytes, and is incorporated herein by reference in its entireties.

### FIELD

The disclosure relates to compositions, methods of making terpenes, methods of making cells, methods of culturing cells, and kits for making terpenes.

### BACKGROUND

Terpenes are five-carbon isoprene derivatives that constitute the largest class of natural products and are widely used as fuels, medicines, and fragrances (1, 2). However, terpene yields from natural biological sources are often low, and chemical synthesis is challenging due to their structural complexity. Engineering microbes, especially bakers' yeast, for sustainable terpene production has achieved considerable success in the past decade (3, 4). Terpene biosynthesis in yeast relies on the mevalonate (MVA) pathway, which produces the universal terpene precursors isopentyl pyrophosphate (IPP) and dimethylallyl pyrophosphate (DMAPP) (FIG. 1A).

The production of terpenes from engineered microbes contributes markedly to the bioeconomy by providing essential medicines, sustainable materials, and renewable fuels. The mevalonate pathway leading to the synthesis of terpene precursors has been extensively targeted for engineering. Nevertheless, the importance of individual pathway enzymes to the overall pathway flux and final terpene yield is less known, especially enzymes that are thought to be non-rate-limiting.

Engineered yeast strains for terpene production usually overexpresses MVA pathway genes to provide sufficient IPP and DMAPP for producing a wide range of terpenes in yeast Saccharomyces cerevisiae (5). In recent works, all seven genes of the MVA pathway were overexpressed from the yeast genome to increase concentrations of IPP and DMAPP and subsequently increased the titer of specific terpenes (6-15). The seven genes were usually expressed from strong promoters, and there has been limited attention to balancing the expression of each gene. Unbalanced expression of pathway genes may lead to the accumulation of intermediates that inhibit enzyme activities through feedback regulations (16). Combinatorial screening of the MVA pathway genes expressed from promoters with various strengths can help identify the optimal expression of each enzyme for maximized pathway flux and terpene production. Such effort can also reveal the in vivo contribution of each gene in the MVA pathway, especially the five non-rate-limiting enzymes. While there is a consensus that HMG-CoA reductase Hmg1p and IPP isomerase Idi1p are bottlenecks (17-21), varying information exists regarding the relative contribution of the other five MVA pathway genes (22-29). Moreover, creating a yeast platform strain with increased terpene precursors can shorten the strain development process to support the high-titer production of terpenes. A platform strain is a genetically engineered microbe that provides abundant precursors for producing various products (30). Developing a platform strain eliminates repetitive engineering of the same precursor pathway for different target molecules. Several yeast platform strains have been developed to access precursors for alkaloids and aromatics (31-35), but no such platform strain exists for terpenes. Therefore, there is an ongoing and unmet need for a yeast platform strain that can be used to produce any terpene once compound-specific downstream modifications are incorporated. The disclosure is pertinent to this need.

### SUMMARY

In some embodiments, the disclosure relates to a composition comprising a modified yeast cell. In some embodiments, the modified yeast cell comprises open reading frames encoding ERG8, ERG10, ERG12, ERG13, and ERG19, and a first regulatory sequence of weak-strength, medium-strength or high-strength operably linked to the open reading frame encoding ERG12. In some embodiments, the first regulatory sequence is of weak strength. In some embodiments, the first regulatory sequence is of medium strength. In some embodiments, the first regulatory sequence is of high-strength. In some embodiments, the yeast cell further comprises one or both of an open reading frame encoding tHMG1 and an open reading frame encoding IDI1. In some embodiments, the yeast cell further comprises one or more of: a second regulatory sequence operably linked to the open reading frame encoding ERG8, a third regulatory sequence operably linked to the open reading frame encoding ERG10, a fourth regulatory sequence operably linked to the open reading frame encoding ERG13, and a fifth regulatory sequence operably linked to the open reading frame encoding ERG19. In some embodiments, the first regulatory sequence, the second regulatory sequence, the third regulatory sequence, the fourth regulatory sequence, and the fifth regulatory sequence are each high-strength promoters. In some embodiments, the first regulatory sequence, the second regulatory sequence, the third regulatory sequence, the fourth regulatory sequence, and the fifth regulatory sequence are independently selected from a promoter comprising a nucleic acid sequence comprising at least about 72% sequence identity to SEQ ID NO: 1, SEQ ID NO: 2, SEQ ID NO: 3, SEQ ID NO: 4, SEQ ID NO: 5, SEQ ID NO: 6, or SEQ ID NO: 7. In some embodiments, the first regulatory sequence, the second regulatory sequence, the third regulatory sequence, the fourth regulatory sequence, and the fifth regulatory sequence are independently selected from: pTDH3, pCCW12, pPGK1, pHHF2, pTEF1, pTEF2, and pHHF1. In some embodiments, the first regulatory sequence, the second regulatory sequence, the third regulatory sequence, the fourth regulatory sequence, and the fifth regulatory sequence are each medium-strength promoters. In some embodiments, the first regulatory sequence, the second regulatory sequence, the third regulatory sequence, the fourth regulatory sequence, and the fifth regulatory sequence are independently selected from a promoter comprising a nucleic acid sequence that comprises at least about 72% sequence to SEQ ID NO: 8, SEQ ID NO: 9, SEQ ID NO: 10, SEQ ID NO: 11, SEQ ID NO: 12. In some embodiments, the first regulatory sequence, the second regulatory sequence, the third regulatory sequence, the fourth regulatory sequence, and the fifth regulatory sequence are independently selected from pRPL18B, pHTB2, pALD6, pPAB1, and pRET2. In some embodiments, the first regulatory sequence, the second regulatory sequence, the third regulatory sequence, the fourth regulatory sequence, and the fifth regulatory sequence are each weak-strength promoters. In some embodiments, the first regulatory sequence, the second regulatory sequence, the third regulatory sequence, the fourth regulatory sequence, and the fifth regulatory sequence are independently selected from a promoter comprising a nucleic acid sequence that comprises at least about 72% sequence to SEQ ID NO: 13, SEQ ID NO: 14, SEQ ID NO: 15, SEQ ID NO: 16, SEQ ID NO: 17. In some embodiments, the first regulatory sequence, the second regulatory sequence, the third regulatory sequence, the fourth regulatory sequence, and the fifth regulatory sequence are independently selected from pPOP6, pRNR2, pPSP2, pRAD27, and pREV1. In some embodiments, the first regulatory sequence is selected from a promoter comprising a nucleic acid sequence having at least about 72% sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17; and the second regulatory sequence, the third regulatory sequence, the fourth regulatory sequence, and the fifth regulatory sequence are each independently selected from a promoter comprising a nucleic acid sequence comprising at least about 72% sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17. In some embodiments, the modified yeast cell is free of modification of any of yeast genes: LPP1, DPP1, HO, ERG1, ANT1, IDP2, IDP3, Cit2, ACS1, ACL1, ACL2, Met15, RHR2, NADH-HMGR, ERG9, GPD1, and GPD2. In some embodiments, the yeast cell further comprises one, two, or three regulatory sequences operably linked to the open reading frame encoding ERG8. In some embodiments, the yeast cell further comprises one, two, or three regulatory sequences operably linked to the open reading frame encoding ERG10. In some embodiments, the yeast cell further comprises one, two, or three regulatory sequences operably linked to the open reading frame encoding ERG13. In some embodiments, the yeast cell further comprises one, two, or three regulatory sequences operably linked to the open reading frame encoding ERG19. In some embodiments, the yeast cell further comprises one or more of a sixth regulatory sequence operably linked to the open reading frame encoding ERG12 and seventh regulatory sequence operably linked to the open reading frame encoding ERG12. In some embodiments, a culture of the modified yeast cell has about a 94-fold, about a 60-fold, and about a 35-fold improved titer of monoterpene geraniol, sesquiterpene α-humulene, and triterpene squalene, respectively, over a culture of wild type yeast cell. In some embodiments, the composition further comprises a terpene and a culture medium. In some embodiments, the terpene is at least about 10 mg/L to about 20 mg/L in the culture medium. In some embodiments, the ERG8, ERG10, ERG12, ERG13, and ERG19 expression levels in the yeast cell at a ratio of about 2.8 ERG8:about 1.0 ERG10:about 2.1 ERG12:about 1.3 ERG13:about 4.5 ERG19.

In some embodiments, the disclosure relates to a method of making a terpene. In some embodiments, the method comprises inoculating a growth medium with a yeast cell, the yeast cell comprising open reading frames encoding ERG8, ERG10, ERG12, ERG13, ERG19, tHMG1, and IDI1; and a first regulatory sequence of weak-strength, medium-strength or high-strength operably linked to the open reading frame encoding ERG12. In some embodiments, the growth medium is synthetic-defined medium plus an antibiotic. In some embodiments, the growth medium is glucose medium or oleate medium.

In some embodiments, the method further comprises incubating the yeast cell in the growth medium. In some embodiments, the method further comprises isolating a plurality of yeast cells from the tissue culture medium after the incubating the plurality of cells. In some embodiments, the method further comprises disrupting the membrane of the yeast cells. In some embodiments, the method further comprises collecting the liquid phase after the step of disrupting. In some embodiments, the method further comprises drying the liquid phase. In some embodiments, the method comprises dissolving the dried product from the step of drying the liquid phase in a solvent.

In some embodiments, the disclosure relates to a kit comprising a nucleic acid molecule. In some embodiments, the nucleic acid molecule comprises a nucleic acid sequence comprising an open reading frame encoding ERG12 and a first regulatory sequence of weak-strength, medium-strength or high-strength operably linked to the open reading frame encoding ERG12. In some embodiments, the kit further comprises a yeast cell.

### BRIEF DESCRIPTION OF THE DRAWINGS

The patent or application file contains at least one drawing executed in color. Copies of this patent or patent application publication with color drawings will be provided by the Office upon request and payment of the necessary fee.

The following detailed description of embodiments of the present invention will be better understood when read in conjunction with the appended drawings. For the purpose of illustrating the invention, there are shown in the drawings certain embodiments. It is understood, however, that the invention is not limited to the precise arrangements and instrumentalities shown. In the drawings:

FIGS. 1A-1C illustrate overexpressing the complete MVA pathway led to increased geraniol production. (FIG. 1A) The MVA pathway leads to geraniol production. Proteins in blue were overexpressed MVA enzymes. Erg10p: acetoacetyl-CoA thiolase; Erg13p: 3-hydroxy-3-methylglutaryl-CoA (HMG-CoA) synthase; tHmg1p: truncated HMG-CoA reductase without the regulatory domain; Erg12p: mevalonate kinase; Erg8p: phosphomevalonate kinase Erg19p: mevalonate pyrophosphate decarboxylase; Idi1p: isopentenyldiphosphate isomerase; Erg20wwp: Erg20p (F96W, N127W) mutant acting as a geranyl pyrophosphate (GPP) synthase; tObGES: truncated geraniol synthase from Ocimum basilicum. IPP: isopentyl pyrophosphate; DMAPP: dimethylallyl pyrophosphate. (FIG. 1B) Schematic showing the genomic integration of seven MVA pathway genes and the tObGES-Erg20wwp fusion protein expressed episomally from a strong constitutive promoter (pPYK001). The two proteins are fused together with a “GSG” linker. (FIG. 1C) Geraniol yield in engineered strains (MVAc1, MVAc2, MVAc3, and MVAc4). “c” indicates that genes are localized to the cytosol. Fold increase compared to the wild type at each time point is noted at the top of each bar. Data represent the average±SD of three independent biological replicates.

FIGS. 2A and 2B illustrate construction and screening of the combinatorial yeast MVA library with varying promoter strengths. (FIG. 2A) A diploid library of 243 strains, each having tHMG1 and IDI1 under strong promoters and ERG13, ERG12, ERG19, ERG10, and ERG8 under a unique combination of strong, medium, or weak promoters integrated into the genome. The tObGES-ERG20^{ww} fusion protein was expressed from a plasmid. Color intensity represents promoter strength. The strains were cultured in 96-deep-well plates, and the geraniol produced was quantified using a fluorescence-based assay. (FIG. 2B) Heat map showing relative promoter strengths and the corresponding fluorescence normalized to OD₆₀₀ of the wild type and the 243 strains. The top ten strains with the highest fluorescence readings are marked with an asterisk. Data represent the average of three independent biological replicates.

FIGS. 3A-3L: Random Forests were used to assess the importance and dependence of the MVA enzymes. (FIG. 3A) Variable importance from a random forest predicting readout. Enzymes are ranked according to increases in node purity, a measure of performance. (FIGS. 3B-F) Partial dependence plots show the predicted geraniol readout values as a function of enzyme expression for ERG19, ERG13, ERG12, ERG10, and ERG8. The blue tick marks represent the promoter strengths within the data, and the remaining curve was generated through interpolation. (FIGS. 3G-L) Two-way partial dependence plots for the interactions between ERG12 and the other four pathway enzymes, as well as the interactions between ERG19 and ERG13, and ERG8 and ERG10.

FIGS. 4A-4D: Creating the MVA platform strain by overexpressing the MVA pathway in both cytosol and peroxisomes. (FIG. 4A) The diploid strain (MVAplatform) was created by mating the haploid MVAc4 and haploid MVAp4. (FIG. 4B) Growth (OD₆₀₀) of the engineered MVAc4, MVAp4, and MVA platform strains and their wildtype counterparts. (FIG. 4C) Geraniol titer and OD₆₀₀ of engineered MVAc4, MVAp4, and MVA platform strains with tObGES-ERG20^{ww} in either the cytosol (‘C’) or peroxisomes (‘P’). (FIG. 4D) Geraniol yield in the above strains. Data represent the average±SD of three independent biological replicates.

FIGS. 5A-5E illustrate production of α-humulene and squalene using the MVA platform strain (FIG. 5A) Pathway for α-humulene and squalene production. ZSS1 encodes an α-humulene synthase from Zingiber zerumbet; ERG9 encodes a squalene synthase in S. cerevisiae; ERG1 encodes a squalene epoxidase in S. cerevisiae. (FIG. 5B) Episomal constructs express ERG20 and ZSS1 either separately or as a fusion protein with a ‘GSG’ linker. (FIG. 5C) α-Humulene production and growth (OD₆₀₀) of the wildtype (WT) and the engineered MVA platform expressing ERG20 and ZSS1. (FIG. 5D) Episomal constructs express ERG20 and ERG9 separately or as a fusion gene with a “GSG” linker. (FIG. 5E) Squalene production and growth (OD₆₀₀) of WT and the engineered MVA platform with ERG20 and ERG9. Data represent the average±SD of three independent biological replicates.

FIGS. 6A and 6B illustrate assembly and integration of multi-gene (MG) plasmids. Schematic depicting the (FIG. 6A) assembly of transcription units (TU) from part plasmids and (FIG. 6B) assembly and integration of multi-gene plasmid at a target locus using CRISPR-Cas9.

FIG. 7 illustrates a mating strategy used to prepare the combinatorial library. A haploid gal1Δ strain in the CEN.PK2-1C (MATa) background was streaked out in vertical streaks and a haploid rox1Δ gal80Δ strain in the CEN.PK2-1D (MATα) background was streaked out in horizontal streaks. Diploid colonies formed at the junctions of the streaks were used for constructing the combinatorial strain library.

FIGS. 8A-8C illustrate standard curves used for quantifying geraniol concentrations using the geraniol dehydrogenase assays. The standard curves correspond to (FIG. 8A) in FIG. 1C, (FIG. 8B) in FIG. 2B, and (FIG. 8C) in FIG. 4C.

FIGS. 9A-9C illustrate validating geraniol quantification by GC-MS. (FIG. 9A) The chromatogram (TIC) and MS spectrum of the authentic geraniol standard (12.5 mg/L). (FIG. 9B) Geraniol produced (⅕^{th} dilution in hexane) by the MVA platform strain transformed with pGAL1-tObGES-ERG20^{ww} and cultured in the YPD media. (FIG. 9C) The standard curve was used to quantify geraniol from the wild type and the MVA platform strain bearing pGAL1-tObGES-ERG20^{ww} by GC-MS. The table below shows that geraniol quantified by GeDH assay and GC-MS yield similar results.

FIG. 10 illustrates the effect of fusing versus separating tObGES and ERG20^{WW} on geraniol production. Geraniol produced by the strain MVAc4 when transformed with the empty vector, or with the tObGES and ERG20^{ww} separate, or with the tObGES-ERG20^{ww} fused. *: p<0.05. Data represent the average±SD of three independent biological replicates.

FIGS. 11A and 11B illustrate geraniol productivity in stepwise engineered strains. (FIG. 11A) Geraniol titer (mg/L) and (FIG. 11B) OD₆₀₀ of WT, MVAc1, MVAc2, MVAc3 and MVAc4 strains. Data represent the average±SD of three independent biological replicates.

FIGS. 12A-12C illustrate qRT-PCR to evaluate the levels of the MVA pathway gene expression in strains α1, β5, and γ9. (FIG. 12A) Correlation of fold change in gene expression over wild-type (WT) and promoter strengths determined in Lee et al. (3). (FIG. 12B) Fold change in gene expression compared to WT for HMG1 and tHMG1. (FIG. 12C) Fold change in gene expression compared to WT for the genes ERG10, ERG13, ERG12, ERG8, ERG19, and IDI1 driven by promoters of different strengths. Data represent the average±SD of three independent biological replicates.

FIGS. 13A-13D illustrate two-way partial dependence plots for the interactions between (FIG. 13A) ERG10 and ERG19, (FIG. 13B) ERG8 and ERG19, (FIG. 13C) ERG10 and ERG13, and (FIG. 13D) ERG8 and ERG13.

FIG. 14 illustrates local importance of pathway enzymes in the top ten geraniol-producing strains.

FIG. 15 illustrates Individual Conditional Expectation (ICE) plots for the top 10 ranked strains. Each line represents a strain, and the profiles capture the predicted changes in geraniol titer when the enzyme abundance is set at a given value. The red line indicates the average value over the strain set. ICE plots are shown for ERG19, ERG13, ERG12, ERG10, and ERG8. The ticks on the x-axis represent the values we have within this strain set, and interpolation is performed between the values.

FIGS. 16A and 16B illustrate that mevalonate is diffusible across peroxisomal membranes. (FIG. 16A) MVAc-p has the top half (ERG10-tHMG1) of the MVA pathway localized to the cytosol and the bottom half (ERG12-IDI) localized to the peroxisome. MVAp-c has the top half of the pathway in the peroxisome and the bottom half of the pathway in the cytosol. MVAc4 and MVAp4 have the entire MVA pathway localized to either the cytosol or the peroxisome, respectively. (FIG. 16B) Geraniol production normalized to growth (OD₆₀₀) by MVAc4 and MVAp-c transformed with tObGES-ERG20^{ww} in the cytosol (“C”), and MVAp4 and MVAc-p transformed with tObGES-ERG20^{ww} in peroxisome (“P”). *: p<0.05. Data represent the average±SD of three independent biological replicates.

FIG. 17 illustrates geraniol production in minimal media. Geraniol production normalized to growth (OD₆₀₀) by strains WT (CEN.PK2), MVAc4, and MVA platform transformed with the pYTK001_tObGES-ERG20^{ww} in the cytosol (“C”) and MVAp4 and MVA platform transformed with the pYTK001_tObGES-ERG20^{ww} in peroxisome (“P”). Data represent the average±SD of three independent biological replicates.

FIG. 18 illustrates localizing two complete MVA pathways into either the cytosol or peroxisomes produced less geraniol compared to the MVA platform strain. Geraniol production normalized to growth (OD₆₀₀) by strains including the MVA platform, MVA cyto*2 transformed with tObGES-ERG20^{ww} in the cytosol (“C”), and MVA platform and MVA per*2 transformed with tObGES-ERG20^{ww}-SKL in peroxisomes (“P”). Data represent the average±SD of three independent biological replicates. *: p<0.05.

FIGS. 19A-19C illustrate geraniol and citronellol quantification. (FIG. 19A) Quantification of geraniol and citronellol produced by the MVA platform strain transformed with tObGES-ERG20^{ww}. (FIG. 19B) GC/MS chromatograms of authentic citronellol (9.92 min), geraniol (10.11 min), and geranyl acetate (10.99 min) standard (6.25 mg/L each), and the MS spectrum of citronellol. (FIG. 19C) Citronellol and geraniol produced (⅓^{rd} dilution in hexane) by the MVA platform strain transformed with pGAL1-tObGES-ERG20^{ww} and the MS spectrum of citronellol produced in cells.

FIG. 20 illustrates geraniol production in YPO and YPD media by the strains MVAp4 and MVA platform transformed with tObGES-ERG20^{ww}-SKL in peroxisome (“P”). Data represent the average±SD of three independent biological replicates.

FIGS. 21A-21C are from Lee et al. (3) and illustrate characterization of promoters. (FIG. 21A) The relative strength of 19 constitutive promoters is consistent across two coding sequences, mRuby2 and Venus. Three promoters (strong pTDH3, medium pRPL18B, and weak pREV1) that are highlighted. The horizontal and vertical bars represent the range of four biological replicates, and the intersection represents the median value. (inset) A third fluorescent protein, mTurquoise2, was also tested, and a larger plot can be found in FIGS. 22A and 22B. (FIG. 21B) The mating-type-specific promoter, pMFA1, is only active in the MATa haploid; pMFα2 is only active in MATa haploids; neither promoter is active in the opposite haploid or in the diploid. The expression level of pRPL18B in the three strains is shown for reference. The height of the bars represents the median value of four biological replicates, and the error bars show the range. (FIG. 21C) Galactose induction of pGAL1 increases expression from background levels up to the highest expressing constitutive promoter, pTDH3. All solid line data were collected from a Δgal2 strain. The dashed line shows a much more sensitive response to galactose induction in a wild type strain. Points represent the median value of four biological replicates, and error bars show the range.

FIGS. 22A and 22B are from Lee et al. (3) illustrate the relative strength of 19 constitutive promoters driving three fluorescent proteins. (FIG. 22A) mRuby2 vs mTurquoise2. (FIG. 22B) mTurquoise2 vs Venus. Venus vs mRuby2 is shown in FIG. XA. The horizontal and vertical bars represent the range of four biological replicates, and the intersection represents the median value.

### DETAILED DESCRIPTION

Certain terminology is used in the following description for convenience only and is not limiting. The words “right,” “left,” “top,” and “bottom” designate directions in the drawings to which reference is made.

In some embodiments, the disclosure includes each genetic modification described herein alone, and in all combinations. Any genetic modifications may comprise, consist essentially of, or consist of the described modifications. All methods for making modified yeast, and making and isolating terpenes, as described herein are encompassed by the disclosure. The modified yeast may be any type of yeast. The disclosure includes diploid yeast, and haploid yeast that can be mated to produce the described modified yeast. The disclosure includes the modified yeast, and cell cultures comprising the modified yeast. Cell culture media that comprises produced terpenes is included. Kits that comprise the modified yeast, and optionally plasmids that encode a selected terpene synthesis protein, which optionally may comprise any prenyltransferase, any terpene synthase, and a combination thereof, are also included.

This disclosure provides, among other embodiments, a combinatorial library of 243 stable transgenic strains with each of the five non-rate-limiting MVA pathway genes under three different promoters. Machine learning algorithms revealed that ERG12 encoding the mevalonate kinase is the most critical gene, apart from HMG1 and IDI, that contributes significantly to the productivity of the MVA pathway. The disclosure provides a universal yeast platform for producing any terpenes by dual-targeting the MVA pathway in both the cytosol and peroxisomes. The dual-targeting revealed that some MVA pathway intermediates, including mevalonate and IPP/DMAPP, are diffusible between cytosol and peroxisomes. The platform strain produced about 94-fold higher monoterpene geraniol, about 60-fold higher sesquiterpene α-humulene, and about 35-fold higher triterpene squalene compared to the wild-type control.

### Definitions

Unless defined otherwise, technical and scientific terms used herein have the same meaning as commonly understood by one of ordinary skill in the art to which this invention belongs. For example, Singleton et al., Dictionary of Microbiology and Molecular Biology 2nd ed., J. Wiley & Sons (New York, NY 1994), provide one skilled in the art with a general guide to many of the terms used in the present application. Additionally, the practice of the present invention will employ, unless otherwise indicated, conventional techniques of molecular biology (including recombinant techniques), microbiology, cell biology, and biochemistry, which are within the skill of the art. Such techniques are explained fully in the literature, such as, “Molecular Cloning: A Laboratory Manual”, 2nd edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology”, 4th edition (D. M. Weir & C. C. Blackwell, eds., Blackwell Science Inc., 1987); “Gene Transfer Vectors for Mammalian Cells” (J. M. Miller & M. P. Calos, eds., 1987); “Current Protocols in Molecular Biology” (F. M. Ausubel et al., eds., 1987); and “PCR: The Polymerase Chain Reaction”, (Mullis et al., eds., 1994).

As used in the present disclosure and claims, the singular forms “a.” “an,” and “the” include plural forms unless the context clearly dictates otherwise.

It is understood that wherever embodiments are described herein with the language “comprising” otherwise analogous embodiments described in terms of “consisting of” and/or “consisting essentially of” are also provided. It is also understood that wherever embodiments are described herein with the language “consisting essentially of” otherwise analogous embodiments described in terms of “consisting of” are also provided.

The term “about” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods. For recitation of numeric ranges herein, each intervening number therebetween with the same degree of precision is explicitly contemplated. For example, for the range of from about 6 to about 9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, “either,” “one of,” “only one of,” or “exactly one of” “Consisting essentially of,” when used in the claims, shall have its ordinary meaning as used in the field of patent law.

The term “substantially free of” as used herein refers to a composition that only has trace or negligible amounts of the substance to which it refers. In some embodiments, substantially free means that the composition comprises only about 0.1%, 0.2%, 0.3% 0.4% or 0.5% of the substance to which it refers. In some embodiments, substantially free means that the composition comprises less than about 1.0% of the substance to which it refers relative to the number or mass of substances in the compositions and confers no biological effect to the compositions.

The term “culture vessel” as used herein is defined as any vessel suitable for growing, culturing, cultivating, proliferating, propagating, or otherwise similarly manipulating cells. In some embodiments, the cells yeast cells. In some embodiments, the culture vessel is made out of biocompatible plastic and/or glass.

The term “exposing” as used herein refers to bringing a disclosed compound and a cell in direct or indirect contact, in such a manner that the compound can affect the activity of the cell (e.g., a yeast cell.). Directly this can occur by physical contact between the disclosed compound and the cell by interacting with the cell itself, or indirectly this can occur by interacting with another molecule, co-factor, factor, or protein on which the activity of the cell is dependent. In some embodiments, the activity of the cell in response to the compound or molecule is production of a terpene.

The terms “polynucleotide,” “oligonucleotide” and “nucleic acid” are used interchangeably throughout and include DNA molecules (e.g., cDNA or genomic DNA), RNA molecules (e.g., mRNA), analogs of the DNA or RNA generated using nucleotide analogs (e.g., peptide nucleic acids and non-naturally occurring nucleotide analogs), and hybrids thereof. The nucleic acid molecule can be single-stranded or double-stranded. In some embodiments, the nucleic acid molecules of the disclosure comprise a contiguous open reading frame encoding a protein, or a fragment thereof, as described herein. “Nucleic acid” or “oligonucleotide” or “polynucleotide” as used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions. Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine Nucleic acids may be obtained by chemical synthesis methods or by recombinant methods. A nucleic acid generally contains phosphodiester bonds, although, in some embodiments, nucleic acid analogs may be included that may have at least one different linkage, e.g., phosphoramidate, phosphorothioate, phosphorodithioate, or O-methylphosphoroamidite linkages and peptide nucleic acid backbones and linkages. Other analog nucleic acids include those with positive backbones; non-ionic backbones, and non-ribose backbones, including those described in U.S. Pat. Nos. 5,235,033 and 5,034,506, which are incorporated by reference in their entireties. Nucleic acids containing one or more non-naturally occurring or modified nucleotides are also included within one definition of nucleic acids. The modified nucleotide analog may be located for example at the 5′-end and/or the 3′-end of the nucleic acid molecule. Representative examples of nucleotide analogs may be selected from sugar- or backbone-modified ribonucleotides. It should be noted, however, that also nucleobase-modified ribonucleotides, i.e. ribonucleotides, containing a non-naturally occurring nucleobase instead of a naturally occurring nucleobase such as uridines or cytidines modified at the 5-position, e.g. 5-(2-amino)propyl uridine, 5-bromo uridine; adenosines and guanosines modified at the 8-position, e.g. 8-bromo guanosine; deaza nucleotides, e.g. 7-deaza-adenosine; O- and N-alkylated nucleotides, e.g. N6-methyl adenosine are suitable. The 2′-OH-group may be replaced by a group selected from H, OR, R, halo, SH, SR, NH.sub.2, NHR, N.sub.2 or CN, wherein R is C.sub.1-C.sub.6 alkyl, alkenyl or alkynyl and halo is F, Cl, Br or I. Modified nucleotides also include nucleotides conjugated with cholesterol through, e.g., a hydroxyprolinol linkage as described in Krutzfeldt et al., Nature (Oct. 30, 2005), Soutschek et al., Nature 432:173-178 (2004), and U.S. Patent Publication No. 20050107325, which are incorporated herein by reference in their entireties. Modified nucleotides and nucleic acids may also include locked nucleic acids (LNA), as described in U.S. Patent No. 20020115080, which is incorporated herein by reference. Additional modified nucleotides and nucleic acids are described in U.S. Patent Publication No. 20050182005, which is incorporated herein by reference in its entirety.

As used herein, the term “nucleic acid molecule” comprises one or more nucleotide sequences that encode one or more proteins. In some embodiments, a nucleic acid molecule comprises initiation and termination signals operably linked to regulatory elements including a promoter and polyadenylation signal capable of directing expression in the cells of the individual to whom the nucleic acid molecule is administered. In some embodiments, the nucleic acid molecule also is a plasmid comprising one or more nucleotide sequences that encode one or a plurality of neoantigens. In some embodiments, the disclosure relates to a pharmaceutical composition comprising a first, second, third or more nucleic acid molecules, each of which encoding one or a plurality of neoantigens and at least one of each plasmid comprising one or more of the Formulae disclosed herein.

“Coding sequence” or “encoding nucleic acid” as used herein may mean refers to a nucleic acid (RNA, DNA, or RNA/DNA hybrid molecule) that comprises a nucleotide sequence which encodes a protein. The coding sequence may further include initiation and termination signals operably linked to regulatory elements including a promoter and polyadenylation signal capable of directing expression in the cells in which the nucleic acid is contained.

“Open reading frame” as used herein refers to nucleic acid sequence encoding a product between a start site and stop site. The transcript, in some embodiments, encodes an amino acid sequence and the start site is a start codon. In some embodiments, the stop site is a stop codon. The transcript, in some embodiments, includes exons and introns. The transcript, in some embodiments, is free of introns.

“Complement” or “complementary” as used herein may mean a nucleic acid may mean Watson-Crick (e.g., A-T/U and C-G) or Hoogsteen base pairing between nucleotides or nucleotide analogs of nucleic acid molecules.

The terms “polypeptide,” “peptide,” and “protein” are used interchangeably herein to refer to polymers of amino acids of any length. The polymer may be linear or branched, it may comprise modified amino acids, and it may be interrupted by non-natural amino acids or chemical groups that are not amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

As used herein, “conservative” amino acid substitutions may be defined as set out in Tables A, B, or C below. The vaccines, compositions, pharmaceutical compositions and method may comprise nucleic acid sequences comprising one or more conservative substitutions. In some embodiments, the vaccines, compositions, pharmaceutical compositions and methods comprise nucleic acid sequences that retain from about 70% sequence identity to about 99% sequences identity to the sequence identification numbers disclosed herein but comprise one or more conservative substitutions. Conservative substitutions of the present disclosure include those wherein conservative substitutions (from either nucleic acid or amino acid sequences) have been introduced by modification of polynucleotides encoding polypeptides. Amino acids can be according to physical properties and contribution to secondary and tertiary protein structure. A conservative substitution is recognized in the art as a substitution of one amino acid for another amino acid that has similar properties. In some embodiments, the conservative substitution is recognized in the art as a substitution of one nucleic acid for another nucleic acid that has similar properties, or, when encoded, has similar binding affinities to its target. Exemplary conservative substitutions are set out in Table A.

TABLE A
Conservative Substitutions I

| Side Chain Characteristics | |
|---|---|
| Aliphatic | Amino Acid |
| Non-polar | GAPILVF |
| Polar-uncharged | CSTMNQ |
| Polar-charged | DEKR |
| Aromatic | HFWY |
| Other | NQDE |

Alternately, conservative amino acids can be grouped as described in Lehninger, (Biochemistry, Second Edition; Worth Publishers, Inc. NY, N.Y. (1975), pp. 71-77) as set forth in Table B.

TABLE B
Conservative Substitutions II

| Side Chain Characteristic | Amino Acid |
|---|---|
| (hydrophobic) | Non-polar |
| Aliphatic: | ALIVP |
| Aromatic: | FWY |
| Sulfur-containing: | M |
| Borderline: | GY |
| Uncharged-polar | |
| Hydroxyl: | STY |
| Amides: | NQ |
| Sulfhydryl: | C |
| Borderline: | GY |
| Negatively Charged (Acidic): | DE |

Alternately, exemplary conservative substitutions are set out in Table B.

TABLE B
Conservative Substitutions III

| Original | Exemplary |
|---|---|
| Residue | Substitution |
| Ala (A) | Val Leu Ile Met |
| Arg (R) | Lys His |
| Asn (N) | Gln |
| Asp (D) | Glu |
| Cys (C) | Ser Thr |
| Gln (Q) | Asn |
| Glu (E) | Asp |
| Gly (G) | Ala Val Leu Pro |
| His (H) | Lys Arg |
| Ile (I) | Leu Val Met Ala Phe |
| Leu (L) | Ile Val Met Ala Phe |
| Lys (K) | Arg His |
| Met (M) | Leu Ile Val Ala |
| Phe (F) | Trp Tyr Ile |
| Pro (P) | Gly Ala Val Leu Ile |
| Ser (S) | Thr |
| Thr (T) | Ser |
| Trp (W) | Tyr Phe Ile |
| Tyr (Y) | Trp Phe Thr Ser |
| Val (V) | Ile Leu Met Ala |

The “percent identity” of two polynucleotide or two polypeptide sequences is determined by comparing the sequences. “Identical” or “identity” as used herein in the context of two or more nucleic acids or amino acid sequences, means that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be calculated manually or by using a computer sequence algorithm such as BLAST or BLAST 2.0. Briefly, the BLAST algorithm, which stands for Basic Local Alignment Search Tool is suitable for determining sequence similarity. Software for performing BLAST analyses is publicly available through the National Center for Biotechnology Information (http://www.ncbi.nlm.nih.gov). This algorithm involves first identifying high scoring sequence pair (HSPs) by identifying short words of length within a query sequence that either match or satisfy some positive-valued threshold score T when aligned with a word of the same length in a database sequence. T is referred to as the neighborhood word score threshold (Altschul et al., 1997). These initial neighborhood word hits act as seeds for initiating searches to find HSPs containing them. The word hits are extended in both directions along each sequence for as far as the cumulative alignment score can be increased. Extension for the word hits in each direction are halted when: 1) the cumulative alignment score falls off by the quantity X from its maximum achieved value; 2) the cumulative score goes to zero or below, due to the accumulation of one or more negative-scoring residue alignments; or 3) the end of either sequence is reached. The Blast algorithm parameters W, T and X determine the sensitivity and speed of the alignment. The Blast program uses as defaults a word length (W) of 11, the BLOSUM62 scoring matrix (see Henikoff et al., Proc. Natl. Acad. Sci. USA, 1992, 89, 10915-10919, which is incorporated herein by reference in its entirety) alignments (B) of 50, expectation (E) of 10, M=5, N=4, and a comparison of both strands. The BLAST algorithm (Karlin et al., Proc. Natl. Acad. Sci. USA, 1993, 90, 5873-5787, which is incorporated herein by reference in its entirety) and Gapped BLAST perform a statistical analysis of the similarity between two sequences. One measure of similarity provided by the BLAST algorithm is the smallest sum probability (P(N)), which provides an indication of the probability by which a match between two nucleotide sequences would occur by chance. For example, a nucleic acid is considered similar to another if the smallest sum probability in comparison of the test nucleic acid to the other nucleic acid is less than about 1, less than about 0.1, less than about 0.01, and less than about 0.001.

Two single-stranded polynucleotides are “the complement” of each other if their sequences can be aligned in an anti-parallel orientation such that every nucleotide in one polynucleotide is opposite its complementary nucleotide in the other polynucleotide, without the introduction of gaps, and without unpaired nucleotides at the 5′ or the 3′ end of either sequence. A polynucleotide is “complementary” to another polynucleotide if the two polynucleotides can hybridize to one another under moderately stringent conditions. Thus, a polynucleotide can be complementary to another polynucleotide without being its complement.

The phrase “stringent hybridization conditions” or “stringent conditions” as used herein is meant to refer to conditions under which a nucleic acid molecule will hybridize another nucleic acid molecule, but to no other sequences. Stringent conditions are sequence-dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present in excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g. 10 to 50 nucleotides) and at least about 600C for longer probes, primers or oligonucleotides. Stringent conditions may also be achieved with the addition of destabilizing agents, such as formamide.

By “substantially identical” is meant nucleic acid molecule (or polypeptide) exhibiting at least 50% identity to a reference amino acid sequence (for example, any one of the amino acid sequences described herein) or nucleic acid sequence (for example, any one of the nucleic acid sequences described herein). Preferably, such a sequence is at least about 60%, about 80% or about 85%, and about 90%, about 95% or about 99% identical at the amino acid level or nucleic acid to the sequence used for comparison.

“Operably linked” as used herein may mean that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function. As used herein, a coding sequence and regulatory sequences are said to be “operably” joined when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably linked to a coding sequence if the promoter region were capable of effecting transcription of that DNA sequence such that the resulting transcript can be translated into the desired protein or polypeptide.

When the nucleic acid molecule that encodes any of the enzymes of the claimed invention is expressed in a cell, a variety of transcription control sequences (e.g., promoter/enhancer sequences) can be used to direct its expression. The promoter can be a native promoter, i.e., the promoter of the gene in its endogenous context, which provides normal regulation of expression of the gene. In some embodiments the promoter can be constitutive, i.e., the promoter is unregulated allowing for continual transcription of its associated gene. A variety of conditional promoters also can be used, such as promoters controlled by the presence or absence of a molecule.

A nucleotide sequence is “operably linked” to a regulatory sequence if the regulatory sequence affects the expression (e.g., the level, timing, or location of expression) of the nucleotide sequence. A “regulatory sequence” is a nucleic acid that affects the expression (e.g., the level, timing, or location of expression) of a nucleic acid to which it is operably linked. The regulatory sequence can, for example, exert its effects directly on the regulated nucleic acid, or through the action of one or more other molecules (e.g., polypeptides that bind to the regulatory sequence and/or the nucleic acid). Examples of regulatory sequences include promoters, enhancers and other expression control elements (e.g., polyadenylation signals). Further examples of regulatory sequences are described in, for example, Goeddel, 1990, Gene Expression Technology: Methods in Enzymology 185, Academic Press, San Diego, Calif. and Baron et al., 1995, Nucleic Acids Res. 23:3605-06.

“Promoter” as used herein may mean a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which can be located as much as several thousand base pairs from the start site of transcription.

The term “fragment” is meant to be a portion of a polypeptide or nucleic acid molecule, such as, but not limiting to, a truncation mutant. This portion contains, preferably, at least about 5%, 10%, 20%, 30%, 40%, 50%, 60%, 70%, 80%, 90%, 95% of the entire length of the reference nucleic acid molecule or polypeptide. A fragment may contain about 5, about 10, about 20, about 30, about 40, about 50, about 60, about 70, about 80, about 90, about 100, about 200, about 300, about 400, about 500, about 600, about 700, about 800, about 900, or about 1000 or more nucleotides or amino acids of a nucleotide or amino acid sequence, respectively, upon which it is based.

The term “functional variant” a polypeptide or nucleic acid sequence, or a portion or fragment thereof, having sufficient identity and/or sufficient length and/or sufficient structure to confer a biological activity that is the same, substantially similar, or similar to the full-length polypeptide or nucleic acid upon which the fragment is based. In some embodiments, “biological activity” means that the functional variant participates in metabolism as to support terpene biosynthesis. In some embodiments, “biological activity” is measured as set forth in examples herein of producing a terpene. In some embodiments, a variant is a portion of a full-length or wild-type nucleic acid sequence that encodes any one of the amino acid sequences disclosed herein, and said portion encodes a polypeptide of a certain length and/or structure that is less than full-length but encodes a domain that is still biologically functional as compared to the full-length or wild-type protein. In such embodiments, the variant may retain at least about 99%, at least about 98%, at least about 97%, at least about 96%, at least about 95%, at least about 94%, at least about 93%, at least about 92%, at least about 91%, or at least about 90% sequence identity to the wild-type or given sequence upon which the sequence is derived. In some embodiments, a variant may retain at least about 85%, at least about 80%, at least about 75%, at least about 72%, at least about 70%, at least about 65%, or at least about 60% sequence identity to the wild-type sequence upon which the sequence is derived.

As used herein, the term “genetic construct” is meant to refer to the DNA or RNA molecules that comprise a nucleotide sequence that encodes protein. The coding sequence includes initiation and termination signals operably linked to regulatory elements including a promoter and polyadenylation signal capable of directing expression in the cells of the individual to whom the nucleic acid molecule is administered.

The term “hybridize” as used herein is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

The term “isolated” as used herein means that the nucleic acid molecule, polynucleotide or polypeptide or fragment, variant, or derivative thereof has been essentially removed from other biological materials with which it is naturally associated, or essentially free from other biological materials derived, e.g., from a recombinant host cell that has been genetically engineered to express the polypeptide of the disclosure.

The term “polypeptide” encompasses two or more naturally or non-naturally-occurring amino acids joined by a covalent bond (e.g., an amide bond). Polypeptides as described herein include full-length proteins (e.g., fully processed pro-proteins or full-length synthetic polypeptides) as well as shorter amino acid sequences (e.g., fragments of naturally-occurring proteins or synthetic polypeptide fragments).

As used herein, the terms “high” and “strong” related to the strength of a promoter are synonymous.

### Nucleic Acids

In some embodiments, the disclosure relates to open reading frames of a yeast gene operably linked to a one or more regulatory sequence. In some embodiments, one or more of the regulatory sequences is a promoter. A list of promoters and their nucleic acid sequences is provided in the below Promoter Table. The list of promoters and nucleic acid sequences in the Promoter Table are non-limiting examples of promoters of embodiments herein. In some embodiments, one or more of the promoters are independently selected from pTDH3, pCCW12, pHHF2, pRPL18B, pPOP6, pPGK1, pHTB2, pRNR2, pTEF2, pPAB1, pPSP2, pTEF1, pALD6, pRAD27, pHHF1, pRET2, and pREV1. In some embodiments, the one or more promoters independently comprise a nucleic acid sequence selected from one comprising, consisting essentially of, or consisting of a sequence having at least about 70%, at least about 72%, at least about 75%, at least about 80%, at least about 85%, at least about 90%, at least about 91%, at least about 92%, at least about 93%, at least about 94%, at least about 95%, at least about 95%, at least about 96%, at least about 97%, at least about 98%, at least about 99% identity to the sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, or SEQ ID NO: 17.

Promoter Table

| | Relative | | | |
|---|---|---|---|---|
| | promoter strength | | | |
| | quantified using | | | |
| | a fluorescent | | | |
| | protein | SEQ ID | | Designated |
| Promoter | (a.u.)* | NO | Nucleic Acid Sequence | strength |
| pTDH3 | 30.75 | 1 | cagttcgagtttatcattatcaatactgccatttcaaagaatacgtaaataattaatagtagt | Strong |
| | | | gattttcctaactttatttagtcaaaaaattagccttttaattctgctgtaacccgtacatgcc | |
| | | | caaaatagggggcgggttacacagaatatataacatcgtaggtgtctgggtgaacagtt | |
| | | | tattcctggcatccactaaatataatggagcccgctttttaagctggcatccagaaaaaaa | |
| | | | aagaatcccagcaccaaaatattgttttcttcaccaaccatcagttcataggtccattctctt | |
| | | | agcgcaactacagagaacaggggcacaaacaggcaaaaaacgggcacaacctcaa | |
| | | | tggagtgatgcaacctgcctggagtaaatgatgacacaaggcaattgacccacgcatg | |
| | | | tatctatctcattttcttacaccttctattaccttctgctctctctgatttggaaaaagctgaaa | |
| | | | aaaaaggttgaaaccagttccctgaaattattcccctacttgactaataagtatataaaga | |
| | | | cggtaggtattgattgtaattctgtaaatctatttcttaaacttcttaaattctacttttatagtta | |
| | | | gtcttttttttagttttaaaacaccaagaacttagtttcgaataaacacacataaacaaacaa | |
| | | | aagatct | |
| pCCW12 | 24.6 | 2 | cacccatgaaccacacggttagtccaaaaggggcagttcagattccagatgcgggaat | Strong |
| | | | tagcttgctgccaccctcacctcactaacgctgcggtgtgcggatacttcatgctatttata | |
| | | | gacgcgcgtgtcggaatcagcacgcgcaagaaccaaatgggaaaatcggaatgggt | |
| | | | ccagaactgctttgagtgctggctattggcgtctgatttccgttttgggaatcctttgccgc | |
| | | | gcgcccctctcaaaactccgcacaagtcccagaaagcgggaaagaaataaaacgcc | |
| | | | accaaaaaaaaaaaaataaaagccaatcctcgaagcgtgggggtaggccctggatta | |
| | | | tcccgtacaagtatttctcaggagtaaaaaaaccgtttgttttggaatttcccatttcgcgg | |
| | | | ccacctacgccgctatctttgcaacaactatctgcgataactcagcaaattttgcatattcg | |
| | | | tgttgcagtattgcgataatgggagtcttacttccaacataacggcagaaagaaatgtga | |
| | | | gaaaattttgcatcctttgcctccgttcaagtatataaagtcggcatgcttgataatctttctt | |
| | | | tccatcctacattgttctaattattcttattctcctttattctttcctaacataccaagaaattaat | |
| | | | cttctgtcattcgcttaaacactatatcaataaagatc | |
| pPGK1 | 11.01 | 3 | gatcttgttttatatttgttgtaaaaagtagataattacttccttgatgatctgtaaaaaagag | Strong |
| | | | aaaaagaaagcatctaagaacttgaaaaactacgaattagaaaagaccaaatatgtattt | |
| | | | cttgcattgaccaatttatgcaagtttatatatatgtaaatgtaagtttcacgaggttctacta | |
| | | | aactaaaccacccccttggttagaagaaaagagtgtgtgagaacaggctgttgttgtca | |
| | | | cacgattcggacaattctgtttgaaagagagagagtaacagtacgatcgaacgaacttt | |
| | | | gctctggagatcacagtgggcatcatagcatgtggtactaaaccctttcccgccattcca | |
| | | | gaaccttcgattgcttgttacaaaacctgtgagccgtcgctaggaccttgttgtgtgacga | |
| | | | aattggaagctgcaatcaataggatgacaggaagtcgagcgtgtctgggttttttcagttt | |
| | | | tgttctttttgcaaacaaatcacgagcgacggtaatttctttctcgataagaggccacgtg | |
| | | | ctttatgagggtaacatcaattcaagaaggagggaaacacttcctttttctggccctgata | |
| | | | atagtatgagggtgaagccaaaataaaggattcgcgcccaaatcggcatctttaaatgc | |
| | | | aggtatgcgatagttcctcactctttccttactcac | |
| pHHF2 | 9.01 | 4 | tgtggagtgtttgcttggattctttagtaaaaggggaagaacagttggaagggccaaagt | Strong |
| | | | ggaagtcacaaaacagtggtcctatataaaagaacaagaaaaagattatttatatacaac | |
| | | | tgcggtcacaagaagcaacgcgagagagcacaacacgctgttatcacgcaaactatgt | |
| | | | tttgacaccgagccatagccgtgattgtgcgtcacattgggcgataatgaacgctaaatg | |
| | | | accaactcccatccgtaggagccccttagggcgtgccaatagtttcacgcgcttaatgc | |
| | | | gaagtgctcggaacggacaactgtggtcgtttggcaccgggaaagtggtactagacc | |
| | | | gagagtttcgcatttgtatggcaggacgttctgggagcttcgcgtctaaagctttttcggg | |
| | | | cgcgaaatgcagaccagaccagaacaaaacaactgacaagaaggcgtttaatttaata | |
| | | | tgttgttcactcgcgcctgggctgttgttattcggctagatacatacgtgtttgtgcgtatgt | |
| | | | agttatatcatatataagtatattaggatgaggcggtgaaagagattttttttttttcgcttaat | |
| | | | ttattcttttctctatcttttttcctacatcttgttcaaaagagtagcaaaaacaacaatcaata | |
| | | | caataaaataagatct | |
| pTEF1 | 8.85 | 5 | ccttgccaacagggagttcttcagagacatggaggctcaaaacgaaattattgacagcc | Strong |
| | | | tagacatcaatagtcatacaacagaaagcgaccacccaactttggctgataatagcgtat | |
| | | | aaacaatgcatactttgtacgttcaaaatacaatgcagtagatatatttatgcatattacata | |
| | | | taatacatatcacataggaagcaacaggcgcgttggacttttaattttcgaggaccgcga | |
| | | | atccttacatcacacccaatcccccacaagtgatcccccacacaccatagcttcaaaatg | |
| | | | tttctactccttttttactcttccagattttctcggactccgcgcatcgccgtaccacttcaaa | |
| | | | acacccaagcacagcatactaaatttcccctctttcttcctctagggtgtcgttaattaccc | |
| | | | gtactaaaggtttggaaaagaaaaaagacaccgcctcgtttctttttcttcgtcgaaaaag | |
| | | | gcaataaaaatttttatcacgtttctttttcttgaaaatttttttttttgatttttttctctttcgatga | |
| | | | cctcccattgatatttaagttaataaacggtcatcaatttctcaagtttcagtttcatttttcttg | |
| | | | ttctattacaactttttttacttcttgctcattagaaagaaagcatagcaatctaatctaagtttt | |
| | | | aattacaaaagatc | |
| pTEF2 | 7.77 | 6 | ttgataggtcaagatcaatgtaaacaattactttgttatgtagagtttttttagctacctatatt | Strong |
| | | | ccaccataacatcaatcatgcggttgctggtgtatttaccaataatgtttaatgtatatatat | |
| | | | atatatatatatggggccgtatacttacatatagtagatgtcaagcgtaggcgcttcccct | |
| | | | gccggctgtgagggcgccataaccaaggtatctatagaccgccaatcagcaaactacc | |
| | | | tccgtacattcatgttgcacccacacatttatacacccagaccgcgacaaattacccata | |
| | | | aggttgtttgtgacggcgtcgtacaagagaacgtgggaactttttaggctcaccaaaaa | |
| | | | agaaagaaaaaatacgagttgctgacagaagcctcaagaaaaaaaaaattcttcttcga | |
| | | | ctatgctggaggcagagatgatcgagccggtagttaactatatatagctaaattggttcc | |
| | | | atcaccttcttttctggtgtcgctccttctagtgctatttctggcttttcctatttttttttttccattt | |
| | | | ttctttctctctttctaatatataaattctcttgcattttctatttttctctctatctattctacttgttt | |
| | | | attcccttcaaggtttttttttaaggagtacttgtttttagaatatacggtcaacgaactataat | |
| | | | taactaaacagatc | |
| pHHF1 | 4.81 | 7 | tcttggggccttaccaccagtggactttcttgctgtttgctttgttctggccattgtttgcgttt | Strong |
| | | | atatatttatgttagatgtttttcttattaactagaaagaaagaatataaaaggttgaggaaa | |
| | | | gagatgtatcccgaagaatacacagtcttttatatatgtatttcaacaaggagccgtggag | |
| | | | ggtactaaaaagaaaaatcgcccgggcatttcgttatcttccacgctaaaagtcaagga | |
| | | | gagatattacggccaggatcgcaaaggtgcagagcaaggaaatgtgagaaattgtga | |
| | | | gaacgataatgtatgggacaatgcgaaaatgtgagaacgagagcaaaaatcttttttgta | |
| | | | tctccccgccgaatttggaaaccgcgttctgaaaacttcgcatcttcacatagtaaaactg | |
| | | | ttccgagcgcttctccccataatggttagtggtaaaaaccgaagttgtttactttagcaaat | |
| | | | gcccgcgaatacggtggtaaattgccacccccccttccccattcattgggtaaagacca | |
| | | | atttgatggataaattggttgtggaaaaggtctaattctttttcctataaataccgagatatttt | |
| | | | ttctatatgatggtttccgtcgcattattgtactctatagtactaaagcaacaaacaaaaac | |
| | | | aagcaacaaatataatatagtaaaatagatc | |
| pRPL18B | 3 | 8 | aagaggatgtccaatattttttttaaggaataaggatacttcaagactagattcccccctgc | Medium |
| | | | attcccatcagaaccgtaaaccttggcgctttccttgggaagtattcaagaagtgccttgt | |
| | | | ccggtttctgtggctcacaaaccagcgcgcccgatatggctttcttttcacttatgaatgta | |
| | | | ccagtacgggacaattagaacgctcctgtaacaatctctttgcaaatgtggggttacattc | |
| | | | taaccatgtcacactgctgacgaaattcaaagtaaaaaaaaatgggaccacgtcttgag | |
| | | | aacgatagattttctttattttacattgaacagtcgttgtctcagcgcgctttatgttttcattca | |
| | | | tacttcatattataaaataacaaaagaagaatttcatattcacgcccaagaaatcaggctg | |
| | | | ctttccaaatgcaattgacacttcattagccatcacacaaaactctttcttgctggagcttct | |
| | | | tttaaaaaagacctcagtacaccaaacacgttacccgacctcgttattttacgacaactat | |
| | | | gataaaattctgaagaaaaaataaaaaaattttcatacttcttgcttttatttaaaccattgaa | |
| | | | tgatttcttttgaacaaaactacctgtttcaccaaaggaaatagaaagaaaaaatcaatta | |
| | | | gaagaaaacaaaaaacaaaagatc | |
| pHTB2 | 2.85 | 9 | tatatattaaatttgctcttgttctgtactttcctaattcttatgtaaaaagacaagaatttatga | Medium |
| | | | tactatttaataacaaaaaactacctaagaaaagcatcatgcagtcgaaattgaaatcga | |
| | | | aaagtaaaactttaacggaacatgtttgaaattctaagaaagcatacatcttcatcccttat | |
| | | | atatagagttatgtttgatattagtagtcatgttgtaatctctggcctaagtatacgtaacga | |
| | | | aaatggtagcacgtcgcgtttatggcccccaggttaatgtgttctctgaaattcgcatcac | |
| | | | tttgagaaataatgggaacaccttacgcgtgagctgtgcccaccgcttcgcctaataaa | |
| | | | gcggtgttctcaaaatttctccccgttttcaggatcacgagcgccatctagttctggtaaa | |
| | | | atcgcgcttacaagaacaaagaaaagaaacatcgcgtaatgcaacagtgagacacttg | |
| | | | ccgtcatatataaggttttggatcagtaaccgttatttgagcataacacaggtttttaaatat | |
| | | | attattatatatcatggtatatgtgtaaaatttttttgctgactggttttgtttatttatttagcttttt | |
| | | | aaaaattttactttcttcttgttaattttttctgattgctctatactcaaaccaacaacaacttac | |
| | | | tctacaactaagatc | |
| pALD6 | 2.28 | 10 | taagggcatgatagaattggattatgtaaaaggtgaagataccattgtagaagcaacca | Medium |
| | | | gcacgtcgccgtggctgatgaagtctcctcttgcccgggccgcagaaaagaggggca | |
| | | | gtggcctgtttttcgacataaatgaggggcatggccagcaccaagacgtcattgttgcat | |
| | | | atggcgtatccaagccgaaacggcgctcgcctcatccccacgggaataaggcagccg | |
| | | | acaaaagaaaaacgaccgaaaaggaaccagaaagaaaaaagagggtgggcgcgc | |
| | | | cgcggacgtgtaaaaagatatgcatccagcttctatatcgctttaactttaccgttttgggc | |
| | | | atcgggaacgtatgtaacattgatctcctcttgggaacggtgagtgcaacgaatgcgat | |
| | | | atagcaccgaccatgtgggcaaattcgtaataaattcggggtgagggggattcaagac | |
| | | | aagcaaccttgttagtcagctcaaacagcgatttaacggttgagtaacacatcaaaacac | |
| | | | cgttcgaggtcaagcctggcgtgtttaacaagttcttgatatcatatataaatgtaataaga | |
| | | | agtttggtaatattcaattcgaagtgttcagtcttttacttctcttgttttatagaagaaaaaac | |
| | | | atcaagaaacatctttaacatacacaaacacatactatcagaatacaagatc | |
| pPAB1 | 1.69 | 11 | aaggcaagcccagaaaaatatcgcaagcacctttggtcttacagtgccaacttttggcct | Medium |
| | | | gccgacgttaagagtacaaagctgatggcaatgtacgacaagataacagagtctcaaa | |
| | | | agaagtgaaacaatttttcttcaccacattttccattgttccttccccccataactataaacgt | |
| | | | atttatgtatatatatttgcgtgtaagtgtgtgtactatagggcaccgtaaagtaataatgctt | |
| | | | aattagttactactatgaccatataagaggtcatactgtatgaagccacaaagcagatag | |
| | | | atcaatcatgtttaacgaaaactgttaatcgaagattatttctttttttttttctctttcctttttac | |
| | | | aaagaaaattttttttgcgctttttgccatcaccatcgcaagttctgggacaattgttctcttt | |
| | | | cgctccagttccaaggaaagaggtttctgttttacttaatagaaagtgtcatcttgtattttat | |
| | | | atctcttctttcttgtgtaaaattctttagttttgattttgtatttttaggacagtgagctacgaa | |
| | | | gtaacatttttacttaataaccgtttgaagcatagagcaggccctggtatcaccacctaat | |
| | | | atctggctttttattcaataaaaactcaaaaaaaaaaatccaaaaaaaactaaaaaaccaa | |
| | | | taaaaataaaagatc | |
| pRET2 | 1.53 | 12 | acgatggcttcttatctcacttcaatagtactttccaccggttatacttccggcttttccctatt | Medium |
| | | | aatacaagctacaatttcaatgggtggcaaataatgtgtagaatagaaaataagccgac | |
| | | | agggtaataaagaaaatttttagaaaaaaaaggttagatggcttatttaagttacaggcta | |
| | | | gcgaaaaaaggaacttcagggcaagtaaagtgtttgattgggcactagcatggcttata | |
| | | | aaggcgagcaattgtcgaaactaattaatgttgtacggactattgctgtcatctcgtggta | |
| | | | aatgcgtgttccaggtcgaatactacttgcacacaggcgagcggggccccataaaagt | |
| | | | gttgccgatttgttaagttgtcttttcggtttttctactctgttattccttacttccctttttaagaa | |
| | | | ctctttttatccttcatttaggatcttgcacgtttccgcctcatcacttgaattaaaacatgtct | |
| | | | ctgtcagtaaaccttggcgtttctattgttcttcatagttcaacttttattattacccgccctgc | |
| | | | gcgtttacatttttccagcaacagccagcgaaaaattagaaaatctggttgttgacacctc | |
| | | | aagaacaagggcaattagcctcagcgtcgaatatagatcatattagaatacctatagctc | |
| | | | catcaaaagaaatacacaagatc | |
| pPOP6 | 1.06 | 13 | ttcgtgctttgtgataaagtgtttcacgtcatccgacatgacttcgtagttatggactgaact | Weak |
| | | | gtgtggtgaggttccatgatttcttaggtccagcagatacatgtctcttcccaatttcttgtta | |
| | | | aggttacggccaatgcttcggttgttgagcttgttaccgaataagccgtgaagtatgataa | |
| | | | taggtggtcttggcttcccttcatccccagtttttactgcatctctcttgattatgtcatatgaa | |
| | | | aggtccagtgggacttgcttttgttgcagcacctttgctaatgaatgaaaggcacatagtg | |
| | | | actgcttaaaaatgcaggaacttaaattattccgaatggtattttgtctcacatatattgtcc | |
| | | | catactgtgccaagatcccggctttacccagtatcatcattgtaccgttaccaattctcctc | |
| | | | gtatatcacggttagtttttaaacctcggggtgacgtttactattggcgtactaatatattctt | |
| | | | attttcttttcttttttgttggcagtttcaagcaacacatgtactggataaccaacccccgca | |
| | | | cgctcttggaaaaaattgagaaggcatcggacacttgctgatgagtatttcgaaaaattc | |
| | | | catgaagatgaggccaagattgtttggaagagattgaaaagaagaagaagaaaaaaa | |
| | | | gataaaagcaaatcaaaagatc | |
| pRNR2 | 1.06 | 14 | agtcgaacaagaagcaggcaaagtttagagcactgcccctccgcactcaaaaaagaa | Weak |
| | | | aaaactaggaggaaaataaaattctcaaccacacaaacacataaacacatacaaatac | |
| | | | aaatacaagcttatttacttgacatcgcgcgatcttccactattcagcgccgtccgccctct | |
| | | | ctcgtgttttttgtttacgcgacaactatgcgaaatccggagcaacgggcaaccgtttgg | |
| | | | ggaaagaccacacccacgcgcgatcgccatggcaacgaggtcgcacacgccccac | |
| | | | acccagacctccctgcgagcgggcatgggtacaatgtccccgttgccacagacacca | |
| | | | cttcgtagcacagcgcagagcgtagcgtgttgttgctgctgacaaaagaaaatttttctta | |
| | | | gcaaagcaaaggaggggaagcacgggcagatagcaccgtaccatacccttggaaac | |
| | | | tcgaaatgaacgaagcaggaaatgagagaatgagagttttgtaggtatatatagcggta | |
| | | | gtgtttgcgcgttaccatcatcttctggatctatctattgttcttttcctcatcactttcccctttt | |
| | | | tcgctcttcttcttgtcttttatttctttcttttttttaattgttccctcgattggctatctaccaaag | |
| | | | aatccaaacttaatacacgtatttatttgtccaattaccagatc | |
| pPSP2 | 0.91 | 15 | tgacccaacatcagatgacccaaggtccacctcttattaaaggacgtttgatccttcgac | Weak |
| | | | accatggctctgttgaacttttatctgagagaggaaaaaaaggaaggaaaaaaaagaa | |
| | | | gaaacttcctttatttatttgtcttaaccacaacacacaatgcaataagatgcaatataatat | |
| | | | caaagccaatatcttatgttgctgatcctgagaaggaatatatacaatttatgtagtaaaat | |
| | | | accttttcttctgcgagttgcaagaaatagaaaagactccgattgcgcatcgccagaata | |
| | | | aaatttcacaaccacactttttggctgaactttttattacctgattaaacagagagagaaaa | |
| | | | ggtagaggtcaaaattttttaagcaaaactaaaaaagatgcaaaatcacgtgctgaaaat | |
| | | | ctaacataagggttaagattagagttttataggacttgttttgtaatatttcaaatacgagct | |
| | | | aaccctactgatttcaattaggtctaatttagggttgagctgcactgaaatttcggaaatttt | |
| | | | gggttattttaaatgagacagaagaactacagagatacgttcttcagactttaaagcttatc | |
| | | | tccacaaagaattggtcaagaaatcatcctagaaaaacacgtttgctcactcgatcttaat | |
| | | | cacatagagtgctggaacgggaagaaagatc | |
| pRAD27 | 0.91 | 16 | ccttgtgaaattgcaaatatggtgatttgaaacgtttcctagtgcagcaggatcacagata | Weak |
| | | | acgtgtaaagggcttagcagttgataatcctctctagttaagacctaaacaaaatgctgtc | |
| | | | actaaccgtagtattaaatgacacactttggtgactttcgttaatggggatgtggtagtgg | |
| | | | ccattgccaataaacaaaaagaacagggaaagaagtagaaagtgatataagtttgcttg | |
| | | | ccacttttcgtttttcacgaaaaaaacaggcgaaaaaaaatgctagacaagtacccggct | |
| | | | gaatcacacctcgttaacagtgactttcggtgacagatacccgattgggcacccggctg | |
| | | | gtaagttatgatagaaagccaacgctgtactattggcttagctatggcaatattttgattatc | |
| | | | agctagttttattaacgttataattagtgtaaccagtttttcatctatttcatttatttcatttattta | |
| | | | ctttaattgcagatccccctaacgcgtttaaagcttttattcactagcttatgtattttttatag | |
| | | | gaaacgcgacgcgtaacatcgcgcaaatgaaggttttgatgtattataatgaggtattctt | |
| | | | ccttatatacatcgatgaaaagcgttgacagcatacattggaaagaaataggaaacgga | |
| | | | caccggaagaaaaaatagatc | |
| pREV1 | 0.86 | 17 | gtgttgttatccgatacaaccggatatttttcttttaatgagtctaaaccgtgatagcttcag | Weak |
| | | | gttaatacaatcaaaaaaagctcaaatattcttttaatgccgcgttcacagattccaattga | |
| | | | atacaactaggtagttcattatatgaagcctttgctactatttttcactatagtctgccttcac | |
| | | | cttaatgcagacatccacatattttaatcactttaaaataaaaaggaagatatattagaagc | |
| | | | tatgatccaatctgtaagccagattaaaattcacgaactcttctttcatttgaattgaatgctt | |
| | | | tgagttggggtagattatcgcaaattactcatcacatttattgactacgaacttgctgatgtc | |
| | | | ctttttttatttatatttttcttcagtgaagcgattttttttttacacagaccaagacggaaaaaa | |
| | | | gtagctaaggaagaaaacaaaatcatgaaaaaaatgtgaagtgatcatgcacatcgcat | |
| | | | caacttaaacattggcttagagatatatagagttagagtttacggcaacctttaagcacca | |
| | | | ataccttttggcatagtctaaagacctggttcttaattttaaacaaatttaactaaagatttcc | |
| | | | ctatcaaagaagtaacgagttgacagattttctcaaaataaatcgatactgcatttctagg | |
| | | | catatccagcgagatc | |

In some embodiments, (1) a high-strength promoter results in at least about 5.5-fold greater expression compared to pREV1 in otherwise identical constructs and conditions, (2) a medium-strength promoter results in at least about 1.5-fold expression but less than about 5.5-fold expression compared to pREV1 in otherwise identical constructs and conditions, and (3) a weak-strength promoter results in less than about 1.5-fold expression compared to pREV1 in otherwise identical constructs and conditions.

In some embodiments, (1) a high-strength promoter is a promoter that will result in a level of expression about equal to or greater than the level of expression of pHHF1 in the constructs and assay of Example 6, (2) a medium-strength promoter is a promoter that will result in a level of expression about equal to or greater than pRET2 but less than the level of expression of pHHF1 in the constructs and assay of Example 6, and (3) a weak-strength promoter is a promoter that will result in a level of expression less than the level of expression of pRET2 in the constructs and assay of Example 6. In some embodiments, (1) a high-strength promoter is a promoter that will result in a level of expression about equal to or greater than the level of expression of pHHF1 in the assay of Example 7, (2) a medium-strength promoter is a promoter that will result in a level of expression greater than pPOP6 but less than the level of expression of pHHF1 in the of Example 6, and (3) a weak-strength promoter is a promoter that will result in a level of expression less than the level of expression of pPOP6 in the assay of Example 6.

In some embodiments, a yeast gene operably linked to one or more regulatory sequence is selected from ERG8, ERG10, ERG12, ERG13, ERG19, tHMG1, or IDI1. A list of genes and their nucleic acid sequences is provided in the below Gene Table. The list of genes and nucleic acid sequences in the Gene Table are non-limiting examples of genes of embodiments herein. A list of amino acid sequences encoded by genes herein is provided in the below Amino Acid Sequence Table. The list of amino acid sequences in the Amino Acid Sequence Table are non-limiting examples of amino acid sequences encoded by genes of embodiments herein.

In some embodiments, the open reading frame of the yeast gene comprises, consists essentially of, or consists of nucleic acid sequence comprising, consisting essentially of, or consisting of one having at least about 70%, at least about 72%, at least about 75%, at least about 80%, at least about 85%, at least about 90%, at least about 91%, at least about 92%, at least about 93% at least about 94%, at least about 95%, at least about 95%, at least about 96%, at least about 97%, at least about 98%, at least about 99% identity to the sequence of SEQ ID NO: 18, SEQ TD NO: 19, SEQ TD NO: 20, SEQ ID NO: 21, SEQ ID NO: 22, SEQ ID NO: 23, or SEQ ID NO: 24.

Gene Table

| | SEQ | Acc. No. | |
|---|---|---|---|
| Gene | ID NO | (SGD) | Nucleic Acid Sequence |
| ERG8 | 18 | S000004833 | atgtcagagttgagagccttcagtgccccagggaaagcgttactagctggtggatatttagttttagatacaaa |
| | | | atatgaagcatttgtagtcggattatcggcaagaatgcatgctgtagcccatccttacggttcattgcaagggtc |
| | | | tgataagtttgaagtgcgtgtgaaaagtaaacaatttaaagatggggagtggctgtaccatataagtcctaaaa |
| | | | gtggcttcattcctgtttcgataggcggatctaagaaccctttcattgaaaaagttatcgctaacgtatttagctac |
| | | | tttaaacctaacatggacgactactgcaatagaaacttgttcgttattgatattttctctgatgatgcctaccattct |
| | | | caggaggatagcgttaccgaacatcgtggcaacagaagattgagttttcattcgcacagaattgaagaagttc |
| | | | ccaaaacagggctgggctcctcggcaggtttagtcacagttttaactacagctttggcctccttttttgtatcgga |
| | | | cctggaaaataatgtagacaaatatagagaagttattcataatttagcacaagttgctcattgtcaagctcaggg |
| | | | taaaattggaagcgggtttgatgtagcggcggcagcatatggatctatcagatatagaagattcccacccgca |
| | | | ttaatctctaatttgccagatattggaagtgctacttacggcagtaaactggcgcatttggttgatgaagaagact |
| | | | ggaatattacgattaaaagtaaccatttaccttcgggattaactttatggatgggcgatattaagaatggttcaga |
| | | | aacagtaaaactggtccagaaggtaaaaaattggtatgattcgcatatgccagaaagcttgaaaatatataca |
| | | | gaactcgatcatgcaaattctagatttatggatggactatctaaactagatcgcttacacgagactcatgacgat |
| | | | tacagcgatcagatatttgagtctcttgagaggaatgactgtacctgtcaaaagtatcctgaaatcacagaagtt |
| | | | agagatgcagttgccacaattagacgttcctttagaaaaataactaaagaatctggtgccgatatcgaacctcc |
| | | | cgtacaaactagcttattggatgattgccagaccttaaaaggagttcttacttgcttaatacctggtgctggtggt |
| | | | tatgacgccattgcagtgattactaagcaagatgttgatcttagggctcaaaccgctaatgacaaaagattttct |
| | | | aaggttcaatggctggatgtaactcaggctgactggggtgttaggaaagaaaaagatccggaaacttatcttg |
| | | | ataaataa |
| ERG10 | 19 | S000005949 | atgtctcagaacgtttacattgtatcgactgccagaaccccaattggttcattccagggttctctatcctccaaga |
| | | | cagcagtggaattgggtgctgttgctttaaaaggcgccttggctaaggttccagaattggatgcatccaaggat |
| | | | tttgacgaaattatttttggtaacgttctttctgccaatttgggccaagctccggccagacaagttgctttggctgc |
| | | | cggtttgagtaatcatatcgttgcaagcacagttaacaaggtctgtgcatccgctatgaaggcaatcattttggg |
| | | | tgctcaatccatcaaatgtggtaatgctgatgttgtcgtagctggtggttgtgaatctatgactaacgcaccatac |
| | | | tacatgccagcagcccgtgcgggtgccaaatttggccaaactgttcttgttgatggtgtcgaaagagatgggtt |
| | | | gaacgatgcgtacgatggtctagccatgggtgtacacgcagaaaagtgtgcccgtgattgggatattactag |
| | | | agaacaacaagacaattttgccatcgaatcctaccaaaaatctcaaaaatctcaaaaggaaggtaaattcgac |
| | | | aatgaaattgtacctgttaccattaagggatttagaggtaagcctgatactcaagtcacgaaggacgaggaac |
| | | | ctgctagattacacgttgaaaaattgagatctgcaaggactgttttccaaaaagaaaacggtactgttactgcc |
| | | | gctaacgcttctccaatcaacgatggtgctgcagccgtcatcttggtttccgaaaaagttttgaaggaaaagaa |
| | | | tttgaagcctttggctattatcaaaggttggggtgaggccgctcatcaaccagctgattttacatgggctccatct |
| | | | cttgcagttccaaaggctttgaaacatgctggcatcgaagacatcaattctgttgattactttgaattcaatgaag |
| | | | ccttttcggttgtcggtttggtgaacactaagattttgaagctagacccatctaaggttaatgtatatggtggtgct |
| | | | gttgctctaggtcacccattgggttgttctggtgctagagtggttgttacactgctatccatcttacagcaagaag |
| | | | gaggtaagatcggtgttgccgccatttgtaatggtggtggtggtgcttcctctattgtcattgaaaagatatga |
| ERG12 | 20 | S000004821 | atgtcattaccgttcttaacttctgcaccgggaaaggttattatttttggtgaacactctgctgtgtacaacaagcc |
| | | | tgccgtcgctgctagtgtgtctgcgttgagaacctacctgctaataagcgagtcatctgcaccagatactattga |
| | | | attggacttcccggacattagctttaatcataagtggtccatcaatgatttcaatgccatcaccgaggatcaagt |
| | | | aaactcccaaaaattggccaaggctcaacaagccaccgatggcttgtctcaggaactcgttagtcttttggatc |
| | | | cgttgttagctcaactatccgaatccttccactaccatgcagcgttttgtttcctgtatatgtttgtttgcctatgccc |
| | | | ccatgccaagaatattaagttttctttaaagtctactttacccatcggtgctgggttgggctcaagcgcctctattt |
| | | | ctgtatcactggccttagctatggcctacttgggggggttaataggatctaatgacttggaaaagctgtcagaa |
| | | | aacgataagcatatagtgaatcaatgggccttcataggtgaaaagtgtattcacggtaccccttcaggaataga |
| | | | taacgctgtggccacttatggtaatgccctgctatttgaaaaagactcacataatggaacaataaacacaaaca |
| | | | attttaagttcttagatgatttcccagccattccaatgatcctaacctatactagaattccaaggtctacaaaagat |
| | | | cttgttgctcgcgttcgtgtgttggtcaccgagaaatttcctgaagttatgaagccaattctagatgccatgggtg |
| | | | aatgtgccctacaaggcttagagatcatgactaagttaagtaaatgtaaaggcaccgatgacgaggctgtaga |
| | | | aactaataatgaactgtatgaacaactattggaattgataagaataaatcatggactgcttgtctcaatcggtgttt |
| | | | ctcatcctggattagaacttattaaaaatctgagcgatgatttgagaattggctccacaaaacttaccggtgctg |
| | | | gtggcggcggttgctctttgactttgttacgaagagacattactcaagagcaaattgacagcttcaaaaagaaa |
| | | | ttgcaagatgattttagttacgagacatttgaaacagacttggggggactggctgctgtttgttaagcgcaaaa |
| | | | aatttgaataaagatcttaaaatcaaatccctagtattccaattatttgaaaaaaaactaccacaaagcaacaaa |
| | | | ttgacgatctattattgccaggaaacacgaatttaccatggacttcataa |
| ERG13 | 21 | S000004595 | atgaaactctcaactaaactttgttggtgtggtattaaaggaagacttaggccgcaaaagcaacaacaattaca |
| | | | caatacaaacttgcaaatgactgaactaaaaaaacaaaagaccgctgaacaaaaaaccagacctcaaaatgt |
| | | | cggtattaaaggtatccaaatttacatcccaactcaatgtgtcaaccaatctgagctagagaaatttgatggcgt |
| | | | ttctcaaggtaaatacacaattggtctgggccaaaccaacatgtcttttgtcaatgacagagaagatatctactc |
| | | | gatgtccctaactgttttgtctaagttgatcaagagttacaacatcgacaccaacaaaattggtagattagaagt |
| | | | cggtactgaaactctgattgacaagtccaagtctgtcaagtctgtcttgatgcaattgtttggtgaaaacactga |
| | | | cgtcgaaggtattgacacgcttaatgcctgttacggtggtaccaacgcgttgttcaactctttgaactggattga |
| | | | atctaacgcatgggatggtagagacgccattgtagtttgcggtgatattgccatctacgataagggtgccgca |
| | | | agaccaaccggtggtgccggtactgttgctatgtggatcggtcctgatgctccaattgtatttgactctgtaaga |
| | | | gcttcttacatggaacacgcctacgatttttacaagccagatttcaccagcgaatatccttacgtcgatggtcatt |
| | | | tttcattaacttgttacgtcaaggctcttgatcaagtttacaagagttattccaagaaggctatttctaaagggttg |
| | | | gttagcgatcccgctggttcggatgctttgaacgttttgaaatatttcgactacaacgttttccatgttccaacctg |
| | | | taaattggtcacaaaatcatacggtagattactatataacgatttcagagccaatcctcaattgttcccagaagtt |
| | | | gacgccgaattagctactcgcgattatgacgaatctttaaccgataagaacattgaaaaaacttttgttaatgttg |
| | | | ctaagccattccacaaagagagagttgcccaatctttgattgttccaacaaacacaggtaacatgtacaccgc |
| | | | atctgtttatgccgcctttgcatctctattaaactatgttggatctgacgacttacaaggcaagcgtgttggtttattt |
| | | | tcttacggttccggtttagctgcatctctatattcttgcaaaattgttggtgacgtccaacatattatcaaggaatta |
| | | | gatattactaacaaattagccaagagaatcaccgaaactccaaaggattacgaagctgccatcgaattgaga |
| | | | gaaaatgcccatttgaagaagaacttcaaacctcaaggttccattgagcatttgcaaagtggtgtttactacttg |
| | | | accaacatcgatgacaaatttagaagatcttacgatgttaaaaaataa |
| ERG19 | 22 | S000005326 | atgaccgtttacacagcatccgttaccgcacccgtcaacatcgcaacccttaagtattgggggaaaagggac |
| | | | acgaagttgaatctgcccaccaattcgtccatatcagtgactttatcgcaagatgacctcagaacgttgacctct |
| | | | gcggctactgcacctgagtttgaacgcgacactttgtggttaaatggagaaccacacagcatcgacaatgaa |
| | | | agaactcaaaattgtctgcgcgacctacgccaattaagaaaggaaatggaatcgaaggacgcctcattgccc |
| | | | acattatctcaatggaaactccacattgtctccgaaaataactttcctacagcagctggtttagcttcctccgctg |
| | | | ctggctttgctgcattggtctctgcaattgctaagttataccaattaccacagtcaacttcagaaatatctagaata |
| | | | gcaagaaaggggtctggttcagcttgtagatcgttgtttggcggatacgtggcctgggaaatgggaaaagct |
| | | | gaagatggtcatgattccatggcagtacaaatcgcagacagctctgactggcctcagatgaaagcttgtgtcc |
| | | | tagttgtcagcgatattaaaaaggatgtgagttccactcagggtatgcaattgaccgtggcaacctccgaacta |
| | | | tttaaagaaagaattgaacatgtcgtaccaaagagatttgaagtcatgcgtaaagccattgttgaaaaagatttc |
| | | | gccacctttgcaaaggaaacaatgatggattccaactctttccatgccacatgtttggactctttccctccaatatt |
| | | | ctacatgaatgacacttccaagcgtatcatcagttggtgccacaccattaatcagttttacggagaaacaatcgt |
| | | | tgcatacacgtttgatgcaggtccaaatgctgtgttgtactacttagctgaaaatgagtcgaaactctttgcattta |
| | | | tctataaattgtttggctctgttcctggatgggacaagaaatttactactgagcagcttgaggctttcaaccatca |
| | | | atttgaatcatctaactttactgcacgtgaattggatcttgagttgcaaaaggatgttgccagagtgattttaactc |
| | | | aagtcggttcaggcccacaagaaacaaacgaatctttgattgacgcaaagactggtctaccaaaggaataa |
| tHMG1 | 23 | S000004540 | atgccagttttaaccaataaaacagtcatttctggatcgaaagtcaaaagtttatcatctgcgcaatcgagctcat |
| | | | caggaccttcatcatctagtgaggaagatgattcccgcgatattgaaagcttggataagaaaatacgtccttta |
| | | | gaagaattagaagcattattaagtagtggaaatacaaaacaattgaagaacaaagaggtcgctgccttggttat |
| | | | tcacggtaagttacctttgtacgctttggagaaaaaattaggtgatactacgagagcggttgcggtacgtagga |
| | | | aggctctttcaattttggcagaagctcctgtattagcatctgatcgtttaccatataaaaattatgactacgaccgc |
| | | | gtatttggcgcttgttgtgaaaatgttataggttacatgcctttgcccgttggtgttataggccccttggttatcgat |
| | | | ggtacatcttatcatataccaatggcaactacagagggttgtttggtagcttctgccatgcgtggctgtaaggca |
| | | | atcaatgctggcggtggtgcaacaactgttttaactaaggatggtatgacaagaggcccagtagtccgtttccc |
| | | | aactttgaaaagatctggtgcctgtaagatatggttagactcagaagagggacaaaacgcaattaaaaaagct |
| | | | tttaactctacatcaagatttgcacgtctgcaacatattcaaacttgtctagcaggagatttactcttcatgagattt |
| | | | agaacaactactggtgacgcaatgggtatgaatatgatttctaaaggtgtcgaatactcattaaagcaaatggt |
| | | | agaagagtatggctgggaagatatggaggttgtctccgtttctggtaactactgtaccgacaaaaaaccagct |
| | | | gccatcaactggatcgaaggtcgtggtaagagtgtcgtcgcagaagctactattcctggtgatgttgtcagaa |
| | | | aagtgttaaaaagtgatgtttccgcattggttgagttgaacattgctaagaatttggttggatctgcaatggctgg |
| | | | gtctgttggtggatttaacgcacatgcagctaatttagtgacagctgttttcttggcattaggacaagatcctgca |
| | | | caaaatgttgaaagttccaactgtataacattgatgaaagaagtggacggtgatttgagaatttccgtatccatg |
| | | | ccatccatcgaagtaggtaccatcggtggtggtactgttctagaaccacaaggtgccatgttggacttattagg |
| | | | tgtaagaggcccgcatgctaccgctcctggtaccaacgcacgtcaattagcaagaatagttgcctgtgccgtc |
| | | | ttggcaggtgaattatccttatgtgctgccctagcagccggccatttggttcaaagtcatatgacccacaacag |
| | | | gaaacctgctgaaccaacaaaacctaacaatttggacgccactgatataaatcgtttgaaagatgggtccgtc |
| | | | acctgcattaaatcctaa |
| IDI1 | 24 | S000006038 | atgactgccgacaacaatagtatgccccatggtgcagtatctagttacgccaaattagtgcaaaaccaaacac |
| | | | ctgaagacattttggaagagtttcctgaaattattccattacaacaaagacctaatacccgatctagtgagacgt |
| | | | caaatgacgaaagcggagaaacatgtttttctggtcatgatgaggagcaaattaagttaatgaatgaaaattgt |
| | | | attgttttggattgggacgataatgctattggtgccggtaccaagaaagtttgtcatttaatggaaaatattgaaa |
| | | | agggtttactacatcgtgcattctccgtctttattttcaatgaacaaggtgaattacttttacaacaaagagccact |
| | | | gaaaaaataactttccctgatctttggactaacacatgctgctctcatccactatgtattgatgacgaattaggttt |
| | | | gaagggtaagctagacgataagattaagggcgctattactgcggcggtgagaaaactagatcatgaattagg |
| | | | tattccagaagatgaaactaagacaaggggtaagtttcactttttaaacagaatccattacatggcaccaagca |
| | | | atgaaccatggggtgaacatgaaattgattacatcctattttataagatcaacgctaaagaaaacttgactgtca |
| | | | acccaaacgtcaatgaagttagagacttcaaatgggtttcaccaaatgatttgaaaactatgtttgctgacccaa |
| | | | gttacaagtttacgccttggtttaagattatttgcgagaattacttattcaactggtgggagcaattagatgaccttt |
| | | | ctgaagtggaaaatgacaggcaaattcatagaatgctataa |

In some embodiments, the open reading frame of the yeast gene comprises, consists essentially of, or consists of nucleic acid sequence selected from one comprising, consisting essentially of, or consisting of one encoding an amino acid sequence having at least about 70%, at least about 72%, at least about 75%, at least about 80%, at least about 85%, at least about 90%, at least about 91%, at least about 92%, at least about 93%, at least about 94%, at least about 95%, at least about 95%, at least about 96%, at least about 97%, at least about 98%, at least about 99% identity to the sequence of SEQ TD NO: 25, SEQ ID NO: 26, SEQ ID NO: 27, SEQ ID NO: 28, SEQ ID NO: 29, SEQ ID NO: 30, or SEQ ID NO: 31.

Amino Acid Sequence Table

| Amino Acid | SEQ | Acc. No. | |
|---|---|---|---|
| Sequence | ID NO | (SGD) | Amino Acid Sequence |
| ERG8 | 25 | S000004833 | MSELRAFSAPGKALLAGGYLVLDTKYEAFVVGLSARMHAVA |
| | | | HPYGSLQGSDKFEVRVKSKQFKDGEWLYHISPKSGFIPVSIGG |
| | | | SKNPFIEKVIANVFSYFKPNMDDYCNRNLFVIDIFSDDAYHSQE |
| | | | DSVTEHRGNRRLSFHSHRIEEVPKTGLGSSAGLVTVLTTALAS |
| | | | FFVSDLENNVDKYREVIHNLAQVAHCQAQGKIGSGFDVAAA |
| | | | AYGSIRYRRFPPALISNLPDIGSATYGSKLAHLVDEEDWNITIK |
| | | | SNHLPSGLTLWMGDIKNGSETVKLVQKVKNWYDSHMPESLK |
| | | | IYTELDHANSRFMDGLSKLDRLHETHDDYSDQIFESLERNDCT |
| | | | CQKYPEITEVRDAVATIRRSFRKITKESGADIEPPVQTSLLDDC |
| | | | QTLKGVLTCLIPGAGGYDAIAVITKQDVDLRAQTANDKRFSK |
| | | | VQWLDVTQADWGVRKEKDPETYLDK |
| ERG10 | 26 | S000005949 | MSQNVYIVSTARTPIGSFQGSLSSKTAVELGAVALKGALAKVP |
| | | | ELDASKDFDEIIFGNVLSANLGQAPARQVALAAGLSNHIVAST |
| | | | VNKVCASAMKAIILGAQSIKCGNADVVVAGGCESMTNAPYY |
| | | | MPAARAGAKFGQTVLVDGVERDGLNDAYDGLAMGVHAEKC |
| | | | ARDWDITREQQDNFAIESYQKSQKSQKEGKFDNEIVPVTIKGF |
| | | | RGKPDTQVTKDEEPARLHVEKLRSARTVFQKENGTVTAANAS |
| | | | PINDGAAAVILVSEKVLKEKNLKPLAIIKGWGEAAHQPADFT |
| | | | WAPSLAVPKALKHAGIEDINSVDYFEFNEAFSVVGLVNTKILK |
| | | | LDPSKVNVYGGAVALGHPLGCSGARVVVTLLSILQQEGGKIG |
| | | | VAAICNGGGGASSIVIEKI |
| ERG12 | 27 | S000004821 | MSLPFLTSAPGKVIIFGEHSAVYNKPAVAASVSALRTYLLISES |
| | | | SAPDTIELDFPDISFNHKWSINDFNAITEDQVNSQKLAKAQQA |
| | | | TDGLSQELVSLLDPLLAQLSESFHYHAAFCFLYMFVCLCPHAK |
| | | | NIKFSLKSTLPIGAGLGSSASISVSLALAMAYLGGLIGSNDLEK |
| | | | LSENDKHIVNQWAFIGEKCIHGTPSGIDNAVATYGNALLFEKD |
| | | | SHNGTINTNNFKFLDDFPAIPMILTYTRIPRSTKDLVARVRVLV |
| | | | TEKFPEVMKPILDAMGECALQGLEIMTKLSKCKGTDDEAVET |
| | | | NNELYEQLLELIRINHGLLVSIGVSHPGLELIKNLSDDLRIGSTK |
| | | | LTGAGGGGCSLTLLRRDITQEQIDSFKKKLQDDFSYETFETDL |
| | | | GGTGCCLLSAKNLNKDLKIKSLVFQLFENKTTTKQQIDDLLLP |
| | | | GNTNLPWTS |
| ERG13 | 28 | S000004595 | MKLSTKLCWCGIKGRLRPQKQQQLHNTNLQMTELKKQKTAE |
| | | | QKTRPQNVGIKGIQIYIPTQCVNQSELEKFDGVSQGKYTIGLGQ |
| | | | TNMSFVNDREDIYSMSLTVLSKLIKSYNIDTNKIGRLEVGTETL |
| | | | IDKSKSVKSVLMQLFGENTDVEGIDTLNACYGGTNALFNSLN |
| | | | WIESNAWDGRDAIVVCGDIAIYDKGAARPTGGAGTVAMWIG |
| | | | PDAPIVFDSVRASYMEHAYDFYKPDFTSEYPYVDGHFSLTCY |
| | | | VKALDQVYKSYSKKAISKGLVSDPAGSDALNVLKYFDYNVF |
| | | | HVPTCKLVTKSYGRLLYNDFRANPQLFPEVDAELATRDYDES |
| | | | LTDKNIEKTFVNVAKPFHKERVAQSLIVPTNTGNMYTASVYA |
| | | | AFASLLNYVGSDDLQGKRVGLFSYGSGLAASLYSCKIVGDVQ |
| | | | HIIKELDITNKLAKRITETPKDYEAAIELRENAHLKKNFKPQGSI |
| | | | EHLQSGVYYLTNIDDKFRRSYDVKK |
| ERG19 | 29 | S000005326 | MTVYTASVTAPVNIATLKYWGKRDTKLNLPTNSSISVTLSQD |
| | | | DLRTLTSAATAPEFERDTLWLNGEPHSIDNERTQNCLRDLRQL |
| | | | RKEMESKDASLPTLSQWKLHIVSENNFPTAAGLASSAAGFAA |
| | | | LVSAIAKLYQLPQSTSEISRIARKGSGSACRSLFGGYVAWEMG |
| | | | KAEDGHDSMAVQIADSSDWPQMKACVLVVSDIKKDVSSTQG |
| | | | MQLTVATSELFKERIEHVVPKRFEVMRKAIVEKDFATFAKET |
| | | | MMDSNSFHATCLDSFPPIFYMNDTSKRIISWCHTINQFYGETIV |
| | | | AYTFDAGPNAVLYYLAENESKLFAFIYKLFGSVPGWDKKFTT |
| | | | EQLEAFNHQFESSNFTARELDLELQKDVARVILTQVGSGPQET |
| | | | NESLIDAKTGLPKE |
| tHMG1 | 30 | S000004540 | MPVLTNKTVISGSKVKSLSSAQSSSSGPSSSSEEDDSRDIESLDK |
| | | | KIRPLEELEALLSSGNTKQLKNKEVAALVIHGKLPLYALEKKL |
| | | | GDTTRAVAVRRKALSILAEAPVLASDRLPYKNYDYDRVFGAC |
| | | | CENVIGYMPLPVGVIGPLVIDGTSYHIPMATTEGCLVASAMRG |
| | | | CKAINAGGGATTVLTKDGMTRGPVVRFPTLKRSGACKIWLDS |
| | | | EEGQNAIKKAFNSTSRFARLQHIQTCLAGDLLFMRFRTTTGDA |
| | | | MGMNMISKGVEYSLKQMVEEYGWEDMEVVSVSGNYCTDKK |
| | | | PAAINWIEGRGKSVVAEATIPGDVVRKVLKSDVSALVELNIAK |
| | | | NLVGSAMAGSVGGFNAHAANLVTAVFLALGQDPAQNVESSN |
| | | | CITLMKEVDGDLRISVSMPSIEVGTIGGGTVLEPQGAMLDLLG |
| | | | VRGPHATAPGTNARQLARIVACAVLAGELSLCAALAAGHLV |
| | | | QSHMTHNRKPAEPTKPNNLDATDINRLKDGSVTCIKS |
| IDI1 | 31 | S000006038 | MTADNNSMPHGAVSSYAKLVQNQTPEDILEEFPEIIPLQQRPN |
| | | | TRSSETSNDESGETCFSGHDEEQIKLMNENCIVLDWDDNAIGA |
| | | | GTKKVCHLMENIEKGLLHRAFSVFIFNEQGELLLQQRATEKIT |
| | | | FPDLWTNTCCSHPLCIDDELGLKGKLDDKIKGAITAAVRKLDH |
| | | | ELGIPEDETKTRGKFHFLNRIHYMAPSNEPWGEHEIDYILFYKI |
| | | | NAKENLTVNPNVNEVRDFKWVSPNDLKTMFADPSYKFTPWF |
| | | | KIICENYLFNWWEQLDDLSEVENDRQIHRML |

In some embodiments, the disclosure relates to an isolated nucleic acid molecule comprising a combination of any one or more regulatory element sequence herein with any one or more gene sequence herein.

In some embodiments, the disclosure relates to one or more nucleic acid molecules comprising one or more open reading frames herein. In some embodiments, the disclosure relates to at least one of a first nucleic acid molecule comprising an open reading frame for the ERG8 gene or a functional variant thereof, a second nucleic acid molecule comprising an open reading frame for the ERG10 gene or a functional variant thereof, a third nucleic acid molecule comprising an open reading frame for the ERG12 gene or a functional variant thereof, a fourth nucleic acid molecule comprising an open reading frame for the ERG13 or a functional variant thereof, a fifth nucleic acid molecule comprising an open reading frame for the ERG19 or a functional variant thereof, a sixth nucleic acid molecule comprising an open reading frame for the tHMG1 gene or a functional variant thereof, and a seventh nucleic acid molecule comprising an open reading frame for the IDI1 gene or a functional variant thereof, wherein each of the first, second, third, fourth, fifth, sixth, and seventh open reading frames are operably linked to one or more regulatory element. In some embodiments, the one or more regulatory element comprises at least one promoter independently selected from pTDH3 or a functional variant thereof, pCCW12 or a functional variant thereof, pHHF2 or a functional variant thereof, pRPL18B or a functional variant thereof, pPOP6 or a functional variant thereof, pPGK1 or a functional variant thereof, pHTB2 or a functional variant thereof, pRNR2 or a functional variant thereof, pTEF2, pPAB1 or a functional variant thereof, pPSP2 or a functional variant thereof, pTEF1 or a functional variant thereof, pALD6 or a functional variant thereof, pRAD27 or a functional variant thereof, pHHF1 or a functional variant thereof, pRET2 or a functional variant thereof, and pREV1 or a functional variant thereof. In some embodiments, the one or more regulatory element are independently selected and comprises a nucleic acid sequence comprising at least about 70%, at least about 72%, at least about 75%, at least about 80%, at least about 85%, at least about 90%, at least about 91%, at least about 92%, at least about 93%, at least about 94%, at least about 95%, at least about 95%, at least about 96%, at least about 97%, at least about 98%, at least about 99%, or about 100% identity to the sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, or SEQ ID NO: 17. In some embodiments, the ERG8 gene or a functional variant thereof, the ERG10 gene or a functional variant thereof, the ERG12 gene or a functional variant thereof, the ERG13 or a functional variant thereof, the ERG19 or a functional variant thereof, the tHMG1 gene or a functional variant thereof, and the IDI1 gene or a functional variant thereof comprise a nucleic acid sequence comprising at least about 70%, at least about 72%, at least about 75%, at least about 80%, at least about 85%, at least about 90%, at least about 91%, at least about 92%, at least about 93%, at least about 94%, at least about 95%, at least about 95%, at least about 96%, at least about 97%, at least about 98%, at least about 99%, or about 100% identity to the sequence of SEQ ID NO: 18, SEQ ID NO: 19, SEQ ID NO: 20, SEQ ID NO: 21, SEQ ID NO: 22, SEQ ID NO: 23, or SEQ ID NO: 24, respectively. In some embodiments, the ERG8 gene or a functional variant thereof, the ERG10 gene or a functional variant thereof, the ERG12 gene or a functional variant thereof, the ERG13 gene or a functional variant thereof, the ERG19 gene or a functional variant thereof, the tHMG1 gene or a functional variant thereof, and the IDI1 gene or a functional variant thereof comprise a nucleic acid sequence encoding an amino acid sequence comprising at least about 70%, at least about 72%, at least about 75%, at least about 80%, at least about 85%, at least about 90%, at least about 91%, at least about 92%, at least about 93%, at least about 94%, at least about 95%, at least about 95%, at least about 96%, at least about 97%, at least about 98%, at least about 99%, or about 100% identity to the sequence of SEQ ID NO: 25, SEQ ID NO: 26, SEQ ID NO: 27, SEQ ID NO: 28, SEQ ID NO: 29, SEQ ID NO: 30, or SEQ ID NO: 31, respectively. In some embodiments, the at least one of a first, second, third, fourth, fifth, sixth, and seventh nucleic acid molecule comprises a plurality of the first, second, third, fourth, fifth, sixth, and seventh nucleic acid molecules. In some embodiments, the at least one of a first, second, third, fourth, fifth, sixth, and seventh nucleic acid molecule comprises all of the first, second, third, fourth, fifth, sixth, and seventh nucleic acid molecules.

In some embodiments, the disclosure relates to one to seven nucleic acid molecules. Combined, the one to seven nucleic acid molecules comprise at least the open reading frames for the ERG8 gene or a functional variant thereof, the ERG10 gene or a functional variant thereof, the ERG12 gene or a functional variant thereof, the ERG13 or a functional variant thereof, and the ERG19 or a functional variant thereof, each open reading frame operably linked to one or more regulatory element. In some embodiments, the one to seven nucleic acid molecules further comprise the open reading frame for the tHMG1 gene or a functional variant thereof, and the open reading frame for the IDI1 gene or a functional variant thereof, each open reading frame operably linked to one or more regulatory element. The open reading frames and regulatory elements, in some embodiments, are as described above.

### Vectors

In some embodiments, the disclosure relates to a vector comprising any one or more nucleic acid herein. In some embodiments, a vector herein further comprises at least one of a yeast origin of replication, one or more selection markers, one or more resistance markers. In some embodiments, the yeast origin of replication is selected from Up, YRp, YCp, or YEp. In some embodiments, the one or more section markers are selected from HIS3, URA3, LYS2, LEU2, TRP1, MET15, ura4+, leu1+, and ade6+. In some embodiments, the one or more resistance markers are selected from kan(r), KanMX3, kanMX4, or open reading frames conferring resistance to the antibiotics hygromycin B (hph), nourseothricin (nat), or G418.

Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989. Cells are genetically engineered by the introduction into the cells of heterologous DNA (RNA). That heterologous DNA (RNA) is placed under operable control of transcriptional elements to permit the expression of the heterologous DNA in the host cell. Heterologous expression of genes associated with the invention, for production of a terpenoid, such as taxadiene, is demonstrated in the Examples section using a modified yeast cell.

A nucleic acid molecule that encodes an enzyme associated with the terpene synthesis can be introduced into a cell or cells using methods and techniques that are standard in the art. For example, nucleic acid molecules can be introduced by standard protocols such as transformation including chemical transformation and electroporation, transduction, particle bombardment, etc. Expressing the nucleic acid molecule encoding the enzymes of the claimed invention also may be accomplished by integrating the nucleic acid molecule into the genome.

In some embodiments one or more genes associated with the invention is expressed recombinantly in a modified yeast cell disclosed herein. Yeast cells according to the invention can be cultured in media of any type (rich or minimal) and any composition. As would be understood by one of ordinary skill in the art, routine optimization would allow for use of a variety of types of media. The selected medium can be supplemented with various additional components. Some non-limiting examples of supplemental components include glucose, antibiotics, an inducible promoter for gene induction, ATCC Trace Mineral Supplement, and glycolate. Similarly, other aspects of the medium, and growth conditions of the cells of the invention may be optimized through routine experimentation. For example, pH and temperature are non-limiting examples of factors which can be optimized. In some embodiments, factors such as choice of media, media supplements, and temperature can influence production levels of terpenes, such as menthol. In some embodiments the concentration and amount of a supplemental component may be optimized. In some embodiments, how often the media is supplemented with one or more supplemental components, and the amount of time that the media is cultured before harvesting a terpene, such as menthol, is optimized.

According to aspects of the invention, high titers of a terpenoid (such as but not limited to menthol), are produced through the recombinant expression of genes as described herein, in a cell expressing components of the known metabolic pathway, and one or more downstream genes for the production of a terpene (or related compounds) from the products of the metabolic pathway. As used herein “high titer” refers to a titer in the milligrams per liter (mg per liter of culture medium) scale. The titer produced for a given product will be influenced by multiple factors including choice of media. In some embodiments, the total titer of a terpene or derivative is at least about 1 mg per liter of culture medium. In some embodiments, the total terpenoid or derivative titer is at least about 10 mg per liter of culture medium. In some embodiments, the total terpenoid or derivative titer is at least about 250 mg per liter of culture medium. For example, the total terpenoid or derivative titer can be at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 75, 80, 85, 90, 95, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900 or more than about 900 mg per liter of culture medium including any intermediate values. In some embodiments, the total terpenoid or derivative titer can be at least about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, or more than 5.0 grams per liter of culture medium including any intermediate values.

In some embodiments, the total terpene titer is at least about 1 mg per liter of culture medium. In some embodiments, the total titer is at least about 10 mg per liter of culture medium. In some embodiments, the total terpene titer is at least about 50 mg per liter of culture medium. For example, the total terpene titer can be at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, or more than about 70 mg per liter of culture medium including any intermediate values.

### Compositions

In some embodiments, the disclosure relates to a composition comprising any one or more nucleic acid herein. In some embodiments, the composition further comprises a cell, such as a yeast cell. In some embodiments, the cell comprises any one or more nucleic acid molecules and/or open reading frames disclosed herein. In other embodiments, the cell is a fungal cell such as a yeast cell, e.g., Saccharomyces spp., Schizosaccharomyces spp., Pichia spp., Paffia spp., Kluyveromyces spp., Candida spp., Talaromyces spp., Brettanomyces spp., Pachysolen spp., Debaryomyces spp., Yarrowia spp., and industrial polyploid yeast strains. In some embodiments, the yeast strain is a S. cerevisiae strain or a Yarrowia spp. Strain.

In some embodiments, the disclosure relates to a composition comprising any one or more vectors herein. In some embodiments, the composition further comprises a yeast cell.

In some embodiments, the disclosure relates to a composition comprising one or more strains listed in Example 9. In some embodiments, the composition further comprises at least one terpene. The at least one terpene, in some embodiments, is as described below. In some embodiments, the composition further comprises a culture medium.

In some embodiments, the disclosure relates to a composition comprising a modified yeast cell. In some embodiments, the modified yeast cell comprises any one or more nucleic acid herein. In some embodiments, the modified yeast cell comprises any one or more vector herein. In some embodiments, the modified yeast cell comprises any one or more amino acid sequence herein.

In some embodiments, the disclosure relates to a composition comprising a modified yeast cell. In some embodiments, the modified yeast cell comprises open reading frames encoding ERG8, ERG10, ERG12, ERG13, and ERG19, and a first regulatory sequence of weak-strength, medium-strength or high-strength operably linked to the open reading frame encoding ERG12. In some embodiments, the yeast cell further comprises one or both of an open reading frame encoding tHMG1 and an open reading frame encoding IDI1.

In some embodiments, an open reading frame herein comprises a nucleic acid sequence encoding one of ERG8, ERG10, ERG12, ERG13, ERG19, tHMG1, or IDI1. In some embodiments, the yeast cell comprises a nucleic acid molecule comprising each of the open reading frames. In some embodiments, the composition yeast cell comprises a plurality of nucleic acid molecules, and two or more of the plurality of nucleic acid molecules comprise one or more of the open reading frames. In some embodiments, a nucleic acid molecule herein is a yeast chromosome. In some embodiments, a nucleic acid molecule herein is a vector.

In some embodiments, the yeast cell further comprises one or more of: a second regulatory sequence operably linked to the open reading frame encoding ERG8, a third regulatory sequence operably linked to the open reading frame encoding ERG10, a fourth regulatory sequence operably linked to the open reading frame encoding ERG13, and a fifth regulatory sequence operably linked to the open reading frame encoding ERG19.

In some embodiments, the first regulatory sequence, the second regulatory sequence, the third regulatory sequence, the fourth regulatory sequence, and the fifth regulatory sequence are each high-strength promoters. In some embodiments, the first regulatory sequence, the second regulatory sequence, the third regulatory sequence, the fourth regulatory sequence, and the fifth regulatory sequence are independently selected from a promoter comprising a nucleic acid sequence comprising at least about 70%, at least about 72%, at least about 75%, at least about 80%, at least about 85%, at least about 90%, at least about 91%, at least about 92%, at least about 93%, at least about 94%, at least about 95%, at least about 95%, at least about 96%, at least about 97%, at least about 98%, at least about 99%, or about 100% sequence identity to SEQ ID NO: 1, SEQ ID NO: 2, SEQ ID NO: 3, SEQ ID NO: 4, SEQ ID NO: 5, SEQ ID NO: 6, or SEQ ID NO: 7. In some embodiments, the first regulatory sequence, the second regulatory sequence, the third regulatory sequence, the fourth regulatory sequence, and the fifth regulatory sequence are independently selected from: pTDH3, pCCW12, pPGK1, pHHF2, pTEF1, pTEF2, and pHHF1

In some embodiments, the first regulatory sequence, the second regulatory sequence, the third regulatory sequence, the fourth regulatory sequence, and the fifth regulatory sequence are each medium-strength promoters. In some embodiments, the first regulatory sequence, the second regulatory sequence, the third regulatory sequence, the fourth regulatory sequence, and the fifth regulatory sequence are independently selected from a promoter comprising a nucleic acid sequence that comprises at least about 70%, at least about 72%, at least about 75%, at least about 80%, at least about 85%, at least about 90%, at least about 91%, at least about 92%, at least about 93%, at least about 94%, at least about 95%, at least about 95%, at least about 96%, at least about 97%, at least about 98%, at least about 99%, or about 100% sequence identity to SEQ ID NO: 8, SEQ ID NO: 9, SEQ ID NO: 10, SEQ ID NO: 11, or SEQ ID NO: 12. In some embodiments, the first regulatory sequence, the second regulatory sequence, the third regulatory sequence, the fourth regulatory sequence, and the fifth regulatory sequence are independently selected from pRPL18B, pHTB2, pALD6, pPAB1, and pRET2. In some embodiments, the first regulatory sequence, the second regulatory sequence, the third regulatory sequence, the fourth regulatory sequence, and the fifth regulatory sequence are independently selected from a promoter comprising a nucleic acid sequence that comprises at least about 70%, at least about 72%, at least about 75%, at least about 80%, at least about 85%, at least about 90%, at least about 91%, at least about 92%, at least about 93%, at least about 94%, at least about 95%, at least about 95%, at least about 96%, at least about 97%, at least about 98%, at least about 99%, or about 100% sequence identity to pRPL18B, pHTB2, pALD6, pRNR1, pPAB1, pRET2, and pSAC6. In some embodiments, the first regulatory sequence, the second regulatory sequence, the third regulatory sequence, the fourth regulatory sequence, and the fifth regulatory sequence are independently selected from pRPL18B, pHTB2, pALD6, pRNR1, pPAB1, pRET2, and pSAC6. In some embodiments, the first regulatory sequence, the second regulatory sequence, the third regulatory sequence, the fourth regulatory sequence, and the fifth regulatory sequence are each weak-strength promoters. In some embodiments, the first regulatory sequence, the second regulatory sequence, the third regulatory sequence, the fourth regulatory sequence, and the fifth regulatory sequence are independently selected from a promoter comprising a nucleic acid sequence that comprises at least about 70%, at least about 72%, at least about 75%, at least about 80%, at least about 85%, at least about 90%, at least about 91%, at least about 92%, at least about 93%, at least about 94%, at least about 95%, at least about 95%, at least about 96%, at least about 97%, at least about 98%, at least about 99%, or about 100% sequence identity to SEQ ID NO: 13, SEQ ID NO: 14, SEQ ID NO: 15, SEQ ID NO: 16, or SEQ ID NO: 17. In some embodiments, the first regulatory sequence, the second regulatory sequence, the third regulatory sequence, the fourth regulatory sequence, and the fifth regulatory sequence are independently selected from pPOP6, pRNR2, pPSP2, pRAD27, and pREV1.

In some embodiments, the first regulatory sequence is selected from a promoter comprising a nucleic acid sequence having at least about 70%, at least about 72%, at least about 75%, at least about 80%, at least about 85%, at least about 90%, at least about 91%, at least about 92%, at least about 93%, at least about 94%, at least about 95%, at least about 95%, at least about 96%, at least about 97%, at least about 98%, at least about 99%, or about 100% sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12 SEQ ID NO: 13, SEQ ID NO: 14, SEQ ID NO: 15, SEQ ID NO: 16, or SEQ ID NO: 17, and the second regulatory sequence, the third regulatory sequence, the fourth regulatory sequence, and the fifth regulatory sequence are each independently selected from a promoter comprising a nucleic acid sequence comprising at least about 70%, at least about 72%, at least about 75%, at least about 80%, at least about 85%, at least about 90%, at least about 91%, at least about 92%, at least about 93%, at least about 94%, at least about 95%, at least about 95%, at least about 96%, at least about 97%, at least about 98%, at least about 99%, or about 100% sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, or SEQ ID NO: 17.

In some embodiments, the modified yeast cell is free of modification of one or more yeast genes selected from LPP1, DPP1, HO, ERG1, ANT1, IDP2, IDP3, Cit2, ACS1, ACL1, ACL2, Met15, RHR2, NADH-HMGR, ERG9, GPD1, and GPD2. In some embodiments, the modified yeast cell is free of modification of a plurality of yeast genes selected from LPP1, DPP1, HO, ERG1, ANT1, IDP2, IDP3, Cit2, ACS1, ACL1, ACL2, Met15, RHR2, NADH-HMGR, ERG9, GPD1, and GPD2. In some embodiments, the modified yeast cell is free of modification of the yeast genes LPP1, DPP1, HO, ERG1, ANT1, IDP2, IDP3, Cit2, ACS1, ACL1, ACL2, Met15, RHR2, NADH-HMGR, ERG9, GPD1, and GPD2.

In some embodiments, the modified yeast cell further comprises one, two, or three regulatory sequences operably linked to the open reading frame encoding ERG8, when present, one, two, or three regulatory sequences operably linked to the open reading frame encoding ERG10, when present, one, two, or three regulatory sequences operably linked to the open reading frame encoding ERG13, when present, and one, two, or three regulatory sequences operably linked to the open reading frame encoding ERG19, when present, and one or more of a sixth regulatory sequence operably linked to the open reading frame encoding ERG12, when present, and seventh regulatory sequence operably linked to the open reading frame encoding ERG12 when present.

In some embodiments, the composition further comprises a culture of the modified yeast cell comprising about a 94-fold, about a 60-fold, and/or about a 35-fold improved titer of monoterpene geraniol, sesquiterpene α-humulene, and triterpene squalene, respectively, over a culture of wild type yeast cell.

In some embodiments, the composition further comprises at least one terpene. In some embodiments, the composition further comprises a culture medium. In some embodiments, the composition further comprises at least one terpene and a culture medium. In some embodiments, the terpene is at least about 10 mg/L to about 20 mg/L of culture medium.

In some embodiments, the at least one terpene is selected from monoterpenes, sesquiterpenes, diterpenes, triterpenes, tertaterpenes, polyterpenes.

In some embodiments, at least one terpene comprises at least one monoterpene selected from α-Phellandrene, grandisol, thujone, artemisia alcohol, yomogi alcohol, yomogone, myrcene, carvone, dihydrocarvone, dihydrocarvyl acetate, carvoloxide, ascaridole, chrysanthemic acid, chrysanthemone, chrysanthemol, chrysanthenyl acetate, borneol, camphor, linalool oxide, γ-terpinene, limonenol, limonene, limonene-1,2-diol, limonene oxide, safranal, citral, geraniol, citronellal, sabinene, phellandrene, phellandrene epoxide, piperitone oxide, eucalyptol, pinocarveol, 1,4-cineole, phellandral, cryptone, fenchone, fenchol, fenchyl formate, ipsdienol, ipsenol, sabina ketone, sabinol, linalool, lavandulol, myrcenol acetate, lavandulyl acetate, dihydromyrcenol, α-terpinene, terpinene-4-ol, terpinene-1-ol, melilotal, isopulegol, menthol, carvomenthenol, carvomenthyl acetate, mintlactone, menthenol, carvomenthol, isocarvomenthol, piperitenone, piperitenone oxide, piperitone piperitol, piperityl acetate, isopiperitone, piperitylacetone, menthofuran, pulegone, eucarvone, dihydrocarveol, isopulegyl acetate, carveol formate, carveol, carveol acetate, carvenone, isodihydrocarveol, carveol methyl ether, myrtenol, myrtenyl acetate, myrtenal, myrtenyl formate, carvacrol, α-thujene, carvacrol methyl ester, origanol, perillyl alcohol, dihydroperillyl alcohol, perillic acid, perillaldehyde, dihydroperillic acid, dihydroperillol, isoperillyl alcohol, camphene, α-terpineol, terpineol acetate, sobrerol, α-pinene, isoterpinolene, nopol, pinanediol, nopinone, terpinolene, pinocarvone, nerol, citronellol, rose oxide, rosefuran, β-pinene, verbenone, salvylene, salviol, teresantalol, santolinatriene, tagetone, dihydrotagetone, carvotanacetone, thujenol, thuj-3-en-10-al, α-Thujenal, thujyl alcohol, thujol, isoborneol, cymenol, thymol, sabinene hydrate, methylthymol, cymenene, p-cymene, umbelluone, verbenol, verbenol oxide, and verbenone.

In some embodiments, at least one terpene comprises at least one sesquiterpene selected from Chamazulene, acorenone, acora-3,5-diene, β-acoradiene, africanol, selinene, ishwarane, artemisinin, asteriscanolide, oppositol, axamide, spathulenol, botrydial, guaiadiene, guaiol, ylangene, elemol, elematol, sativene, isosativene, capsidiol, himachalane, cedrol, cedrene, nootkatone, farnesol, bergamotene, quinol, silphinene, furanoeudesma-1,3-diene, copaene, β-eudesmol, α-bulnesene, cuparene, curcumene, β-elemene, furanodiene, xanthorrhizol, zedoarol, isocyperol, carotol, daucene, isodaucene, dendrolasin, dictamnol, yahazunol, drimane, polygodial, furodysinin, eremophilone, eremoligenol, aromadendrene, globulol, reidin, gossonorol, gossypol, guaiene, quaianine, hedycaryol, helminthosporal, helminthosprol, helminthogermacrene, α-humulene, alantolactone, widdrol, junenol, widdrane, junipene, junicedranol, kickxin, lactarorufin, ledol, lepidozene, lepisantine, lepidozenol, maalioxide, marasmene, guaiazulene, α-bisabolol, viridiflorol, jatamansone, kusunol, illudin, oplopanone, petasol, longifolene, nerolidol, patchouli alcohol, patchoulol, premnaspirodiene, prezizaene, prezizanol, salvial-4(14)-en-1-one, α-santalol, costunolide, dehydrocostus lactone, dehydrocostuslactone, furanoeremophilane, caryolane, clovane, neoclovene, β-caryophyllene, parthenolide, thapsigargin, occidentalol, thujopsene, hibaene, modhephene, upial, valencianes, valerenic acid, valeranone, valerenal, valerianol, kessane, valerendial, germacrene D, cadinene, cadinol, bicyclogermacrene, isoledene, neomeranol, oxymaalioxide, cubenol, α-vetivone, zizaene, zizanene, khusimol, rotundone, warburganal, africanene, muzigadial, xanthinol, zingiberenol, zingiberene, and zerumbone.

In some embodiments, at least one terpene comprises at least one diterpene selected from 6β,7β-Dihydroxy-12-methylroyleanone, 7β-hydroxyroyleanone, 6β-hydroxyroyleanone, 6β,7β-dihydroxyroyleanone, 7β-acetoxy-6β-hydroxyroyleanone, 7β-acetoxy-6β-hydroxy-12-o-methylroyleanone, coleon-u-quinone, demethylinuroyleanol, coleon V, ar-abietatriene, 17-hydroxyjolkinolide B, plectranthroyleanone B, plectranthroyleanone C, sugiol, 6,7-dehydroferruginol, ferruginol, eupholides F, eupholides G, eupholides H, 14α-hydroxy-17-al-ent-abieta-7(8),11(12),13(15)-trien-16,12-olide, horminone, 7α-acetoxy-6β-hydroxyroyleanone, scordidesin A, teucrin A, ballodiolic acid A, ballodiolic acid B, (−)-polyalthic acid, kaurenoic acid, (1R*,2E,4R*,7E,10S*,11S*,12R*)-10,18-diacetoxydolabella-2,7-dien-6-one, stachatranone B, atranone Q, ent-beyer-15-en-18-o-malonate, ent-beyer-15-en-18-o-succinate, ent-beyer-15-en-18-o-oxalate, (5S,7R,8S,9R,10S,12R)-7,8-dihydroxycleroda-3,13(16),14-triene-17,12; 18,19-diolide, (7R,8S,9R,12R)-7-hydroxy-5,10-seco-neo-cleroda-1 (10),2,4,13 (16),14-pentaene-17,12; 18,19-diolide, tilifodiolide, (5R,7R,8S,9R,10R,12R)-7-hydroxycleroda-1,3,13(16),14-tetraene-17,12;18,19-diolide, splendidin C, galdosol, (5S,7R,8R,9R,10S,12R)-7,8-dihydroxycleroda-3,13(16),14-tri-ene-17,12;18,19-diolide, psathyrellins A, psathyrellins B, psathyrellins C, harzianol I, emindole SB, paspalitrem C, 6-hydroxylpaspalinine, paspaline, 3-deoxo-4b-deoxypaxilline, PC-M6, drechmerin A, drechmerin C, drechmerin G, terpendole I, penijanthine C, penijanthine D, drechmerin, terpendole L, cladosporine A, akhdarenol, virescenol B, 19-acetoxy-7,15-isopimaradien-3β-ol, 17-hydroxy-ent-kaur-15-en-18-oic acid, acidanticopalic acid, 8(17)-labden-15-ol, anticopalol, labda-8(17),13-dien-15-oic acid, 8(17),11(Z),13(E)-trien-15,18-dioic acid, coleonol B, forskolin, cuceolatins A, cuceolatins B, cuceolatins C, 8(17),12,14-labda-trien-18-oic acid, vitexilactone, andrographolide, libertellenone A, eutypellenoid B, sandaracopimarinol, icacinlactone B, cryptotanshinone, ebractenoid Q, euphorin A, macfarlandin D, macfarlandin G, carmichaedine, sinchiangensine A, lipodeoxyaconitine, heterophylline A, heterophylline B, condelphine, koninginol A, koninginol B, conidiogenone C, conidiogenone D, conidiogenone G, psathyrelloic acid, psathyrins A, psathyrins B, smirnotine A, smirnotine B, jolkinolide B, jolkinolide A, 17-hydroxyjolkinolide B, 17-acetoxyjolkinolide B, prostratin, langduin A, 13-o-acetylphorbol, 12-deoxyphorbol 13-palmitate, ingenol-6,7-epoxy-3-tetradecanoate, ingenol-3-myristinate, ingenol 3-palmitate, ent-1β,3β,16β, 17-tetrahydroxyatisane, ent-1β,3α,16β, 17-tetrahydroxyatisane, ent-kaurane-3-oxo-16β, 17-acetonide, phylloquinone, colforsin, vitamin A, menadione, alitretinoin, tretinoin, paclitaxel, docetaxel, carboxyatractyloside, 4-oxoretinol, anhydrovitamin A, N-ethylretinamide, ecabet, paclitaxel docosahexaenoic acid, AI-850, paclitaxel trevatide, ginkgolide A, ginkgolide-C, ginkgolide-J, cabazitaxel, gibberellic acid, gibberellin A4, ortataxel, tesetaxel, menatetrenone, salvinorin A, milataxel, steviolbioside, BMS-188797, BMS-184476, larotaxel, menaquinone 7, motretinide, paclitaxel poliglumex, 13-cis-12-(3′-carboxyphenyl)retinoic acid, menadiol diphosphate, menaquinone 6, rebaudioside A, menaquinone, simotaxel, menadione bisulfite, isosteviol, stevioside, tanshinone I phorbol 12-myristate 13-acetate diester, TPI-287, paclitaxel ceribate, transcrocetinate, aphidicolin, ANG1005, and oridonin.

In some embodiments, at least one terpene comprises at least one triterpene selected from Cucurbitacin E, taikugausins A, taikugausins B, taikugausins C, taikugausins D, taikugausins E, kuguacins II-VI, kaguacin X, citriodora A, hemsleypenside B, cucurbitacin I, cucurbitacin Q, 2-deoxycucurbitacin D, 25-acetylcucurbitacin F, cucurbitacin D, cucurbitacin B, cucurbitacin D, cucurbitacin E, cucurbitacin I, 23,24-dihydro-cucurbitacin F, 23,24-dihydro-25-acetylcucurbitacin F, 23,24-dihydro-cucurbitacin B, cucurbitacin B, cucurbitacin B, balsaminapentanol, balsaminol A, balsaminol B, cucurbalsaminol B, cabraleadiol, cabraleahydroxylactone, cabralealactone, eichlerialactone, methyl antcinate B, zhankuic acid A, zhankuic acid C, netzahualcoyonol tigenone, celastrol, pristimerin, celastrol, fridelin, fridelin-1-3-dione,15α-acetyl-dehydrosulphurenic acid, sulphurenic acid, meliavolkenin, melianin B, melianin C, meliavolkinin, betulinic acid, botulin, lupeol, remangilones A, remangilones C, 3β,23,28-trihydroxy-12-oleanene 23-caffeate, 3β,23,28-trihydroxy-12-oleanene 3β-caffeate, oleanolic acid, masticadienonic acid, masticadienolic acid, 3-α-hydroxy-masticadienolic acid, 24,25S-dihydro-masticadienoic acid, ursolic acid, promolic acid, 2-oxopromolic acid, 3-o-acetyl promolic acid, α-amyrine, ursolic acid, cis- and trans-3-o-p-hydroxycinnamoyl ursolic acid, 2α-hydroxyursolic acid, 3β-trans-p-coumaroyloxy-2α-hydroxyolean-12-en-28-oic acid, 2α-hydroxyursolic acid, uncarinic acid C, uncarinic acid D, uncarinic acid E, 9,19-cycloart-23-ene-3β,25-diol, 9,19-Cycloart-25-ene-3β,24-diol, bryonolic acid, AECHL-1, glycyrrhizic acid, ginsenosides, Ibrexafungerp, squalene, carbenoxolone, bardoxolone methyl, ginsenoside C, ginsenoside Rb1, ginsenoside Rg1, squalane, betulinic Acid, lupeol, bardoxolone, enoxolone, acetoxolone, asiatic acid, ginsenoside B2, beta-escin, escin, pristimerin, omaveloxolone, bevirimat, botulin, celastrol, ginsenoside Rd, and ginsenoside Rg3.

In some embodiments, at least one terpene comprises at least one tertraterpene and/or polyterpene selected from β-Carotene, lycopene, lutein, zeaxanthin, astaxanthin, canthaxanthin, fucoxanthin, bixin, capsanthin, crocetin, staphyloxanthin, spirilloxanthin, bacterioruberin, peridinin, violaxanthin, neoxanthin, diadinoxanthin, alloxanthin, torulene, spheroidene, oscillaxanthin, myxoxanthophyll, siphonaxanthin, pectenolone, echinenone, phoenicoxanthin, rhodoxanthin, rubixanthin, phytoene, phytofluene, α-carotene, γ-carotene, cryptoxanthin, capsorubin, thermozeaxinthin, saproxanthin, flexixanthin, neurosporaxanthin, torularhodin, auroxanthin, lactucaxanthin, okenone, isorenieratene, sarcinaxanthin, decaprenoxanthin, mutatochrome, retinal, retinoic acid, crocin, picrocrocin, antheraxanthin, dinoxanthin, monadoxanthin, prasinoxanthin, loroxanthin, diatoxanthin, heteroxanthin, trollixanthin, mytiloxanthin, trikentriorhodin, astacene, idoxanthin, crustaxanthin, plectaniaxanthin, phillipsiaxanthin, eutreptiellanone, pyrrhoxanthin, mimulaxanthin, mactraxanthin, phleixanthophyll, lutein dipalmitate, zeaxanthin dipalmitate, astaxanthin diester, fucoxanthin palmitate, capsanthin dipalmitate, dehydroretinol, β-apocarotenal, citranaxanthin, rhodopinal, spheroidenol, ionone, β-cyclocitral, safranal, damascenone, megastigmatrienone, synechoxanthin, caloxanthin, nostoxanthin, chlorobactene, hydroxypyrrhoxanthin, renierapurpurin, siphonein, peridininol, okenirone, spheroidenethiol, thiothece-474, ζ-carotene, mutatoxanthin, citraurin, tetrahydrolycopene, keto-α-carotene, 3-Hydroxyechinenone, 4-ketozeaxanthin, adonixanthin, aleuriaxanthin, anhydrolutein, azafrinone, bacterial vioxanthin, β-cryptoxanthin-5,6-epoxide, β-doradexanthin, celaxanthin, corynexanthin, cryptoflavin, deepoxineoxanthin, deinoxanthin, deoxylutein, diketospirilloxanthin, echinenone-4-oxide, epilutein, erythroxanthin, flexixanthin-3-glucoside, foliachrome, gazaniaxanthin, hydroxyspirilloxanthin, isocryptoxanthin, isorenieratene-3-glucoside, ketospirilloxanthin, latochrome, leprotene, lycoxanthin, marennine, methoxyneurosporene, micrococcin, myxobactin, neochrome, nephrocytol, neurosporaxanthin-β-D-glucoside, nonaprenoxanthin, OH-chlorobactene, oscillol, paracentrone, pectenol, pentaxanthin, persicaxanthin, phillisiaxanthin-β-glucoside, physalien, pipixanthin, plectaniaxanthin-6′-epoxide, prolycopene, pyrrhoxanthininol, rhodopin, rhodopinol, rubichrome, sarcinene, siphonaxanthin-3′-glucoside, spheroidenone-hydroxy, spirilloxanthin-20-al, sulcatoxanthin, taraxanthin, thiothixin, triophaxanthin, valencene, vaucheriaxanthin, warmingone, xanthophyllomyces, zeaxanthin-β-diglucoside, α-cryptoxanthin, α-doradecin, β-isorenieratene, β-monadoxanthin, β-zeacarotene, γ-cryptoxanthin, δ-carotene, ε-carotene, and ζ-carotene-glucoside.

In some embodiments, the composition comprises ERG8, ERG10, ERG12, ERG13, and ERG19 expression levels in the modified yeast cell at a ratio of about 2.8 ERG8:about 1.0 ERG10:about 2.1 ERG12:about 1.3 ERG13:about 4.5 ERG19. In some embodiments, the ratio of ERG12:tHMG1:IDI1 expression levels in the yeast cell is about 2.1 ERG12:about 18 tHMG1:about 12 IDI1. In some embodiment, the level of expression is measured as qRT-PCR fold change of gene expression over wild-type as outline in the below examples.

In some embodiments, the composition comprises ERG8, ERG10, ERG12, ERG13, and ERG19 expression levels in the yeast cell at a ratio of about 2.6 ERG8:about 2.6 ERG10:about 2.0 ERG12:about 1.0 ERG13:about 3.4 ERG19. In some embodiments, the ratio of ERG12:tHMG1:IDI1 expression levels in the yeast cell is about 2.0 ERG12:about 18 tHMG1:about 12 IDI1. In some embodiments, the yeast cell comprises ERG8, ERG10, ERG12, ERG13, and ERG19 expression levels at any ratio outlined in the below examples when the promoter for each is independently selected from a strong-, medium-, or weak-strength promoter. In some embodiment, the level of expression is measured as qRT-PCR fold change of gene expression over wild-type as outline in the below examples.

In some embodiments, the first regulatory sequence is selected from pTDH3, pCCW12, pPGK1, pHHF2, pTEF1, pTEF2, pHHF1, pRPL18B, pHTB2, pALD6, pRNR1, pPAB1, pRET2, pSAC6, pPOP6, pRNR2, pPSP2, pRAD27, or pREV1. In some embodiments, the second regulatory sequence, the third regulatory sequence, the fourth regulatory sequence, and the fifth regulatory sequence are each independently selected from pTDH3, pCCW12, pPGK1, pHHF2, pTEF1, pTEF2, pHHF1, pRPL18B, pHTB2, pALD6, pRNR1, pPAB1, pRET2, or pSAC6, pRNR2, pPOP6, pRAD27, pPSP2, and pREV1. In some embodiments, the first regulatory sequence is selected from pTDH3, pCCW12, pPGK1, pHHF2, pTEF1, pTEF2, pHHF1, pRPL18B, pHTB2, pALD6, pRNR1, pPAB1, pRET2, or pSAC6, pPOP6, pRNR2, pPSP2, pRAD27, or pREV1, and the second regulatory sequence, the third regulatory sequence, the fourth regulatory sequence, and the fifth regulatory sequence are each independently selected from pTDH3, pCCW12, pPGK1, pHHF2, pTEF1, pTEF2, pHHF1, pRPL18B, pHTB2, pALD6, pRNR1, pPAB1, pRET2, or pSAC6, pRNR2, pPOP6, pRAD27, pPSP2, and pREV1.

In some embodiments, the disclosure relates to a yeast culture comprising one or more modified yeast cells herein. In some embodiments, the one or more modified yeast cells comprises one or more nucleic acid molecules, wherein the one or more nucleic acid molecules comprise the open reading frames disclosed herein, each nucleic acid molecule comprising a regulatory sequence operably linked to at least one of the open reading frames.

In some embodiments, the disclosure relates to a composition comprising a modified yeast comprising or consisting of the following genomic modifications: gal1Δ::pPGK1-ERG13-tPGK1, pTEF2-ERG12-tΔDH1, pHHF1-ERG19-tCYC1, LEU2; gal80Δ::pTEF1-ERG8-tSSA1, pCCW12-IDI1-tENO2, TRP1; rox1Δ::pHHF2-ERG10-SKL-tENO1, pTDH3-tHMG1-SKL-tTDH1, URA3; gal1Δ::pPGK1-ERG13-SKL-tPGK1, pTEF2-ERG12-SKL-tΔDH1, pHHF1-ERG19-SKL-tCYC1, LEU2; gal80Δ::pTEF2-ERG8-SKL-tSSA1, pCCW12-IDI1-SKL-tENO2, pTEF1-HygR-tTEF1, wherein each A represents a deletion, wherein each :: represents a genomic insertion which may be a deletion or replacement of the preceding deleted locus; wherein each lowercase “p” represents a promoter; wherein each lowercase “t” signifies a transcription terminator, and wherein each SKL represents a peroxisome localization signal. In some embodiments, the modifications do not comprise a modification of any of yeast genes: LPP1, DPP1, HO, ERG1, ANT1, IDP2, IDP3, Cit2, ACS1, ACL1, ACL2, Met15, RHR2, NADH-HMGR, ERG9, GPD1, and GPD2. In some embodiments, the genomic modifications consist of the modifications in this paragraph. In some embodiments, the disclosure relates to a yeast cell culture comprising the modified yeast of this paragraph.

The disclosure relates to a library cells, each cell comprising a modified yeast cell disclosed herein.

### Methods

In some embodiments, the disclosure relates to a method of culturing at least one modified yeast cell herein to produce a population of modified yeast cells. The at least one modified yeast cell, in some embodiments, is any one modified yeast cell described herein. The at least one modified yeast cell, in some embodiments, is a plurality of any two or more modified yeast cells described herein. The modified yeast cell(s) may be selected from any described herein. The modified yeast cell(s) may be selected from Example 9. In some embodiment, the method comprises inoculating a growth medium with a modified yeast cell herein. In some embodiments, the methods comprise a step of providing a culture vessel with at least one vessel into which culture medium is contained; and then a step of inoculating the culture medium with the one or more modified yeast cells disclosed herein. In some embodiments, the method further comprises incubating the inoculated growth medium. In some embodiments, the incubating comprises exposing the inoculated growth medium to a temperature suitable for growth of the modified yeast cell into the population of modified yeast cells. In some embodiments, the temperature is about 20° C. to about 35° C. In some embodiments, the temperature is about 30° C. In some embodiments, the incubating further comprises agitating the inoculated growth medium. In some embodiments, the agitation is shaking at about 150 to about 250 rpm. In some embodiments, the agitation is about 200 rpm. In some embodiments, the incubating comprises a time of about 8 to about 16 hours. In some embodiments, the time is about 12 hours. In some embodiments, the method further comprises inoculating another volume of growth medium with a portion of the population of modified yeast cells. In an embodiment, the population of modified yeast cells has an OD600 of about 0.1 when inoculating the another volume. In some embodiments, the method further comprises incubating the another volume of growth medium to obtain a second population of modified yeast cells. In some embodiment the conditions for incubating the another volume of growth medium are similar or the same as for the prior step of incubating. In some embodiments, the conditions for incubating the another volume of growth medium include batch culture, batch fermentation, or continuous fermentation. The growth medium may be any described herein or know to the skilled artisan. In some embodiments, the growth medium is synthetic-defined medium plus an antibiotic. In some embodiments, the growth medium is glucose medium or oleate medium.

In some embodiments, the disclosure relates to a method of making a terpene. In some embodiments, the method of making a terpene comprises steps of a method of culturing as described herein. In some embodiments, the method comprises inoculating a growth medium with a modified yeast cell, the modified yeast cell comprising open reading frames encoding ERG8, ERG10, ERG12, ERG13, and ERG19 and a first regulatory sequence of medium-strength or high-strength operably linked to the open reading frame encoding ERG12. The growth medium may be any described herein or known to the skilled artisan. In some embodiments, the growth medium is synthetic-defined medium plus an antibiotic. In some embodiments, the growth medium is glucose medium or oleate medium. In some embodiments, the method further comprises incubating the yeast cell in the growth medium. In some embodiments, the method further comprises isolating a plurality of yeast cells from the culture medium after the incubating the plurality of cells, disrupting the membrane of the yeast cells, and collecting the liquid phase after the step of disrupting. In some embodiments, the method further comprises drying the liquid phase. In some embodiments, the method further comprises creating the modified yeast cell. In some embodiments, creating the modified yeast cell comprises transforming a yeast cell with a nucleic acid or vector herein.

In some embodiments, the method comprises transforming a cell culture comprising modified yeast herein with at least one plasmid that encodes at least one selected terpene synthesis protein, such that the modified yeast produces the selected terpene synthesis protein and produces the selected terpene. In some embodiments, the at least one selected terpene synthesis protein optionally comprises a prenyltransferase, a terpene synthase, or a combination thereof. In some embodiments, the method further comprises isolating the selected terpene from the modified yeast. In some embodiments, the selected terpene is a mono-, sesqui-, or triterpene.

In some embodiments, the disclosure provides a method for making a product containing a terpene or terpene derivative. The methods according to this aspect comprise increasing terpene production in a cell that produces one or more terpenes by controlling the accumulation of metabolites or byproducts of known reactions producing the terpenes in the cell or in a culture of the cells. While some methods of isolating a terpene are generally known and disclosed in U.S. patent application Ser. No. 17/314,561, which is incorporated by reference in its entirety, methods of this disclosure relate to culturing one or more cells disclosed herein to the desired volume of culture medium, separating liquid and solid fractions from the culture, isolating the culture medium if the cell is secreting the terpene or isolating the solid fraction of cells if the terpene is contained within the modified yeast cell; and, if the terpene is contained within the cells, disrupting the cell membrane to release the cytoplasm containing the terpene; and collecting the solution fraction of the isolated cells to purify the terpene.

In some embodiments, the product is a food product, food additive, beverage, chewing gum, candy, or oral care product. In such embodiments, the terpene or derivative may be a flavor enhancer or sweetener. In some embodiments, the product is a food preservative.

In various embodiments, the product is a fragrance product, a cosmetic, a cleaning product, or a soap. In such embodiments, the terpene or derivative may be a fragrance.

In still other embodiments, the product is a vitamin or nutritional supplement.

In some embodiments, the product is a solvent, cleaning product, lubricant, or surfactant.

In some embodiments, the product is a pharmaceutical, and the terpene or derivative is an active pharmaceutical ingredient.

In some embodiments, the terpene or derivative is polymerized, and the resulting polymer may be elastomeric.

In some embodiments, the product is an insecticide, pesticide or pest control agent, and the terpene or derivative is an active ingredient. In some embodiments, the product is a cosmetic or personal care product, and the terpene or derivative is not a fragrance.

Downstream enzymes for the production of such terpenes and derivatives are known.

For example, the terpene may be alpha-sinensal, and which may be synthesized through a pathway comprising one or more of farnesyl diphosphate synthase (e.g., AAK63847.1) and valencene synthase (e.g., AF441124_1).

In other embodiments, the terpene is beta-Thujone, and which may be synthesized through a pathway comprising one or more of Geranyl pyrophosphate synthase (e.g., AAN01134.1, ACA21458.2) and (+)-sabinene synthase (e.g., AF051901.1).

In other embodiments, the terpene is Camphor, which may be synthesized through a pathway comprising one or more of Geranyl pyrophosphate synthase (e.g., AAN01134.1, ACA21458.2), (−)-borneol dehydrogenase (e.g., GU253890.1), and bornyl pyrophosphate synthase (e.g., AF051900).

In certain embodiments, the one or more terpenes include Carveol or Carvone, which may be synthesized through a pathway comprising one or more of Geranyl pyrophosphate synthase (e.g., AAN01134.1, ACA21458.2), 4S-limonene synthase (e.g., AAC37366.1), limonene-6-hydroxylase (e.g., AAQ18706.1, AAD44150.1), and carveol dehydrogenase (e.g., AAU20370.1, ABR15424.1).

In some embodiments, the one or more terpenes comprise Cineole, which may be synthesized through a pathway comprising one or more of Geranyl pyrophosphate synthase (e.g., AAN01134.1, ACA21458.2) and 1,8-cineole synthase (e.g., AF051899).

In some embodiments, the one or more terpenes includes Citral, which may be synthesized through a pathway comprising one or more of Geranyl pyrophosphate synthase (e.g., AAN01134.1, ACA21458.2), geraniol synthase (e.g., HM807399, GU136162, AY362553), and geraniol dehydrogenase (e.g., AY879284).

In still other embodiments, the one or more terpenes includes Cubebol, which is synthesized through a pathway comprising one or more of farnesyl diphosphate synthase (e.g., AAK63847.1), and cubebol synthase (e.g., CQ813505.1).

The one or more terpenes may include Limonene, and which may be synthesized through a pathway comprising one or more of Geranyl pyrophosphate synthase (e.g., AAN01134.1, ACA21458.2), and limonene synthase (e.g., EF426463, JN388566, HQ636425).

The one or more terpenes may include Menthone or Menthol, which may be synthesized through a pathway comprising one or more of Geranyl pyrophosphate synthase (e.g., AAN01134.1, ACA21458.2), limonene synthase (e.g., EF426463, JN388566, HQ636425), (−)-limonene-3-hydroxylase (e.g., EF426464, AY622319), (−)-isopiperitenol dehydrogenase (e.g., EF426465), (−)-isopiperitenone reductase (e.g., EF426466), (+)-cis-isopulegone isomerase, (−)-menthone reductase (e.g., EF426467), and for Menthol (−)-menthol reductase (e.g., EF426468).

In some embodiments, the one or more terpenes comprise myrcene, which may be synthesized through a pathway comprising one or more of Geranyl pyrophosphate synthase (e.g., AAN01134.1, ACA21458.2) and myrcene synthase (e.g., U87908, AY195608, AF271259).

The one or more terpenes may include Nootkatone, which may be synthesized through a pathway comprising one or more of farnesyl diphosphate synthase (e.g., AAK63847.1), and Valancene synthase (e.g., CQ813508, AF441124_1).

The one or more terpenes may include Sabinene hydrate, which may be synthesized through a pathway comprising one or more of Geranyl pyrophosphate synthase (e.g., AAN01134.1, ACA21458.2), and sabinene synthase (e.g., 081193.1).

The one or more terpenes may include Steviol or steviol glycoside, and which may be synthesized through a pathway comprising one or more of geranylgeranylpyrophosphate synthase (e.g., AF081514), ent-copalyl diphosphate synthase (e.g., AF034545.1), ent-kaurene synthase (e.g., AF097311.1), ent-kaurene oxidase (e.g., DQ200952.1), and kaurenoic acid 13-hydroxylase (e.g., EU722415.1). For steviol glycoside, the pathway may further include UDP-glycosyltransferases (UGTs) (e.g., AF515727.1, AY345983.1, AY345982.1, AY345979.1, AAN40684.1, ACE87855.1).

The one or more terpenes may include Thymol, which may be synthesized through a pathway comprising one or more of Geranyl pyrophosphate synthase (e.g., AAN01134.1, ACA21458.2), limonene synthase (e.g., EF426463, JN388566, HQ636425), (−)-limonene-3-hydroxylase (e.g., EF426464, AY622319), (−)-isopiperitenol dehydrogenase (e.g., EF426465), and (−)-isopiperitenone reductase (e.g., EF426466).

The one or more terpenes may include Valencene, which may be synthesized through a pathway comprising one or more of farnesyl diphosphate synthase (e.g., AAK63847.1), and Valancene synthase (e.g., CQ813508, AF441124_1).

In some embodiments, the one or more terpenes includes one or more of alpha, beta and γ-humulene, which may be synthesized through a pathway comprising one or more of farnesyl diphosphate synthase (e.g., AAK63847.1), and humulene synthase (e.g., U92267.1).

In some embodiments, the one or more terpenes includes (+)-borneol, which may be synthesized through a pathway comprising one or more of Geranyl pyrophosphate synthase (e.g., AAN01134.1, ACA21458.2), and bornyl pyrophosphate synthase (e.g., AF051900).

The one or more terpenes may comprise 3-carene, which may be synthesized through a pathway comprising one or more of Geranyl pyrophosphate synthase (e.g., AAN01134.1, ACA21458.2), and 3-carene synthase (e.g., HQ336800).

In some embodiments, the one or more terpenes include 3-Oxo-alpha-Ionone or 4-oxo-beta-ionone, which may be synthesized through a pathway comprising carotenoid cleavage dioxygenase (e.g., ABY60886.1, BAJ05401.1).

In some embodiments, the one or more terpenes include alpha-terpinolene, which may be synthesized through a pathway comprising one or more of Geranyl pyrophosphate synthase (e.g., AAN01134.1, ACA21458.2), and alpha-terpineol synthase (e.g., AF543529).

In some embodiments, the one or more terpenes include alpha-thujene, which may be synthesized through a pathway comprising one or more of Geranyl pyrophosphate synthase (e.g., AAN01134.1, ACA21458.2), and alpha-thujene synthase (e.g., AEJ91555.1).

In some embodiments, the one or more terpenes include Farnesol, which may be synthesized through a pathway comprising one or more of farnesyl diphosphate synthase (e.g., AAK63847.1), and Farnesol synthase (e.g., AF529266.1, DQ872159.1).

In some embodiments, the one or more terpenes include Fenchone, which may be synthesized through a pathway comprising one or more of Geranyl pyrophosphate synthase (e.g., AAN01134.1, ACA21458.2), and (−)-endo-fenchol cyclase (e.g., AY693648).

In some embodiments, the one or more terpenes include gamma-Terpinene, which may be synthesized through a pathway comprising one or more of Geranyl pyrophosphate synthase (e.g., AAN01134.1, ACA21458.2), and terpinene synthase (e.g., AB110639).

In some embodiments, the one or more terpenes include Geraniol, which may be synthesized through a pathway comprising one or more of Geranyl pyrophosphate synthase (e.g., AAN01134.1, ACA21458.2), and geraniol synthase (e.g. HM807399, GU136162, AY362553).

In still other embodiments, the one or more terpenes include ocimene, which may be synthesized through a pathway comprising one or more of Geranyl pyrophosphate synthase (e.g., AAN01134.1, ACA21458.2), and beta-ocimene synthase (e.g., EU194553.1).

In certain embodiments, the one or more terpenes include Pulegone, which may be synthesized through a pathway comprising one or more of Geranyl pyrophosphate synthase (e.g., AAN01134.1, ACA21458.2), and pinene synthase (e.g., HQ636424, AF543527, U87909).

In certain embodiments, the one or more terpenes includes Sabinene, which may be synthesized through a pathway comprising one or more of Geranyl pyrophosphate synthase (e.g., AAN01134.1, ACA21458.2), and sabinene synthase (e.g., HQ336804, AF051901, DQ785794).

### Kits

In some embodiments, the disclosure relates to a kit comprising at least one nucleic acid molecule. In some embodiments, the at least one nucleic acid molecule is selected from any nucleic acid molecule herein. In some embodiments, the at least one nucleic acid molecule comprises a nucleic acid molecule comprising a nucleic acid sequence comprising an open reading frame encoding ERG12 and a first regulatory sequence of weak-strength, medium-strength or high-strength operably linked to the open reading frame encoding ERG12. In some embodiments, the kit comprising one or more plasmids that encode one or more terpene synthesis proteins. In some embodiments, the kit further comprises a yeast cell. In some embodiments, the kit further comprises a growth medium. The growth medium may be any known to the skilled artisan. In some embodiments, the growth medium is synthetic-defined medium plus an antibiotic. In some embodiments, the growth medium is glucose medium or oleate medium. In some embodiments, the growth medium is dried. In some embodiments, the kit further comprises instructions for transforming the yeast cell with the at least one nucleic acid molecule to create a modified yeast cell. In some embodiments, the kit further comprises instructions for producing a terpene from the modified yeast cell.

In some embodiments, the disclosure relates to a kit comprising at least one modified yeast cell. In some embodiments, the kit further comprises a growth medium. In some embodiments, the growth medium is glucose medium or oleate medium. In some embodiments, the growth medium is dried. In some embodiments, the kit further comprises instructions for producing a terpene or terpenes from the at least one modified yeast cell.

All citations and references used in the aforementioned sections and Examples, including patent applications and journal articles are incorporated herein by reference in their entireties.

TABLE of Yeast Nucleic Acid Sequences Referenced Above.

| | |
|---|---|
| LPP1 | ATGATCTCTGTCATGGCGGATGAGAAACATAAGGAGTATTTTAAGCTATACTACTTTCAGTACATGATAATTGGTC |
| | TATGTACGATATTATTCCTCTATTCGGAGATATCCCTGGTACCTAGGGGCCAAAACATCGAATTTAGTCTTGATGA |
| | CCCCAGTATATCAAAACGTTATGTACCTAACGAACTCGTGGGCCCACTAGAATGTTTGATTTTGAGTGTTGGACTG |
| | AGTAACATGGTCGTCTTCTGGACCTGCATGTTTGACAAGGACTTACTGAAGAAGAATAGAGTAAAGAGACTAAGA |
| | GAGAGGCCGGACGGAATCTCGAACGATTTTCACTTCATGCATACTAGCATTCTATGTCTGATGCTGATTATAAGCA |
| | TAAATGCTGCCCTAACAGGCGCCTTAAAGTTGATTATAGGAAACTTGAGGCCTGACTTTGTTGATAGATGTATACC |
| | TGACCTCCAAAAGATGAGTGATTCAGATTCTTTGGTTTTTGGCTTGGACATTTGCAAGCAGACTAACAAATGGATT |
| | CTATACGAAGGCTTAAAAAGCACTCCAAGCGGACATTCAAGTTTCATAGTCAGTACCATGGGCTTTACATATCTTT |
| | GGCAAAGGGTTTTCACCACACGCAATACAAGAAGTTGCATTTGGTGCCCTTTATTAGCTCTAGTAGTAATGGTTTC |
| | AAGGGTTATCGATCACAGACATCATTGGTACGATGTTGTCTCTGGAGCTGTTCTAGCATTTTTAGTCATTTATTGTT |
| | GCTGGAAATGGACATTTACAAACTTGGCGAAAAGAGACATACTTCCTTCACCGGTTAGTGTTTAG |
| DPP1 | ATGAACAGAGTTTCGTTTATTAAAACGCCTTTCAACATAGGGGCGAAATGGAGATTAGAAGATGTCTTTTTGCTCA |
| | TTATCATGATACTTCTTAACTACCCAGTGTATTACCAACAACCGTTCGAACGTCAGTTTTACATTAACGATCTCACT |
| | ATATCGCATCCTTATGCGACAACTGAACGTGTAAATAACAACATGTTGTTTGTTTATAGTTTTGTCGTGCCATCTTT |
| | AACCATATTGATAATTGGTTCCATTTTGGCCGATAGAAGACATTTGATTTTTATTTTGTACACATCTCTCCTTGGTT |
| | TATCACTCGCTTGGTTCAGTACGAGTTTCTTTACAAACTTCATCAAGAATTGGATTGGAAGACTAAGACCAGATTT |
| | TCTAGATCGTTGCCAACCTGTTGAAGGCTTGCCATTGGACACTTTATTTACTGCAAAAGATGTGTGTACGACTAAG |
| | AATCACGAACGTCTGTTGGATGGGTTTAGGACAACTCCGTCAGGTCATTCAAGTGAAAGCTTTGCAGGACTGGGT |
| | TATTTGTACTTCTGGCTATGTGGGCAACTTTTGACTGAATCACCGTTGATGCCTTTATGGAGAAAAATGGTGGCCT |
| | TTCTACCACTGTTAGGAGCTGCACTAATTGCTCTATCCAGAACTCAAGATTACAGACATCATTTCGTCGATGTAAT |
| | TTTAGGGTCTATGTTGGGTTATATAATGGCACACTTTTTCTACAGAAGAATCTTCCCACCCATTGATGATCCTCTTC |
| | CGTTCAAACCATTGATGGACGATTCAGATGTCACCCTGGAGGAAGCAGTCACCCATCAGAGGATCCCGGATGAGG |
| | AATTACATCCTTTGTCCGATGAAGGTATGTAA |
| HO | ATGCTTTCTGAAAACACGACTATTCTGATGGCTAACGGTGAAATTAAAGACATCGCAAACGTCACGGCTAACTCTT |
| | ACGTTATGTGCGCAGATGGCTCCGCTGCCCGCGTCATAAATGTCACACAGGGCTATCAGAAAATCTATAATATAC |
| | AGCAAAAAACCAAACACAGAGCTTTTGAAGGTGAACCTGGTAGGTTAGATCCCAGGCGTAGAACAGTTTATCAGC |
| | GTCTTGCATTACAATGTACTGCAGGTCATAAATTGTCAGTCAGGGTCCCTACCAAACCACTGTTGGAAAAAAGTG |
| | GTAGAAATGCCACCAAATATAAAGTGAGATGGAGAAATCTGCAGCAATGTCAGACGCTTGATGGTAGGATAATA |
| | ATAATTCCAAAAAACCATCATAAGACATTCCCAATGACAGTTGAAGGTGAGTTTGCCGCAAAACGCTTCATAGAA |
| | GAAATGGAGCGCTCTAAAGGAGAATATTTCAACTTTGACATTGAAGTTAGAGATTTGGATTATCTTGATGCTCAAT |
| | TGAGAATTTCTAGCTGCATAAGATTTGGTCCAGTACTCGCAGGAAATGGTGTTTTATCTAAATTTCTCACTGGACG |
| | TAGTGACCTTGTAACTCCTGCTGTAAAAAGTATGGCTTGGATGCTTGGTCTGTGGTTAGGTGACAGTACAACAAAA |
| | GAGCCAGAAATCTCAGTAGATAGCTTGGATCCTAAGCTAATGGAGAGTTTAAGAGAAAATGCGAAAATCTGGGGT |
| | CTCTACCTTACGGTTTGTGACGATCACGTTCCGCTACGTGCCAAACATGTAAGGCTTCATTATGGAGATGGTCCAG |
| | GGGATCTTGATGGAGAGAAGCAAATCCCTGAATTTATGTACGGCGAGCATATAGAAGTTCGTGAAGCATTCTTAG |
| | ATGAAAACAGGAAGACAAGGAATTTGAGGAAAAATAATCCATTCTGGAAAGCTGTCACAATTTTAAAGTTTAAAA |
| | CCGGCTTGATCGACTCAGATGGGTACGTTGTGAAAAAGGGCGAAGGCCCTGAATCTTATAAAATAGCAATTCAAA |
| | CTGTTTATTCATCCATTATGGACGGAATTGTCCATATTTCAAGATCTCTTGGTATGTCAGCTACTGTGACGACCAGG |
| | TCAGCTAGGGAGGAAATCATTGAAGGAAGAAAAGTCCAATGTCAATTTACATACGACTGTAATGTTGCTGGGGGA |
| | ACAACTTCACAGAATGTTTTGTCATATTGTCGAAGTGGTCACAAAACAAGAGAAGTTCCGCCAATTATAAAAAGG |
| | GAACCCGTATATTTCAGCTTCACGGATGATTTCCAGGGTGAGAGTACTGTATATGGGCTTACGATAGAAGGCCAT |
| | AAAAATTTCTTGCTTGGCAACAAAATAGAAGTGAAATCATGTCGAGGCTGCTGTGTGGGAGAACAGCTTAAAATA |
| | TCACAAAAAAAGAATCTAAAACACTGTGTTGCTTGTCCCAGAAAGGGAATCAAGTATTTTTATAAAGATTGGAGT |
| | GGTAAAAATCGAGTATGTGCTAGATGCTATGGAAGATACAAATTCAGCGGTCATCACTGTATAAATTGCAAGTAT |
| | GTACCAGAAGCACGTGAAGTGAAAAAGGCAAAAGACAAAGGCGAAAAATTGGGCATTACGCCCGAAGGTTTGCC |
| | AGTTAAAGGACCAGAGTGTATAAAATGTGGCGGAATCTTACAGTTTGATGCTGTCCGCGGGCCTCATAAGAGTTG |
| | TGGTAACAACGCAGGTGCGCGCATCTGCTAA |
| ERG1 | ATGTCTGCTGTTAACGTTGCACCTGAATTGATTAATGCCGACAACACAATTACCTACGATGCGATTGTCATCGGTG |
| | CTGGTGTTATCGGTCCATGTGTTGCTACTGGTCTAGCAAGAAAGGGTAAGAAAGTTCTTATCGTAGAACGTGACTG |
| | GGCTATGCCTGATAGAATTGTTGGTGAATTGATGCAACCAGGTGGTGTTAGAGCATTGAGAAGTCTGGGTATGAT |
| | TCAATCTATCAACAACATCGAAGCATATCCTGTTACCGGTTATACCGTCTTTTTCAACGGCGAACAAGTTGATATT |
| | CCATACCCTTACAAGGCCGATATCCCTAAAGTTGAAAAATTGAAGGACTTGGTCAAAGATGGTAATGACAAGGTC |
| | TTGGAAGACAGCACTATTCACATCAAGGATTACGAAGATGATGAAAGAGAAAGGGGTGTTGCTTTTGTTCATGGT |
| | AGATTCTTGAACAACTTGAGAAACATTACTGCTCAAGAGCCAAATGTTACTAGAGTGCAAGGTAACTGTATTGAG |
| | ATATTGAAGGATGAAAAGAATGAGGTTGTTGGTGCCAAGGTTGACATTGATGGCCGTGGCAAGGTGGAATTCAAA |
| | GCCCACTTGACATTTATCTGTGACGGTATCTTTTCACGTTTCAGAAAGGAATTGCACCCAGACCATGTTCCAACTG |
| | TCGGTTCTTCGTTTGTCGGTATGTCTTTGTTCAATGCTAAGAATCCTGCTCCTATGCACGGTCACGTTATTCTTGGT |
| | AGTGATCATATGCCAATCTTGGTTTACCAAATCAGTCCAGAAGAAACAAGAATCCTTTGTGCTTACAACTCTCCAA |
| | AGGTCCCAGCTGATATCAAGAGTTGGATGATTAAGGATGTCCAACCTTTCATTCCAAAGAGTCTACGTCCTTCATT |
| | TGATGAAGCCGTCAGCCAAGGTAAATTTAGAGCTATGCCAAACTCCTACTTGCCAGCTAGACAAAACGACGTCAC |
| | TGGTATGTGTGTTATCGGTGACGCTCTAAATATGAGACATCCATTGACTGGTGGTGGTATGACTGTCGGTTTGCAT |
| | GATGTTGTCTTGTTGATTAAGAAAATAGGTGACCTAGACTTCAGCGACCGTGAAAAGGTTTTGGATGAATTACTAG |
| | ACTACCATTTCGAAAGAAAGAGTTACGATTCCGTTATTAACGTTTTGTCAGTGGCTTTGTATTCTTTGTTCGCTGCT |
| | GACAGCGATAACTTGAAGGCATTACAAAAAGGTTGTTTCAAATATTTCCAAAGAGGTGGCGATTGTGTCAACAAA |
| | CCCGTTGAATTTCTGTCTGGTGTCTTGCCAAAGCCTTTGCAATTGACCAGGGTTTTCTTCGCTGTCGCTTTTTACAC |
| | CATTTACTTGAACATGGAAGAACGTGGTTTCTTGGGATTACCAATGGCTTTATTGGAAGGTATTATGATTTTGATC |
| | ACAGCTATTAGAGTATTCACCCCATTTTTGTTTGGTGAGTTGATTGGTTAA |
| ANT1 | ATGTTAACTCTAGAGTCTGCATTAACTGGCGCTGTGGCTTCGGCAATGGCCAATATTGCAGTTTATCCGCTGGATT |
| | TATCGAAGACGATCATTCAGTCACAAGTATCTCCTTCTTCAAGTGAGGATAGTAACGAAGGTAAAGTTTTGCCCAA |
| | TAGGAGATATAAGAATGTTGTAGATTGCATGATAAACATATTCAAAGAAAAGGGTATTTTGGGTCTGTATCAAGG |
| | TATGACAGTCACTACGGTGGCCACATTTGTCCAGAATTTTGTTTATTTCTTTTGGTACACATTTATCAGAAAGTCCT |
| | ACATGAAACATAAGCTGTTAGGACTGCAATCACTGAAAAACCGCGATGGTCCTATCACACCTTCTACGATTGAAG |
| | CATAACTGCTTTTTGGAAAGGTTTAAGAACAGGTTTAGCATTGACGATAAATCCTTCCATCACATATGCCTCTTTTC |
| | AATTGGTACTTGGGGTAGCAGCTGCCAGTATATCGCAACTTTTTACTAGTCCCATGGCTGTGGTAGCTACAAGACA |
| | ACAAACAGTCCATTCTGCAGAGTCTGCCAAATTTACCAACGTTATTAAGGACATTTACCGTGAAAATAATGGGGA |
| | AAAGACTTAAAGAAGTTTTTTTCCATGACCATTCCAACGATGCTGGCAGTTTGTCAGCAGTGCAAAATTTCATTTT |
| | GGGTGTCCTTTCCAAGATGATTTCGACTCTAGTTACGCAACCCTTGATTGTCGCTAAAGCAATGCTTCAAAGCGCT |
| | GGCTCTAAATTCACTACTTTCCAAGAAGCGCTACTATACTTGTACAAAAATGAAGGGTTAAAATCTCTTTGGAAGG |
| | GAGTTCTTCCTCAATTGACAAAGGGTGTCATTGTGCAAGGTCTGTTGTTTGCTTTCAGAGGAGAATTGACAAAATC |
| | TTTAAAGAGGCTAATATTCTTGTACTCTTCTTTTTTCCTAAAGCACAACGGACAACGCAAGCTGGCTTCCACTTGA |
| IDP2 | ATGACAAAGATTAAGGTAGCTAACCCCATTGTGGAAATGGACGGCGATGAGCAAACAAGAATAATCTGGCATTTA |
| | ATCAGGGACAAGTTAGTCTTGCCCTATCTTGACGTTGATTTGAAGTACTACGATCTTTCCGTGGAGTATCGTGACC |
| | AGACTAATGATCAAGTAACTGTGGATTCTGCCACCGCGACTTTAAAGTATGGAGTAGCTGTCAAATGCGCGACTA |
| | TTACACCCGATGAGGCAAGGGTCGAGGAATTTCATTTGAAAAAGATGTGGAAATCTCCAAATGGTACTATTAGAA |
| | ACATTTTGGGTGGTACAGTGTTCAGAGAACCTATTATTATCCCTAGAATTCCAAGGCTAGTTCCTCAATGGGAGAA |
| | GCCCATCATCATTGGGAGACACGCATTCGGCGATCAGTACAAAGCTACCGATGTAATAGTCCCTGAAGAAGGCGA |
| | GTTGAGGCTTGTTTATAAATCCAAGAGCGGAACTCATGATGTAGATCTGAAGGTATTTGACTACCCAGAACATGG |
| | TGGGGTTGCCATGATGATGTACAACACTACAGATTCGATCGAAGGGTTTGCGAAGGCCTCCTTTGAATTGGCCATT |
| | GAAAGGAAGTTACCATTATATTCCACTACTAAGAATACTATTTTGAAGAAGTATGATGGTAAATTCAAAGATGTTT |
| | TCGAAGCCATGTATGCTAGAAGTTATAAAGAGAAGTTTGAATCCCTTGGCATCTGGTACGAGCACCGTTTAATTGA |
| | TGATATGGTGGCCCAAATGTTGAAATCTAAAGGTGGATACATAATTGCCATGAAAAATTACGACGGTGACGTAGA |
| | ATCAGATATTGTTGCACAAGGATTTGGCTCCTTGGGGTTAATGACATCTGTGTTGATTACCCCGGACGGTAAAACC |
| | TTTGAAAGCGAAGCCGCCCACGGTACAGTAACAAGACATTTTAGACAGCATCAGCAAGGAAAGGAGACGTCAAC |
| | AAATTCCATTGCATCAATTTTCGCGTGGACTAGAGGTATTATTCAAAGGGGTAAACTTGATAATACTCCAGATGTA |
| | GTTAAGTTCGGCCAAATATTGGAAAGCGCTACGGTAAATACAGTGCAAGAAGATGGAATCATGACTAAAGATTTG |
| | GCGCTCATTCTCGGTAAGTCTGAAAGATCCGCTTATGTCACTACCGAGGAGTTCATTGACGCGGTGGAATCTAGAT |
| | TGAAAAAAGAGTTCGAGGCAGCTGCATTGTAA |
| IDP3 | ATGAGTAAAATTAAAGTTGTTCATCCCATCGTGGAAATGGACGGTGATGAGCAGACAAGAGTTATTTGGAAACTT |
| | ATCAAAGAAAAATTGATATTGCCATATTTAGATGTGGATTTAAAATACTATGACCTTTCAATCCAAGAGCGTGATA |
| | GGACTAATGATCAAGTAACAAAGGATTCTTCTTATGCTACCCTAAAATATGGGGTTGCTGTCAAATGTGCCACTAT |
| | AACACCCGATGAGGCAAGAATGAAAGAATTTAACCTTAAAGAAATGTGGAAATCTCCAAATGGAACAATCAGAA |
| | ACATCCTAGGTGGAACTGTATTTAGAGAACCCATCATTATTCCAAAAATACCTCGTCTAGTCCCTCACTGGGAGAA |
| | ACCTATAATTATAGGCCGTCATGCTTTTGGTGACCAATATAGGGCTACTGACATCAAGATTAAAAAAGCAGGCAA |
| | ACTAAGGTTACAGTTTAGCTCAGATGACGGTAAAGAAAACATCGATTTAAAGGTTTATGAATTTCCTAAAAGTGG |
| | TGGGATCGCAATGGCAATGTTTAATACAAATGATTCCATTAAAGGGTTCGCAAAGGCATCCTTCGAATTAGCTCTC |
| | AAAAGAAAACTACCGTTATTCTTTACAACCAAAAACACTATTCTGAAAAATTATGATAATCAGTTCAAACAAATTT |
| | TCGATAATTTGTTCGATAAAGAATATAAGGAAAAGTTTCAGGCTTTAAAAATAACGTACGAGCATCGTTTGATTG |
| | ATGATATGGTAGCACAGATGCTAAAATCAAAGGGCGGGTTTATAATCGCCATGAAGAATTATGATGGCGATGTCC |
| | AGTCTGACATTGTGGCACAAGGATTTGGGTCTCTTGGTTTAATGACGTCCATATTGATTACACCTGATGGTAAAAC |
| | GTTTGAAAGCGAGGCTGCCCATGGTACGGTGACCAGACATTTTAGAAAACATCAAAGAGGCGAAGAAACATCAA |
| | CAAATTCAATAGCCTCAATATTTGCCTGGACAAGGGCAATTATACAAAGAGGAAAATTAGACAATACAGATGATG |
| | TTATAAAATTTGGAAACTTACTAGAAAAGGCTACTTTGGACACAGTTCAAGTGGGCGGAAAAATGACCAAGGATT |
| | TAGCATTGATGCTTGGAAAGACTAATAGATCATCATATGTAACCACAGAAGAGTTTATTGATGAAGTTGCCAAGA |
| | GGCTTCAAAACATGATGCTCAGCTCCAATGAAGACAAGAAAGGTATGTGCAAACTATAA |
| CIT2 | ATGACAGTTCCTTATCTAAATTCAAACAGAAATGTTGCATCATATTTACAATCAAATTCAAGCCAAGAAAAGACTC |
| | TAAAAGAGAGATTTAGCGAAATCTACCCCATCCATGCTCAAGATGTAAGGCAATTCGTTAAAGAGCATGGCAAAA |
| | CTAAAATTAGCGATGTTCTATTAGAACAGGTATATGGTGGTATGAGAGGTATTCCAGGGAGCGTATGGGAAGGTT |
| | CCGTTTTGGACCCAGAAGACGGTATTCGTTTCAGAGGTCGTACGATCGCCGACATTCAAAAGGACCTGCCCAAGG |
| | CAAAAGGAAGCTCACAACCACTACCAGAAGCTCTCTTTTGGTTATTGCTAACTGGCGAGGTTCCAACTCAAGCGC |
| | AAGTTGAAAACTTATCAGCTGATCTAATGTCAAGATCGGAACTACCTAGTCATGTCGTTCAACTTTTGGATAATTT |
| | ACCAAAGGACTTACACCCAATGGCTCAATTCTCTATTGCTGTAACTGCCTTGGAAAGCGAGTCAAAGTTTGCTAAG |
| | GCTTATGCTCAAGGAATTTCCAAGCAAGATTATTGGAGTTATACTTTTGAAGATTCACTAGACTTGCTGGGTAAAT |
| | ATTATGCTAAAAATCTGGTCAACTTGATTGGTTCTAAGGATGAAGATTTCGTGGACTTGATGAGACTTTATTTAAC |
| | CATTCATTCGGATCACGAAGGTGGTAATGTATCTGCACATACATCCCATCTTGTGGGCTCAGCACTATCATCACCT |
| | TGCCAGTTATTGCAGCTAAAATTTATCGTAATGTATTCAAAGATGGCAAAATGGGTGAAGTGGACCCAAATGCCG |
| | TATCTGTCCCTTGCATCAGGTTTGAACGGGTTGGCTGGCCCACTTCATGGGCGTGCTAATCAAGAAGTACTAGAAT |
| | GGTTATTTGCACTTAAAGAAGAGGTAAATGATGACTACTCTAAAGATACGATCGAAAAATATTTATGGGATACTC |
| | TAAACTCAGGAAGAGTCATTCCCGGTTATGGTCATGCTGTGCTAAGGAAAACTGATCCTCGTTATATGGCTCAGCG |
| | TAAGTTTGCCATGGACCATTTTCCAGATTATGAATTATTCAAGTTAGTTTCATCAATATACGAGGTAGCACCTGGC |
| | GTATTGACTGAACATGGTAAAACCAAAAATCCATGGCCAAATGTAGATGCTCACTCTGGTGTCTTATTACAATATT |
| | ATGGACTAAAAGAATCTTCTTTCTATACCGTTTTATTTGGCGTTTCAAGGGCATTTGGTATTCTTGCTCAATTGATC |
| | ACTGATAGGGCCATCGGTGCTTCCATTGAAAGGCCAAAGTCCTATTCTACTGAGAAATACAAGGAATTGGTCAAA |
| | AACATTGAAAGCAAACTATAG |
| ACL1 | ATGTCAGCGAAATCCATTCACGAGGCCGACGGCAAGGCCCTGCTCGCACACTTTCTGTCCAAGGCGCCCGTGTGG |
| | GCCGAGCAGCAGCCCATCAACACGTTTGAAATGGGCACACCCAAGCTGGCGTCTCTGACGTTCGAGGACGGCGTG |
| | GCCCCCGAGCAGATCTTCGCCGCCGCTGAAAAGACCTACCCCTGGCTGCTGGAGTCCGGCGCCAAGTTTGTGGCC |
| | AAGCCCGACCAGCTCATCAAGCGACGAGGCAAGGCCGGCCTGCTGGTACTCAACAAGTCGTGGGAGGAGTGCAA |
| | GCCCTGGATCGCCGAGCGGGCCGCCAAGCCCATCAACGTGGAGGGCATTGACGGAGTGCTGCGAACGTTCCTGGT |
| | CGAGCCCTTTGTGCCCCACGACCAGAAGCACGAGTACTACATCAACATCCACTCCGTGCGAGAGGGCGACTGGAT |
| | CCTCTTCTACCACGAGGGAGGAGTCGACGTCGGCGACGTGGACGCCAAGGCCGCCAAGATCCTCATCCCCGTTGA |
| | CATTGAGAACGAGTACCCCTCCAACGCCACGCTCACCAAGGAGCTGCTGGCACACGTGCCCGAGGACCAGCACCA |
| | GACCCTGCTCGACTTCATCAACCGGCTCTACGCCGTCTACGTCGATCTGCAGTTTACGTATCTGGAGATCAACCCC |
| | CTGGTCGTGATCCCCACCGCCCAGGGCGTCGAGGTCCACTACCTGGATCTTGCCGGCAAGCTCGACCAGACCGCA |
| | GAGTTTGAGTGCGGCCCCAAGTGGGCTGCTGCGCGGTCCCCCGCCGCTCTGGGCCAGGTCGTCACCATTGACGCC |
| | GGCTCCACCAAGGTGTCCATCGACGCCGGCCCCGCCATGGTCTTCCCCGCTCCTTTCGGTCGAGAGCTGTCCAAGG |
| | AGGAGGCGTACATTGCGGAGCTCGATTCCAAGACCGGAGCTTCTCTGAAGCTGACTGTTCTCAATGCCAAGGGCC |
| | GAATCTGGACCCTTGTGGCTGGTGGAGGAGCCTCCGTCGTCTACGCCGACGCCATTGCGTCTGCCGGCTTTGCTGA |
| | CGAGCTCGCCAACTACGGCGAGTACTCTGGCGCTCCCAACGAGACCCAGACCTACGAGTACGCCAAAACCGTACT |
| | GGATCTCATGACCCGGGGCGACGCTCACCCCGAGGGCAAGGTACTGTTCATTGGCGGAGGAATCGCCAACTTCAC |
| | CCAGGTTGGATCCACCTTCAAGGGCATCATCCGGGCCTTCCGGGACTACCAGTCTTCTCTGCACAACCACAAGGTG |
| | AAGATTTACGTGCGACGAGGCGGTCCCAACTGGCAGGAGGGTCTGCGGTTGATCAAGTCGGCTGGCGACGAGCTG |
| | AATCTGCCCATGGAGATTTACGGCCCCGACATGCACGTGTCGGGTATTGTTCCTTTGGCTCTGCTTGGAAAGCGGC |
| | CCAAGAATGTCAAGCCTTTTGGCACCGGACCTTCTACTGAGGCTTCCACTCCTCTCGGAGTTTAA |
| ACL2 | ATGTCTGCCAACGAGAACATCTCCCGATTCGACGCCCCTGTGGGCAAGGAGCACCCCGCCTACGAGCTCTTCCAT |
| | AACCACACACGATCTTTCGTCTATGGTCTCCAGCCTCGAGCCTGCCAGGGTATGCTGGACTTCGACTTCATCTGTA |
| | AGCGAGAGAACCCCTCCGTGGCCGGTGTCATCTATCCCTTCGGCGGCCAGTTCGTCACCAAGATGTACTGGGGCA |
| | CCAAGGAGACTCTTCTCCCTGTCTACCAGCAGGTCGAGAAGGCCGCTGCCAAGCACCCCGAGGTCGATGTCGTGG |
| | TCAACTTTGCCTCCTCTCGATCCGTCTACTCCTCTACCATGGAGCTGCTCGAGTACCCCCAGTTCCGAACCATCGCC |
| | ATTATTGCCGAGGGTGTCCCCGAGCGACGAGCCCGAGAGATCCTCCACAAGGCCCAGAAGAAGGGTGTGACCATC |
| | ATTGGTCCCGCTACCGTCGGAGGTATCAAGCCCGGTTGCTTCAAGGTTGGAAACACCGGAGGTATGATGGACAAC |
| | ATTGTCGCCTCCAAGCTCTACCGACCCGGCTCCGTTGCCTACGTCTCCAAGTCCGGAGGAATGTCCAACGAGCTGA |
| | ACAACATTATCTCTCACACCACCGACGGTGTCTACGAGGGTATTGCTATTGGTGGTGACCGATACCCTGGTACTAC |
| | CTTCATTGACCATATCCTGCGATACGAGGCCGACCCCAAGTGTAAGATCATCGTCCTCCTTGGTGAGGTTGGTGGT |
| | GTTGAGGAGTACCGAGTCATCGAGGCTGTTAAGAACGGCCAGATCAAGAAGCCCATCGTCGCTTGGGCCATTGGT |
| | ACTTGTGCCTCCATGTTCAAGACTGAGGTTCAGTTCGGCCACGCCGGCTCCATGGCCAACTCCGACCTGGAGACTG |
| | CCAAGGCTAAGAACGCCGCCATGAAGTCTGCTGGCTTCTACGTCCCCGATACCTTCGAGGACATGCCCGAGGTCC |
| | TTGCCGAGCTCTACGAGAAGATGGTCGCCAAGGGCGAGCTGTCTCGAATCTCTGAGCCTGAGGTCCCCAAGATCC |
| | CCATTGACTACTCTTGGGCCCAGGAGCTTGGTCTTATCCGAAAGCCCGCTGCTTTCATCTCCACTATTTCCGATGAC |
| | CGAGGCCAGGAGCTTCTGTACGCTGGCATGCCCATTTCCGAGGTTTTCAAGGAGGACATTGGTATCGGCGGTGTC |
| | ATGTCTCTGCTGTGGTTCCGACGACGACTCCCCGACTACGCCTCCAAGTTTCTTGAGATGGTTCTCATGCTTACTGC |
| | TGACCACGGTCCCGCCGTATCCGGTGCCATGAACACCATTATCACCACCCGAGCTGGTAAGGATCTCATTTCTTCC |
| | CTGGTTGCTGGTCTCCTGACCATTGGTACCCGATTCGGAGGTGCTCTTGACGGTGCTGCCACCGAGTTCACCACTG |
| | CCTACGACAAGGGTCTGTCCCCCCGACAGTTCGTTGATACCATGCGAAAGCAGAACAAGCTGATTCCTGGTATTG |
| | GCCATCGAGTCAAGTCTCGAAACAACCCCGATTTCCGAGTCGAGCTTGTCAAGGACTTTGTTAAGAAGAACTTCCC |
| | CTCCACCCAGCTGCTCGACTACGCCCTTGCTGTCGAGGAGGTCACCACCTCCAAGAAGGACAACCTGATTCTGAA |
| | CGTTGACGGTGCTATTGCTGTTTCTTTTGTCGATCTCATGCGATCTTGCGGTGCCTTTACTGTGGAGGAGACTGAGG |
| | ACTACCTCAAGAACGGTGTTCTCAACGGTCTGTTCGTTCTCGGTCGATCCATTGGTCTCATTGCCCACCATCTCGAT |
| | CAGAAGCGACTCAAGACCGGTCTGTACCGACATCCTTGGGACGATATCACCTACCTGGTTGGCCAGGAGGCTATC |
| | CAGAAGAAGCGAGTCGAGATCAGCGCCGGCGACGTTTCCAAGGCCAAGACTCGATCATAG |
| MET17 | ATGCCATCTCATTTCGATACTGTTCAACTACACGCCGGCCAAGAGAACCCTGGTGACAATGCTCACAGATCCAGA |
| | GCTGTACCAATTTACGCCACCACTTCTTATGTTTTCGAAAACTCTAAGCATGGTTCGCAATTGTTTGGTCTAGAAGT |
| | TCCAGGTTACGTCTATTCCCGTTTCCAAAACCCAACCAGTAATGTTTTGGAAGAAAGAATTGCTGCTTTAGAAGGT |
| | GGTGCTGCTGCTTTGGCTGTTTCCTCCGGTCAAGCCGCTCAAACCCTTGCCATCCAAGGTTTGGCACACACTGGTG |
| | ACAACATCGTTTCCACTTCTTACTTATACGGTGGTACTTATAACCAGTTCAAAATCTCGTTCAAAAGATTTGGTATC |
| | GAGGCTAGATTTGTTGAAGGTGACAATCCAGAAGAATTCGAAAAGGTCTTTGATGAAAGAACCAAGGCTGTTTAT |
| | TTGGAAACCATTGGTAATCCAAAGTACAATGTTCCGGATTTTGAAAAAATTGTTGCAATTGCTCACAAACACGGTA |
| | TTCCAGTTGTCGTTGACAACACATTTGGTGCCGGTGGTTACTTCTGTCAGCCAATTAAATACGGTGCTGATATTGT |
| | AACACATTCTGCTACCAAATGGATTGGTGGTCATGGTACTACTATCGGTGGTATTATTGTTGACTCTGGTAAGTTC |
| | CCATGGAAGGACTACCCAGAAAAGTTCCCTCAATTCTCTCAACCTGCCGAAGGATATCACGGTACTATCTACAAT |
| | GAAGCCTACGGTAACTTGGCATACATCGTTCATGTTAGAACTGAACTATTAAGAGATTTGGGTCCATTGATGAACC |
| | CATTTGCCTCTTTCTTGCTACTACAAGGTGTTGAAACATTATCTTTGAGAGCTGAAAGACACGGTGAAAATGCATT |
| | GAAGTTAGCCAAATGGTTAGAACAATCCCCATACGTATCTTGGGTTTCATACCCTGGTTTAGCATCTCATTCTCAT |
| | CATGAAAATGCTAAGAAGTATCTATCTAACGGTTTCGGTGGTGTCTTATCTTTCGGTGTAAAAGACTTACCAAATG |
| | CCGACAAGGAAACTGACCCATTCAAACTTTCTGGTGCTCAAGTTGTTGACAATTTAAAGCTTGCCTCTAACTTGGC |
| | CAATGTTGGTGATGCCAAGACCTTAGTCATTGCTCCATACTTCACTACCCACAAACAATTAAATGACAAAGAAAA |
| | GTTGGCATCTGGTGTTACCAAGGACTTAATTCGTGTCTCTGTTGGTATCGAATTTATTGATGACATTATTGCAGACT |
| | TCCAGCAATCTTTTGAAACTGTTTTCGCTGGCCAAAAACCATGA |
| GPP1 | ATGCCTTTGACCACAAAACCTTTATCTTTGAAAATCAACGCCGCTCTATTCGATGTTGACGGTACCATCATCATCTC |
| | TCAACCAGCCATTGCTGCTTTCTGGAGAGATTTCGGTAAAGACAAGCCTTACTTCGATGCCGAACACGTTATTCAC |
| | ATCTCTCACGGTTGGAGAACTTACGATGCCATTGCCAAGTTCGCTCCAGACTTTGCTGATGAAGAATACGTTAACA |
| | AGCTAGAAGGTGAAATCCCAGAAAAGTACGGTGAACACTCCATCGAAGTTCCAGGTGCTGTCAAGTTGTGTAATG |
| | CTTTGAACGCCTTGCCAAAGGAAAAATGGGCTGTCGCCACCTCTGGTACCCGTGACATGGCCAAGAAATGGTTCG |
| | ACATTTTGAAGATCAAGAGACCAGAATACTTCATCACCGCCAATGATGTCAAGCAAGGTAAGCCTCACCCAGAAC |
| | CATACTTAAAGGGTAGAAACGGTTTGGGTTTCCCAATTAATGAACAAGACCCATCCAAATCTAAGGTTGTTGTCTT |
| | TGAAGACGCACCAGCTGGTATTGCTGCTGGTAAGGCTGCTGGCTGTAAAATCGTTGGTATTGCTACCACTTTCGAT |
| | TTGGACTTCTTGAAGGAAAAGGGTTGTGACATCATTGTCAAGAACCACGAATCTATCAGAGTCGGTGAATACAAC |
| | GCTGAAACCGATGAAGTCGAATTGATCTTTGATGACTACTTATACGCTAAGGATGACTTGTTGAAATGGTAA |
| NADH- | ATGACTGGTAAGACCGGTCATATTGATGGTTTGAACTCCAGAATCGAAAAGATGAGAGATTTGGATCCAGCTCAA |
| HMGR | AGATTGGTTAGAGTTGCTGAAGCTGCTGGTTTGGAACCAGAAGCTATTTCTGCTTTGGCTGGTAATGGTGCTTTGC |
| | CATTGTCTTTGGCTAATGGTATGATCGAAAACGTCATCGGTAAGTTCGAATTGCCATTGGGTGTTGCTACTAATTT |
| | CACTGTTAACGGTAGAGACTACTTGATTCCAATGGCTGTTGAAGAACCATCTGTTGTTGCTGCTGCTTCTTATATG |
| | GCTAGAATTGCTAGAGAAAACGGTGGTTTTACTGCTCATGGTACTGCTCCATTGATGAGAGCACAAATTCAAGTTG |
| | TTGGTTTGGGTGATCCAGAAGGTGCTAGACAAAGATTATTGGCTCATAAGGCTGCTTTTATGGAAGCTGCAGATGC |
| | TGTTGATCCAGTTTTGGTTGGTTTAGGTGGTGGTTGTAGAGATATCGAAGTTCACGTTTTTAGAGATACTCCAGTTG |
| | GTGCCATGGTTGTCTTGCATTTGATAGTTGATGTTAGAGATGCTATGGGTGCTAACACTGTTAATACCATGGCTGA |
| | AAGATTGGCTCCAGAAGTTGAAAGAATTGCTGGTGGTACTGTTAGATTGAGGATCTTGTCTAATTTGGCCGATTTG |
| | GAGGTATGGTTGAAGCTTGTGCTTTAGCTATCGTTGATCCATATAGAGCTGCTACTCATAACAAGGGTATTATGAA |
| | CGGTATCGATCCAGTTGTTGTTGCCACTGGTAATGATTGGAGAGCTATTGAAGCTGGTGCACATGCTTATGCTGCT |
| | AGATTAGTTAGAGCCAGAGTTGAATTGGCTCCTGAAACTTTGACTACTCAAGGTTATGATGGTGCTGATGTTGCTA |
| | AGAACTGGTCATTATACTTCATTGACCAGATGGGAATTAGCCAACGATGGTAGATTGGTTGGTACTATTGAATTGC |
| | CTTTGGCCTTGGGTTTAGTAGGTGGTGCTACAAAAACTCATCCAACTGCTAGAGCTGCATTGGCTTTGATGCAAGT |
| | TGAAACTGCTACTGAATTGGCACAAGTTACTGCTGCTGTAGGTTTGGCTCAAAACATGGCTGCTATTAGAGCTTTG |
| | GCTACTGAAGGTATTCAAAGGGGTCACATGACTTTACATGCTAGAAACATTGCTATTATGGCTGGTGCTACTGGTG |
| | CAGATATTGATAGAGTTACTAGAGTTATTGTCGAAGCCGGTGATGTTTCTGTTGCAAGAGCTAAACAAGTTTTGGA |
| | GAACACCTAA |
| ERG9 | ATGGGAAAGCTATTACAATTGGCATTGCATCCGGTCGAGATGAAGGCAGCTTTGAAGCTGAAGTTTTGCAGAACA |
| | CCGCTATTCTCCATCTATGATCAGTCCACGTCTCCATATCTCTTGCACTGTTTCGAACTGTTGAACTTGACCTCCAG |
| | ATCGTTTGCTGCTGTGATCAGAGAGCTGCATCCAGAATTGAGAAACTGTGTTACTCTCTTTTATTTGATTTTAAGGG |
| | CTTTGGATACCATCGAAGACGATATGTCCATCGAACACGATTTGAAAATTGACTTGTTGCGTCACTTCCACGAGAA |
| | ATTGTTGTTAACTAAATGGAGTTTCGACGGAAATGCCCCCGATGTGAAGGACAGAGCCGTTTTGACAGATTTCGA |
| | ATCGATTCTTATTGAATTCCACAAATTGAAACCAGAATATCAAGAAGTCATCAAGGAGATCACCGAGAAAATGGG |
| | TAATGGTATGGCCGACTACATCTTAGATGAAAATTACAACTTGAATGGGTTGCAAACCGTCCACGACTACGACGT |
| | GTACTGTCACTACGTAGCTGGTTTGGTCGGTGATGGTTTGACCCGTTTGATTGTCATTGCCAAGTTTGCCAACGAA |
| | TCTTTGTATTCTAATGAGCAATTGTATGAAAGCATGGGTCTTTTCCTACAAAAAACCAACATCATCAGAGATTACA |
| | ATGAAGATTTGGTCGATGGTAGATCCTTCTGGCCCAAGGAAATCTGGTCACAATACGCTCCTCAGTTGAAGGACTT |
| | CATGAAACCTGAAAACGAACAACTGGGGTTGGACTGTATAAACCACCTCGTCTTAAACGCATTGAGTCATGTTAT |
| | CGATGTGTTGACTTATTTGGCCGGTATCCACGAGCAATCCACTTTCCAATTTTGTGCCATTCCCCAAGTTATGGCCA |
| | TTGCAACCTTGGCTTTGGTATTCAACAACCGTGAAGTGCTACATGGCAATGTAAAGATTCGTAAGGGTACTACCTG |
| | CTATTTAATTTTGAAATCAAGGACTTTGCGTGGCTGTGTCGAGATTTTTGACTATTACTTACGTGATATCAAATCTA |
| | AATTGGCTGTGCAAGATCCAAATTTCTTAAAATTGAACATTCAAATCTCCAAGATCGAACAGTTTATGGAAGAAA |
| | TGTACCAGGATAAATTACCTCCTAACGTGAAGCCAAATGAAACTCCAATTTTCTTGAAAGTTAAAGAAAGATCCA |
| | GATACGATGATGAATTGGTTCCAACCCAACAAGAAGAAGAGTACAAGTTCAATATGGTTTTATCTATCATCTTGTC |
| | CGTTCTTCTTGGGTTTTATTATATATACACTTTACACAGAGCGTGA |
| GPD1 | ATGTCTGCTGCTGCTGATAGATTAAACTTAACTTCCGGCCACTTGAATGCTGGTAGAAAGAGAAGTTCCTCTTCTG |
| | TTTCTTTGAAGGCTGCCGAAAAGCCTTTCAAGGTTACTGTGATTGGATCTGGTAACTGGGGTACTACTATTGCCAA |
| | GGTGGTTGCCGAAAATTGTAAGGGATACCCAGAAGTTTTCGCTCCAATAGTACAAATGTGGGTGTTCGAAGAAGA |
| | GATCAATGGTGAAAAATTGACTGAAATCATAAATACTAGACATCAAAACGTGAAATACTTGCCTGGCATCACTCT |
| | ACCCGACAATTTGGTTGCTAATCCAGACTTGATTGATTCAGTCAAGGATGTCGACATCATCGTTTTCAACATTCCA |
| | CATCAATTTTTGCCCCGTATCTGTAGCCAATTGAAAGGTCATGTTGATTCACACGTCAGAGCTATCTCCTGTCTAA |
| | AGGGTTTTGAAGTTGGTGCTAAAGGTGTCCAATTGCTATCCTCTTACATCACTGAGGAACTAGGTATTCAATGTGG |
| | TGCTCTATCTGGTGCTAACATTGCCACCGAAGTCGCTCAAGAACACTGGTCTGAAACAACAGTTGCTTACCACATT |
| | CCAAAGGATTTCAGAGGCGAGGGCAAGGACGTCGACCATAAGGTTCTAAAGGCCTTGTTCCACAGACCTTACTTC |
| | CACGTTAGTGTCATCGAAGATGTTGCTGGTATCTCCATCTGTGGTGCTTTGAAGAACGTTGTTGCCTTAGGTTGTG |
| | GTTTCGTCGAAGGTCTAGGCTGGGGTAACAACGCTTCTGCTGCCATCCAAAGAGTCGGTTTGGGTGAGATCATCA |
| | GATTCGGTCAAATGTTTTTCCCAGAATCTAGAGAAGAAACATACTACCAAGAGTCTGCTGGTGTTGCTGATTTGAT |
| | CACCACCTGCGCTGGTGGTAGAAACGTCAAGGTTGCTAGGCTAATGGCTACTTCTGGTAAGGACGCCTGGGAATG |
| | TGAAAAGGAGTTGTTGAATGGCCAATCCGCTCAAGGTTTAATTACCTGCAAAGAAGTTCACGAATGGTTGGAAAC |
| | ATGTGGCTCTGTCGAAGACTTCCCATTATTTGAAGCCGTATACCAAATCGTTTACAACAACTACCCAATGAAGAAC |
| | CTGCCGGACATGATTGAAGAATTAGATCTACATGAAGATTAG |
| GPD2 | ATGCTTGCTGTCAGAAGATTAACAAGATACACATTCCTTAAGCGAACGCATCCGGTGTTATATACTCGTCGTGCAT |
| | ATAAAATTTTGCCTTCAAGATCTACTTTCCTAAGAAGATCATTATTACAAACACAACTGCACTCAAAGATGACTGC |
| | TCATACTAATATCAAACAGCACAAACACTGTCATGAGGACCATCCTATCAGAAGATCGGACTCTGCCGTGTCAAT |
| | TGTACATTTGAAACGTGCGCCCTTCAAGGTTACAGTGATTGGTTCTGGTAACTGGGGGACCACCATCGCCAAAGTC |
| | ATTGCGGAAAACACAGAATTGCATTCCCATATCTTCGAGCCAGAGGTGAGAATGTGGGTTTTTGATGAAAAGATC |
| | GGCGACGAAAATCTGACGGATATCATAAATACAAGACACCAGAACGTTAAATATCTACCCAATATTGACCTGCCC |
| | CATAATCTAGTGGCCGATCCTGATCTTTTACACTCCATCAAGGGTGCTGACATCCTTGTTTTCAACATCCCTCATCA |
| | ATTTTTACCAAACATAGTCAAACAATTGCAAGGCCACGTGGCCCCTCATGTAAGGGCCATCTCGTGTCTAAAAGG |
| | GTTCGAGTTGGGCTCCAAGGGTGTGCAATTGCTATCCTCCTATGTTACTGATGAGTTAGGAATCCAATGTGGCGCA |
| | CTATCTGGTGCAAACTTGGCACCGGAAGTGGCCAAGGAGCATTGGTCCGAAACCACCGTGGCTTACCAACTACCA |
| | AAGGATTATCAAGGTGATGGCAAGGATGTAGATCATAAGATTTTGAAATTGCTGTTCCACAGACCTTACTTCCACG |
| | TCAATGTCATCGATGATGTTGCTGGTATATCCATTGCCGGTGCCTTGAAGAACGTCGTGGCACTTGCATGTGGTTT |
| | CGTAGAAGGTATGGGATGGGGTAACAATGCCTCCGCAGCCATTCAAAGGCTGGGTTTAGGTGAAATTATCAAGTT |
| | CGGTAGAATGTTTTTCCCAGAATCCAAAGTCGAGACCTACTATCAAGAATCCGCTGGTGTTGCAGATCTGATCACC |
| | ACCTGCTCAGGCGGTAGAAACGTCAAGGTTGCCACATACATGGCCAAGACCGGTAAGTCAGCCTTGGAAGCAGA |
| | AAAGGAATTGCTTAACGGTCAATCCGCCCAAGGGATAATCACATGCAGAGAAGTTCACGAGTGGCTACAAACATG |
| | TGAGTTGACCCAAGAATTCCCATTATTCGAGGCAGTCTACCAGATAGTCTACAACAACGTCCGCATGGAAGACCT |
| | ACCGGAGATGATTGAAGAGCTAGACATCGATGACGAATAG |

### EXAMPLES

The following examples illustrate particular non-limiting embodiments.

To investigate the individual contribution of the five non-rate-limiting enzymes in the mevalonate pathway, we created a combinatorial library of 243 Saccharomyces cerevisiae strains, each having an extra copy of the mevalonate pathway integrated into the genome and expressing the non-rate-limiting enzymes from a unique combination of promoters. High-throughput screening combined with machine learning algorithms revealed that the mevalonate kinase, Erg12p, stands out as the critical enzyme that influences product titer. ERG12 is ideally expressed from a medium-strength promoter which is the ‘sweet spot’ resulting in high product yield. Additionally, a platform strain was created by targeting the mevalonate pathway to both the cytosol and peroxisomes. The dual localization synergistically increased terpene production and implied that some mevalonate pathway intermediates, such as mevalonate, IPP, and DMAPP, are diffusible across peroxisome membranes. The platform strain resulted in 94-fold, 60-fold, and 35-fold improved titer of monoterpene geraniol, sesquiterpene α-humulene, and triterpene squalene, respectively. The terpene platform strain will serve as a chassis for producing any terpenes and terpene derivatives.

### 2. Materials and Methods

2.1 Strains and growth media: S. cerevisiae strains used to construct the engineered strains, CEN.PK2-1C (MATa; his3D1; leu2-3_112; ura3-52; trp1-289; MAL2-8c; SUC2), CEN.PK2-1D (MATa; his3D1; leu2-3_112; ura3-52; trp1-289; MAL2-8c; SUC2) and CEN.PK2 (MATa/a; his3D1 his3D1; leu2-3_112 leu2-3_112; ura3-52 ura3-52; trp1-289 trp1-289; MAL2-8c/MAL2-8c; SUC2 SUC2), were acquired from Euroscarf, Germany. E. coli strain DH5α was used for cloning and plasmid propagation.

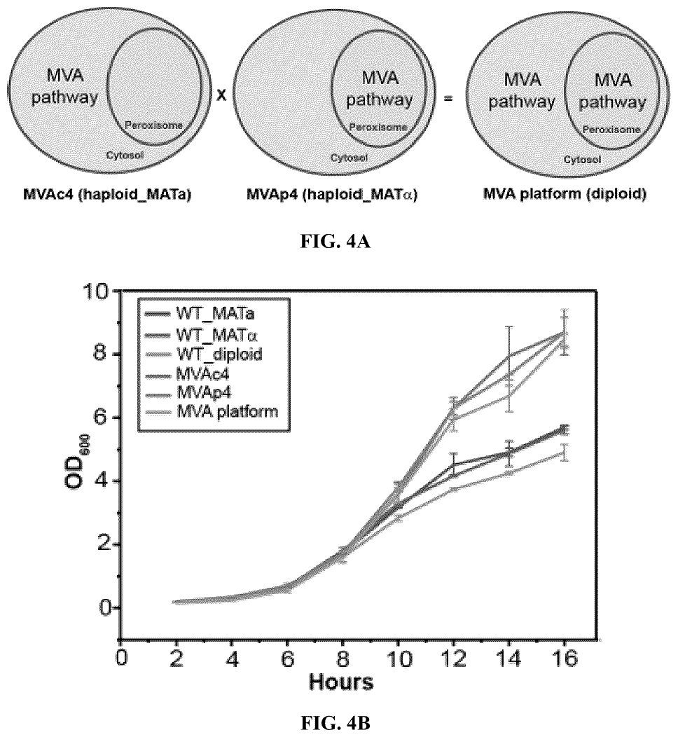

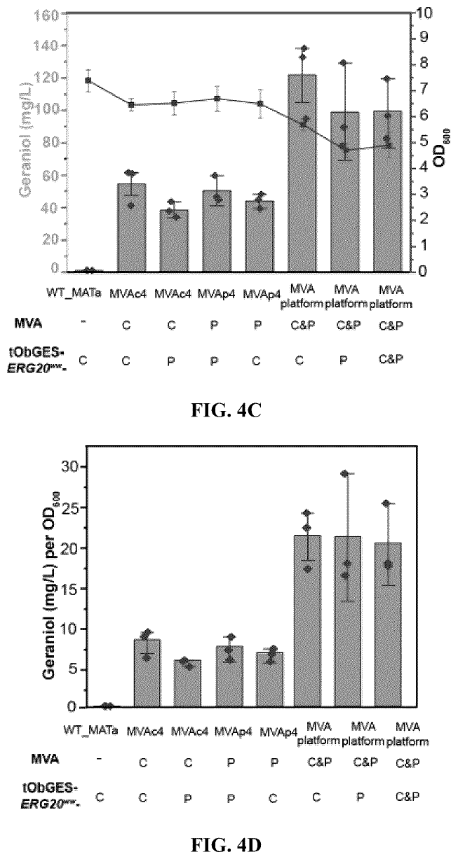

E. coli cells were grown on Luria-Bertani (LB) plates with appropriate antibiotics. Yeast synthetic dropout media used for integrations, mating, and culturing contained 0.67% (w/v) yeast nitrogen base without amino acids (Difco, Franklin Lakes, NJ), 2% (w/v) dextrose (Fisher Scientific, Waltham, MA), 0.07% (w/v) synthetic complete amino acid mix (CSM) without certain amino acids (Sunrise Science, Knoxville, TN). SD+400 μg/ml G418 (pH=7) (Goldbio, St. Louis, MO), which selects for the plasmid, was used for seed culture preparation. YPD (1% yeast extract, 2% peptone, and 2% dextrose) without antibiotic selection was used for preparing the growth curves in FIG. 4B. YPD+200 μg/ml G418 was used for compound production (36).

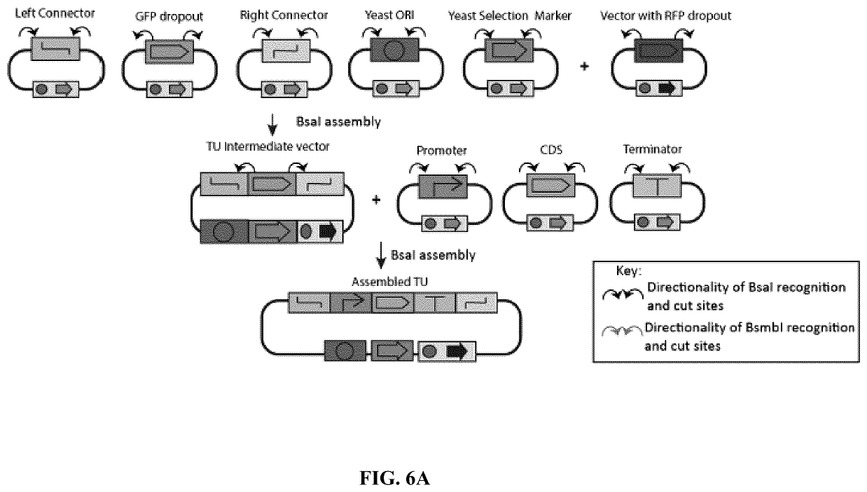

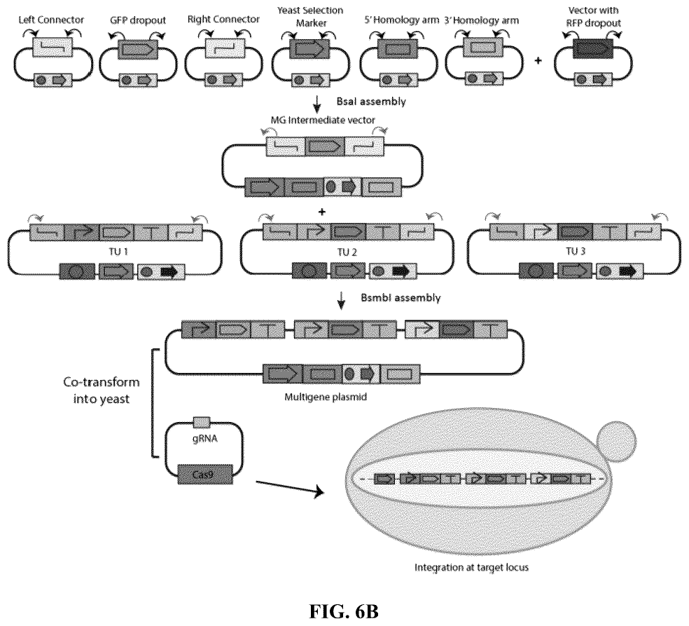

2.2 Gene synthesis, PCR, and Cloning: The ERG20^{WW}, tObGES, ZSS1, and CdGeDH genes were codon-optimized and synthesized by IDT (Newark, NJ). PCR amplification was performed using the Phusion High Fidelity DNA Polymerase (NEB, Ipswich, MA) according to the manufacturer's protocol. Gibson assembly (37) was used to clone the sgRNAs into the pCAS (70) plasmid for CRISPR-guided genomic integration. Golden Gate assembly (38) was performed to assemble all the other constructs. The sequences of all part plasmids were confirmed using Sanger sequencing (GeneWiz, South Plainfield, NJ). A schematic outlining the general strategy for cloning the multi-gene plasmids is outlined in FIGS. 6A and 6B. All the constructs created and primers used are listed in Tables 3-10.

2.3 Strain construction: Yeast competent cells were co-transformed with the NotI digested and linearized multi-gene (39) and pCAS-sgRNA (40) plasmids using the Frozen-EZ yeast transformation II kit (Zymo Research, Irvine, CA) according to the manufacturer's protocol. The transformed cells were plated on appropriate dropout media for selection and incubated at 30° C. for two days and 37° C. for an additional day to facilitate genomic integration (40). Two pairs of diagnostic primers were used to confirm each integration by polymerase-chain reactions (PCR) using the GoTaqGreen DNA polymerase (Promega, Madison, WI). For further confirmation of each gene in two-gene inserts at ROX1 and GAL80 loci, primers were designed such that the forward and reverse primers bind to the first and the second gene, respectively. For three gene inserts at the GAL1 locus, an additional pair of forward and reverse primers bind to the second and third genes, respectively. All the primers used are listed in Table 10.

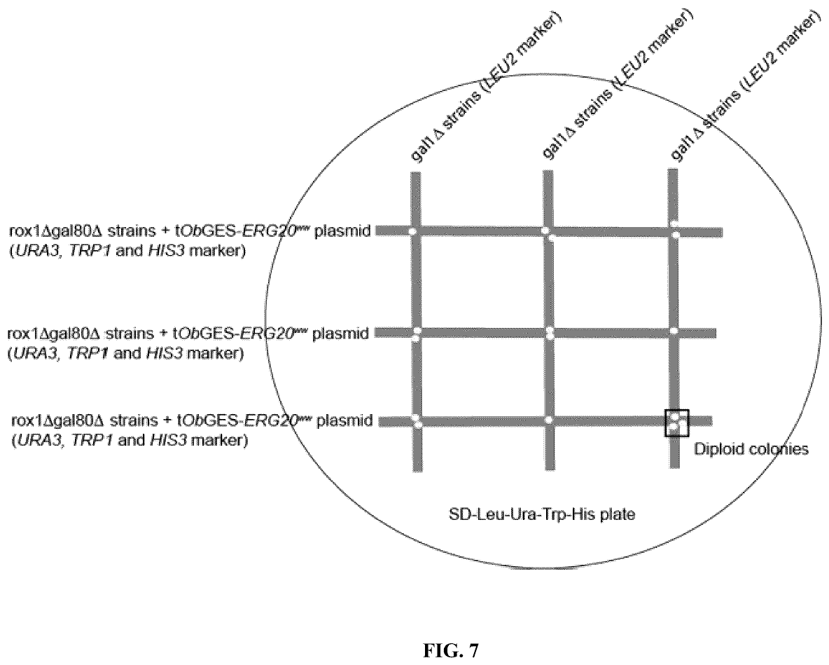

2.4 Mating of yeast strains: 243 library strains: One colony was picked from each of the 27 GAL1Δ and 9 ROX1ΔGAL80Δ+tObGES-ERG20^{ww} strains from their respective dropout plates (SD-Leu and SD-Ura-Trp-His) and streaked out in vertical and horizontal lines respectively on an SD-Leu-Ura-Trp-His plate followed by incubating at 30° C. for two days (see schematic in FIG. 7). Colonies growing at the intersection of the streaks were further streaked out on a fresh SD-Leu-Ura-Trp-His plate and incubated at 30° C. overnight. They were then screened with diagnostic and gene-specific primers to confirm the integration. For the MVA platform strain, one colony from MVAc4 and MVAp4 were streaked out as above on an SD-Leu-Ura-Trp+200 μg/ml Hygromycin (Goldbio, St. Louis, MO) plate and incubated and screened as mentioned above.

### 2.5 Geraniol Production and Quantification:

2.5.1 Geraniol production: For geraniol production from strains CEN.PK2-1C and MVAc1-MVAc4, yeast colonies transformed with the pPYK1-tObGES-ERG20^{ww} plasmids were grown overnight in 5 ml SD-His at 30° C. with shaking at 200 rpm. The overnight culture was inoculated at an initial OD₆₀₀ of 0.1 into fresh SD-His and grown at 30° C. with shaking at 200 rpm for 48 hours. 1 ml of the culture was collected at 12, 24, and 48 hours and was pelleted at 16,000×g for 1 min, and 50 μl of the supernatant was used to quantify geraniol using the geraniol dehydrogenase (GeDH) assay (41).

For library screening, seed cultures were set up with three replicates of each wildtype CEN.PK2 and 243 strains by inoculating three colonies of each strain into 200 μl SD-Leu-Ura-Trp-His media in 96-well plates. The overnight culture was inoculated at an initial OD₆₀₀ of ˜0.1 into fresh SD-Leu-Ura-Trp-His media in 96-deep-well plates; each well has 500 ul culture. The deep-well plates were incubated at 30° C. with shaking at 400 rpm for 12 hours. The plates were centrifuged at 3,220×g for 5 mins, and 50 μl of the supernatant was used for the GeDH assay.

For geraniol production from the wildtype CEN.PK2-1C, MVAc4, MVAp4, and MVA platform strains, yeast colonies transformed with either pGAL1-tObGES-ERG20^{ww} or tObGES-ERG20^{ww}-SKL were grown overnight in 5 ml SD+400 μg/ml G418 (pH=7). The overnight culture was inoculated at an initial OD₆₀₀ of 0.1 into fresh YPD+200 μg/ml G418 and grown at 30° C. with shaking at 200 rpm for 24 hours. 1 ml of the culture was collected and pelleted at 16,000×g for 1 min, and 50 μl of the supernatant was used to quantify geraniol using the GeDH assay.

2.5.2 Geraniol dehydrogenase assay: CdGeDH gene from Castellaniella defragrans, encoding the geraniol dehydrogenase, was cloned in the pET-24 vector by Gibson assembly (75). Protein purification and the assay were performed with slight modifications from the protocol described in Lin et al. 2018 (41). Briefly, pET-24_CdGeDH with a C-terminal his-tag was transformed into E. coli (BL21), a single colony was inoculated for seed culture overnight and diluted 50-fold in a scaled-up culture, grown at 37° C. till OD₆₀₀ of 0.6, then 0.1 mM of IPTG (Goldbio, St. Louis, MO) was added, followed by grown at 16° C. for 24 hours. The culture was centrifuged at 3220×g for 20 mins, the supernatant was discarded, and the pellet was resuspended in lysis buffer (50 mM Tris pH=7.5, 5 mM imidazole, and 1 mM phenylmethylsulfonyl fluoride) and 1 mg/ml lysozyme (Sigma Aldrich, St. Louis, MO). Cells were lysed with a sonicator (Misonix, Farmingdale, NY) for 2 min with 10 s pulses. Proteins were purified using a Ni-NTA column (Qiagen, Germantown, MD). Unbound proteins were eliminated with wash buffer (50 mM Tris pH-7.5, 40 mM imidazole), and GeDH protein was eluted with elution buffer (50 mM Tris pH-7.5, 250 mM imidazole). The purify of the resulting CdGeDH enzyme was routinely examined by protein gel electrophoresis.

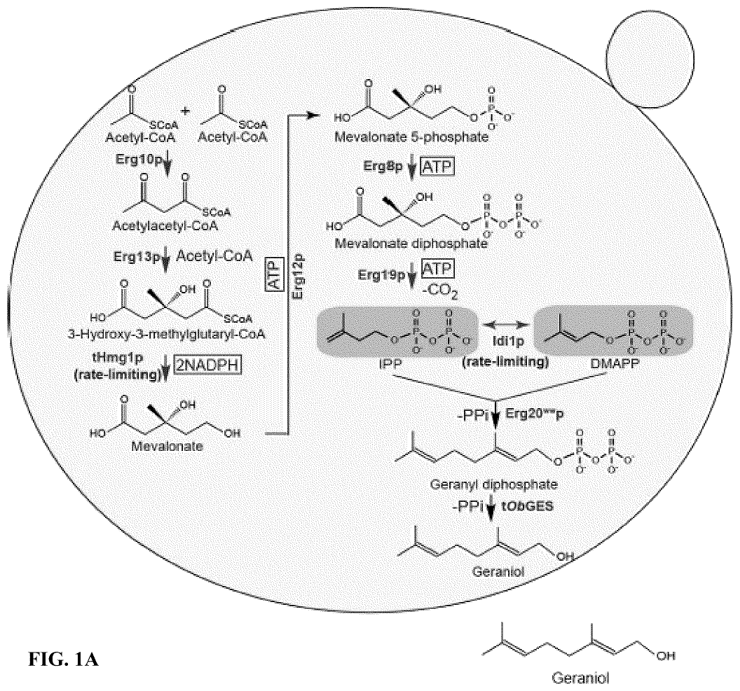

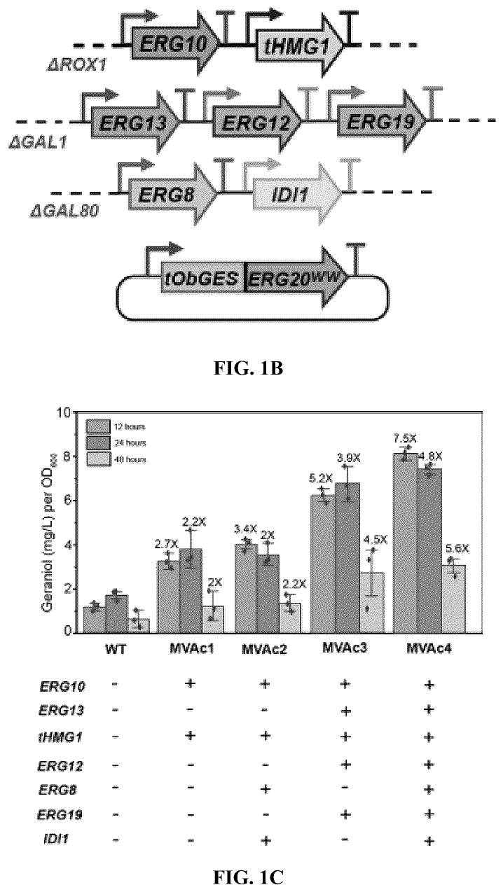

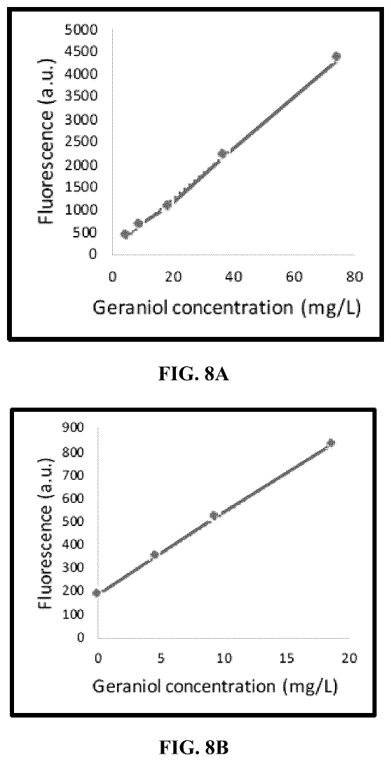

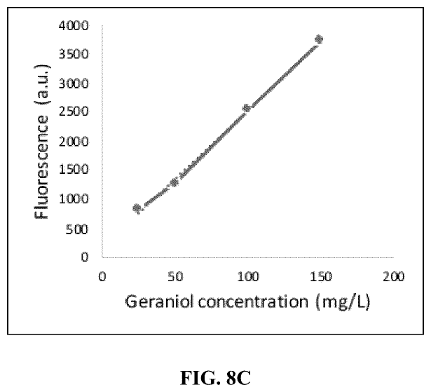

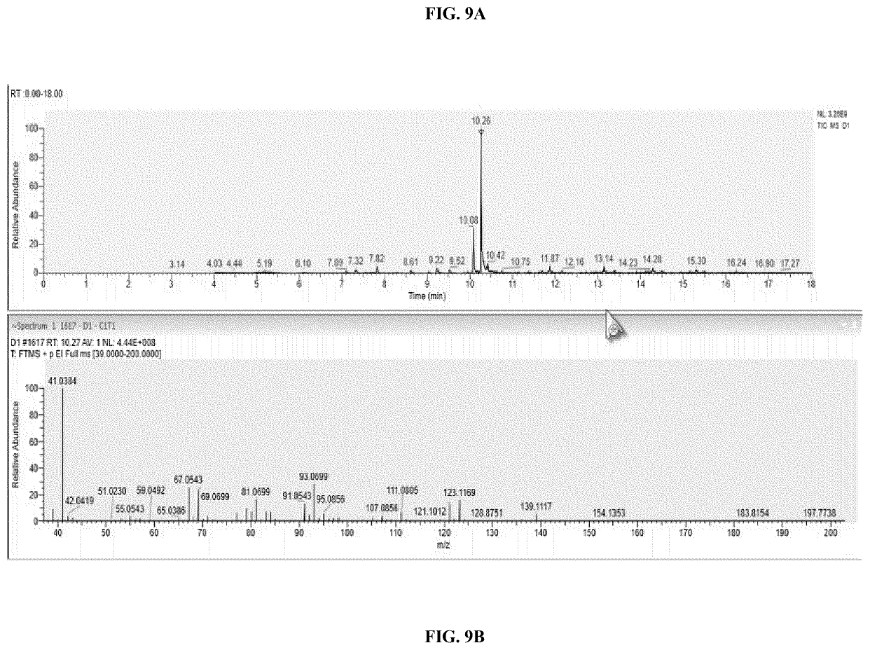

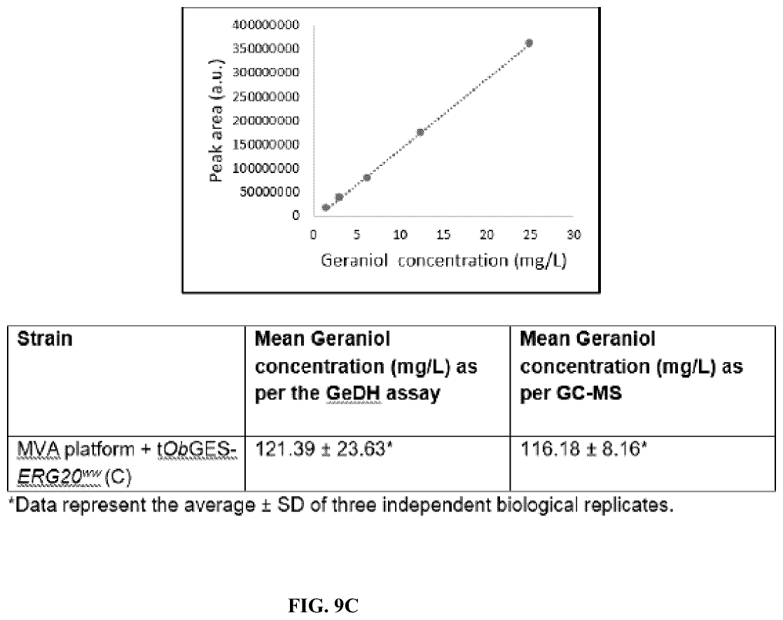

For the GeDH assay, 50 μl of the spent media was mixed with 50 μl of a prepared reaction mix such that the final mixture contained: 100 mM Tris-HCl (pH 8.0), 2 mM nicotinamide adenine dinucleotide (NAD⁺) (Goldbio, St. Louis, MO), 2 mM resazurin sodium salt (Acros Organics, Belgium), 0.002 U purified geraniol dehydrogenase, and 1 U diaphorase (Sigma Aldrich, St. Louis, MO). To prepare geraniol standard curve, 10× of each geraniol concentration was prepared by dissolving authentic geraniol standard (Acros Organics, Belgium) in acetone. Next, the 10× concentrations were diluted and added to the reaction mix such that the final geraniol concentration is 1×. The geraniol standard curves used for FIGS. 1A, 2B, and 4C are shown in FIGS. 8A-8C. Each reaction was incubated at room temperature for 45 min, and fluorescence was recorded at the excitation and emission of 530 nm and 590 nm, respectively, using a Tecan Spark microplate reader (Morrisville, NC). The geraniol concentrations of MVA platform+ tObGES-ERG20^{ww} were confirmed using gas chromatography coupled with mass spectrometry (GC-MS) (FIGS. 9A-9C).

2.6 Terpene quantification using GC-MS: For geraniol, citronellol, and geranyl acetate extraction, 1 ml culture was centrifuged at 16,000×g for 1 min, 500 μl of the supernatant was mixed with 500 μl hexane and shaken in a plate shaker at the highest speed for 10 min, followed by centrifugation at 16,000×g for 2 mins. 500 μl of the hexane layer was diluted five folds in hexane and used for GC-MS. For α-humulene extraction, 1 ml culture was centrifuged at 16,000×g for 1 min, and 500 μl of the supernatant was mixed with 500 μl ethyl acetate and shaken in a plate shaker at the highest speed for 10 min followed by centrifugation at 16,000×g for 2 mins. 500 μl of the ethyl acetate layer was collected for GC-MS. For squalene extraction, 1 ml culture was centrifuged at 16,000×g for 1 min. The supernatant was discarded, and the pellet was dissolved in 200 μl ethyl acetate, followed by homogenizing with 100 mg of 0.5 mm glass beads in a Bullet Blender® tissue homogenizer at the highest setting for 10 mins at 4° C. 300 μl ethyl acetate was then added to the sample, and the sample was further vortexed and centrifuged at 16,000×g for 2 mins. 500 μl of the hexane layer was collected for GC-MS.

Terpenes were detected using a Thermo Trace 1300 Gas Chromatograph and Thermo Q-Exactive™ Orbitrap Mass Spectrometer (Waltham, MA). 5 μL geraniol-containing samples, 2 μL α-humulene-, or squalene-containing samples were injected into a Thermo Scientific TraceGOLD TG-5SILMS column (30 m long, 0.25 mm inner diameter, 0.25 m film thickness) using helium as the carrier gas (1 ml/min). The injector was held at 200° C. For geraniol, citronellol, and geranyl acetate analysis, the oven was held at 40° C. for 4 mins, followed by ramping up to 280° C. at a rate of 20° C./min and then holding at 280° C. for 2 mins. The mass range monitored was 39-200 m/z in the positive ion mode. Geraniol eluted at 10.24 mins, citronellol at 9.93 mins, and geranyl acetate at 10.99 mins. For α-humulene, the oven was held at 80° C. for 3 mins, followed by ramping up to 180° C. at a rate of 15° C./min and further ramping to 240° C. at the rate of 10° C./min, holding for 1 min. The mass range monitored was 50-250 M/Z in the positive ion mode. α-humulene eluted at 9.7 mins. For squalene, the oven was held at 80° C. for 3 mins, followed by ramping up to 180° C. at a rate of 15° C./min and further ramping to 310° C. at 20° C./min and then holding at 280° C. for 1 min. The mass range monitored was 50-450 m/z in the positive ion mode. Squalene eluted at 16.8 mins. The MS transfer line was at 250° C., and the source temperature was 200° C. The resolution was set to 60,000. The MS was set to monitor total ion counts.

Peak areas for geraniol, α-humulene, and squalene were quantified using the Xcalibur™ software (Thermo Fisher, Waltham, MA). Absolute sample concentrations were calculated from a standard curve of authentic geraniol (Acros Organics, Belgium), citronellol (Acros Organics, Belgium), geranyl acetate (Thermo Scientific, Waltham, MA), α-humulene (Millipore Sigma, Burlington, MA), and squalene (TCI America, Portland, OR) standards. To prepare standard curves, geraniol, citronellol, and geranyl acetate were diluted in hexane and squalene and α-Humulene standards in ethyl acetate. Geraniol and squalene standards were diluted over a range of 1.56-25 mg/L, citronellol 1.06-6.25 mg/L, and α-Humulene 0.531-12.5 mg/L. Ions of m/z values 123.1168±5 ppm, 138.1403±5 ppm, 136.1247±5 ppm, 93.0698±5 ppm, and 121.1012±5 ppm were used for quantifying the peak area for geraniol, citronellol, geranyl acetate, α-humulene, and squalene, respectively.

Statistical methods: A random forest (RF) (42) was used to fit predictive models for geraniol production. Briefly, RFs construct ensembles of Classification and Regression Trees (CART) (43) from bootstrap replications of the data. Each CART model is a decision tree that creates a prediction of geraniol, and the final prediction is based on aggregation over the ensemble. Models were fit based on out-of-bag estimation (44), which prevents overfitting.

Tree-based models such as RFs are particularly useful when interactions are expected between variables, in this case, the MVA pathway enzymes, and for delineating the role and importance of the individual variables (44) in the prediction of the outcome, geraniol titer. Another strength of the RF is that it implements bootstrap resampling of the data (45), accounting for uncertainty in the population, and is ideal for a smaller sample size of this type. The bootstrap replication datasets are generated by resampling the observations (strains) with replacement and are the same size as the original dataset. The output is an ensemble of prediction models aggregated to produce a prediction for each observation. The accuracy of the RF was estimated using a simple residual sum of squares (RSS) loss function averaged over out-of-bag (OOB) samples (46) in the ensemble to produce a mean squared error (MSE). Using the GOB error estimate eliminates the requirement for a set-aside test set (42). Notably, by nature of the resampling, not all the observations are present in each bootstrap replication. OOB error leverages this for estimation by aggregating only over the predictors in the ensemble for which an observation was not randomly selected in the bootstrap, which inherently avoids overfitting (42). OOB estimation is an effective alternative for smaller datasets that may be sensitive to training and testing splits or fold assignments in cross-validation.

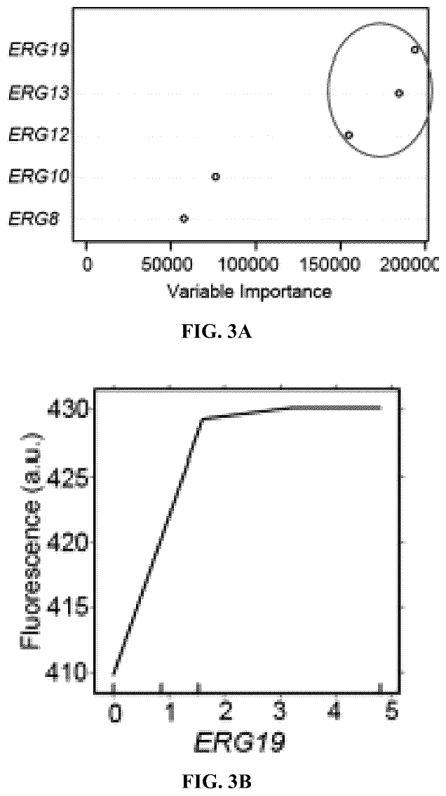

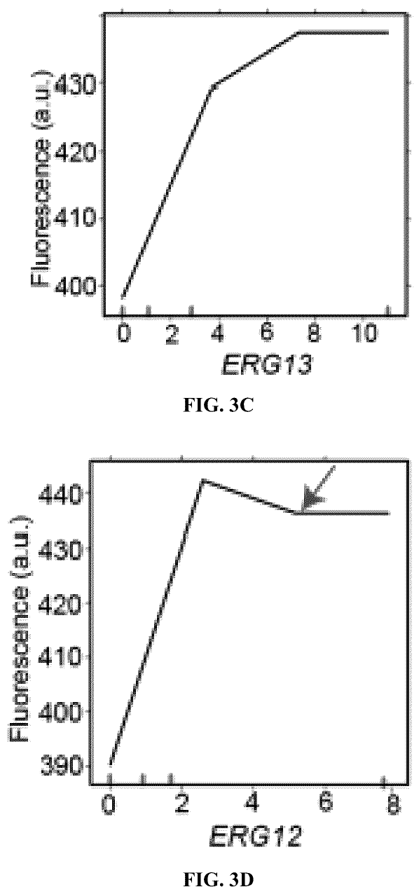

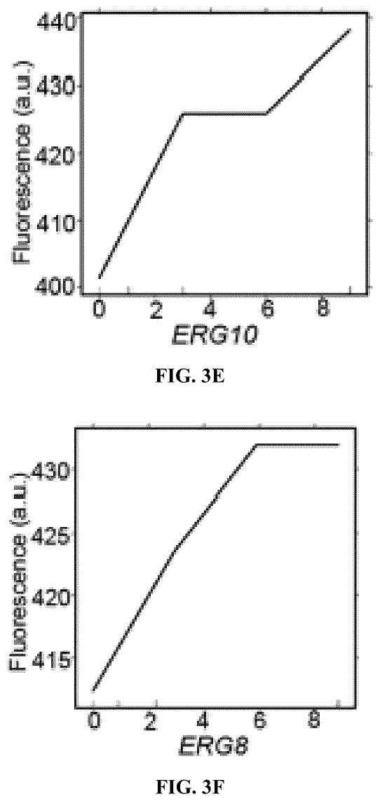

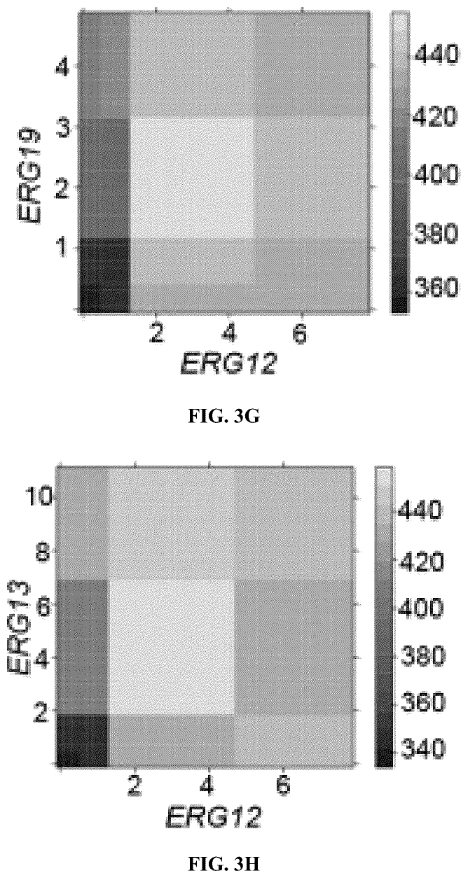

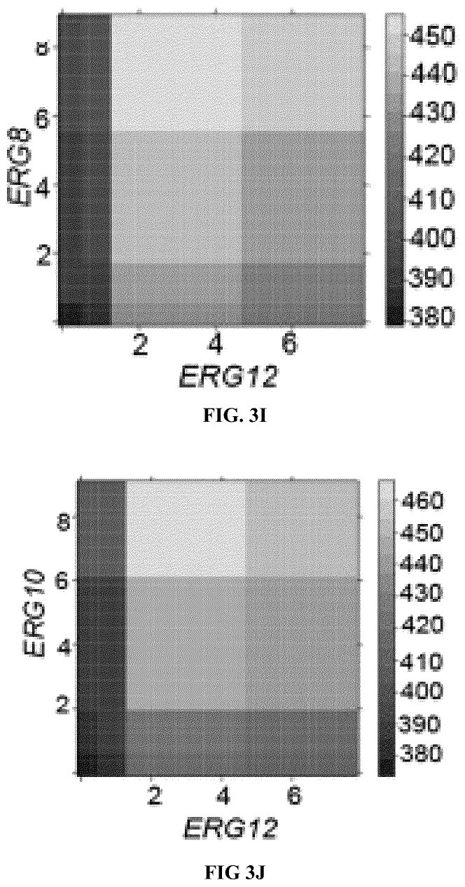

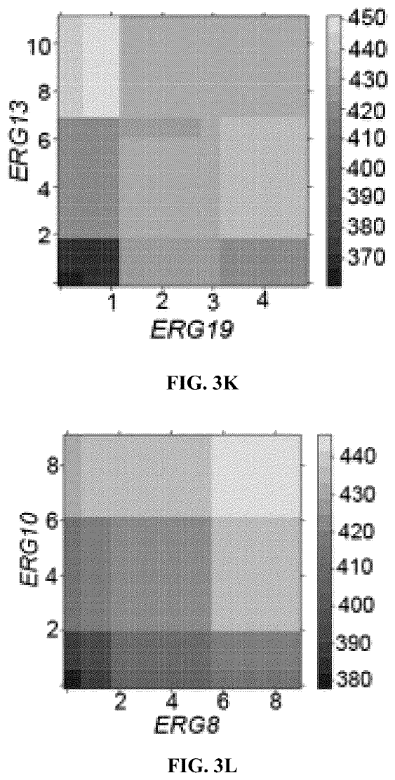

Variable importance (42, 46) measures were used to prioritize the enzymes according to their contribution to the predictive accuracy of the outcome. Importance is measured by increases in node purity that serves as a surrogate for the performance of the random forest. High increases in node purity indicate that the predictive strength of the model shows high levels of improvement when the enzyme is included in the random forest, and its elimination from the data set would considerably degrade the predictive strength (FIG. 3A).

Partial Dependence Plots (PDP) are a popular technique for visualizing the contribution of variables to an outcome and the relationships between pairs of variables and an outcome (47, 48). Using the variable importance measure as a prioritization, we examined the impact of the five MVA pathway enzymes on geraniol production and their interactions. PDP profiles were computed using grids created of ten equally spaced values over the support region for each enzyme. Linear interpolation was used to estimate geraniol production in between data points.

Individual Conditional Expectation (ICE) curves (49) were also examined for the highest and lowest-producing strains. ICE curves enable the visualization of the functional relationships between the predicted values of geraniol production and enzyme levels for individual strains and are useful for assessing sensitivity (FIGS. 9A-9C).

Analysis was performed in the R programming language with the “randomForest” (42), “PDP” (48), and “vivo” packages.

### 3 Results

3.1 Sequential Integration of the Complete MVA Pathway into the Yeast Genome

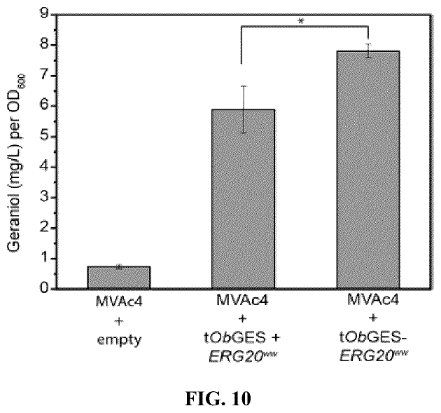

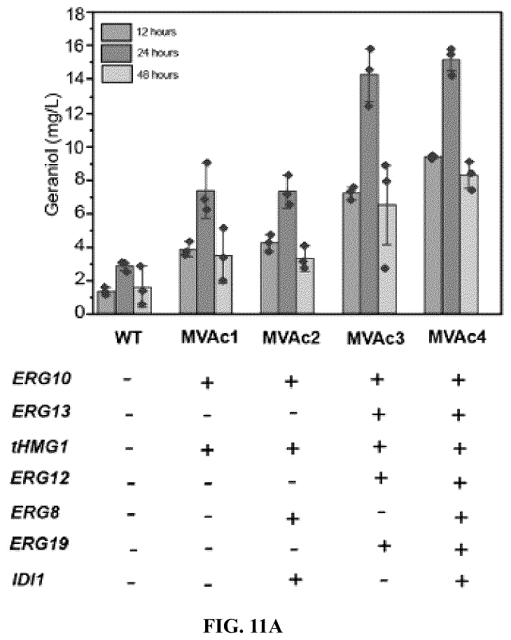

The disclosure provides for genomic integration instead of a plasmid-based system for certain described genes because a preferable platform strain should be genetically stable and not require selective markers during fermentation. An additional copy of all seven MVA pathway genes was integrated sequentially into the yeast genome under the rationale that overexpression of the complete MVA pathway would increase IPP and DMAPP levels. The MVA pathway genes were inserted into three genomic loci, GAL80, GAL1, and ROX1 (FIG. 1B, Table 1). GAL80 and GAL1 deletions allowed gene expression under galactose-inducible promoters when glucose was the sole carbon source (11). ROX1 was disrupted to boost the MVA pathway by alleviating transcriptional repression (50). Each MVA pathway gene was expressed from a unique, strong constitutive promoter to minimize potential homologous recombination (51). The sequentially engineered MVA strains (MVAc1-4) were transformed with a plasmid enabling the production of geraniol, a fragrant monoterpene and a precursor for medicinally important indole alkaloids (52, 53). The fusion protein tObGES-ERG20^{ww} (54, 55) was used for geraniol biosynthesis as fusing geraniol diphosphate synthase (ERG20^{ww}) with geraniol synthase (tObGES) resulted in higher geraniol production than when the two genes are separately expressed (FIG. 10).

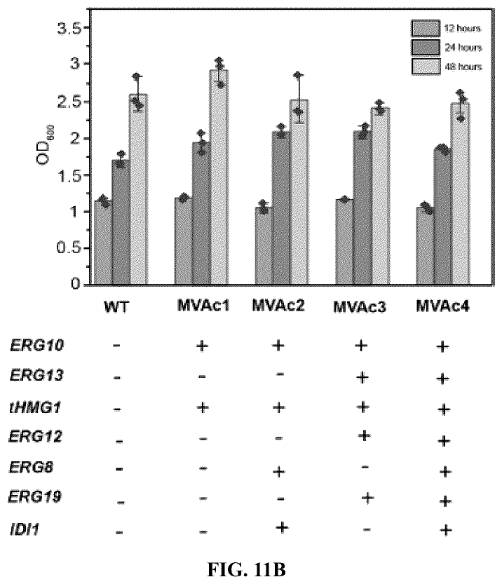

Geraniol yield increased with the increase in the number of overexpressed MVA pathway genes (FIG. 1C). Strain MVAc1 with ERG10 and tHMG1 overexpressed had over 2.5-fold increased geraniol yield after 12 hours of shake-flask cultivation. Strain MVAc2 only showed a marginal increase compared with MVAc1, likely because the excessive mevalonate generated by tHMG1 overexpression was not channeled into the MVA pathway due to the lack of the mevalonate kinase ERG12 in the heterologous pathway. Strain MVAc3 overexpressing five out of the seven MVA pathway genes further increased geraniol yield. MVAc4 with the complete MVA pathway overexpressed had the highest geraniol yield, which is 7.5-fold of the wild type at 12 hours. Geraniol titer was maximum at 24 hours (FIGS. 11A and 11B). Therefore, in addition to the two rate-limiting enzymes, the other five enzymes also play important roles in increasing the MVA pathway productivity.

TABLE 1
List of strains generated for creating the MVA platform strain.

| Strains | Description | Source |
|---|---|---|
| MVAc1 | CEN-PK2-1C; rox1Δ::ERG10-tENO1, | This study |
| | pTDH3-tHMG1-tTDH1, URA3 | |
| MVAc2 | MVAc1; gal80Δ::pTEF1-ERG8-tSSA1, | This study |
| | pCCW12-IDI1-tENO2, TRP1 | |
| MVAc3 | MVAc1; gal1Δ::pPGK1-ERG13-tPGK1, | This study |
| | pTEF2-ERG12-tADH1, pHHF1-ERG19- | |
| | tCYC1, LEU2 | |
| MVAc4 | MVAc3; gal80Δ::pTEF1-ERG8-tSSA1, | This study |
| | pCCW12-IDI1-tENO2, TRP1 | |
| MVAp1 | CEN-PK2-1D; rox1Δ::pHHF2-ERG10- | This study |
| | SKL-tENO1, tHMG1-SKL-tTDH1, URA3 | |
| MVAp2 | MVAp1; gal80Δ::pTEF1-ERG8-SKL-tSSA1, | This study |
| | pCCW12-IDI1-SKL-tENO2, pTEF1-HygR- | |
| | tTEF1 | |
| MVAp3 | MVAp1; gal1Δ::pPGK1-ERG13-SKL-tPGK1, | This study |
| | pTEF2-ERG12-SKL-tADH1, pHHF1-ERG19- | |
| | SKL-tCYC1, LEU2 | |
| MVAp4 | MVAp3; gal80Δ::pTEF1-ERG8-SKL-tSSA1, | This study |
| | pCCW12-IDI1-SKL-tENO2, pTEF1-HygR- | |
| | tTEF1 | |
| MVA | CEN-PK2; rox1Δ::pHHF2-ERG10-tENO1, | This study |
| platform | pTDH3-tHMG1-tTDH1, URA3; gal1Δ:: | |
| | pPGK1-ERG13-tPGK1, pTEF2-ERG12-tADH1, | |
| | pHHF1-ERG19-tCYC1, LEU2; gal80Δ:: | |
| | pTEF1-ERG8-tSSA1, pCCW12-IDI1-tENO2, | |
| | TRP1; rox1Δ::pHHF2-ERG10-SKL-tENO1, | |
| | pTDH3-tHMG1-SKL-tTDH1, URA3; | |
| | gal1Δ::pPGK1-ERG13-SKL-tPGK1, pTEF2- | |
| | ERG12-SKL-tADH1, pHHF1-ERG19-SKL- | |
| | tCYC1, LEY2; gal80Δ::pTEF2-ERG8-SKL- | |
| | tSSA1, pCCW12-IDI1-SKL-tENO2, | |
| | pTEF1-HygR-tTEF1 | |

### 3.2 Creating a Combinatorial Strain Library to Survey the Promoter Space of MVA Pathway Genes

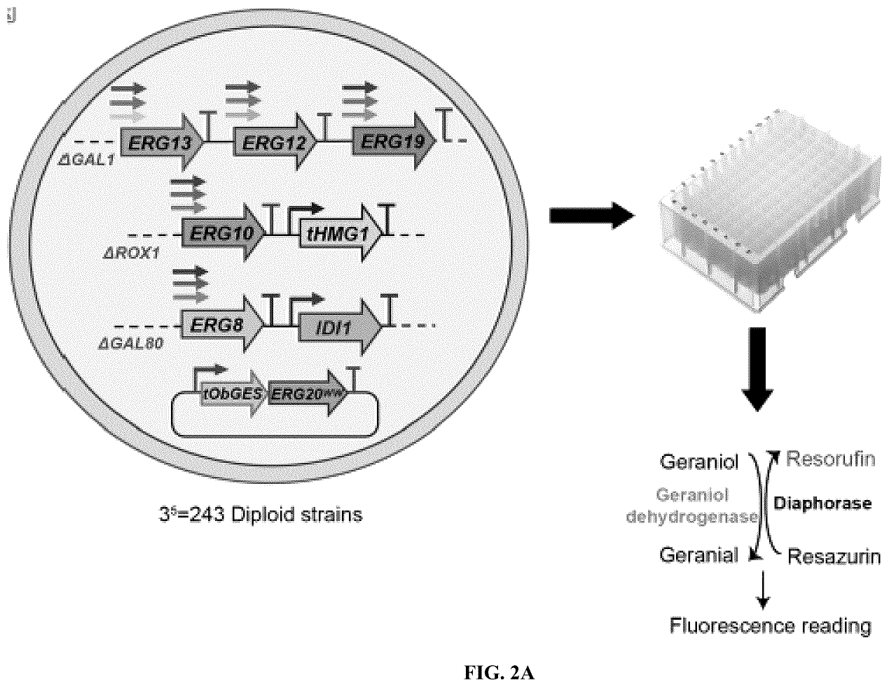

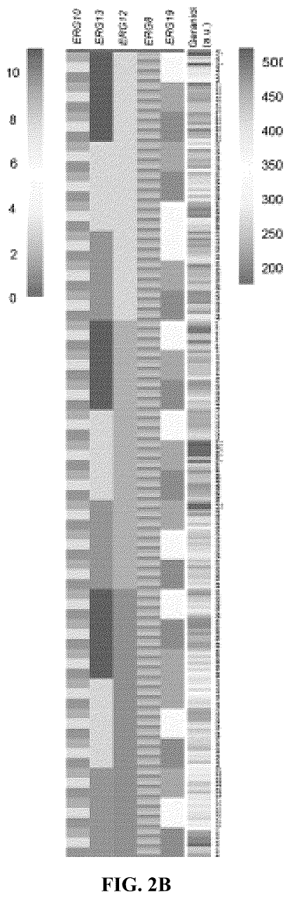

When integrating the complete MVA pathway into the genome, strong yeast promoters are usually used. However, they may not be a preferred set of promoters that maximize pathway productivity. To find the improved promoter combinations of pathway genes and to delineate the contribution of each gene to MVA pathway productivity, we created a combinatorial strain library of 243 diploid strains with varying promoter strengths. The rate-limiting genes tHMG1 and IDI1 were always expressed from a strong promoter since their essentiality to the pathway is well-documented (17-21, 56). Each of the remaining five genes was expressed from a unique combination of strong, medium, or weak promoters, creating 3⁵=243 strains (FIG. 2A). The choice of promoters and their relative expression strengths were based on the extensive characterization of yeast promoters by Lee et al (39) (Table 12).

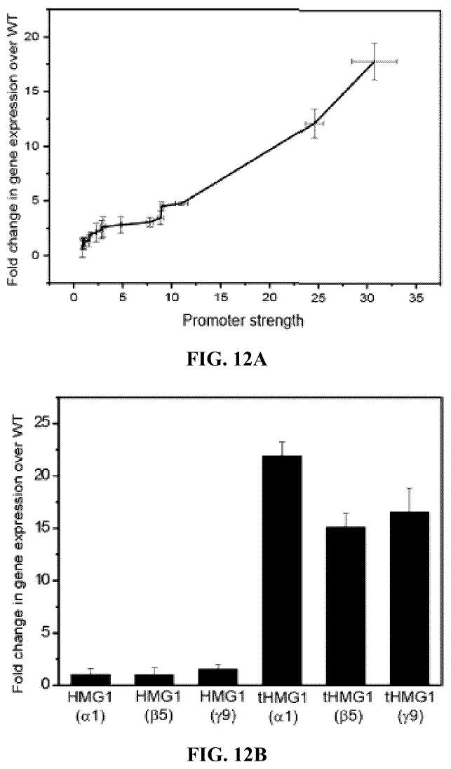

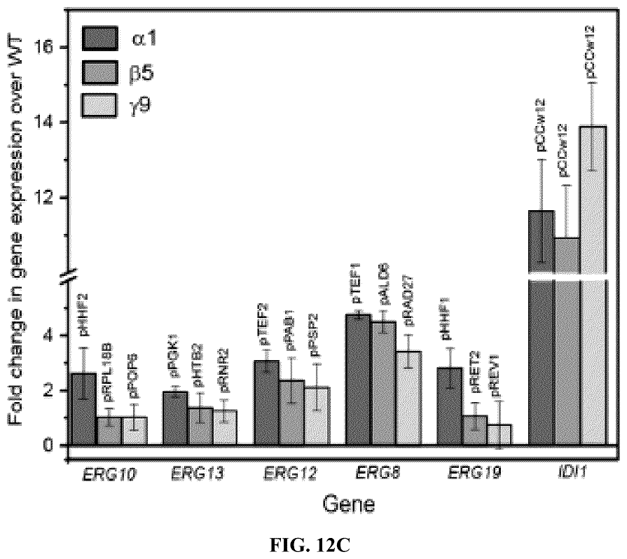

The construction of the combinatorial library was streamlined by mating engineered haploids of opposite mating types. Haploid strains of mating-type MATa overexpressed ERG13, ERG12, and ERG19, each under three different promoters, in the GAL1 locus. 3³=27 of such MATa strains were created (Table 12). Similarly, haploid strains with the opposite MATa mating type overexpressed the other four MVA pathway genes with ERG10 and ERG8 under three different promoters, generating 3²=9 strains (Table 13). These nine strains were also transformed with a plasmid bearing the tObGES-ERG20^{ww} fusion gene for geraniol production. Mating the engineered haploid strains with the opposite mating type generated 3³×3²=243 diploid strains, each containing an extra copy of the seven MVA pathway genes and capable of producing geraniol. The strain library was cultivated in 96-deep-well plates, followed by geraniol quantification using a high-throughput fluorescence-based assay (41). A heat map with the promoter strengths and fluorescence readings of all strains revealed a unique pattern that the strains expressing ERG12 from a medium-strength promoter produced some of the highest amounts of geraniol. Eight out of the top ten geraniol-producing strains had ERG12 expressed from the medium-strength promoter (FIG. 2B). Quantitative real-time PCR verified that transcript levels of overexpressed MVA pathway genes positively correlated with the promoter strengths (FIGS. 12A-12C, Table 11). Quantification of intracellular mevalonate, a critical pathway intermediate, in the strains with all strong promoters (α1), all medium promoters (β5), and all weak promoters (γ9) showed a progressive decrease, as expected. (Table 14).

### 3.3 Applying Machine Learning to the Combinatorial Strain Library

Machine learning was used to investigate the combinatorial library with the primary objective of understanding the impact of each of the five enzymes on the productivity of the MVA pathway. Random forest models (42) were fit to the data in the combinatorial library with the outcome variable as geraniol production. Variable importance measures indicate that the top three enzymes that are critical for predicting geraniol production are Erg19p, the mevalonate pyrophosphate decarboxylase; Erg13p, the HMG-CoA synthase; and Erg12p, the mevalonate kinase (FIG. 3A). In addition to the ranking, we also view the drops in importance as insightful, especially between Erg12p and Erg10p. This large gap secured the role of the top three enzymes as critical for the predictive accuracy of geraniol production in the 243 strains.

Next, we took a closer look at measures of variable importance using Partial Dependence Plots (PDPs) (48) to visualize the contribution of the enzyme levels to geraniol output. PDP of the five enzymes showed the predicted geraniol production when an enzyme was set at a given promoter strength (FIG. 3B-F). Erg13p, Erg19p, and Erg8p showed increased geraniol production when their promoter strengths were increased, eventually leveling off at saturation (FIG. 3B, C&F), as expected. However, a unique role of mevalonate kinase (Erg12p) was apparent from the PDP of ERG12 (FIG. 3D), which showed that a maximum geraniol production was reached within our data when its expression level was moderately low and then decreased with higher promoter strength. Erg10p did not show saturation in the promoter strengths tested.

In the two-enzyme interaction plots (FIG. 3G-L), the role of Erg12p is even more apparent. When the value of ERG12 was in the moderate range, the predicted geraniol output was the highest. This could be due to several reasons, such as feedback inhibitions of Erg12p by pathway intermediates (57-60) and metabolic burden leading to protein aggregation. Therefore, moderate expression of ERG12 most likely strikes the right balance for higher flux through the pathway.

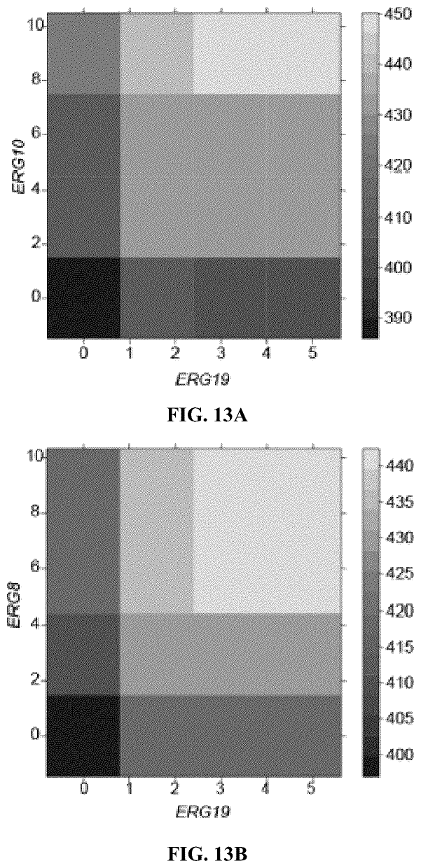

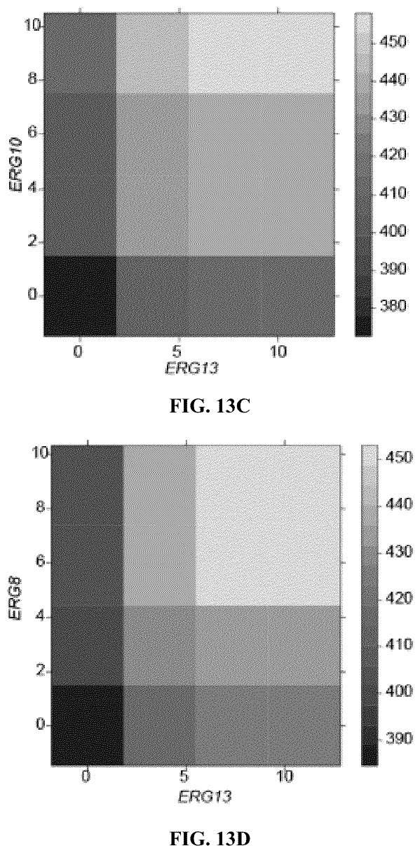

The two-enzyme interaction plot between ERG19 and ERG13 (FIG. 3K) showed the highest geraniol production when the expression of ERG19 was low and ERG13 was high. In the same plot, we also see relatively high predicted readout values when the expression of ERG19 was high and ERG13 was moderate. This reverse balance is likely because when Erg13p is expressed highly, Erg12p might be feedback inhibited due to the increased intermediates downstream of Erg19p (57-60), and lower expression of ERG19 would be more desirable. However, when Erg13p is expressed low, Erg19p must have a higher expression to maximize the pathway productivity since it catalyzes the irreversible step, which releases CO₂ to produce IPP. The rest of the two-enzyme interaction plots are similar to ERG10 and ERG8 interactions (FIG. 3L), where expression of both enzymes led to the highest amount of product, as expected, and are included in FIGS. 13A-13D.

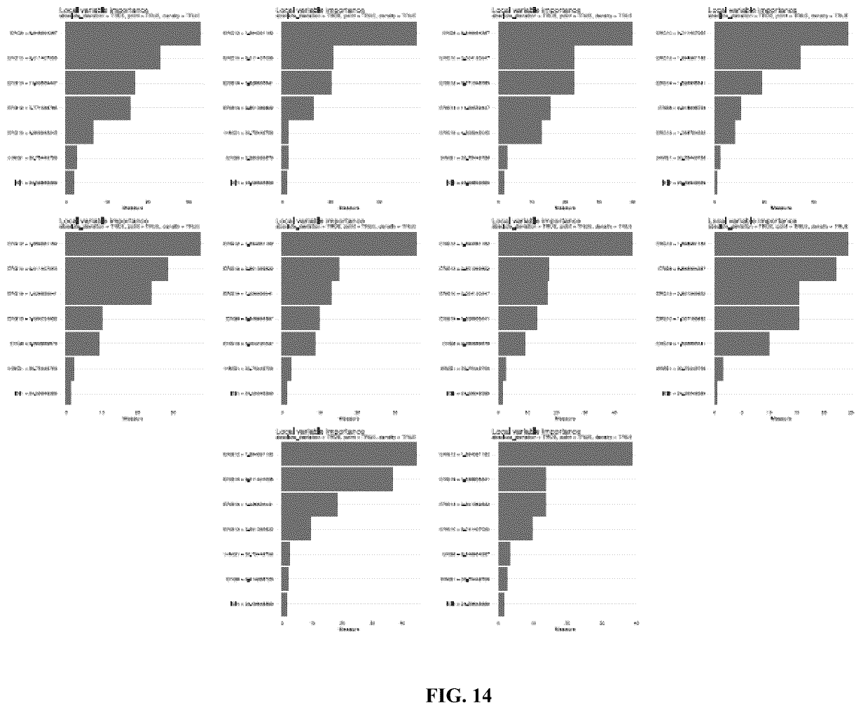

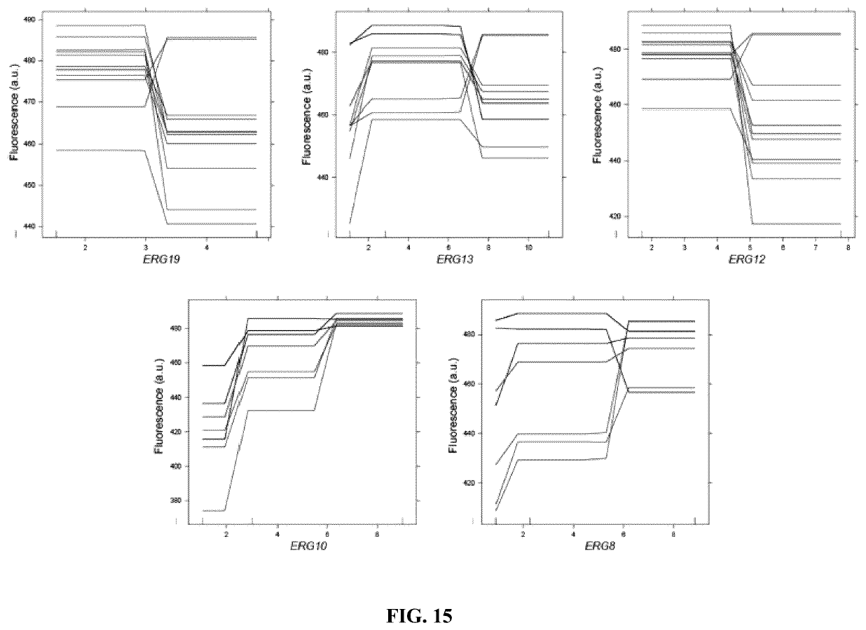

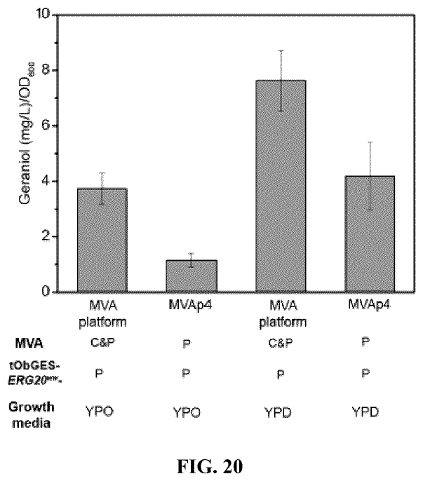

While the global analysis, including data from the entire combinatorial library, provides information in the prediction of geraniol output, the local analysis focuses on the top ten producers. Through the examination of the enzyme profiles and their variable importance of the ten highest geraniol-producing strains, we can gain insights into the role of the individual enzymes in the prediction of high geraniol levels. The local importance of pathway enzymes in the top ten strains supplements the PDP plots and shows a clear pattern where Erg12p comes out as the most important enzyme in seven out of ten strains (Table 2, FIG. 14). In Table 2, there are two instances of ERG12's expression as high (promoter strength=7.77). In both cases, the expression of ERG8, ERG13, and ERG19 is also high. This is also supported in the Individual Conditional Expectation (ICE) curves (49) (FIG. 20), which show that if ERG12's expression is high, other pathway enzymes' expression has to be also high to maximize geraniol production. In the top ten geraniol-producing strains, eight have ERG12 expressed at a moderately low range (promoter strength=1.69), which we found to be a ‘sweet spot.’ When ERG12 is expressed moderately, there are a variety of scenarios that can arise to produce a high amount of geraniol. Indeed, within the eight strains having ERG12 expressed in a moderately low range, seven have Erg12p as the most important enzyme for determining final productivity (Table 2, FIG. 14). In addition, Erg19p has consistently moderate low abundance across the top ten strains when Erg12p is in the sweet spot. Taken together, Erg12p is clearly the most critical enzyme for maximum geraniol production out of the five non-rate-limiting enzymes.

TABLE 2
Top ten strains with the highest level of geraniol. The numbers under each enzyme are
the relative promoter strengths quantified by Lee, et. al (39).

| | | | | | | | Critical |
|---|---|---|---|---|---|---|---|
| Strains | ERG10 | ERG13 | ERG12 | ERG8 | ERG19 | Geraniol (a.u.) | enzymes |
| α1 | 9.01 | 11.01 | 7.77 | 8.85 | 4.81 | 518.85 ± 0.54 | Erg8p |
| β2 | 9.01 | 2.85 | 1.69 | 2.28 | 1.53 | 517.94 ± 13.96 | Erg12p |
| α4 | 3.00 | 11.01 | 7.77 | 8.85 | 4.81 | 516.19 ± 87.54 | Erg8p |
| N3 | 9.01 | 1.06 | 1.69 | 0.91 | 1.53 | 513.53 ± 42.87 | Erg10p |
| N2 | 9.01 | 1.06 | 1.69 | 2.28 | 1.53 | 510.49 ± 11.46 | Erg12p |
| β4 | 3.00 | 2.85 | 1.69 | 8.85 | 1.53 | 509.51 ± 21.59 | Erg12p |
| β5 | 3.00 | 2.85 | 1.69 | 2.28 | 1.53 | 505.28 ± 10.16 | Erg12p |
| β7 | 1.06 | 2.85 | 1.69 | 8.85 | 1.53 | 502.44 ± 15.87 | Erg12p |
| β3 | 9.01 | 2.85 | 1.69 | 0.91 | 1.53 | 502.34 ± 12.10 | Erg12p |
| β1 | 9.01 | 2.85 | 1.69 | 8.85 | 1.53 | 501.19 ± 1.77 | Erg12p |

These local and global measures of variable importance provide complementary information. While the global analysis focuses overall on the variables that are important for predicting readouts of all ranges, the local importance allows us to zoom in on the patterns that give rise to high geraniol production. Not surprisingly, they tell somewhat different stories. Although ranked third in global variable importance, Erg12p is the control point that limits production in the entire pathway and is the most important enzyme when it comes to maximization of geraniol production. The prominent role of Erg12p is likely due to feedback regulations by pathway intermediates (61-64), reduced protein expression, or protein aggregation.

### 3.4 Dual Localization of the MVA Pathway to Both the Cytosol and Peroxisomes:

To further increase geraniol production, we localized the MVA pathway into both the cytosol and peroxisomes. Peroxisomes are an excellent choice for metabolic compartmentalization as they are not essential for cell survival (65). Additionally, fatty acid β-oxidation inside peroxisomes generates a pool of acetyl-CoA, which is the substrate for the MVA pathway (66). A haploid peroxisome strain (MVAp4) was generated by tagging all seven MVA genes with a C-terminal-SKL tripeptide. Similar to the MVAc4 strain, the MVAp4 strain has seven MVA genes integrated into the genome.

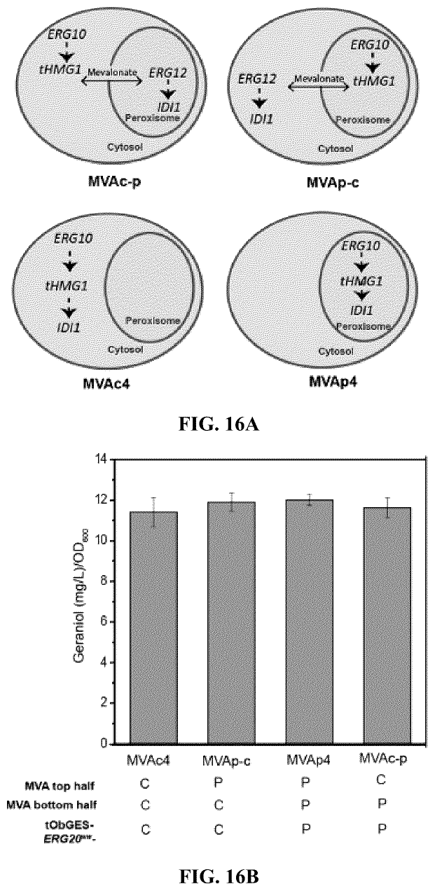

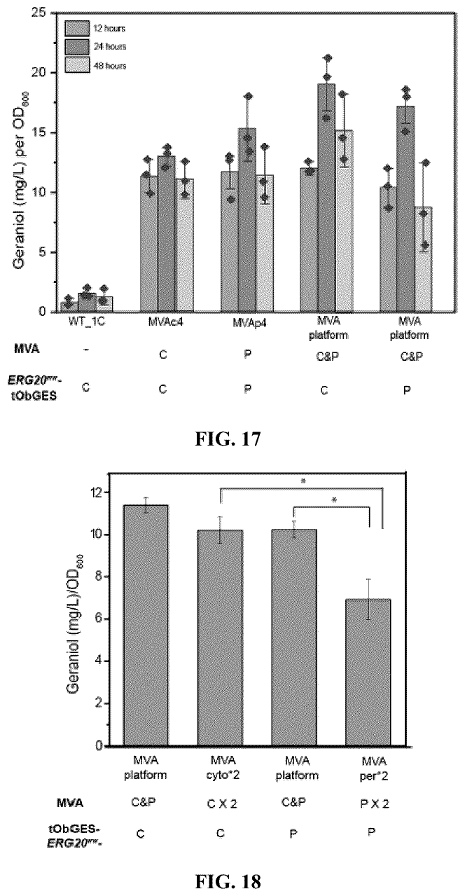

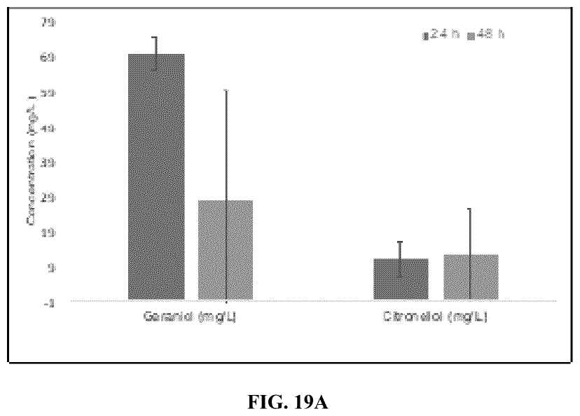

Next, MVAc4 and MVAp4 strains were mated to obtain a diploid strain, creating the MVA platform strain (FIG. 4A). The growth curves of the strains showed that the engineered strains had no growth defect and, in fact, grew significantly faster than the wild-type strains in rich media (FIG. 4B). When transformed with a plasmid bearing tObGES-ERG20^{ww}, the MVA platform strain doubled geraniol titers compared to the haploid strains, indicating that the dual targeting of the MVA pathway significantly increased geraniol production (FIG. 4C). We also generated two control strains, MVA cyto*2 and MVA per*2, in which two copies of the entire MVA pathway were targeted to either the cytosol or peroxisomes (FIG. 18). The MVA platform strain produced comparable amount of geraniol as the MVA cyto*2 strain but higher amount than the MVA per*2 strain. This could be due to the insufficient NADPH inside peroxisomes that limited the MVA pathway productivity. There was no difference in geraniol titers between the strains expressing the MVA pathway in the cytosol (MVAc4) and peroxisomes (MVAp4) (FIGS. 4C, D). Similar results were observed when the same strains were cultured in minimal media (FIG. 17). Expressing the tObGES-ERG20^{ww} in the peroxisome of the cytosolic strain MVAc4 showed only a small drop in geraniol titer compared to the strain with both the fusion protein and the additional MVA pathway localized to the cytosol. Furthermore, when localizing the tObGES-ERG20^{ww} into the cytosol of the peroxisomal strain MVAp4, there was no significant drop in geraniol titer compared to the strain with the fusion protein and the additional MVA pathway localized to the peroxisome. These data indicate that the IPP/DMAPP may diffuse somewhat freely between the cytosol and the peroxisome. To check if the pathway intermediate, mevalonate, is diffusible, two more strains, MVAp-c and MVAc-p were constructed. MVAp-c has the top half of the pathway, from ERG10 to tHMG1, localized to the peroxisome, and the bottom half of the pathway, from ERG12 to IDI1, in the cytosol. Conversely, MVAc-p has the top half of the pathway localized to the cytosol and the bottom half of the pathway in the peroxisome (FIGS. 16A and 16B). There was no difference in geraniol titer among the strains MVAc4 and MVAp-c or MVAp4 and MVAc-p; thus, mevalonate diffuses readily between the cytosol and peroxisome.

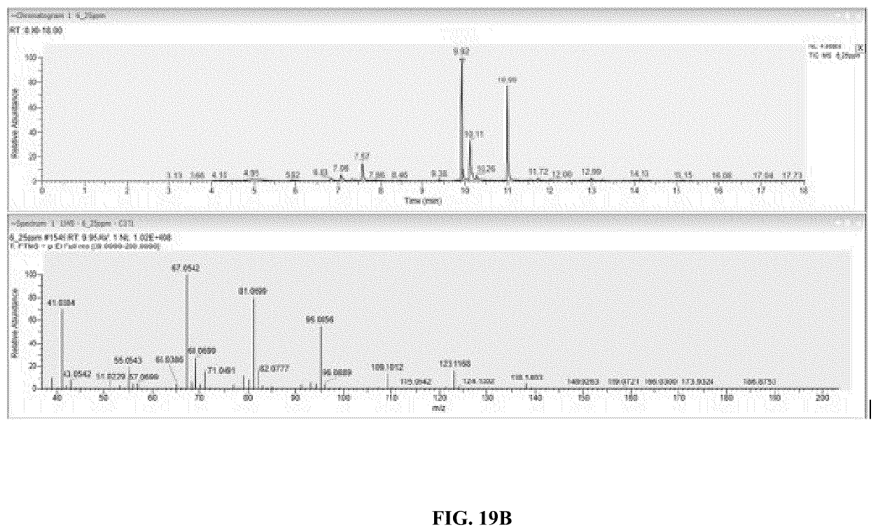

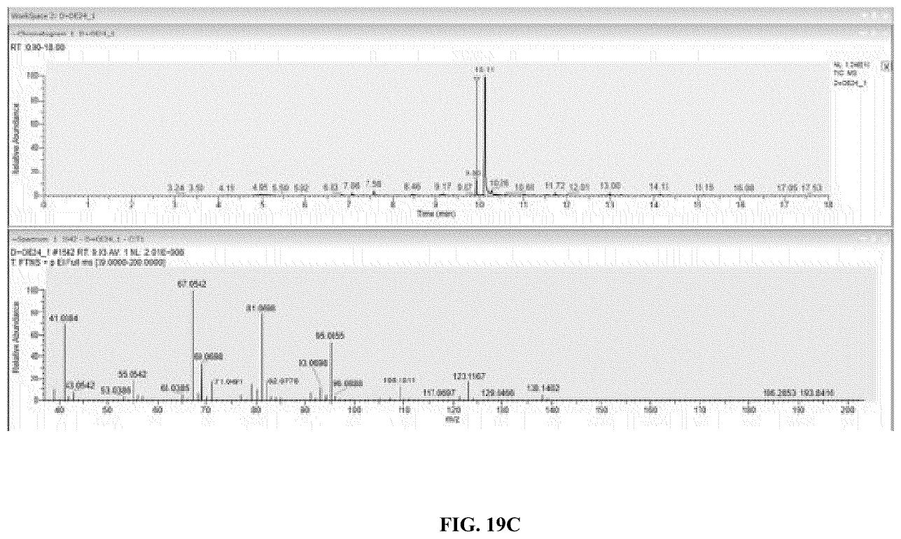

The growth of the engineered strains showed an inversed relationship with geraniol titer, possibly caused by geraniol toxicity to yeast at higher concentrations (67). When normalized by OD₆₀₀, there is an over two-fold increase in geraniol production in the MVA platform strain compared to the haploids (FIG. 4D). When extending the culturing time from 24 to 48 hours, geraniol production decreased significantly (FIGS. 19A-19C). The decrease in geraniol titer could be due to the compound's volatility or the reduced expression of the heterologous MVA pathway genes when glucose has been exhausted during the stationary phase (68). We also detected a minor product, citronellol, which is reduced from geraniol by yeast's native enzymes (FIGS. 19A-19C), whereas another common geraniol derivative, geraniol acetate, was not detected. In an attempt to increase geraniol production, MVAp4 and MVA platform strains were grown in a fatty-acid-based media (YPO) (69). However, the geraniol production in YPO decreased 2-fold compared to the productivity in YPD (FIG. 20). This was likely due to the low activity of promoters for expressing MVA genes in fatty-acid-based media since most of these promoters are from the glycolysis pathway.

3.5 Producing Diverse Terpenes from the MVA Platform Strain

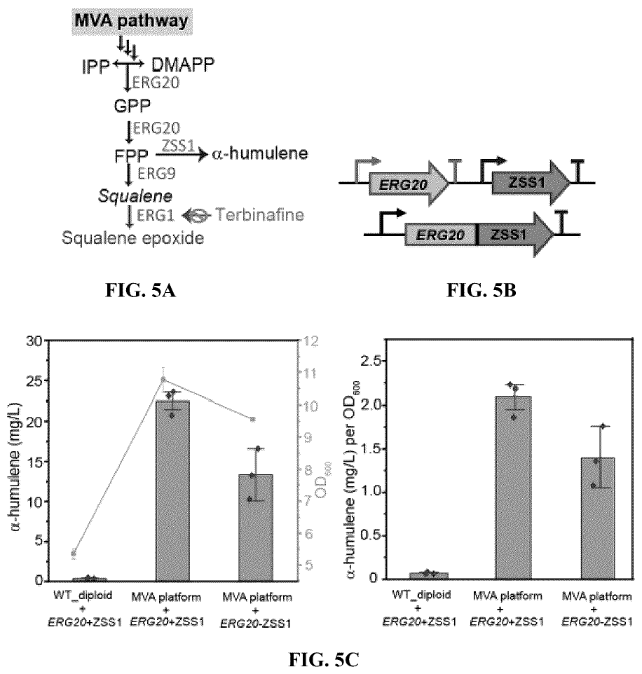

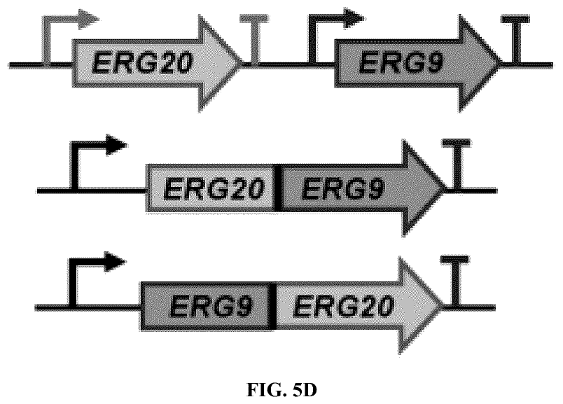

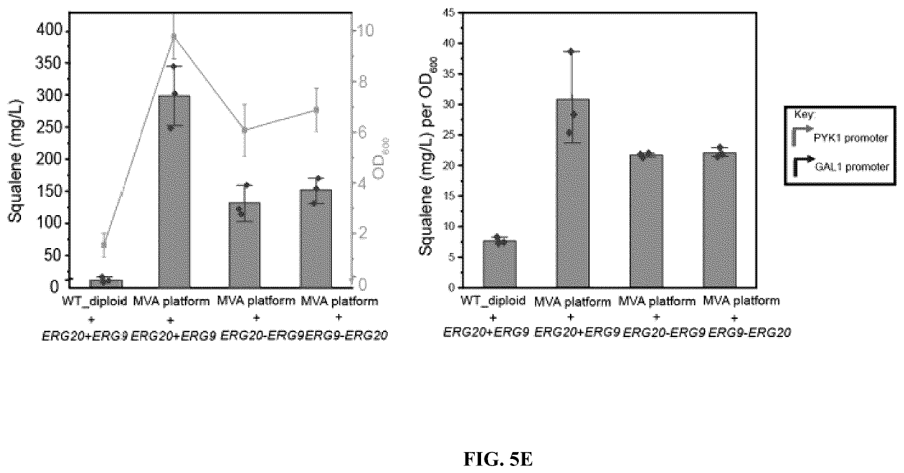

The MVA platform strain can be conveniently leveraged to jumpstart the production of a wide range of terpenes since the users only need to transform a plasmid with the desired prenyltransferase and terpene synthase. To demonstrate the versatility of the MVA platform strain, we next utilized it to produce a sesquiterpene α-humulene and a triterpene, squalene, in addition to the monoterpene geraniol. α-humulene has potential anti-inflammatory properties and acts as a precursor for the anti-cancer drug zerumbone (70, 71), while squalene is used as an emollient in personal care products due to its skin-compatible properties (72). For α-humulene production, the MVA platform strain transformed with a plasmid having ERG20 encoding the FPP synthase and ZSS1 encoding an α-humulene synthase from Zingiber zerumbet (73) produced ˜60-fold more α-humulene than the wild type in 24 hours (FIG. 5A-C). Fusion constructs with ERG20-ZSS1 produced about half of the amount compared with the non-fused counterpart, indicating that the fused enzymes have unfavorable conformational properties. OD₆₀₀ increased with the increase of α-humulene, which is likely due to a parallel increase in squalene, the precursor for ergosterol (13). For squalene production, the MVA platform strain was transformed with a plasmid having ERG20 and ERG9 encoding a squalene synthase. The resulting strain yielded ˜35-fold more squalene than the wild type when grown in the presence of terbinafine, an anti-fungal agent that inhibits Erg1p, which metabolizes squalene to 2,3-oxidosqualene (74) (FIG. 5A, D&E). Fusion constructs of ERG20 and ERG9 produced approximately half the amount of squalene, potentially due to unfavorable protein conformation. The growth of these strains was positively correlated with the amount of squalene produced since squalene is the substrate for ergosterol biosynthesis.

This disclosure provides an analysis of the contribution of individual enzymes to the MVA pathway, which is widely utilized to improve titers of terpenes. Previous studies have highlighted the importance of tHMG1 and IDI1 as rate-limiting enzymes (17-21, 56); however, there is a lack of consensus about the role of the other five enzymes in the pathway (22-29, 57, 58, 62, 64, 75). To clarify the importance of non-rate-limiting enzymes in the MVA pathway, we created a combinatorial yeast library for a comprehensive exploration of the promoter space of each of the five enzymes. Machine learning-guided modeling quantitatively revealed the contribution of each enzyme to product titer and found Erg19, Erg13, and Erg12p as crucial enzymes in determining product yield. The importance of each enzyme in a given pathway cannot be inferred from the Gibbs free energy (ΔG) of the reaction it catalyzes since enzymes act by decreasing the activation energy necessary for reactions to proceed but do not change the overall ΔG of the reactions (76). While monoterpene geraniol was employed as a readout of the MVA pathway, the modeling results are extendable to terpenes with longer chain lengths because all these terpenes require IPP:DMAPP ratio equal or above one, whereas the product ratio of IDI1 at equilibrium is IPP:DMAPP=1:2.2 (77).

We identified the medium expression of Erg12p as the ‘sweet spot’ for optimal terpene yield. A feedback-resistant mevalonate kinase from archaea (59, 60) may be used instead of the native enzyme for further enhancement of the pathway productivity. Further, our analysis of the top ten geraniol-producing strains (Table 2) shows that the strongest combination, α1, expressing all seven MVA pathway genes under strong promoters, indeed maximizes geraniol production, but several pathway genes can be expressed with relatively weaker promoters without significantly reducing the product titer. Seven out of the top ten producers having at least four genes expressed from medium or weak promoters produced comparable geraniol titer as the top strain α1. These conclusions may only apply to the MVA pathway during the exponential phase of growth.

The dual localization of the MVA pathway to both the cytosol and peroxisomes significantly increased geraniol titers (FIG. 4), most likely due to the high abundance of acetyl-CoA and NADPH in the peroxisomes and cytosol, respectively. Interestingly, targeting the MVA pathway into the peroxisome but the prenyltransferase and terpene synthase into the cytosol yielded similar amounts of geraniol. The same observations were made when switching the localization of the overexpressed MVA pathway and the prenyl transferase and geraniol synthase. These results indicate that IPP/DMAPP are diffusible across the peroxisome membrane. Similarly, we've constructed strains MVAc-p and MVAp-c to show that mevalonate can diffuse readily across peroxisome membranes (FIGS. 16A and 16B). Since peroxisome has a single-layer membrane, small molecules can travel across either passively or facilitated by transporters (78). Furthermore, multiple MVA enzymes have been reported to be localized in peroxisomes of plants and animals (79-82), which also supports the diffusion of MVA intermediates between peroxisomes and cytosol. The faster growth of the engineered strains with the MVA pathway overexpressed is likely due to the increased demand for acetyl-CoA, ATP, and NADPH, which results in the accelerated turnover of sugar, lipids, and amino acids in the rich media.

We used the dual localization strategy to create a platform strain as a starting point for the production of terpenes. Although plasmid-based expression for peroxisomal localized genes resulted in a much higher monoterpene production (66), we focused on genomic integration. Users only need to transfer a plasmid carrying the particular prenyltransferase and terpene synthase into the platform strain for the production of target terpenes. To demonstrate the versatility of our platform strain, we used it to produce geraniol, α-humulene, and squalene as representatives of the three classes of terpenes: mono-, sesqui-, and triterpenes. The highest titer in shaking flask culture reported so far for geraniol, α-humulene, and squalene are 523.96 mg/L (19), 160 mg/L (15), and 1.3 g/L (14), respectively. These titers were achieved by introducing compound-specific genetic modifications and optimizing culturing conditions. We did not introduce any additional compound-specific genomic modifications in the platform strain since such modifications will narrow the product scope of the platform, but such modifications are not necessarily excluded from the disclosure. The disclosure includes additional compound-specific genomic modifications to increase the titers of a particular terpene. For example, genes such as ATF1 and OYE2 may be deleted to increase geraniol titer by preventing its metabolism (53). For increasing α-humulene and squalene production, genes encoding non-specific phosphatases such as LPP1 and DPP1 (83-85) may be deleted to prevent the divergence of farnesyl pyrophosphate (FPP) to farnesol. Expressing ERG9 from a weak promoter (71) or tagging it for degradation (15) can lead to higher α-humulene accumulation. Expressing ERG1 under a weak promoter (14) can improve the production of squalene.

### 4.1 Conclusions:

This study elucidated the detailed contribution of the five non-rate-limiting enzymes of the MVA pathway in S. cerevisiae by creating a combinatorial yeast library. Analysis using machine learning algorithms revealed the critical role of Erg12p in determining MVA pathway productivity. A platform strain with dual localization of the MVA pathway into both the cytosol and peroxisomes was created. This strain can be leveraged to produce diverse terpenes. The disclosure regarding the contribution of individual MVA pathway enzymes and the MVA yeast platform created will provides for engineering to produce high titers of any terpene.

### REFERENCES

1. D. W. Christianson, Structural and chemical biology of terpene cyclases. Chem Rev 117, 11570-11648 (2017).
2. M. S. Belcher, J. Mahinthakumar, J. D. Keasling, New frontiers: harnessing pivotal advances in microbial engineering for the biosynthesis of plant-derived terpenes. Curr Opin Biotechnol 65, 88-93 (2020).
3. D. K. Ro et al., Production of the antimalarial drug precursor artemisinic acid in engineered yeast. Nature 440, 940-943 (2006).
4. B. Engels, P. Dahm, S. Jennewein, Metabolic engineering of taxadiene biosynthesis in yeast as a first step towards taxol (paclitaxel) production. Metab Eng 10, 201-206 (2008).
5. G. R. Navale, M. S. Dharne, S. S. Shinde, Metabolic engineering and synthetic biology for isoprenoid production in Escherichia coli and Saccharomyces cerevisiae. Appl Microbiol Biotechnol 105, 457-475 (2021).
6. X. J. Guo et al., Metabolic engineering of Saccharomyces cerevisiae for 7-dehydrocholesterol overproduction. Biotechnol Biofuels 11, 192 (2018).
7. J. Yuan, C. B. Ching, Combinatorial engineering of mevalonate pathway for improved amorpha-4,11-diene production in budding yeast. Biotechnol Bioeng 111, 608-617 (2014).
8. D. A. Yee et al., Engineered mitochondrial production of monoterpenes in Saccharomyces cerevisiae. Metab Eng 55, 76-84 (2019).
9. X. Lv et al., Dual regulation of cytoplasmic and mitochondrial acetyl-CoA utilization for improved isoprene production in Saccharomyces cerevisiae. Nat Commun 7, 12851 (2016).
10. L. Jiang et al., Improved functional expression of cytochrome P450s in Saccharomyces cerevisiae through screening a cDNA library from Arabidopsis thaliana. Front Bioeng Biotechnol 9, 764851 (2021).
11. P. J. Westfall et al., Production of amorphadiene in yeast, and its conversion to dihydroartemisinic acid, precursor to the antimalarial agent artemisinin. Proc Natl Acad Sci USA 109, E111-118 (2012).
12. B. Peng et al., A squalene synthase protein degradation method for improved sesquiterpene production in Saccharomyces cerevisiae. Metab Eng 39, 209-219 (2017).
13. T. Li et al., Metabolic Engineering of Saccharomyces cerevisiae to overproduce squalene. J Agric Food Chem 68, 2132-2138 (2020).
14. G. S. Liu et al., The yeast peroxisome: A dynamic storage depot and subcellular factory for squalene overproduction. Metab Eng 57, 151-161 (2020).
15. C. Zhang, M. Li, G. R. Zhao, W. Lu, Harnessing yeast peroxisomes and cytosol acetyl-Coa for sesquiterpene alpha-humulene production. J Agric Food Chem 68, 1382-1389 (2020).
16. H. M. Sauro, Control and regulation of pathways via negative feedback. J R Soc Interface 14 (2017).
17. J. Y. Han, S. H. Seo, J. M. Song, H. Lee, E. S. Choi, High-level recombinant production of squalene using selected Saccharomyces cerevisiae strains. J Ind Microbiol Biotechnol 45, 239-251 (2018).
18. J. Zhao et al., Dynamic control of ERG20 expression combined with minimized endogenous downstream metabolism contributes to the improvement of geraniol production in Saccharomyces cerevisiae. Microb Cell Fact 16, 17 (2017).
19. G. Z. Jiang et al., Manipulation of GES and ERG20 for geraniol overproduction in Saccharomyces cerevisiae. Metab Eng 41, 57-66 (2017).
20. W. Xie, X. Lv, L. Ye, P. Zhou, H. Yu, Construction of lycopene-overproducing Saccharomyces cerevisiae by combining directed evolution and metabolic engineering. Metab Eng 30, 69-78 (2015).
21. R. Verwaal et al., High-level production of beta-carotene in Saccharomyces cerevisiae by successive transformation with carotenogenic genes from Xanthophyllomyces dendrorhous. Appl Environ Microbiol 73, 4342-4350 (2007).
22. S. Kwak et al., Redirection of the glycolytic flux enhances isoprenoid production in Saccharomyces cerevisiae. Biotechnol J 15, e1900173 (2020).
23. P. Zhou et al., Crystal structure of cytoplasmic acetoacetyl-CoA thiolase from Saccharomyces cerevisiae. Acta Crystallogr F Struct Biol Commun 74, 6-13 (2018).
24. J. McClory, J. T. Lin, D. J. Timson, J. Zhang, M. Huang, Catalytic mechanism of mevalonate kinase revisited, a QM/MM study. Org Biomol Chem 17, 2423-2431 (2019).
25. Z. Hu et al., Improve the production of D-limonene by regulating the mevalonate pathway of Saccharomyces cerevisiae during alcoholic beverage fermentation. J Ind Microbiol Biotechnol 47, 1083-1097 (2020).
26. K. M. Madsen et al., Linking genotype and phenotype of Saccharomyces cerevisiae strains reveals metabolic engineering targets and leads to triterpene hyper-producers. PLoS One 6, e14763 (2011).
27. Z. Yao et al., Enhanced isoprene production by reconstruction of metabolic balance between strengthened precursor supply and improved isoprene synthase in Saccharomyces cerevisiae. ACS Synth Biol 7, 2308-2316 (2018).
28. A. M. Redding-Johanson et al., Targeted proteomics for metabolic pathway optimization: application to terpene production. Metab Eng 13, 194-203 (2011).
29. J. Alonso-Gutierrez et al., Principal component analysis of proteomics (PCAP) as a tool to direct metabolic engineering. Metab Eng 28, 123-133 (2015).
30. J. Nielsen, Bioengineering. Yeast cell factories on the horizon. Science 349, 1050-1051 (2015).
31. Y. Chen, L. Daviet, M. Schalk, V. Siewers, J. Nielsen, Establishing a platform cell factory through engineering of yeast acetyl-CoA metabolism. Metab Eng 15, 48-54 (2013).
32. A. Rodriguez, K. R. Kildegaard, M. Li, I. Borodina, J. Nielsen, Establishment of a yeast platform strain for production of p-coumaric acid through metabolic engineering of aromatic amino acid biosynthesis. Metab Eng 31, 181-188 (2015).
33. N. D. Gold et al., Metabolic engineering of a tyrosine-overproducing yeast platform using targeted metabolomics. Microb Cell Fact 14, 73 (2015).
34. A. Campbell et al., Engineering of a nepetalactol-producing platform strain of Saccharomyces cerevisiae for the production of plant seco-iridoids. ACS Synth Biol 5, 405-414 (2016).
35. M. E. Pyne et al., A yeast platform for high-level synthesis of tetrahydroisoquinoline alkaloids. Nat Commun 11, 3337 (2020).
36. C. E. Vickers, S. F. Bydder, Y. Zhou, L. K. Nielsen, Dual gene expression cassette vectors with antibiotic selection markers for engineering in Saccharomyces cerevisiae. Microb Cell Fact 12, 96 (2013).
37. D. G. Gibson, Enzymatic assembly of overlapping DNA fragments. Methods Enzymol 498, 349-361 (2011).
38. M. Mukherjee, E. Caroll, Z. Q. Wang, Rapid assembly of multi-gene constructs using modular Golden Gate cloning. J Vis Exp 168, e61993 (2021).
39. M. E. Lee, W. C. DeLoache, B. Cervantes, J. E. Dueber, A highly characterized yeast toolkit for modular, multipart assembly. ACS Synth Biol 4, 975-986 (2015).
40. O. W. Ryan et al., Selection of chromosomal DNA libraries using a multiplex CRISPR system. Elife 3, e03703 (2014).
41. J.-L. Lin, H. Ekas, K. Markham, H. S. Alper, An enzyme-coupled assay enables rapid protein engineering for geraniol production in yeast. Biochemical Engineering Journal 139, 95-100 (2018).
42. L. Breiman, Random Forests. Machine Learning 45, 5-32 (2001).
43. L. Breiman, J. H. Friedman, R. A. Olshen, C. J. Stone, Classification and regression trees (Routledge, 2017).
44. L. Breiman, Out-of-bag estimation. (1996).
45. B. Efron, R. LePage, Introduction to bootstrap (Wiley & Sons, New York, 1992).
46. H. T. Friedman J, Tibshirani R, The elements of statistical learning (Springer series in statistics New York, 2001).
47. D. R. Cutler et al., Random forests for classification in ecology. Ecology 88, 2783-2792 (2007).
48. B. M. Greenwell, pdp: an R Package for constructing partial dependence plots. R J. 9, 421 (2017).
49. A. Goldstein, A. Kapelner, J. Bleich, E. Pitkin, Peeking inside the black box: Visualizing statistical learning with plots of individual conditional expectation. J Comput Graph Stat 24, 44-65 (2015).
50. F. A. Trikka et al., Iterative carotenogenic screens identify combinations of yeast gene deletions that enhance sclareol production. Microb Cell Fact 14, 60 (2015).
51. T. L. Orr-Weaver, J. W. Szostak, R. J. Rothstein, Yeast transformation: a model system for the study of recombination. Proc Natl Acad Sci USA 78, 6354-6358 (1981).
52. W. Chen, A. M. Viljoen, Geraniol A review of a commercially important fragrance material. S Afr J Bot 76, 643-651 (2010).
53. S. Brown, M. Clastre, V. Courdavault, S. E. O'Connor, De novo production of the plant-derived alkaloid strictosidine in yeast. Proc Natl Acad Sci USA 112, 3205-3210 (2015).
54. X. Wang et al., Engineering Escherichia coli for production of geraniol by systematic synthetic biology approaches and laboratory-evolved fusion tags. Metab Eng 66, 60-67 (2021).
55. C. Ignea, M. Pontini, M. E. Maffei, A. M. Makris, S. C. Kampranis, Engineering monoterpene production in yeast using a synthetic dominant negative geranyl diphosphate synthase. ACS Synth Biol 3, 298-306 (2014).
56. Y. J. Zhou et al., Modular pathway engineering of diterpene synthases and the mevalonic acid pathway for miltiradiene production. J Am Chem Soc 134, 3234-3241 (2012).
57. J. R. Anthony et al., Optimization of the mevalonate-based isoprenoid biosynthetic pathway in Escherichia coli for production of the anti-malarial drug precursor amorpha-4,11-diene. Metab Eng 11, 13-19 (2009).
58. D. E. Garcia, J. D. Keasling, Kinetics of phosphomevalonate kinase from Saccharomyces cerevisiae. PLoS One 9, e87112 (2014).
59. Y. A. Primak et al., Characterization of a feedback-resistant mevalonate kinase from the archaeon Methanosarcina mazei. Appl Environ Microbiol 77, 7772-7778 (2011).
60. E. Kazieva et al., Characterization of feedback-resistant mevalonate kinases from the methanogenic archaeons Methanosaeta concilii and Methanocella paludicola. Microbiology (Reading) 163, 1283-1291 (2017).
61. D. D. Hinson, K. L. Chambliss, M. J. Toth, R. D. Tanaka, K. M. Gibson, Post-translational regulation of mevalonate kinase by intermediates of the cholesterol and nonsterol isoprene biosynthetic pathways. J Lipid Res 38, 2216-2223 (1997).
62. H. Chen et al., Directed evolution of mevalonate kinase in Escherichia coli by random mutagenesis for improved lycopene. RSC Advances 8, 15021-15028 (2018).
63. Z. Fu, N. E. Voynova, T. J. Herdendorf, H. M. Miziorko, J. J. Kim, Biochemical and structural basis for feedback inhibition of mevalonate kinase and isoprenoid metabolism. Biochemistry 47, 3715-3724 (2008).
64. S. M. Ma et al., Optimization of a heterologous mevalonate pathway through the use of variant HMG-CoA reductases. Metab Eng 13, 588-597 (2011).
65. A. A. Sibirny, Yeast peroxisomes: structure, functions and biotechnological opportunities. FEMS Yeast Res 16 (2016).
66. S. Dusseaux, W. T. Wajn, Y. Liu, C. Ignea, S. C. Kampranis, Transforming yeast peroxisomes into microfactories for the efficient production of high-value isoprenoids. Proc Natl Acad Sci USA 117, 31789-31799 (2020).
67. C. M. Denby et al., Industrial brewing yeast engineered for the production of primary flavor determinants in hopped beer. Nat Commun 9, 965 (2018).
68. B. Peng, T. C. Williams, M. Henry, L. K. Nielsen, C. E. Vickers, Controlling heterologous gene expression in yeast cell factories on different carbon substrates and across the diauxic shift: a comparison of yeast promoter activities. Microb Cell Fact 14, 91 (2015).
69. J. Gerke et al., Production of the fragrance geraniol in peroxisomes of a product-tolerant baker's yeast. Front Bioeng Biotechnol 8, 582052 (2020).
70. E. S. Fernandes et al., Anti-inflammatory effects of compounds alpha-humulene and (−)-trans-caryophyllene isolated from the essential oil of Cordia verbenacea. Eur J Pharmacol 569, 228-236 (2007).
71. C. Zhang et al., Production of sesquiterpene zerumbone from metabolic engineered Saccharomyces cerevisiae. Metab Eng 49, 28-35 (2018).
72. O. Popa, N. E. Babeanu, I. Popa, S. Nita, C. E. Dinu-Parvu, Methods for obtaining and determination of squalene from natural sources. Biomed Res Int 2015, 367202 (2015).
73. S. Alemdar et al., Heterologous expression, purification, and biochemical characterization of alpha-Humulene Synthase from Zingiber zerumbet Smith. Appl Biochem Biotechnol 178, 474-489 (2016).
74. M. Garaiova, V. Zambojova, Z. Simova, P. Griac, I. Hapala, Squalene epoxidase as a target for manipulation of squalene levels in the yeast Saccharomyces cerevisiae. FEMS Yeast Res 14, 310-323 (2014).
75. F. Pojer et al., Structural basis for the design of potent and species-specific inhibitors of 3-hydroxy-3-methylglutaryl CoA synthases. Proc Natl Acad Sci USA 103, 11491-11496 (2006).
76. D. L. Nelson, & Cox, M. M, Lehninger principles of biochemistry (2004).
77. I. P. Street, D. J. Christensen, C. D. Poulter, Hydrogen exchange during the enzyme-catalyzed isomerization of isopentenyl diphosphate and dimethylallyl diphosphate. J Am Chem Soc 112, 8577-8578 (1990).
78. V. D. Antonenkov, S. Mindthoff, S. Grunau, R. Erdmann, J. K. Hiltunen, An involvement of yeast peroxisomal channels in transmembrane transfer of glyoxylate cycle intermediates. Int J Biochem Cell Biol 41, 2546-2554 (2009).
79. G. Guirimand et al., A single gene encodes isopentenyl diphosphate isomerase isoforms targeted to plastids, mitochondria and peroxisomes in Catharanthus roseus. Plant Mol Biol 79, 443-459 (2012).
80. A. J. Simkin et al., Peroxisomal localisation of the final steps of the mevalonic acid pathway in planta. Planta 234, 903-914 (2011).
81. R. Breitling, S. K. Krisans, A second gene for peroxisomal HMG-CoA reductase? A genomic reassessment. J Lipid Res 43, 2031-2036 (2002).
82. M. Sapir-Mir et al., Peroxisomal localization of Arabidopsis isopentenyl diphosphate isomerases suggests that part of the plant isoprenoid mevalonic acid pathway is compartmentalized to peroxisomes. Plant Physiol 148, 1219-1228 (2008).
83. A. Faulkner et al., The LPP1 and DPP1 gene products account for most of the isoprenoid phosphate phosphatase activities in Saccharomyces cerevisiae. J Biol Chem 274, 14831-14837 (1999).
84. L. Albertsen et al., Diversion of flux toward sesquiterpene production in Saccharomyces cerevisiae by fusion of host and heterologous enzymes. Appl Environ Microbiol 77, 1033-1040 (2011).
85. G. Scalcinati et al., Dynamic control of gene expression in Saccharomyces cerevisiae engineered for the production of plant sesquitepene alpha-santalene in a fed-batch mode. Metab Eng 14, 91-103 (2012).

### Example 5: Supplementary Material

Quantitative Real-Time PCR (qRT-PCR):

For RNA extraction, the wildtype strain CEN.PK2 and engineered strains α1, β5, and λ9 transformed with the pPYK001_tObGES-ERG20ww were grown overnight in 5 ml SD-His at 30° C. with shaking at 200 rpm. The overnight culture was inoculated at an initial OD600 of 0.1 into fresh SD-His and grown at 30° C. with shaking at 200 rpm for 12 hours. Total RNA extraction from all the yeast cultures was performed using the YeaStar RNA kit (ZymoResearch, Irvine, CA) as per the manufacturer's instructions. The RNA isolated was converted to cDNA using the iScript™ cDNA synthesis kit (BioRad, Hercules, CA) following the manufacturer's instructions. Primers for qRT-PCR analysis are in Table 10. The qRT-PCR reaction mix consisted of cDNA templates, primers, 2× Universal SYBR green fast qPCR mix (ABClonal, Woburn, MA), and double-distilled water with a final volume of 20 μL. The thermocycling conditions were: denaturation at 95° C. for 3 min, 40 cycles of denaturation at 95° C. for 10 sec, annealing at 55° C. for 30 sec, and extension at 68° C. for 50 secs. A final melting step from 55° C. to 95° C. in 0.5° C. increments for 81 cycles was used to generate melting curves. Three biological replicates and two technical replicates were used to measure each gene's expression. UBC6 was used as the internal reference.

### Geraniol Production in Glucose and Oleate Media:

MVAp4 and MVA platform strains were transformed with the pYTK001_tObGES-ERG20^{ww}-SKL and plated either on SD (0.2% glucose)+400 μg/ml G418 (pH=7) or SO (0.1% oleic acid)+400 μg/ml G418 (pH=7) plates. SD (0.2% glucose) contained 0.67% (w/v) yeast nitrogen base without amino acids, 0.2% (w/v) dextrose, and 0.07% (w/v) synthetic complete amino acid mix (CSM). SO (0.1% oleic acid) contained 0.67% (w/v) yeast nitrogen base without amino acids, 0.1% oleic acid, 0.3% Tween-80, 0.05% dextrose, and 0.07% (w/v) synthetic complete amino acid mix (CSM). Single colonies from each plate were inoculated in 5 ml of either SD+400 μg/ml G418 (pH=7) or SO+400 μg/ml G418 (pH=7) for seed culture preparation. The overnight seed culture was inoculated at an initial OD₆₀₀ of 0.1 into 25 ml of fresh YPD (0.2% glucose)+200 μg/ml G418 or YPO (0.1% oleic acid)+200 μg/ml G418 and grown at 30° C. with shaking at 200 rpm. YPD (0.2% glucose) contained 1% yeast extract, 2% peptone, and 0.2% dextrose whereas YPO (0.1% oleic acid) contained 1% yeast extract, 2% peptone, and 0.1% oleic acid. 0.2% Glucose and 0.1% oleic acid have the same number of carbon atoms. The cultures were grown for 24 hours in YPD (0.2% glucose) and for 72 hours in YPO (0.1% oleic acid). A longer growth period in YPO (0.1% oleic acid) was required because of the slower growth.

### Extraction and Quantification of Mevalonate by Liquid Chromatography-Mass Spectrometry (LC-MS):

The extraction method for MVA metabolites was modified from Kim et al., 2021 (1). Briefly, single colonies of the top ten geraniol producing (Table 2) and the all weak D 9 strains transformed with pPYK1-tObGES-ERG20^{ww} plasmid were inoculated in 5 ml SD-Leu-Ura-Trp-His broth for seed culture preparation. The overnight seed culture was inoculated at an initial OD₆₀₀ of 0.1 into 25 ml fresh SD-Leu-Ura-Trp-His broth and grown at 30° C. with shaking at 200 rpm for 12 hours. Cultures of OD₆₀₀=15 were pelleted, the supernatant discarded, and the pellet was dissolved in 650 μl water: chloroform: methanol (1:2:2). 500 mg glass beads were added, and the cells were disrupted in a Bullet Blender® tissue homogenizer at the highest setting for 10 mins at 4° C. The samples were then centrifuged at 14,000×g for 10 mins at 4° C. 300 μl of the aqueous phase was collected and dried using a SpeedVac™ (Thermo Scientific, Waltham, MA) at the high setting for 4.5 hours. The dried sample was resuspended in 300 μl of acetonitrile: methanol: water (6:1:3) for LC-MS analysis.

A BEH Z-HILIC HPLC column (Atlantis™ PREMIER, Waters, Milford, MA) (1.7 μm particle size, 2.1 mm i.d., 100 mm length) was used for separation on a Thermo Scientific Q-Exactive Focus™ Orbitrap with a 60% mobile phase A containing 10 mM ammonium carbonate and 118.4 mM ammonium hydroxide in acetonitrile:water (60:40) (2) and 40% mobile phase B containing acetonitrile for 8 min at a flow rate of 300 μl min⁻¹. The eluent was analyzed in the negative full-scan mode with an m/z range: 100-400, and mevalonate was detected at an m/z of 147.0668±5 ppm at 1.7 min. Absolute sample concentrations were calculated from a standard curve made from authentic (R)-mevalonic acid lithium salt (Sigma Aldrich, St Louis, MO) dissolved in acetonitrile:methanol:water (6:1:3). An m/z of 147.0668±5 ppm was used for quantitative analysis of mevalonate using the Xcalibur™ software.

TABLE 3
List of part plasmids generated in this study.

| Name | Description |
|---|---|
| pYTK001_ERG10 | ERG10 |
| pYTK001_ERG13 | ERG13 |
| pYTK001_tHMG1 | truncated HMG1 |
| pYTK001_ERG12 | ERG12 |
| pYTK001_ERG8 | ERG8 |
| pYTK001_ERG19 | ERG19 |
| pYTK001_IDI1 | IDI1 |
| pYTK001_tObGES-ERG20^{ww} | Fusion of the truncated |
| | tObGES and ERG20^{ww} |
| pYTK001_tCYC1 | CYC1 terminator |
| pYTK001_ROX1(5′Hom) | 5' homology arm for |
| | integration at the ROX1 locus |
| pYTK001_ROX1(3′Hom) | 3' homology arm for |
| | integration at the ROX1 locus |
| pYTK001_GAL1(5′Hom) | 5' homology arm for |
| | integration at the GAL1 locus |
| pYTK001_GAL1(3′Hom) | 3' homology arm for |
| | integration at the GAL1 locus |
| pYTK001_GAL80(5′Hom) | 5' homology arm for |
| | integration at the GAL80 locus |
| pYTK001_GAL80(3′Hom) | 3' homology arm for |
| | integration at the GAL80 locus |
| pYTK001_TRP1 | Yeast tryptophan selection marker |
| pYTK001_ERG10-SKL | ERG10 with SKL tripeptide |
| | at the C-terminus |
| pYTK001_ERG13-SKL | ERG13 with SKL tripeptide |
| | at the C-terminus |
| pYTK001_tHMG1-SKL | tHMG1 with SKL tripeptide |
| | at the C-terminus |
| pYTK001_ERG12-SKL | ERG12 with SKL tripeptide |
| | at the C-terminus |
| pYTK001_ERG8-SKL | ERG8 with SKL tripeptide |
| | at the C-terminus |
| pYTK001_ERG19-SKL | ERG19 with SKL tripeptide |
| | at the C-terminus |
| pYTK001_IDI1-SKL | IDI1 with SKL tripeptide |
| | at the C-terminus |
| pYTK001_tObGES- | tObGES-ERG20^{ww} fusion with |
| ERG20^{ww}-SKL | SKL tripeptide at the C-terminus |
| pYTK001_ERG20 | ERG20 |
| pYTK001_ZSS1 | ZSS1 |
| pYTK001_ERG20-ZSS1 | Fusion of ERG20 and ZSS1 |
| pYTK001_ERG9 | ERG9 |
| pYTK001_ERG20-ERG9 | Fusion of ERG20 and ERG9 |
| pYTK001_ERG9-ERG20 | Fusion of ERG9 and ERG20 |

TABLE 4
Numbering system of the transcription unit (TU) and
multi-gene (MG) plasmids used in this study.

| 1^{st} | | 2^{nd} | Yeast | 3^{rd} | Left | 4^{th} | Right |
|---|---|---|---|---|---|---|---|
| digit | ORI | digit | selection* | digit | Connector | digit | Connector |
| 1 | CEN | 1 | URA3 | S | ConLS | 1 | ConR1 |
| 2 | 2μ | 2 | LEU2 | 1 | ConL1 | 2 | ConR2 |
| | | 3 | HIS3 | 2 | ConL2 | 6 | ConRE |
| | | 4 | KanR | | | | |
| | | 5 | TRP1 | | | | |
| | | 6 | HygR | | | | |

*For integrative multi-gene plasmids, the first and only digit refers to the yeast selection marker since the integrative plasmids do not have yeast ORI or left and right connecters.

TABLE 5
List of intermediate TU vectors generated in this study.

| Name | Description |
|---|---|
| pTU11S1_ (Inter)_GFP | Intermediate vector for cloning the |
| Dropout | first TU in a multi-gene plasmid |
| pTU1116_(Inter)_GFP | Intermediate vector for cloning the second |
| Dropout | TU in a 2-gene multi-gene plasmid |
| pTU1112_(Inter)_GFP | Intermediate vector for cloning the |
| Dropout | second TU in a 3-gene multi-gene plasmid |
| pTU1126_(Inter)_GFP | Intermediate vector for cloning the |
| Dropout | third TU in a 3-gene multi-gene plasmid |
| pTU13S1_(Inter)_GFP | Low copy intermediate vector |
| Dropout | with HIS3 marker |
| | for cloning the downstream fusion genes |
| pTU23S1_(Inter)_GFP | High copy intermediate vector |
| Dropout | with HIS3 marker |
| | for cloning the downstream fusion genes |
| pTU24S1_(Inter)_GFP | High copy intermediate vector |
| Dropout | with KanR marker |
| | for cloning the downstream fusion genes |

pTU = transcription unit plasmid

TABLE 6
List of TU plasmids generated in this study.

| Name | Description (Promoter-CDS-Terminator) |
|---|---|
| pTU11S1_ERG10s | pHHF2-ERG10-tENO1 |
| pTU11S1_ERG10m | pRPL18B-ERG10-tENO1 |
| pTU11S1_ERG10w | pPOP6-ERG10-tENO1 |
| pTU1116_tHMG1 | pTDH3-tHMG1-tTDH1 |
| pTU11S1_ERG8s | pTEF1-ERG8-tSSA1 |
| pTU11S1_ERG8m | pALD6-ERG8-tSSA1 |
| pTU11S1_ERG8w | pRAD27-ERG8-tSSA1 |
| pTU1116_IDI1 | pCCW12-IDI1-tENO2 |
| pTU11S1_ERG13s | pPGK1-ERG13-tPGK1 |
| pTU11S1_ERG13m | pHTB2-ERG13-tPGK1 |
| pTU11S1_ERG13m | pHTB2-ERG13-tPGK1 |
| pTU11S1_ERG13w | pRNR2-ERG13-tPGK1 |
| pTU1112_ERG12s | pTEF2-ERG12-tADH1 |
| pTU1112_ERG12m | pPAB1-ERG12-tADH1 |
| pTU1112_ERG12w | pPSP2-ERG12-tADH1 |
| pTU1126_ERG19s | pHHF-ERG19-tCYC1 |
| pTU1126_ERG19m | pRET2-ERG19-tCYC1 |
| pTU1126_ERG19w | pREV1-ERG19-tCYC1 |
| pTU13S1_tObGES-ERG20^{ww}1 | pENO1-tObGES-ERG20^{ww}-tTDH2 |
| pTU23S1_tObGES-ERG20^{ww}1 | pENO1-tObGES-ERG20^{ww}-tTDH2 |
| pTU23S1_tObGES-ERG20^{ww}2 | pPDC1-tObGES-ERG20^{ww}-tADH2 |
| pTU23S1_tObGES-ERG20^{ww}3 | pPYK1-tObGES-ERG20^{ww}-tACS2 |
| pTU23S1_tObGES-ERG20^{ww}4 | pGAL1-tObGES-ERG20^{ww}-tCYC1 |
| pTU11S1_ERG10s-SKL | pHHF2-ERG10-SKL-tENO1 |
| pTU1116_tHMG1-SKL | pTDH3-tHMG1-SKL-tTDH1 |
| pTU11S1_ERG8s-SKL | pTEF1-ERG8-SKL-tSSA1 |
| pTU1116_IDI1-SKL | pCCW12-IDI1-SKL-tENO2 |
| pTU11S1_ERG13s-SKL | pPGK1-ERG13-SKL-tPGK1 |
| pTU2223_ERG12s-SKL | pTEF2-ERG12-SKL-tADH1 |
| pTU1126_ERG19s-SKL | pHHF1-ERG19-SKL-tCYC1 |
| pTU24S1_tObGES- | pGAL1-tObGES-ERG20^{ww}-SKL-tCYC1 |
| ERG20^{ww}4-SKL | |
| pTU24S1_ERG20 | pPYK1-ERG20-tACS2 |
| pTU1116_ZSS1 | pGAL1-ZSS1-tCYC1 |
| pTU1116_ERG9 | pGAL1-ERG9-tCYC1 |
| pTU24S1_ERG20-ZSS1 | pGAL1-ERG20-ZSS1-tCYC1 |
| pTU24S1_ERG20-ERG9 | pGAL1-ERG20-ERG9-tCYC1 |
| pTU24S1_ERG9-ERG20 | pGAL1-ERG9-ERG20-tCYC1 |

s = strong,
m = medium,
w = weak

TABLE 7
List of intermediate multi-gene vectors generated in this study.

| Name | Description |
|---|---|
| pMGI1(Inter)_rox1Δ::GFP dropout | Intermediate vector with |
| | homology arms for the ROX1 |
| | locus and selection marker URA3 |
| pMGI2(Inter)_gal1Δ::GFP dropout | Intermediate vector with |
| | homology arms for the GAL1 |
| | locus and selection marker Leu2 |
| pMGI5(Inter)_gal80Δ::GFP dropout | Intermediate vector with |
| | homology arms for the GAL80 |
| | locus and selection marker TRP1 |
| pMGR24(Inter)_GFP dropout | High copy intermediate |
| | vector with KanR marker for |
| | cloning the downstream genes |

pMG = multi-gene plasmid,
I = integrative,
R = replicative

TABLE 8
List of multi-gene plasmids generates in this study.

| | Description |
|---|---|
| Name | (Constituent TUs and target locus) |
| pMGI1_rox1Δ::ERG10s.tHMG1 | ERG10s and tHGM1 |
| | TUs in the ROX1 locus |
| pMGI1_rox1Δ::ERG10m.tHMG1 | ERG10m and tHMG1 |
| | TUs in the ROX1 locus |
| pMGI1_rox1Δ::ERG10w.tHMG1 | ERG10w and tHMG1 |
| | TUs in the ROX1 locus |
| pMGI1_gal 1Δ::ERG13s.ERG12s. | ERG13s, ERG12s and ERG19s |
| ERG19s | TUs in the GAL1 locus |
| pMGI2_gal1Δ::ERG13s.ERG12s. | ERG13s, ERG12s and ERG19m |
| ERG19m | TUs in the GAL1 locus |
| pMGI2_gal1Δ::ERG13s.ERG12s. | ERG13s, ERG12s and ERG19w |
| ERG19w | TUs in the GAL1 locus |
| pMGI2_gal1Δ::ERG13s.ERG12m. | ERG13s, ERG12m and ERG19s |
| ERG19s | TUs in the GAL1 locus |
| pMGI2_gal1Δ::ERG13s.ERG12m. | ERG13s, ERG12m and ERG19m |
| ERG19m | TUs in the GAL1 locus |
| pMGI2_gal1Δ::ERG13s.ERG12m. | ERG13s, ERG12m and ERG19w |
| ERG19w | TUs in the GAL1 locus |
| pMGI2_gal1Δ::ERG13s.ERG12w. | ERG13s, ERG12w and ERG19s |
| ERG19s | TUs in the GAL1 locus |
| pMGI2_gal1Δ::ERG13s.ERG12w. | ERG13s, ERG12w and ERG19m |
| ERG19m | TUs in the GAL1 locus |
| pMGI2_gal1Δ::ERG13s.ERG12w. | ERG13s, ERG12w and ERG19w |
| ERG19w | TUs in the GAL1 locus |
| pMGI2_gal1Δ::ERG13m.ERG12s. | ERG13m, ERG12s and ERG19w |
| ERG19s | TUs in the GAL1 locus |
| pMGI2_gal1Δ::ERG13m.ERG12s. | ERG13m, ERG12s and ERG19m |
| ERG19m | TUs in the GAL1 locus |
| pMGI2_gal1Δ::ERG13m.ERG12s. | ERG13m, ERG12s and ERG19w |
| ERG19w | TUs in the GAL1 locus |
| pMGI2_gal1Δ::ERG13m.ERG12m. | ERG13m, ERG12m and ERG19s |
| ERG19s | TUs in the GAL1 locus |
| pMGI2_gal1Δ:::ERG13m.ERG12m. | ERG13m, ERG12m and ERG19m |
| ERG19m | TUs in the GAL1 locus |
| pMGI2_gal1Δ::ERG13m.ERG12m. | ERG13m, ERG12m and ERG19w |
| ERG19w | TUs in the GAL1 locus |
| pMGI2_gal1Δ::ERG13m.ERG12w. | ERG13m, ERG12w and ERG19s |
| ERG19s | TUs in the GAL1 locus |
| pMGI2_gal1Δ::ERG13m.ERG12w. | ERG13m, ERG12w and ERG19m |
| ERG19m | TUs in the GAL1 locus |
| pMGI2_gal1Δ::ERG13m.ERG12w. | ERG13m, ERG12w and ERG19w |
| ERG19w | TUs in the GAL1 locus |
| pMGI2_gal1Δ::ERG13w.ERG12s. | ERG13w, ERG12s and ERG19s |
| ERG19s | TUs in the GAL1 locus |
| pMGI2_gal1Δ::ERG13w.ERG12s. | ERG13w, ERG12s and ERG19m |
| ERG19m | TUs in the GAL1 locus |
| pMGGI2_gal1Δ::ERG13w.ERG12s. | ERG13w, ERG12s and ERG19w |
| ERG19w | TUs in the GAL1 locus |
| pMGI2_gal1Δ::ERG13w.ERG12m. | ERG13w, ERG12m and ERG19s |
| ERG19s | TUs in the GAL1 locus |
| pMGI2_gal1Δ::ERG13w.ERG12m. | ERG13w, ERG12m and ERG19m |
| ERG19m | TUs in the GAL1 locus |
| pMGI2_gal1Δ::ERG13w.ERG12m. | ERG13w, ERG12m and ERG19w |
| ERG19w | TUs in the tGAL1 locus |
| pMGI2_gal1Δ::ERG13w.ERG12w. | ERG13w, ERG12w and ERG19s |
| ERG19s | TUs in the GAL1 locus |
| pMGI2_gal1Δ::ERG13w.ERG12w. | ERG13w, ERG12w and ERG19m |
| ERG19m | TUs in the GAL1 locus |
| pMGI2_gal1Δ::ERG13w.ERG12w. | ERG13w, ERG121w and ERG19w |
| ERG19w | TUs in the GAL1 locus |
| pMGI5gal80Δ::ERG8s.IDI1 | ERG8 and IDI1 TUs in the |
| | GAL80 locus |
| pMGI5gal80Δ::ERG8m.IDI1 | ERG8m and IDI1 TUs in |
| | the tGAL80 locus |
| pMGI5gal80Δ::ERG8w.IDI1 | ERG8w and IDI1 TUs in |
| | the GAL80 locus |
| pMGR24_ERG20.ZSS1 | ERG20 and ZSS1 TUs in |
| | the GAL80 locus |
| pMGI1_rox1Δ::ERG10s-SKL. | ERG10s-SKL and tHMG1-SKL |
| tHMG1-SKL | TUs in the ROX1 locus |
| pMGI5gal80Δ::ERG8s-SKL.IDI1. | ERG8s-SKL and IDI1-SKL |
| SKL | TUs in the GAL80 locus |
| pMGR24_ERG20.ERG9 | ERG20 and ERG9 TUs |
| pMGR24_ERG9.ERG20 | ERG9 and ERG20 TUs |

TABLE 9
List of pCAS9 plasmids for genomic integrations used in this study.

| Name | Description |
|---|---|
| pCAS_Pphe-BsaI_NAT | tRNA^{Phe} promoter-delta ribozyme-gRNA |
| (pCAS) | cloning site-SNR52t, pRNR2- |
| | Cas9-NLS-CYC1t, NATMX |
| pCAS-ROX1 | gRNA targeting the ROX1 locus cloned at the |
| | gRNA cloning site of pCAS |
| pCAS-GAL1 | gRNA targeting the GAL1 locus cloned at the |
| | gRNA cloning site of pCAS |
| pCAS-GAL80 | gRNA targeting the GAL80 locus cloned at the |
| | gRNA cloning site of pCAS |

TABLE 10
List of primers and DNA oligos used in this study. F: forward primer; R: reverse
primer; dom: domestication; hom: homologous arm; gRNA: guide RNA; gDNA: genomic DNA;
conf: confirmation; rt: real-time PCR.
Primers with Moclo overhangs for cloning in pYTK001:

| | |
|---|---|
| ERG10 F | tttcgtctcgtcggggtctcgtatgtctcagaacgtttacattgtatc |
| ERG10 R | tttcgtctctggtcggtctccggattcatatcttttcaatgacaatagaggaag |
| ERG8F | tttcgtctcgtcggggtctcgtatgtcagagttgagagccttc |
| ERG8R | tttcgtctctggtcggtctccggatttatttatcaagataagtttccggatc |
| ERG12 F | tttcgtctcgtcggggtctcgtatgtcattaccgttcttaacttctg |
| ERG12 R | tttcgtctctggtcggtctccggatttatgaagtccatggtaaattcgtg |
| tHMG1 F | tttcgtctcgtcggggtctcgtatgccagttttaaccaataaaacag |
| tHMG1 R | tttcgtctctggtcggtctccggatttaggatttaatgcaggtgacgg |
| ERG13 F | tttcgtctcgtcggggtctcgtatgaaactctcaactaaactttgttg |
| ERG13 dom R | tttcgtctcatggcgtccctaccatcc |
| ERG13 dom F | tttcgtctccgccattgtagtttgcggtg |
| ERG13 R | tttcgtctctggtcggtctccggatttattttttaacatcgtaagatcttctaaatttgtc |
| ERG19 F | tttcgtctcgtcggggtctcgtatgaccgtttacacagcatcc |
| ERG19 dom R | tttcgtctcttgcagagaccaatgcagcaaagc |
| ERG19 dom F | ttcgtctcctgcaattgctaagttataccaattacc |
| ERG19 R | tttcgtctctggtctcggtctccggatttattcctttggtagaccagtctttg |
| IDI1 F | tttcgtctcgtcggggtctcgtatgactgccgacaacaatag |
| IDI1 dom R | tttcgtctctcatttgaagtctcactagatcg |
| IDI1 dom F | tttcgtctcaaatgacgaaagcggagaaa |
| IDI1 R | tttcgtctctggtcggtctccggatttatagcattctatgaatttgcctgtc |
| ERG10 SKL R | tttcgtctctggtctccggattcataacttagatatcttttcaatgacaatagaggaag |
| ERG13 SKL R | tttcgtctcgggtctccggatttataacttagattttttaacatcgtaagatcttctaaatttg |
| ERG12 SKL R | tttcgtctctggtcggtctccggatttataacttagatgaagtccatggtaaattcgtg |
| ERG8 SKL R | tttcgtctctggtctccggatttataacttagatttatcaagataagtttccggatc |
| ERG19 SKL R | atgcgtctctggtctccggatctataacttagattcctttggtagaccagtctttg |
| tHMG1 SKL R | tttcgtctctggtcggtctccggatttataacttagaggatttaatgcaggtgacgg |
| IDI1 SKL R | tttcgtctctggtctccggatttataacttagatagcattctatgaatttgcctgtc |
| tObGES F | tttcgtctcgtcggggtctcgtatggaagagagttcatcaaagc |
| tObGES fusion R | tgacgtctcgccatgccagaaccttgtgtaaaaaacagggcatcg |
| ERG20WW fusion F | tttcgtctcgatggcttcagaaaaagaaattaggag |
| ERG20WW R | tttcgtctcgggtcggtctcgggatctatttgcttctcttgtaaactttgttc |
| ERG20WW SKL R | tttcgtctctggtctctggatttataacttagatttgcttctcttgtaaactttgttc |
| CYC1 F | tttcgtctcgtcggggtctcgatccgctctaaccgaaaagg |
| CYC1 R | tttcgtctcgggtcggtctcgcagccttcgagcgtcccaaaac |
| ROX1 5′Hom F | tttcgtctcgtcggtctcacaatcggccggtctggc |
| ROX1 5′Hom R | tttcgtctcgggtctcaagggtaagaacctacacacaaaagacaca |
| ROX1 3′Hom F | tttcgtctcgtcggtctcagagtcttctaactatatggtctccagatcttta |
| ROX1 3′Hom R | tttcgtctcgggtctcatcggatgcgtaggggtagttgtg |
| GAL1 5′Hom F | tttcgtctcgtcggtctcacaataaaaattcttactttttttttggatggac |
| GAL1 5′Hom R | tttcgtctcgggtctcaagggaatagatcaaaaatcatcgcttcgc |
| GAL1 3′Hom F | tttcgtctcgtcggtctcagagtgctgcctctgtttgcg |
| GAL1 3′Hom R | tttcgtctcgggtctcatcggaatctcactggagatgttgttaagtag |
| GAL80 5′Hom F | tttcgtctcgtcggtctcacaatggattgcgcttgcctttg |
| GAL80 5′Hom R | tttcgtctcgggtctcaaggggaagttaatacctttaggttggttttcc |
| GAL80 3′Hom F | tttcgtctcgtcggtctcagagttgctgaacgtggggttc |
| GAL80 3′Hom R | tttcgtctcgggtctcatcggcaagtttcaaatctcccttggtac |
| TRP1 F | tttcgtctcgtcggtctcatacaaacgacattactatatatataatataggaagc |
| TRP1 R | tttcgtctcgggtctcgactccgcatctgtgcggtatttc |
| ERG20 F | tttcgtctcgtcggggtctcgtatggcttcagaaaaagaaattagg |
| ERG9 F | tttcgtctcgtcggggtctcgtatgggaaagctattacaattggc |
| ERG9 R | tttcgtctcgggtcggtctccggattcacgctctgtgtaaagtgt |
| ERG20 fusion R | tttcgtctcgccatagaaccaccacctttgcttctcttgtaaactttgttc |
| ZSS1 fusion F | tttcgtctcgatggagcgtcagtcaatgg |
| ERG9 fusion F | tttcgtctcgatgggaaagctattacaattggc |
| ERG9 fusion R | tttcgtctcgccatagaaccaccacccgctctgtgtaaagtgtatatataataaaac |

Oligos containing gRNAs and overhangs for Gibson cloning:

| | |
|---|---|
| ROX1 gRNA F | cgggtggcgaatgggactttattcgtctattaagatcctggttttagagctagaaatagc |
| ROX1 gRNA R | gctatttctagctctaaaaccaggatcttaatagacgaataaagtcccattcgccacccg |
| GAL1 gRNA F | cgggtggcgaatgggactttatatcaaaatcaatagctaagttttagagctagaaatagc |
| GALI gRNAR | gctatttctagctctaaaacttagctattgattttgatataaagtcccattcgccacccg |
| GAL80 gRNA F | cgggtggcgaatgggacttttcgttcgggcgagagtgcgcgttttagagctagaaatagc |
| GAL80 gRNA R | gctatttctagctctaaaacgcgcactctcgcccgaacgaaaagtcccattcgccacccg |

Primers for diagnostic PCR to confirm genomic integrations:

| | |
|---|---|
| ROX1 gDNA 5′ F | cacacactgcgttctcttg |
| Multi-gene R | cagttcagtctagatgcgaattc |
| ROX1 URA3 F | gctaaggtagagggtgaacg |
| ROX1 gDNA 3′ R | ggtttggtatatgaggaatgtgatg |
| GAL1 gDNA 5′ F | gtaactgagctgtcatttatattgaattttc |
| GAL1 LEU2 F | gctgtcgccgaagaag |
| GAL1 gDNA 3′ R | ccctctgatatagctttaagacttga |
| GAL80 gDNA 5′ F | ctacctgactagattttcattttgtttc |
| GAL80 TRP1 F | cgcttagattaaatggcgttattgg |
| GAL80 HYGR F | gaagtactcgccgatagtgg |
| GAL80 gDNA 3′ R | gtaaaggaccagatttgaaatttctg |

Primers for confirmation of identity of each integrated gene:

| | |
|---|---|
| ERG10 conf F | cactgctatccatcttacagc |
| tHMG1 conf R | caaccgctctcgtagtatcac |
| ERG8 conf F | gataaataaatcctaactcgaggcc |
| IDI1 conf R | gcactctcgagttattatagcattc |
| ERG13 conf F | gcaaagtggtgtttactacttg |
| ERG12 conf R | catagctaaggccagtgatac |
| ERG12 conf F | cacgaatttaccatggacttc |
| ERG19 conf R | gtctgcgatttgtactgcc |

Primers for qRT-PCR:

| | |
|---|---|
| UBC6 F | gatacttggaatcctggctgg |
| UBC6 R | gctaatgtcttcttctgatggtctg |
| ERG10 rt F | gtctgtgcatccgctatgaag |
| ERG10 rt R | ctgctggcatgtagtatggtg |
| ERG13 rt F | gatggtagagacgccattgtag |
| ERG13 rt R | gcgtgttccatgtaagaagc |
| HMG1 rt F | aagcagacccgtttgacg |
| HMG1 rt R | tgacccggtcttcctcatg |
| tHMG1 rt F | ccgtatccatgccatccatc |
| tHMG1 rt R | gaatagttgcctgtgccgtc |
| ERG12 rt F | gccatcaccgaggatcaag |
| ERG12 rt R | gctgcatggtagtggaagg |
| ERG8 rt F | gatgatgcctaccattctcagg |
| ERG8 rt R | ctgtgactaaacctgccgag |
| ERG19 rt F | ctgaagatggtcatgattccatgg |
| ERG19 rt R | tgccacggtcaattgcatac |
| IDI1 rt F | ctacatcgtgcattctccgtc |
| IDI1 rt R | cttatcgtctagcttacccttcaaac |

TABLE 11
List of yeast promoters with their designated and relative
strengths (Lee et al, 2015) (3) as well as qRT-PCR validation.

| | | | Relative promoter | qRT-PCR Fold |
|---|---|---|---|---|
| | | | strength quantified | change of gene |
| | Gene | Designated | using a fluorescent | expression over |
| Promoter | expressed | strength | protein (a.u.) * | wild-type |
| pTDH3 | tHMG1 | Strong | 30.75 ± 2.3 | 17.84 ± 1.66** |
| pCCW12 | IDI1 | Strong | 24.60 ± 0.91 | 12.16 ± 1.31** |
| pHHF2 | ERG10 | Strong | 9.01 ± 0.17 | 2.62 ± 0.93 |
| pRPL18B | ERG10 | Medium | 3 ± 0.25 | 1.01 ± 0.32 |
| pPOP6 | ERG10 | Weak | 1.06 ± 0.04 | 1.02 ± 0.47 |
| pPGK1 | ERG13 | Strong | 11.01 ± 0.65 | 1.09 ± 0.19 |
| pHTB2 | ERG13 | Medium | 2.85 ± 0.1 | 1.25 ± 0.53 |
| pRNR2 | ERG13 | Weak | 1.06 ± 0.04 | 1.36 ± 0.4 |
| pTEF2 | ERG12 | Strong | 7.77 ± 0.35 | 1.95 ± 0.4 |
| pPAB1 | ERG12 | Medium | 1.69 ± 0.12 | 2.11 ± 0.83 |
| pPSP2 | ERG12 | Weak | 0.91 ± 0.03 | 2.38 ± 0.85 |
| pTEF1 | ERG8 | Strong | 8.85 ± 0.3 | 2.62 ± 0.15 |
| pALD6 | ERG8 | Medium | 2.28 ± 0.05 | 2.8 ± 0.4 |
| pRAD27 | ERG8 | Weak | 0.91 ± 0.03 | 3.06 ± 0.61 |
| pHHF1 | ERG19 | Strong | 4.81± 0.08 | 3.44 ± 0.72 |
| pRET2 | ERG19 | Medium | 1.53 ± 0.14 | 4.49 ± 0.49 |
| pREV1 | ERG19 | Weak | 0.86 ± 0.02 | 4.75 ± 0.86 |

* Calculated from the raw data for promoter strengths kindly provided by Prof. John Dueber.
**The qRT-PCR fold change of gene expression over wildtype values for pTDH3 and pCCW12 are the mean values of the fold change of gene expression in the all-strong (α1), all-medium (β5), and all-weak (λ9) strains.

TABLE 12
List of the 27 gal1Δ strains in the CEN-PK2-1C background used
for preparing the combinatorial library.

| Name of strains | Description |
|---|---|
| α | gal1Δ::pPGK1-ERG13-tPGK1, pTEF2-ERG12-tADH1, |
| | pHHF1-ERG19-tCYC1 |
| A | gal1Δ::pPGK1-ERG13-tPGK1, pTEF2-ERG12-tADH1, |
| | pRET2-ERG19-tCYC1 |
| B | gal1Δ::pPGK1-ERG13-tPGK1, pTEF2-ERG12-tADH1, |
| | pREV1-ERG19-tCYC1 |
| C | gal1Δ::pHTB2-ERG13-tPGK1, pTEF2-ERG12-tADH1, |
| | pRET2-ERG19-tCYC1 |
| D | gal1Δ::pHTB2-ERG13-tPGK1, pTEF2-ERG12-tADH1, |
| | pREV1-ERG19-tCYC1 |
| E | gal1Δ::pHTB2-ERG13-tPGK1, pTEF2-ERG12-tADH1, |
| | pHHF1-ERG19-tCYC1 |
| F | gal1Δ::pRNR2-ERG13-tPGK1, pTEF2-ERG12-tADH1, |
| | pHHF1-ERG19-tCYC1 |
| G | gal1Δ::pRNR2-ERG13-tPGK1, pTEF2-ERG12-tADH1, |
| | pRET2-ERG19-tCYC1 |
| H | gal1Δ::pRNR2-ERG13-tPGK1, pTEF2-ERG12-tADH1, |
| | pREV1-ERG19-tCYC1 |
| I | gal1Δ::pPGK1-ERG13-tPGK1, pPAB1-ERG12-tADH1, |
| | pHHF1-ERG19-tCYC1 |
| J | gal1Δ::pPGK1-ERG13-tPGK1, pPAB1-ERG12-tADH1, |
| | pRET2-ERG19-tCYC1 |
| K | gal1Δ::pPGK1-ERG13-tPGK1, pPAB1-ERG12-tADH1, |
| | pREV1-ERG19-tCYC1 |
| L | gal1Δ::pHTB2-ERG13-tPGK1, pPAB1-ERG12-tADH1, |
| | pREV1-ERG19-tCYC1 |
| β | gal1Δ::pHTB2-ERG13-tPGK1, pPAB1-ERG12-tADH1, |
| | pRET2-ERG19-tCYC1 |
| M | gal1Δ::pHTB2-ERG13-tPGK1, pPAB1-ERG12-tADH1, |
| | pREV1-ERG19-tCYC1 |
| N | gal1Δ::pRNR2-ERG13-tPGK1, pPAB1-ERG12-tADH1, |
| | pRET2-ERG19-tCYC1 |
| O | gal1Δ::pRNR2-ERG13-tPGK1, pPAB1-ERG12-tADH1, |
| | pHHF1-ERG19-tCYC1 |
| P | gal1Δ::pRNR2-ERG13-tPGK1, pPAB1-ERG12-tADH1, |
| | pREV1-ERG19-tCYC1 |
| Q | gal1Δ::pPGK1-ERG13-tPGK1, pPSP2-ERG12-tADH1, |
| | pHHF1-ERG19-tCYC1 |
| R | gal1Δ::pPGK1-ERG13-tPGK1, pPSP2-ERG12-tADH1, |
| | pREV1-ERG19-tCYC1 |
| S | gal1Δ::pPGK1-ERG13-tPGK1, pPSP2-ERG12-tADH1, |
| | pRET2-ERG19-tCYC1 |
| T | gal1Δ::pHTB2-ERG13-tPGK1, pPSP2-ERG12-tADH1, |
| | pRET2-ERG19-tCYC1 |
| U | gal1Δ::pHTB2-ERG13-tPGK1, pPSP2-ERG12-tADH1, |
| | pHHF1--ERG19-tCYC1 |
| V | gal1Δ::pHTB2-ERG13-tPGK1, pPSP2-ERG12-tADH1, |
| | pREV1--ERG19-tCYC1 |
| W | gal1Δ::pRNR2-ERG13-tPGK1, pPSP2-ERG12-tADH1, |
| | pRET2-ERG19-tCYC1 |
| X | gal1Δ::pRNR2-ERG13-tPGK1, pPSP2-ERG12-tADH1, |
| | pHHF1-ERG19-tCYC1 |
| γ | gal1Δ::pRNR2-ERG13-tPGK1, pPSP2-ERG12-tADH1, |
| | pREV1--ERG19-tCYC1 |

TABLE 13
List of the strains in the CEN-PK2-1D background used for preparing the
combinatorial library. R: rox1; G: gal80.

| Name | Background strain | Description |
|---|---|---|
| R1 | CEN-PK2-1D | rox1Δ::pHHF2-ERG10-tENO1, |
| | | pTDH3-tHMG1-tTDH1 |
| R2 | CEN-PK2-1D | rox1Δ::pRPL18B-ERG10-tENO1, |
| | | pTDH3-tHMG1-tTDH1 |
| R3 | CEN-PK2-1D | rox1Δ::pPOP6-ERG10-tENO1, |
| | | pTDH3-tHMG1-tTDH1 |
| RG1 | R1 | gal80Δ::pTEF1-ERG8-tSSA1, |
| | | pCCW12-IDI1-tENO2 |
| RG2 | R1 | gal80Δ::pALD6-ERG8-tSSA1, |
| | | pCCW12-IDI1-tENO2 |
| RG3 | R1 | gal80Δ::pRAD27-ERG8-tSSA1, |
| | | pCCW12-IDI1-tENO2 |
| RG4 | R2 | gal80Δ::pTEF1-ERG8-tSSA1, |
| | | pCCW12-IDI1-tENO2 |
| RG5 | R2 | gal80Δ::pALD6-ERG8-tSSA1, |
| | | pCCW12-IDI1-tENO2 |
| RG6 | R2 | gal80Δ::pRAD27-ERG8-tSSA1, |
| | | pCCW12-IDI1-tENO2 |
| RG7 | R3 | gal80Δ::pTEF1-ERG8-tSSA1, |
| | | pCCW12-IDI1-tENO2 |
| RG8 | R3 | gal80Δ::pALD6-ERG8-tSSA1, |
| | | pCCW12-IDI1-tENO2 |
| RG9 | R3 | gal80Δ::pRAD27-ERG8-tSSA1, |
| | | pCCW12-IDI1-tENO2 |

TABLE 14
Mevalonate concentration in the top ten geraniol-producing and the γ9 all-weak strains.

| | | | | | | | Mevalonate |
|---|---|---|---|---|---|---|---|
| Strains | ERG10 | ERG13 | ERG12 | ERG8 | ERG19 | Geraniol (a.u.) | (mg/L) |
| α1 (all-strong) | 9.01 | 11.01 | 7.77 | 8.85 | 4.81 | 518.85 ± 0.54 | 22.89 ± 1.59 |
| β2 | 9.01 | 2.85 | 1.69 | 2.28 | 1.53 | 517.94 ± 13.96 | 16.59 ± 1.51 |
| α4 | 3.00 | 11.01 | 7.77 | 8.85 | 4.81 | 516.19 ± 87.54 | 9.28 ± 2.85 |
| N3 | 9.01 | 1.06 | 1.69 | 0.91 | 1.53 | 513.53 ± 42.87 | 11.43 ± 1.46 |
| N2 | 9.01 | 1.06 | 1.69 | 2.28 | 1.53 | 510.49 ± 11.46 | 14.63 ± 1.26 |
| β4 | 3.00 | 2.85 | 1.69 | 8.85 | 1.53 | 509.51 ± 21.59 | 18.89 ± 3.64 |
| β5 (all-medium) | 3.00 | 2.85 | 1.69 | 2.28 | 1.53 | 505.28 ± 10.16 | 11.04 ± 0.67 |
| β7 | 1.06 | 2.85 | 1.69 | 8.85 | 1.53 | 502.44 ± 15.87 | 15.06 ± 3.92 |
| β3 | 9.01 | 2.85 | 1.69 | 0.91 | 1.53 | 502.34 ± 12.10 | 16.25 ± 4.55 |
| β1 | 9.01 | 2.85 | 1.69 | 8.85 | 1.53 | 501.19 ± 1.77 | 7.62 ± 0.87 |
| γ9 (all-weak) | 1.06 | 1.06 | 0.86 | 0.91 | 0.91 | 221.18 ± 6.28 | 7.09 ± 2.9 |

### REFERENCES FOR EXAMPLE 5

1. J. Kim et al., Engineering Saccharomyces cerevisiae for isoprenol production. Metab Eng 64, 154-166 (2021).
2. E. E. K. Baidoo, G. Wang, C. J. Joshua, V. T. Benites, J. D. Keasling, Liquid chromatography and mass spectrometry analysis of isoprenoid intermediates in Escherichia coli. Methods Mol Biol 1859, 209-224 (2019).
3. M. E. Lee, W. C. DeLoache, B. Cervantes, J. E. Dueber, A highly characterized yeast toolkit for modular, multipart assembly. ACS Synth Biol 4, 975-986 (2015).

### Example 6: Promoter Strength

Designated promoter strength was assessed by M. E. Lee, W. C. DeLoache, B. Cervantes, J. E. Dueber, A highly characterized yeast toolkit for modular, multipart assembly. ACS Synth Biol 4, 975-986 (2015), which is incorporated herein by references as if fully set forth.

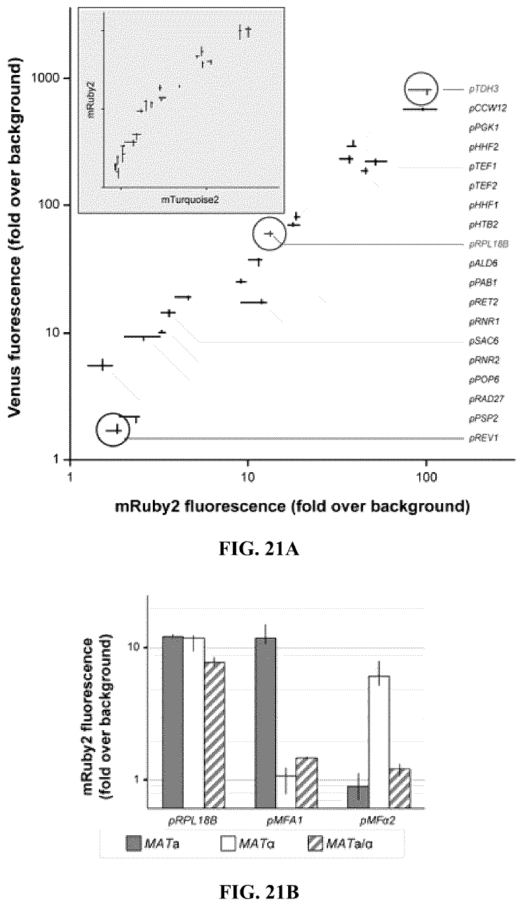

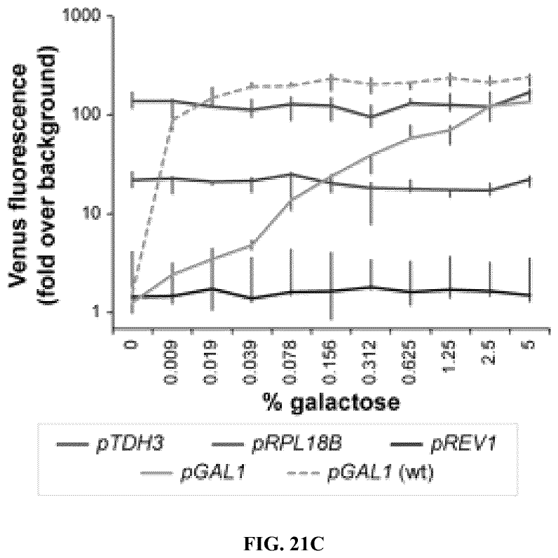

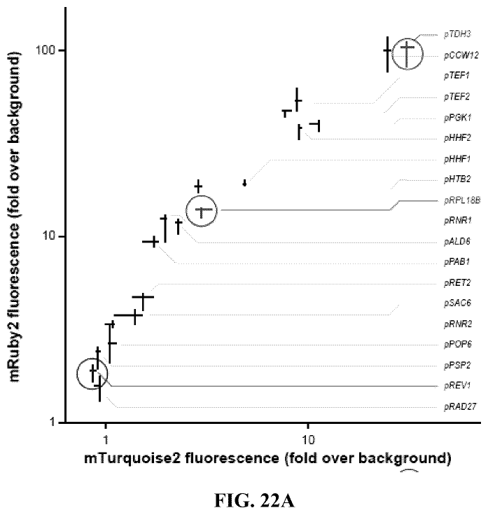

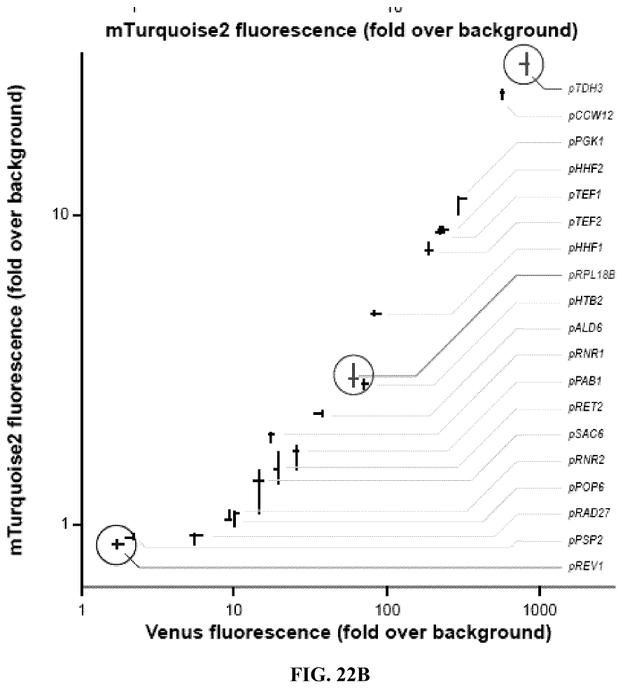

Briefly, Lee et al. characterized the strength of 19 constitutive promoters across two coding sequences, mRuby2 and Venus. As illustrated in FIG. 21A-22B, the relative strength of 19 promoters was consistent across two coding sequences, mRuby2 and Venus. Three promoters (strong pTDH3, medium pRPL18B, and weak pREV1) are highlighted. (FIG. 21A) The horizontal and vertical bars represent the range of four biological replicates, and the intersection represents the median value. (inset) A third fluorescent protein, mTurquoise2, was also tested, and a larger plot can be found in FIGS. 22A and 22B. (FIG. 21B) The mating-type-specific promoter, pMFA1, is only active in the MATa haploid; pMFα2 is only active in MATα haploids; neither promoter is active in the opposite haploid or in the diploid. The expression level of pRPL18B in the three strains is shown for reference. The height of the bars represents the median value of four biological replicates, and the error bars show the range. (FIG. 21C) Galactose induction of pGAL1 increases expression from background levels up to the highest expressing constitutive promoter, pTDH3. All solid line data were collected from a Δgal2 strain. The dashed line shows a much more sensitive response to galactose induction in a wild type strain. Points represent the median value of four biological replicates, and error bars show the range.

It is sometimes useful to have genes under dynamic control, and for this we provide two tools: mating-type-specific and inducible promoters. pMFA1 and pMFα2 were tested by Lee et al. and it was found that they have very close to background levels of fluorescence in both the opposite mating-type haploid and diploid strains and a 6- to 10-fold induction in the appropriate haploid (FIG. 21). Lee et al. also tested pGAL1 in varying concentrations of galactose and observed a 100-fold induction (FIG. 21C). Although the promoter can be used in wild-type strains, the response is very sensitive to low concentrations of galactose; a strain with the GAL2 transporter knocked out should be used for more graded control overexpression. Finally, pCUP1 was tested in varying concentrations of copper (II) sulfate (CuSO₄) and a 55-fold induction was observed. This promoter exhibits leaky expression under basal conditions, with approximately 7-fold fluorescence over background when CuSO₄ is not added to the media. This may be due in part to the CuSO₄ that is present at 250 nM in the yeast nitrogen base commonly used to make defined media.

For these assays, promoter testing constructs were integrated into the URA3 locus of the yeast chromosome. Constitutive promoter, terminator, and degradation tag testing constructs were selected using a Zeocin resistance cassette; mating-type and inducible promoter testing constructs were selected for uracil prototrophy.

Colonies were picked and grown in 500 μL of media in 96-deep-well blocks at 30° C. in an ATR shaker, shaking at 750 rpm until saturated. Cultures were diluted 1:100 in fresh media, grown for 12-16 h, then diluted 1:3 in fresh media, and fluorescence was measured on a TECAN Safire2. For the galactose inductions, the media was switched during the dilution step from 2% dextrose to 2% raffinose with different concentrations of galactose. For the copper inductions, saturated cultures were diluted 1:100 in fresh media with different concentrations of copper (II) sulfate and grown for 18 h.

Excitation and emission wavelengths used to measure fluorescent proteins were mTurquoise2 at 435 nm/478 nm, Venus at 516 nm/530 nm, and mRuby2 at 559 nm/600 nm. Raw fluorescence values were first normalized to the OD600 of the cultures, and then normalized to the background fluorescence of cells not expressing any fluorescent protein. The median log value of biological replicates was calculated and plotted with the range.

As found in Lee et al., (1) the high-strength promoters were pTDH3 (SEQ ID NO: 1), pCCW12 (SEQ ID NO: 2), pPGK1 (SEQ ID NO: 3), pHHF2 (SEQ ID NO: 4), pTEF1 (SEQ ID NO: 5), pTEF2 (SEQ ID NO: 6), and pHHF1 (SEQ ID NO: 7), (2) the medium-strength promoters were pRPL18B (SEQ ID NO: 8), pHTB2 (SEQ ID NO: 9), pALD6 (SEQ ID NO: 10, pPAB1 (SEQ ID NO: 11), pRET2 (SEQ ID NO: 12), and (3) the weak-strength promoters were pPOP6 (SEQ ID NO: 13), pRNR2 (SEQ ID NO: 14), pPSP2 (SEQ ID NO: 15), pRAD27 (SEQ ID NO: 16), and pREV1 (SEQ ID NO: 17).

### Example 7: Quantifying Promoter Strength by a Fluorescent Assay

In order to quantify promoter strengths, a fluorescent protein mTurquoise 2 was cloned downstream of each promoter, and fluorescence was recorded using a plate reader by Dr. John Dueber's group, A highly characterized yeast toolkit for modular, multipart assembly. ACS Synth Biol 4, 975-986 (2015), which is incorporated herein by references as if fully set forth. Specifically, plasmids containing each of the 17 promoters were cloned upstream of a mTurquoise 2. These plasmids also contain a zeocin selective marker. The mTurquoise 2 and the zeocin transcription units were then integrated into the yeast URA3 locus using CRISPR/Cas9 genome editing. Successfully integrated yeast colonies were selected using Zeocin marker in a synthetic medium composed of 2% (w/v) glucose, 0.67% (w/v) yeast nitrogen base, 0.2% (w/v) dropout mix complete without yeast nitrogen base, 0.85% (w/v) MOPS free acid (pH 7.0), 0.1 M dipotassium phosphate, and 100 μg/L Zeocin. A single colony was inoculated in 500 μl of the fresh medium in a 96-deep-well plate at 30° C. with shaking until OD₆₀₀ saturated. Cultures were then diluted 1:100 into fresh medium followed by shaking at 30° C. for an additional 12-16 hours. Cultures were then diluted 1:3, and the fluorescence was recorded using a plate reader with excitation at 435 nm and emission at 478 nm. The fluorescence values were then normalized by OD₆₀₀ cell density values. The folds of normalized fluorescence over the background were then calculated. The final reported folds of fluorescence over the background were the average of four biological replicates.

### Example 8: Production of Terpenes from Engineered Microbes

See Mukherjee, M. et al. “Machine-learning guided elucidation of contribution of individual steps in the mevalonate pathway and construction of a yeast platform strain for terpene production” (2022) Metabolic Engineering 74: 139-149, which is incorporated herein by reference as if fully set forth.

### Example 9: A Combinatorial Library with 243 Engineered Yeast Strains

In the below Strain Table, promoters used to express each genes are listed, as well as the amount of geraniol produced. WT means wild type. A composition, method, or kit herein may comprise one or more of the below listed strains.

| Strain | | | | | | | | |
|---|---|---|---|---|---|---|---|---|
| Name | ERG10 | ERG13 | tHMG1 | ERG12 | ERG8 | ERG19 | IDI1 | Geraniol (a.u.) |
| WT | N/A | N/A | N/A | N/A | N/A | N/A | NA | 196 |
| α1 | pHHF2 | pPGK1 | pTDH3 | pTEF2 | pTEF1 | pHHF1 | pCCW12 | 518.8549102 |
| α2 | pHHF2 | pPGK1 | pTDH3 | pTEF2 | pALD6 | pHHF1 | pCCW12 | 434.5408231 |
| α3 | pHHF2 | pPGK1 | pTDH3 | pTEF2 | pRAD27 | pHHF1 | pCCW12 | 381.7535662 |
| α4 | pRPL18B | pPGK1 | pTDH3 | pTEF2 | pTEF1 | pHHF1 | pCCW12 | 516.1874151 |
| α5 | pRPL18B | pPGK1 | pTDH3 | pTEF2 | pALD6 | pHHF1 | pCCW12 | 409.4631317 |
| α6 | pRPL18B | pPGK1 | pTDH3 | pTEF2 | pRAD27 | pHHF1 | pCCW12 | 389.9199774 |
| α7 | pPOP6 | pPGK1 | pTDH3 | pTEF2 | pTEF1 | pHHF1 | pCCW12 | 394.2614434 |
| α8 | pPOP6 | pPGK1 | pTDH3 | pTEF2 | pALD6 | pHHF1 | pCCW12 | 376.9769225 |
| α9 | pPOP6 | pPGK1 | pTDH3 | pTEF2 | pRAD27 | pHHF1 | pCCW12 | 342.5062647 |
| A 1 | pHHF2 | pPGK1 | pTDH3 | pTEF2 | pTEF1 | pRET2 | pCCW12 | 492.2137222 |
| A 2 | pHHF2 | pPGK1 | pTDH3 | pTEF2 | pALD6 | pRET2 | pCCW12 | 467.3674993 |
| A 3 | pHHF2 | pPGK1 | pTDH3 | pTEF2 | pRAD27 | pRET2 | pCCW12 | 447.2153248 |
| A 4 | pRPL18B | pPGK1 | pTDH3 | pTEF2 | pTEF1 | pRET2 | pCCW12 | 463.4235912 |
| A 5 | pRPL18B | pPGK1 | pTDH3 | pTEF2 | pALD6 | pRET2 | pCCW12 | 461.6800848 |
| A 6 | pRPL18B | pPGK1 | pTDH3 | pTEF2 | pRAD27 | pRET2 | pCCW12 | 469.1169062 |
| A 7 | pPOP6 | pPGK1 | pTDH3 | pTEF2 | pTEF1 | pRET2 | pCCW12 | 433.663831 |
| A 8 | pPOP6 | pPGK1 | pTDH3 | pTEF2 | pALD6 | pRET2 | pCCW12 | 435.1758809 |
| A 9 | pPOP6 | pPGK1 | pTDH3 | pTEF2 | pRAD27 | pRET2 | pCCW12 | 428.3118252 |
| B 1 | pHHF2 | pPGK1 | pTDH3 | pTEF2 | pTEF1 | pREV1 | pCCW12 | 472.7364452 |
| B 2 | pHHF2 | pPGK1 | pTDH3 | pTEF2 | pALD6 | pREV1 | pCCW12 | 454.4233827 |
| B 3 | pHHF2 | pPGK1 | pTDH3 | pTEF2 | pRAD27 | pREV1 | pCCW12 | 443.545539 |
| B 4 | pRPL18B | pPGK1 | pTDH3 | pTEF2 | pTEF1 | pREV1 | pCCW12 | 468.1141506 |
| B 5 | pRPL18B | pPGK1 | pTDH3 | pTEF2 | pALD6 | pREV1 | pCCW12 | 411.8780187 |
| B 6 | pRPL18B | pPGK1 | pTDH3 | pTEF2 | pRAD27 | pREV1 | pCCW12 | 489.2331505 |
| B 7 | pPOP6 | pPGK1 | pTDH3 | pTEF2 | pTEF1 | pREV1 | pCCW12 | 460.222433 |
| B 8 | pPOP6 | pPGK1 | pTDH3 | pTEF2 | pALD6 | pREV1 | pCCW12 | 448.8461625 |
| B 9 | pPOP6 | pPGK1 | pTDH3 | pTEF2 | pRAD27 | pREV1 | pCCW12 | 387.7324145 |
| C1 | pHHF2 | pHTB2 | pTDH3 | pTEF2 | pTEF1 | pRET2 | pCCW12 | 418.6008846 |
| C2 | pHHF2 | pHTB2 | pTDH3 | pTEF2 | pALD6 | pRET2 | pCCW12 | 333.8961362 |
| C3 | pHHF2 | pHTB2 | pTDH3 | pTEF2 | pRAD27 | pRET2 | pCCW12 | 470.5688865 |
| C4 | pRPL18B | pHTB2 | pTDH3 | pTEF2 | pTEF1 | pRET2 | pCCW12 | 437.2225097 |
| C5 | pRPL18B | pHTB2 | pTDH3 | pTEF2 | pALD6 | pRET2 | pCCW12 | 426.3711918 |
| C6 | pRPL18B | pHTB2 | pTDH3 | pTEF2 | pRAD27 | pRET2 | pCCW12 | 456.5264842 |
| C7 | pPOP6 | pHTB2 | pTDH3 | pTEF2 | pTEF1 | pRET2 | pCCW12 | 446.1253697 |
| C8 | pPOP6 | pHTB2 | pTDH3 | pTEF2 | pALD6 | pRET2 | pCCW12 | 466.8726511 |
| C9 | pPOP6 | pHTB2 | pTDH3 | pTEF2 | pRAD27 | pRET2 | pCCW12 | 333.470655 |
| D1 | pHHF2 | pHTB2 | pTDH3 | pTEF2 | pTEF1 | pREV1 | pCCW12 | 449.5036657 |
| D2 | pHHF2 | pHTB2 | pTDH3 | pTEF2 | pALD6 | pREV1 | pCCW12 | 427.6793423 |
| D3 | pHHF2 | pHTB2 | pTDH3 | pTEF2 | pRAD27 | pREV1 | pCCW12 | 410.5783964 |
| D4 | pRPL18B | pHTB2 | pTDH3 | pTEF2 | pTEF1 | pREV1 | pCCW12 | 414.8517034 |
| D5 | pRPL18B | pHTB2 | pTDH3 | pTEF2 | pALD6 | pREV1 | pCCW12 | 387.3014692 |
| D6 | pRPL18B | pHTB2 | pTDH3 | pTEF2 | pRAD27 | pREV1 | pCCW12 | 427.1443337 |
| D7 | pPOP6 | pHTB2 | pTDH3 | pTEF2 | pTEF1 | pREV1 | pCCW12 | 375.4758539 |
| D8 | pPOP6 | pHTB2 | pTDH3 | pTEF2 | pALD6 | pREV1 | pCCW12 | 441.0788448 |
| D9 | pPOP6 | pHTB2 | pTDH3 | pTEF2 | pRAD27 | pREV1 | pCCW12 | 386.4923599 |
| E1 | pHHF2 | pHTB2 | pTDH3 | pTEF2 | pTEF1 | pHHF1 | pCCW12 | 453.9016285 |
| E2 | pHHF2 | pHTB2 | pTDH3 | pTEF2 | pALD6 | pHHF1 | pCCW12 | 465.2358518 |
| E3 | pHHF2 | pHTB2 | pTDH3 | pTEF2 | pRAD27 | pHHF1 | pCCW12 | 489.3750557 |
| E4 | pRPL18B | pHTB2 | pTDH3 | pTEF2 | pTEF1 | pHHF1 | pCCW12 | 475.5346698 |
| E5 | pRPL18B | pHTB2 | pTDH3 | pTEF2 | pALD6 | pHHF1 | pCCW12 | 486.62597 |
| E6 | pRPL18B | pHTB2 | pTDH3 | pTEF2 | pRAD27 | pHHF1 | pCCW12 | 379.9341203 |
| E7 | pPOP6 | pHTB2 | pTDH3 | pTEF2 | pTEF1 | pHHF1 | pCCW12 | 402.816314 |
| E8 | pPOP6 | pHTB2 | pTDH3 | pTEF2 | pALD6 | pHHF1 | pCCW12 | 419.7709405 |
| E9 | pPOP6 | pHTB2 | pTDH3 | pTEF2 | pRAD27 | pHHF1 | pCCW12 | 418.211033 |
| F1 | pHHF2 | pRNR2 | pTDH3 | pTEF2 | pTEF1 | pHHF1 | pCCW12 | 477.3612527 |
| F2 | pHHF2 | pRNR2 | pTDH3 | pTEF2 | pALD6 | pHHF1 | pCCW12 | 426.3983862 |
| F3 | pHHF2 | pRNR2 | pTDH3 | pTEF2 | pRAD27 | pHHF1 | pCCW12 | 480.641194 |
| F4 | pRPL18B | pRNR2 | pTDH3 | pTEF2 | pTEF1 | pHHF1 | pCCW12 | 455.493994 |
| F5 | pRPL18B | pRNR2 | pTDH3 | pTEF2 | pALD6 | pHHF1 | pCCW12 | 470.2409752 |
| F6 | pRPL18B | pRNR2 | pTDH3 | pTEF2 | pRAD27 | pHHF1 | pCCW12 | 446.3741225 |
| F7 | pPOP6 | pRNR2 | pTDH3 | pTEF2 | pTEF1 | pHHF1 | pCCW12 | 445.2569601 |
| F8 | pPOP6 | pRNR2 | pTDH3 | pTEF2 | pALD6 | pHHF1 | pCCW12 | 427.6275317 |
| F9 | pPOP6 | pRNR2 | pTDH3 | pTEF2 | pRAD27 | pHHF1 | pCCW12 | 398.8999412 |
| G1 | pHHF2 | pRNR2 | pTDH3 | pTEF2 | pTEF1 | pRET2 | pCCW12 | 414.9747161 |
| G2 | pHHF2 | pRNR2 | pTDH3 | pTEF2 | pALD6 | pRET2 | pCCW12 | 490.617234 |
| G3 | pHHF2 | pRNR2 | pTDH3 | pTEF2 | pRAD27 | pRET2 | pCCW12 | 458.3581896 |
| G4 | pRPL18B | pRNR2 | pTDH3 | pTEF2 | pTEF1 | pRET2 | pCCW12 | 447.7179198 |
| G5 | pRPL18B | pRNR2 | pTDH3 | pTEF2 | pALD6 | pRET2 | pCCW12 | 446.8349425 |
| G6 | pRPL18B | pRNR2 | pTDH3 | pTEF2 | pRAD27 | pRET2 | pCCW12 | 429.8666205 |
| G7 | pPOP6 | pRNR2 | pTDH3 | pTEF2 | pTEF1 | pRET2 | pCCW12 | 457.6854404 |
| G8 | pPOP6 | pRNR2 | pTDH3 | pTEF2 | pALD6 | pRET2 | pCCW12 | 426.0563354 |
| G9 | pPOP6 | pRNR2 | pTDH3 | pTEF2 | pRAD27 | pRET2 | pCCW12 | 423.1515979 |
| H1 | pHHF2 | pRNR2 | pTDH3 | pTEF2 | pTEF1 | pREV1 | pCCW12 | 483.2568762 |
| H2 | pHHF2 | pRNR2 | pTDH3 | pTEF2 | pALD6 | pREV1 | pCCW12 | 476.2790915 |
| H3 | pHHF2 | pRNR2 | pTDH3 | pTEF2 | pRAD27 | pREV1 | pCCW12 | 472.4230679 |
| H4 | pRPL18B | pRNR2 | pTDH3 | pTEF2 | pTEF1 | pREV1 | pCCW12 | 479.9329446 |
| H5 | pRPL18B | pRNR2 | pTDH3 | pTEF2 | pALD6 | pREV1 | pCCW12 | 423.3626509 |
| H6 | pRPL18B | pRNR2 | pTDH3 | pTEF2 | pRAD27 | pREV1 | pCCW12 | 442.4082618 |
| H7 | pPOP6 | pRNR2 | pTDH3 | pTEF2 | pTEF1 | pREV1 | pCCW12 | 468.4434898 |
| H8 | pPOP6 | pRNR2 | pTDH3 | pTEF2 | pALD6 | pREV1 | pCCW12 | 394.3226328 |
| H9 | pPOP6 | pRNR2 | pTDH3 | pTEF2 | pRAD27 | pREV1 | pCCW12 | 394.8700854 |
| I1 | pHHF2 | pPGK1 | pTDH3 | pPAB1 | pTEF1 | pHHF1 | pCCW12 | 442.6115968 |
| I2 | pHHF2 | pPGK1 | pTDH3 | pPAB1 | pALD6 | pHHF1 | pCCW12 | 468.122392 |
| I3 | pHHF2 | pPGK1 | pTDH3 | pPAB1 | pRAD27 | pHHF1 | pCCW12 | 500.1403618 |
| I4 | pRPL18B | pPGK1 | pTDH3 | pPAB1 | pTEF1 | pHHF1 | pCCW12 | 500.7356134 |
| I5 | pRPL18B | pPGK1 | pTDH3 | pPAB1 | pALD6 | pHHF1 | pCCW12 | 433.0473649 |
| I6 | pRPL18B | pPGK1 | pTDH3 | pPAB1 | pRAD27 | pHHF1 | pCCW12 | 427.3786113 |
| I7 | pPOP6 | pPGK1 | pTDH3 | pPAB1 | pTEF1 | pHHF1 | pCCW12 | 494.9676899 |
| I8 | pPOP6 | pPGK1 | pTDH3 | pPAB1 | pALD6 | pHHF1 | pCCW12 | 439.742717 |
| I9 | pPOP6 | pPGK1 | pTDH3 | pPAB1 | pRAD27 | pHHF1 | pCCW12 | 420.7028482 |
| J1 | pHHF2 | pPGK1 | pTDH3 | pPAB1 | pTEF1 | pRET2 | pCCW12 | 470.080355 |
| J2 | pHHF2 | pPGK1 | pTDH3 | pPAB1 | pALD6 | pRET2 | pCCW12 | 432.869045 |
| J3 | pHHF2 | pPGK1 | pTDH3 | pPAB1 | pRAD27 | pRET2 | pCCW12 | 455.3802193 |
| J4 | pRPL18B | pPGK1 | pTDH3 | pPAB1 | pTEF1 | pRET2 | pCCW12 | 458.9642964 |
| J5 | pRPL18B | pPGK1 | pTDH3 | pPAB1 | pALD6 | pRET2 | pCCW12 | 434.3579597 |
| J6 | pRPL18B | pPGK1 | pTDH3 | pPAB1 | pRAD27 | pRET2 | pCCW12 | 439.139643 |
| J7 | pPOP6 | pPGK1 | pTDH3 | pPAB1 | pTEF1 | pRET2 | pCCW12 | 433.809653 |
| J8 | pPOP6 | pPGK1 | pTDH3 | pPAB1 | pALD6 | pRET2 | pCCW12 | 436.9500231 |
| J9 | pPOP6 | pPGK1 | pTDH3 | pPAB1 | pRAD27 | pRET2 | pCCW12 | 383.1199478 |
| K1 | pHHF2 | pPGK1 | pTDH3 | pPAB1 | pTEF1 | pREV1 | pCCW12 | 475.5051365 |
| K2 | pHHF2 | pPGK1 | pTDH3 | pPAB1 | pALD6 | pREV1 | pCCW12 | 476.6265789 |
| K3 | pHHF2 | pPGK1 | pTDH3 | pPAB1 | pRAD27 | pREV1 | pCCW12 | 465.3839588 |
| K4 | pRPL18B | pPGK1 | pTDH3 | pPAB1 | pTEF1 | pREV1 | pCCW12 | 445.2750025 |
| K5 | pRPL18B | pPGK1 | pTDH3 | pPAB1 | pALD6 | pREV1 | pCCW12 | 431.4845384 |
| K6 | pRPL18B | pPGK1 | pTDH3 | pPAB1 | pRAD27 | pREV1 | pCCW12 | 392.0193642 |
| K7 | pPOP6 | pPGK1 | pTDH3 | pPAB1 | pTEF1 | pREV1 | pCCW12 | 433.2137264 |
| K8 | pPOP6 | pPGK1 | pTDH3 | pPAB1 | pALD6 | pREV1 | pCCW12 | 424.6384639 |
| K9 | pPOP6 | pPGK1 | pTDH3 | pPAB1 | pRAD27 | pREV1 | pCCW12 | 427.7537622 |
| L1 | pHHF2 | pHTB2 | pTDH3 | pPAB1 | pTEF1 | pHHF1 | pCCW12 | 465.4400175 |
| L2 | pHHF2 | pHTB2 | pTDH3 | pPAB1 | pALD6 | pHHF1 | pCCW12 | 456.3614794 |
| L3 | pHHF2 | pHTB2 | pTDH3 | pPAB1 | pRAD27 | pHHF1 | pCCW12 | 457.0726516 |
| L4 | pRPL18B | pHTB2 | pTDH3 | pPAB1 | pTEF1 | pHHF1 | pCCW12 | 456.4654591 |
| L5 | pRPL18B | pHTB2 | pTDH3 | pPAB1 | pALD6 | pHHF1 | pCCW12 | 463.0862448 |
| L6 | pRPL18B | pHTB2 | pTDH3 | pPAB1 | pRAD27 | pHHF1 | pCCW12 | 450.5404776 |
| L7 | pPOP6 | pHTB2 | pTDH3 | pPAB1 | pTEF1 | pHHF1 | pCCW12 | 431.5209431 |
| L8 | pPOP6 | pHTB2 | pTDH3 | pPAB1 | pALD6 | pHHF1 | pCCW12 | 412.6174326 |
| L9 | pPOP6 | pHTB2 | pTDH3 | pPAB1 | pRAD27 | pHHF1 | pCCW12 | 421.8756494 |
| β1 | pHHF2 | pHTB2 | pTDH3 | pPAB1 | pTEF1 | pRET2 | pCCW12 | 501.1932026 |
| β2 | pHHF2 | pHTB2 | pTDH3 | pPAB1 | pALD6 | pRET2 | pCCW12 | 517.9414633 |
| β3 | pHHF2 | pHTB2 | pTDH3 | pPAB1 | pRAD27 | pRET2 | pCCW12 | 502.342742 |
| β4 | pRPL18B | pHTB2 | pTDH3 | pPAB1 | pTEF1 | pRET2 | pCCW12 | 509.5189277 |
| β5 | pRPL18B | pHTB2 | pTDH3 | pPAB1 | pALD6 | pRET2 | pCCW12 | 505.2825402 |
| β6 | pRPL18B | pHTB2 | pTDH3 | pPAB1 | pRAD27 | pRET2 | pCCW12 | 440.5431304 |
| β7 | pPOP6 | pHTB2 | pTDH3 | pPAB1 | pTEF1 | pRET2 | pCCW12 | 502.443358 |
| β8 | pPOP6 | pHTB2 | pTDH3 | pPAB1 | pALD6 | pRET2 | pCCW12 | 414.9491274 |
| β9 | pPOP6 | pHTB2 | pTDH3 | pPAB1 | pRAD27 | pRET2 | pCCW12 | 441.4560147 |
| M1 | pHHF2 | pHTB2 | pTDH3 | pPAB1 | pTEF1 | pREV1 | pCCW12 | 475.9395378 |
| M2 | pHHF2 | pHTB2 | pTDH3 | pPAB1 | pALD6 | pREV1 | pCCW12 | 445.1090082 |
| M3 | pHHF2 | pHTB2 | pTDH3 | pPAB1 | pRAD27 | pREV1 | pCCW12 | 437.127584 |
| M4 | pRPL18B | pHTB2 | pTDH3 | pPAB1 | pTEF1 | pREV1 | pCCW12 | 433.8262371 |
| M5 | pRPL18B | pHTB2 | pTDH3 | pPAB1 | pALD6 | pREV1 | pCCW12 | 475.039272 |
| M6 | pRPL18B | pHTB2 | pTDH3 | pPAB1 | pRAD27 | pREV1 | pCCW12 | 469.2291762 |
| M7 | pPOP6 | pHTB2 | pTDH3 | pPAB1 | pTEF1 | pREV1 | pCCW12 | 461.3952785 |
| M8 | pPOP6 | pHTB2 | pTDH3 | pPAB1 | pALD6 | pREV1 | pCCW12 | 455.6781434 |
| M9 | pPOP6 | pHTB2 | pTDH3 | pPAB1 | pRAD27 | pREV1 | pCCW12 | 422.9415018 |
| N1 | pHHF2 | pRNR2 | pTDH3 | pPAB1 | pTEF1 | pRET2 | pCCW12 | 454.5167276 |
| N2 | pHHF2 | pRNR2 | pTDH3 | pPAB1 | pALD6 | pRET2 | pCCW12 | 510.4869867 |
| N3 | pHHF2 | pRNR2 | pTDH3 | pPAB1 | pRAD27 | pRET2 | pCCW12 | 513.5257601 |
| N4 | pRPL18B | pRNR2 | pTDH3 | pPAB1 | pTEF1 | pRET2 | pCCW12 | 440.9364602 |
| N5 | pRPL18B | pRNR2 | pTDH3 | pPAB1 | pALD6 | pRET2 | pCCW12 | 473.3233065 |
| N6 | pRPL18B | pRNR2 | pTDH3 | pPAB1 | pRAD27 | pRET2 | pCCW12 | 409.7907273 |
| N7 | pPOP6 | pRNR2 | pTDH3 | pPAB1 | pTEF1 | pRET2 | pCCW12 | 407.2001148 |
| N8 | pPOP6 | pRNR2 | pTDH3 | pPAB1 | pALD6 | pRET2 | pCCW12 | 437.2492284 |
| N9 | pPOP6 | pRNR2 | pTDH3 | pPAB1 | pRAD27 | pRET2 | pCCW12 | 315.0361339 |
| O1 | pHHF2 | pRNR2 | pTDH3 | pPAB1 | pTEF1 | pHHF1 | pCCW12 | 423.4519746 |
| O2 | pHHF2 | pRNR2 | pTDH3 | pPAB1 | pALD6 | pHHF1 | pCCW12 | 432.2590417 |
| O3 | pHHF2 | pRNR2 | pTDH3 | pPAB1 | pRAD27 | pHHF1 | pCCW12 | 444.0609661 |
| O4 | pRPL18B | pRNR2 | pTDH3 | pPAB1 | pTEF1 | pHHF1 | pCCW12 | 422.3564398 |
| O5 | pRPL18B | pRNR2 | pTDH3 | pPAB1 | pALD6 | pHHF1 | pCCW12 | 430.9498774 |
| O6 | pRPL18B | pRNR2 | pTDH3 | pPAB1 | pRAD27 | pHHF1 | pCCW12 | 416.5738045 |
| O7 | pPOP6 | pRNR2 | pTDH3 | pPAB1 | pTEF1 | pHHF1 | pCCW12 | 409.9993279 |
| O8 | pPOP6 | pRNR2 | pTDH3 | pPAB1 | pALD6 | pHHF1 | pCCW12 | 385.9100507 |
| O9 | pPOP6 | pRNR2 | pTDH3 | pPAB1 | pRAD27 | pHHF1 | pCCW12 | 391.9396126 |
| P1 | pHHF2 | pRNR2 | pTDH3 | pPAB1 | pTEF1 | pREV1 | pCCW12 | 434.5250837 |
| P2 | pHHF2 | pRNR2 | pTDH3 | pPAB1 | pALD6 | pREV1 | pCCW12 | 418.262363 |
| P3 | pHHF2 | pRNR2 | pTDH3 | pPAB1 | pRAD27 | pREV1 | pCCW12 | 461.8811685 |
| P4 | pRPL18B | pRNR2 | pTDH3 | pPAB1 | pTEF1 | pREV1 | pCCW12 | 420.3509002 |
| P5 | pRPL18B | pRNR2 | pTDH3 | pPAB1 | pALD6 | pREV1 | pCCW12 | 428.4894336 |
| P6 | pRPL18B | pRNR2 | pTDH3 | pPAB1 | pRAD27 | pREV1 | pCCW12 | 433.3411489 |
| P7 | pPOP6 | pRNR2 | pTDH3 | pPAB1 | pTEF1 | pREV1 | pCCW12 | 409.9420939 |
| P8 | pPOP6 | pRNR2 | pTDH3 | pPAB1 | pALD6 | pREV1 | pCCW12 | 421.7857288 |
| P9 | pPOP6 | pRNR2 | pTDH3 | pPAB1 | pRAD27 | pREV1 | pCCW12 | 380.115259 |
| Q1 | pHHF2 | pPGK1 | pTDH3 | pPSP2 | pTEF1 | pHHF1 | pCCW12 | 462.9243199 |
| Q2 | pHHF2 | pPGK1 | pTDH3 | pPSP2 | pALD6 | pHHF1 | pCCW12 | 477.0869969 |
| Q3 | pHHF2 | pPGK1 | pTDH3 | pPSP2 | pRAD27 | pHHF1 | pCCW12 | 434.8438199 |
| Q4 | pRPL18B | pPGK1 | pTDH3 | pPSP2 | pTEF1 | pHHF1 | pCCW12 | 423.2766734 |
| Q5 | pRPL18B | pPGK1 | pTDH3 | pPSP2 | pALD6 | pHHF1 | pCCW12 | 403.9730438 |
| Q6 | pRPL18B | pPGK1 | pTDH3 | pPSP2 | pRAD27 | pHHF1 | pCCW12 | 418.0310848 |
| Q7 | pPOP6 | pPGK1 | pTDH3 | pPSP2 | pTEF1 | pHHF1 | pCCW12 | 377.024716 |
| Q8 | pPOP6 | pPGK1 | pTDH3 | pPSP2 | pALD6 | pHHF1 | pCCW12 | 413.3834125 |
| Q9 | pPOP6 | pPGK1 | pTDH3 | pPSP2 | pRAD27 | pHHF1 | pCCW12 | 461.6338981 |
| R1 | pHHF2 | pPGK1 | pTDH3 | pPSP2 | pTEF1 | pREV1 | pCCW12 | 480.2765582 |
| R2 | pHHF2 | pPGK1 | pTDH3 | pPSP2 | pALD6 | pREV1 | pCCW12 | 484.4369198 |
| R3 | pHHF2 | pPGK1 | pTDH3 | pPSP2 | pRAD27 | pREV1 | pCCW12 | 483.9910082 |
| R4 | pRPL18B | pPGK1 | pTDH3 | pPSP2 | pTEF1 | pREV1 | pCCW12 | 459.6871068 |
| R5 | pRPL18B | pPGK1 | pTDH3 | pPSP2 | pALD6 | pREV1 | pCCW12 | 470.9392406 |
| R6 | pRPL18B | pPGK1 | pTDH3 | pPSP2 | pRAD27 | pREV1 | pCCW12 | 449.7841523 |
| R7 | pPOP6 | pPGK1 | pTDH3 | pPSP2 | pTEF1 | pREV1 | pCCW12 | 448.0531834 |
| R8 | pPOP6 | pPGK1 | pTDH3 | pPSP2 | pALD6 | pREV1 | pCCW12 | 469.3834147 |
| R9 | pPOP6 | pPGK1 | pTDH3 | pPSP2 | pRAD27 | pREV1 | pCCW12 | 468.0717234 |
| S1 | pHHF2 | pPGK1 | pTDH3 | pPSP2 | pTEF1 | pRET2 | pCCW12 | 435.9135131 |
| S2 | pHHF2 | pPGK1 | pTDH3 | pPSP2 | pALD6 | pRET2 | pCCW12 | 386.6212719 |
| S3 | pHHF2 | pPGK1 | pTDH3 | pPSP2 | pRAD27 | pRET2 | pCCW12 | 427.8869201 |
| S4 | pRPL18B | pPGK1 | pTDH3 | pPSP2 | pTEF1 | pRET2 | pCCW12 | 403.7716265 |
| S5 | pRPL18B | pPGK1 | pTDH3 | pPSP2 | pALD6 | pRET2 | pCCW12 | 430.6500449 |
| S6 | pRPL18B | pPGK1 | pTDH3 | pPSP2 | pRAD27 | pRET2 | pCCW12 | 381.6576054 |
| S7 | pPOP6 | pPGK1 | pTDH3 | pPSP2 | pTEF1 | pRET2 | pCCW12 | 420.2558556 |
| S8 | pPOP6 | pPGK1 | pTDH3 | pPSP2 | pALD6 | pRET2 | pCCW12 | 354.0818936 |
| S9 | pPOP6 | pPGK1 | pTDH3 | pPSP2 | pRAD27 | pRET2 | pCCW12 | 400.2516817 |
| T1 | pHHF2 | pHTB2 | pTDH3 | pPSP2 | pTEF1 | pRET2 | pCCW12 | 409.5993801 |
| T2 | pHHF2 | pHTB2 | pTDH3 | pPSP2 | pALD6 | pRET2 | pCCW12 | 398.9217484 |
| T3 | pHHF2 | pHTB2 | pTDH3 | pPSP2 | pRAD27 | pRET2 | pCCW12 | 361.3764126 |
| T4 | pRPL18B | pHTB2 | pTDH3 | pPSP2 | pTEF1 | pRET2 | pCCW12 | 413.4821306 |
| T5 | pRPL18B | pHTB2 | pTDH3 | pPSP2 | pALD6 | pRET2 | pCCW12 | 333.5966993 |
| T6 | pRPL18B | pHTB2 | pTDH3 | pPSP2 | pRAD27 | pRET2 | pCCW12 | 372.1194899 |
| T7 | pPOP6 | pHTB2 | pTDH3 | pPSP2 | pTEF1 | pRET2 | pCCW12 | 409.8139805 |
| T8 | pPOP6 | pHTB2 | pTDH3 | pPSP2 | pALD6 | pRET2 | pCCW12 | 419.3790213 |
| T9 | pPOP6 | pHTB2 | pTDH3 | pPSP2 | pRAD27 | pRET2 | pCCW12 | 400.1225642 |
| U1 | pHHF2 | pHTB2 | pTDH3 | pPSP2 | pTEF1 | pHHF1 | pCCW12 | 461.9529033 |
| U2 | pHHF2 | pHTB2 | pTDH3 | pPSP2 | pALD6 | pHHF1 | pCCW12 | 468.4005072 |
| U3 | pHHF2 | pHTB2 | pTDH3 | pPSP2 | pRAD27 | pHHF1 | pCCW12 | 462.3418469 |
| U4 | pRPL18B | pHTB2 | pTDH3 | pPSP2 | pTEF1 | pHHF1 | pCCW12 | 464.6720725 |
| U5 | pRPL18B | pHTB2 | pTDH3 | pPSP2 | pALD6 | pHHF1 | pCCW12 | 429.2381552 |
| U6 | pRPL18B | pHTB2 | pTDH3 | pPSP2 | pRAD27 | pHHF1 | pCCW12 | 381.7243825 |
| U7 | pPOP6 | pHTB2 | pTDH3 | pPSP2 | pTEF1 | pHHF1 | pCCW12 | 433.0161172 |
| U8 | pPOP6 | pHTB2 | pTDH3 | pPSP2 | pALD6 | pHHF1 | pCCW12 | 416.6001715 |
| U9 | pPOP6 | pHTB2 | pTDH3 | pPSP2 | pRAD27 | pHHF1 | pCCW12 | 404.9922743 |
| V1 | pHHF2 | pHTB2 | pTDH3 | pPSP2 | pTEF1 | pREV1 | pCCW12 | 421.3705848 |
| V2 | pHHF2 | pHTB2 | pTDH3 | pPSP2 | pALD6 | pREV1 | pCCW12 | 422.6214473 |
| V3 | pHHF2 | pHTB2 | pTDH3 | pPSP2 | pRAD27 | pREV1 | pCCW12 | 435.8909075 |
| V4 | pRPL18B | pHTB2 | pTDH3 | pPSP2 | pTEF1 | pREV1 | pCCW12 | 447.6789821 |
| V5 | pRPL18B | pHTB2 | pTDH3 | pPSP2 | pALD6 | pREV1 | pCCW12 | 381.243258 |
| V6 | pRPL18B | pHTB2 | pTDH3 | pPSP2 | pRAD27 | pREV1 | pCCW12 | 411.6025295 |
| V7 | pPOP6 | pHTB2 | pTDH3 | pPSP2 | pTEF1 | pREV1 | pCCW12 | 272.7760743 |
| V8 | pPOP6 | pHTB2 | pTDH3 | pPSP2 | pALD6 | pREV1 | pCCW12 | 438.0537457 |
| V9 | pPOP6 | pHTB2 | pTDH3 | pPSP2 | pRAD27 | pREV1 | pCCW12 | 315.0214592 |
| W 1 | pHHF2 | pRNR2 | pTDH3 | pPSP2 | pTEF1 | pRET2 | pCCW12 | 386.3302112 |
| W 2 | pHHF2 | pRNR2 | pTDH3 | pPSP2 | pALD6 | pRET2 | pCCW12 | 378.6835101 |
| W 3 | pHHF2 | pRNR2 | pTDH3 | pPSP2 | pRAD27 | pRET2 | pCCW12 | 400.8307201 |
| W 4 | pRPL18B | pRNR2 | pTDH3 | pPSP2 | pTEF1 | pRET2 | pCCW12 | 376.0581519 |
| W 5 | pRPL18B | pRNR2 | pTDH3 | pPSP2 | pALD6 | pRET2 | pCCW12 | 426.731007 |
| W 6 | pRPL18B | pRNR2 | pTDH3 | pPSP2 | pRAD27 | pRET2 | pCCW12 | 431.7291039 |
| W 7 | pPOP6 | pRNR2 | pTDH3 | pPSP2 | pTEF1 | pRET2 | pCCW12 | 397.9680037 |
| W 8 | pPOP6 | pRNR2 | pTDH3 | pPSP2 | pALD6 | pRET2 | pCCW12 | 445.6893788 |
| W 9 | pPOP6 | pRNR2 | pTDH3 | pPSP2 | pRAD27 | pRET2 | pCCW12 | 385.3375456 |
| X 1 | pHHF2 | pRNR2 | pTDH3 | pPSP2 | pTEF1 | pHHF1 | pCCW12 | 405.4243384 |
| X 2 | pHHF2 | pRNR2 | pTDH3 | pPSP2 | pALD6 | pHHF1 | pCCW12 | 377.3324828 |
| X 3 | pHHF2 | pRNR2 | pTDH3 | pPSP2 | pRAD27 | pHHF1 | pCCW12 | 406.5262469 |
| X 4 | pRPL18B | pRNR2 | pTDH3 | pPSP2 | pTEF1 | pHHF1 | pCCW12 | 381.5856461 |
| X 5 | pRPL18B | pRNR2 | pTDH3 | pPSP2 | pALD6 | pHHF1 | pCCW12 | 399.9937835 |
| X 6 | pRPL18B | pRNR2 | pTDH3 | pPSP2 | pRAD27 | pHHF1 | pCCW12 | 407.8301152 |
| X 7 | pPOP6 | pRNR2 | pTDH3 | pPSP2 | pTEF1 | pHHF1 | pCCW12 | 419.8333741 |
| X 8 | pPOP6 | pRNR2 | pTDH3 | pPSP2 | pALD6 | pHHF1 | pCCW12 | 374.5296281 |
| X 9 | pPOP6 | pRNR2 | pTDH3 | pPSP2 | pRAD27 | pHHF1 | pCCW12 | 387.5125876 |
| γ1 | pHHF2 | pRNR2 | pTDH3 | pPSP2 | pTEF1 | pREV1 | pCCW12 | 337.3292773 |
| γ2 | pHHF2 | pRNR2 | pTDH3 | pPSP2 | pALD6 | pREV1 | pCCW12 | 215.1025068 |
| γ3 | pHHF2 | pRNR2 | pTDH3 | pPSP2 | pRAD27 | pREV1 | pCCW12 | 215.1826088 |
| γ4 | pRPL18B | pRNR2 | pTDH3 | pPSP2 | pTEF1 | pREV1 | pCCW12 | 197.4239299 |
| γ5 | pRPL18B | pRNR2 | pTDH3 | pPSP2 | pALD6 | pREV1 | pCCW12 | 196.4894027 |
| γ6 | pRPL18B | pRNR2 | pTDH3 | pPSP2 | pRAD27 | pREV1 | pCCW12 | 175.4587541 |
| γ7 | pPOP6 | pRNR2 | pTDH3 | pPSP2 | pTEF1 | pREV1 | pCCW12 | 260.17997 |
| γ8 | pPOP6 | pRNR2 | pTDH3 | pPSP2 | pALD6 | pREV1 | pCCW12 | 246.187419 |
| γ9 | pPOP6 | pRNR2 | pTDH3 | pPSP2 | pRAD27 | pREV1 | pCCW12 | 221.1876575 |

The references cited throughout this application, are incorporated for all purposes apparent herein and in the references themselves as if each reference was fully set forth. For the sake of presentation, specific ones of these references are cited at particular locations herein. A citation of a reference at a particular location indicates a manner(s) in which the teachings of the reference are incorporated. However, a citation of a reference at a particular location does not limit the manner in which all of the teachings of the cited reference are incorporated for all purposes.

It is understood, therefore, that this invention is not limited to the particular embodiments disclosed, but is intended to cover all modifications which are within the spirit and scope of the invention as defined by the appended claims; the above description; and/or shown in the attached drawings.

1. A composition comprising a modified yeast cell comprising:
(i) open reading frames encoding ERG8, ERG10, ERG12, ERG13, and ERG19; and
(ii) a first regulatory sequence of weak-strength, medium-strength or high-strength operably linked to the open reading frame encoding ERG12.

2. The composition of claim 1, wherein the modified yeast cell further comprises one or both of an open reading frame encoding tHMG1 and an open reading frame encoding IDI.

3. The composition of claim 1, wherein the modified yeast cell further comprises one or more of: a second regulatory sequence operably linked to the open reading frame encoding ERG8, a third regulatory sequence operably linked to the open reading frame encoding ERG10, a fourth regulatory sequence operably linked to the open reading frame encoding ERG13, and a fifth regulatory sequence operably linked to the open reading frame encoding ERG19.

4. The composition of claim 3, wherein the first regulatory sequence, the second regulatory sequence, the third regulatory sequence, the fourth regulatory sequence, and the fifth regulatory sequence are each high-strength promoters.

5. The composition of claim 4, wherein the first regulatory sequence, the second regulatory sequence, the third regulatory sequence, the fourth regulatory sequence, and the fifth regulatory sequence are independently selected from a promoter comprising a nucleic acid sequence comprising at least about 72% sequence identity to SEQ ID NO: 1, SEQ ID NO: 2, SEQ ID NO: 3, SEQ ID NO: 4, SEQ ID NO: 5, SEQ ID NO: 6, or SEQ ID NO: 7; or the first regulatory sequence, the second regulatory sequence, the third regulatory sequence, the fourth regulatory sequence, and the fifth regulatory sequence are independently selected from: pTDH3, pCCW12, pPGK1, pHHF2, pTEF1, pTEF2, and pHHF1.

6. The composition of claim 3, wherein the first regulatory sequence, the second regulatory sequence, the third regulatory sequence, the fourth regulatory sequence, and the fifth regulatory sequence are each medium-strength promoters.

7. The composition of claim 6, wherein the first regulatory sequence, the second regulatory sequence, the third regulatory sequence, the fourth regulatory sequence, and the fifth regulatory sequence are independently selected from a promoter comprising a nucleic acid sequence that comprises at least about 72% sequence to SEQ ID NO: 8, SEQ ID NO: 9, SEQ ID NO: 10, SEQ ID NO: 11, or SEQ ID NO: 12; or the first regulatory sequence, the second regulatory sequence, the third regulatory sequence, the fourth regulatory sequence, and the fifth regulatory sequence are independently selected from pRPL18B, pHTB2, pALD6, pPAB1, and pRET2.

8. The composition of claim 3, wherein the first regulatory sequence is selected from a promoter comprising a nucleic acid sequence having at least about 72% sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, and the second regulatory sequence, the third regulatory sequence, the fourth regulatory sequence, and the fifth regulatory sequence are each independently selected from a promoter comprising a nucleic acid sequence comprising at least about 72% sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, or SEQ ID NO: 17.

9. The composition of claim 3, wherein the first regulatory sequence, the second regulatory sequence, the third regulatory sequence, the fourth regulatory sequence, and the fifth regulatory sequence are each weak-strength promoters.

10. The composition of claim 9, wherein the first regulatory sequence, the second regulatory sequence, the third regulatory sequence, the fourth regulatory sequence, and the fifth regulatory sequence are independently selected from a promoter comprising a nucleic acid sequence that comprises at least about 72% sequence to SEQ ID NO: 13, SEQ ID NO: 14, SEQ ID NO: 15, SEQ ID NO: 16, or SEQ ID NO: 17; or the first regulatory sequence, the second regulatory sequence, the third regulatory sequence, the fourth regulatory sequence, and the fifth regulatory sequence are independently selected from pPOP6, pRNR2, pPSP2, pRAD27, and pREV1.

11. The composition of claim 3, wherein the first regulatory sequence is selected from a promoter comprising a nucleic acid sequence having at least about 72% sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, or SEQ ID NO: 17; and the second regulatory sequence, the third regulatory sequence, the fourth regulatory sequence, and the fifth regulatory sequence are each independently selected from a promoter comprising a nucleic acid sequence comprising at least about 72% sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, or SEQ ID NO: 17.

12. The composition of claim 3, wherein the modified yeast cell is free of modification of any of yeast genes: LPP1, DPP1, HO, ERG1, ANT1, IDP2, IDP3, Cit2, ACS1, ACL1, ACL2, Met15, RHR2, NADH-HMGR, ERG9, GPD1, and GPD2.

13. The composition of claim 1, wherein the modified yeast cell further comprises one, two, or three regulatory sequences operably linked to the open reading frame encoding ERG8, one, two, or three regulatory sequences operably linked to the open reading frame encoding ERG10, one, two, or three regulatory sequences operably linked to the open reading frame encoding ERG13, and one, two, or three regulatory sequences operably linked to the open reading frame encoding ERG19, and one or more of a sixth regulatory sequence operably linked to the open reading frame encoding ERG12 and seventh regulatory sequence operably linked to the open reading frame encoding ERG12.

14. The composition of claim 1, wherein a culture of the modified yeast cell has about a 94-fold, about a 60-fold, and about a 35-fold improved titer of monoterpene geraniol, sesquiterpene α-humulene, and triterpene squalene, respectively, over a culture of wild type yeast cell.

15. The composition of claim 1, further comprising a terpene and a culture medium; wherein the terpene is at least about 10 mg/L to about 20 mg/L of culture medium.

16. A method of making a terpene comprising: inoculating a growth medium with a modified yeast cell, the modified yeast cell comprising open reading frames encoding ERG8, ERG10, ERG12, ERG13, ERG19, tHMG1, IDI and a first regulatory sequence of weak-strength, medium-strength or high-strength operably linked to the open reading frame encoding ERG12.

17. The method of claim 14, wherein the growth medium is synthetic-defined medium plus an antibiotic.

18. The method of claim 14, wherein the growth medium is glucose medium or oleate medium.

19. The method of claim 14 further comprising incubating the modified yeast cell in the growth medium.

20. The method of claim 17 further comprising isolating a plurality of modified yeast cells from the culture medium after the incubating the plurality of cells, disrupting the membrane of the modified yeast cells, and collecting the liquid phase after the step of disrupting.

21. The method of claim 18 further comprising drying the liquid phase.

22. A kit comprising a nucleic acid molecule comprising nucleic acid sequence comprising an open reading frame encoding ERG12 and a first regulatory sequence of weak-strength, medium-strength or high-strength operably linked to the open reading frame encoding ERG12.

23. The kit of claim 20 further comprising a yeast cell.


SEQUENCE LISTING (145 sequences)

SEQ ID NO: 1 - DNA, 686 bp, Saccharomyces cerevisiae
  source 1..686: mol_type = genomic DNA
cagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaagatct

SEQ ID NO: 2 - DNA, 705 bp, Saccharomyces cerevisiae
  source 1..705: mol_type = genomic DNA
cacccatgaaccacacggttagtccaaaaggggcagttcagattccagatgcgggaattagcttgctgccaccctcacctcactaacgctgcggtgtgcggatacttcatgctatttatagacgcgcgtgtcggaatcagcacgcgcaagaaccaaatgggaaaatcggaatgggtccagaactgctttgagtgctggctattggcgtctgatttccgttttgggaatcctttgccgcgcgcccctctcaaaactccgcacaagtcccagaaagcgggaaagaaataaaacgccaccaaaaaaaaaaaaataaaagccaatcctcgaagcgtgggtggtaggccctggattatcccgtacaagtatttctcaggagtaaaaaaaccgtttgttttggaatttcccatttcgcggccacctacgccgctatctttgcaacaactatctgcgataactcagcaaattttgcatattcgtgttgcagtattgcgataatgggagtcttacttccaacataacggcagaaagaaatgtgagaaaattttgcatcctttgcctccgttcaagtatataaagtcggcatgcttgataatctttctttccatcctacattgttctaattattcttattctcctttattctttcctaacataccaagaaattaatcttctgtcattcgcttaaacactatatcaataaagatc

SEQ ID NO: 3 - DNA, 705 bp, Saccharomyces cerevisiae
  source 1..705: mol_type = genomic DNA
gatcttgttttatatttgttgtaaaaagtagataattacttccttgatgatctgtaaaaaagagaaaaagaaagcatctaagaacttgaaaaactacgaattagaaaagaccaaatatgtatttcttgcattgaccaatttatgcaagtttatatatatgtaaatgtaagtttcacgaggttctactaaactaaaccacccccttggttagaagaaaagagtgtgtgagaacaggctgttgttgtcacacgattcggacaattctgtttgaaagagagagagtaacagtacgatcgaacgaactttgctctggagatcacagtgggcatcatagcatgtggtactaaaccctttcccgccattccagaaccttcgattgcttgttacaaaacctgtgagccgtcgctaggaccttgttgtgtgacgaaattggaagctgcaatcaataggatgacaggaagtcgagcgtgtctgggttttttcagttttgttctttttgcaaacaaatcacgagcgacggtaatttctttctcgataagaggccacgtgctttatgagggtaacatcaattcaagaaggagggaaacacttcctttttctggccctgataatagtatgagggtgaagccaaaataaaggattcgcgcccaaatcggcatctttaaatgcaggtatgcgatagttcctcactctttccttactcac

SEQ ID NO: 4 - DNA, 682 bp, Saccharomyces cerevisiae
  source 1..682: mol_type = genomic DNA
tgtggagtgtttgcttggattctttagtaaaaggggaagaacagttggaagggccaaagtggaagtcacaaaacagtggtcctatataaaagaacaagaaaaagattatttatatacaactgcggtcacaagaagcaacgcgagagagcacaacacgctgttatcacgcaaactatgttttgacaccgagccatagccgtgattgtgcgtcacattgggcgataatgaacgctaaatgaccaactcccatccgtaggagccccttagggcgtgccaatagtttcacgcgcttaatgcgaagtgctcggaacggacaactgtggtcgtttggcaccgggaaagtggtactagaccgagagtttcgcatttgtatggcaggacgttctgggagcttcgcgtctaaagctttttcgggcgcgaaatgcagaccagaccagaacaaaacaactgacaagaaggcgtttaatttaatatgttgttcactcgcgcctgggctgttgttattcggctagatacatacgtgtttgtgcgtatgtagttatatcatatataagtatattaggatgaggcggtgaaagagattttttttttttcgcttaatttattcttttctctatcttttttcctacatcttgttcaaaagagtagcaaaaacaacaatcaatacaataaaataagatct

SEQ ID NO: 5 - DNA, 705 bp, Saccharomyces cerevisiae
  source 1..705: mol_type = genomic DNA
ccttgccaacagggagttcttcagagacatggaggctcaaaacgaaattattgacagcctagacatcaatagtcatacaacagaaagcgaccacccaactttggctgataatagcgtataaacaatgcatactttgtacgttcaaaatacaatgcagtagatatatttatgcatattacatataatacatatcacataggaagcaacaggcgcgttggacttttaattttcgaggaccgcgaatccttacatcacacccaatcccccacaagtgatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagacaccgcctcgtttctttttcttcgtcgaaaaaggcaataaaaatttttatcacgtttctttttcttgaaaatttttttttttgatttttttctctttcgatgacctcccattgatatttaagttaataaacggtcatcaatttctcaagtttcagtttcatttttcttgttctattacaactttttttacttcttgctcattagaaagaaagcatagcaatctaatctaagttttaattacaaaagatc

SEQ ID NO: 6 - DNA, 705 bp, Saccharomyces cerevisiae
  source 1..705: mol_type = genomic DNA
ttgataggtcaagatcaatgtaaacaattactttgttatgtagagtttttttagctacctatattccaccataacatcaatcatgcggttgctggtgtatttaccaataatgtttaatgtatatatatatatatatatatggggccgtatacttacatatagtagatgtcaagcgtaggcgcttcccctgccggctgtgagggcgccataaccaaggtatctatagaccgccaatcagcaaactacctccgtacattcatgttgcacccacacatttatacacccagaccgcgacaaattacccataaggttgtttgtgacggcgtcgtacaagagaacgtgggaactttttaggctcaccaaaaaagaaagaaaaaatacgagttgctgacagaagcctcaagaaaaaaaaaattcttcttcgactatgctggaggcagagatgatcgagccggtagttaactatatatagctaaattggttccatcaccttcttttctggtgtcgctccttctagtgctatttctggcttttcctatttttttttttccatttttctttctctctttctaatatataaattctcttgcattttctatttttctctctatctattctacttgtttattcccttcaaggtttttttttaaggagtacttgtttttagaatatacggtcaacgaactataattaactaaacagatc

SEQ ID NO: 7 - DNA, 705 bp, Saccharomyces cerevisiae
  source 1..705: mol_type = genomic DNA
tcttggggccttaccaccagtggactttcttgctgtttgctttgttctggccattgtttgcgtttatatatttatgttagatgtttttcttattaactagaaagaaagaatataaaaggttgaggaaagagatgtatcccgaagaatacacagtcttttatatatgtatttcaacaaggagccgtggagggtactaaaaagaaaaatcgcccgggcatttcgttatcttccacgctaaaagtcaaggagagatattacggccaggatcgcaaaggtgcagagcaaggaaatgtgagaaattgtgagaacgataatgtatgggacaatgcgaaaatgtgagaacgagagcaaaaatcttttttgtatctccccgccgaatttggaaaccgcgttctgaaaacttcgcatcttcacatagtaaaactgttccgagcgcttctccccataatggttagtggtaaaaaccgaagttgtttactttagcaaatgcccgcgaatacggtggtaaattgccacccccccttccccattcattgggtaaagaccaatttgatggataaattggttgtggaaaaggtctaattctttttcctataaataccgagatattttttctatatgatggtttccgtcgcattattgtactctatagtactaaagcaacaaacaaaaacaagcaacaaatataatatagtaaaatagatc

SEQ ID NO: 8 - DNA, 705 bp, Saccharomyces cerevisiae
  source 1..705: mol_type = genomic DNA
aagaggatgtccaatattttttttaaggaataaggatacttcaagactagattcccccctgcattcccatcagaaccgtaaaccttggcgctttccttgggaagtattcaagaagtgccttgtccggtttctgtggctcacaaaccagcgcgcccgatatggctttcttttcacttatgaatgtaccagtacgggacaattagaacgctcctgtaacaatctctttgcaaatgtggggttacattctaaccatgtcacactgctgacgaaattcaaagtaaaaaaaaatgggaccacgtcttgagaacgatagattttctttattttacattgaacagtcgttgtctcagcgcgctttatgttttcattcatacttcatattataaaataacaaaagaagaatttcatattcacgcccaagaaatcaggctgctttccaaatgcaattgacacttcattagccatcacacaaaactctttcttgctggagcttcttttaaaaaagacctcagtacaccaaacacgttacccgacctcgttattttacgacaactatgataaaattctgaagaaaaaataaaaaaattttcatacttcttgcttttatttaaaccattgaatgatttcttttgaacaaaactacctgtttcaccaaaggaaatagaaagaaaaaatcaattagaagaaaacaaaaaacaaaagatc

SEQ ID NO: 9 - DNA, 704 bp, Saccharomyces cerevisiae
  source 1..704: mol_type = genomic DNA
tatatattaaatttgctcttgttctgtactttcctaattcttatgtaaaaagacaagaatttatgatactatttaataacaaaaaactacctaagaaaagcatcatgcagtcgaaattgaaatcgaaaagtaaaactttaacggaacatgtttgaaattctaagaaagcatacatcttcatcccttatatatagagttatgtttgatattagtagtcatgttgtaatctctggcctaagtatacgtaacgaaaatggtagcacgtcgcgtttatggcccccaggttaatgtgttctctgaaattcgcatcactttgagaaataatgggaacaccttacgcgtgagctgtgcccaccgcttcgcctaataaagcggtgttctcaaaatttctccccgttttcaggatcacgagcgccatctagttctggtaaaatcgcgcttacaagaacaaagaaaagaaacatcgcgtaatgcaacagtgagacacttgccgtcatatataaggttttggatcagtaaccgttatttgagcataacacaggtttttaaatatattattatatatcatggtatatgtgtaaaatttttttgctgactggttttgtttatttatttagctttttaaaaattttactttcttcttgttaattttttctgattgctctatactcaaaccaacaacaacttactctacaactaagatc

SEQ ID NO: 10 - DNA, 705 bp, Saccharomyces cerevisiae
  source 1..705: mol_type = genomic DNA
taagggcatgatagaattggattatgtaaaaggtgaagataccattgtagaagcaaccagcacgtcgccgtggctgatgaagtctcctcttgcccgggccgcagaaaagaggggcagtggcctgtttttcgacataaatgaggggcatggccagcaccaagacgtcattgttgcatatggcgtatccaagccgaaacggcgctcgcctcatccccacgggaataaggcagccgacaaaagaaaaacgaccgaaaaggaaccagaaagaaaaaagagggtgggcgcgccgcggacgtgtaaaaagatatgcatccagcttctatatcgctttaactttaccgttttgggcatcgggaacgtatgtaacattgatctcctcttgggaacggtgagtgcaacgaatgcgatatagcaccgaccatgtgggcaaattcgtaataaattcggggtgagggggattcaagacaagcaaccttgttagtcagctcaaacagcgatttaacggttgagtaacacatcaaaacaccgttcgaggtcaagcctggcgtgtttaacaagttcttgatatcatatataaatgtaataagaagtttggtaatattcaattcgaagtgttcagtcttttacttctcttgttttatagaagaaaaaacatcaagaaacatctttaacatacacaaacacatactatcagaatacaagatc

SEQ ID NO: 11 - DNA, 705 bp, Saccharomyces cerevisiae
  source 1..705: mol_type = genomic DNA
aaggcaagcccagaaaaatatcgcaagcacctttggtcttacagtgccaacttttggcctgccgacgttaagagtacaaagctgatggcaatgtacgacaagataacagagtctcaaaagaagtgaaacaatttttcttcaccacattttccattgttccttccccccataactataaacgtatttatgtatatatatttgcgtgtaagtgtgtgtactatagggcaccgtaaagtaataatgcttaattagttactactatgaccatataagaggtcatactgtatgaagccacaaagcagatagatcaatcatgtttaacgaaaactgttaatcgaagattatttctttttttttttctctttcctttttacaaagaaaattttttttgcgctttttgccatcaccatcgcaagttctgggacaattgttctctttcgctccagttccaaggaaagaggtttctgttttacttaatagaaagtgtcatcttgtattttatatctcttctttcttgtgtaaaattctttagttttgattttgtatttttaggacagtgagctacgaagtaacatttttacttaataaccgtttgaagcatagagcaggccctggtatcaccacctaatatctggctttttattcaataaaaactcaaaaaaaaaaatccaaaaaaaactaaaaaaccaataaaaataaaagatc

SEQ ID NO: 12 - DNA, 705 bp, Saccharomyces cerevisiae
  source 1..705: mol_type = genomic DNA
acgatggcttcttatctcacttcaatagtactttccaccggttatacttccggcttttccctattaatacaagctacaatttcaatgggtggcaaataatgtgtagaatagaaaataagccgacagggtaataaagaaaatttttagaaaaaaaaggttagatggcttatttaagttacaggctagcgaaaaaaggaacttcagggcaagtaaagtgtttgattgggcactagcatggcttataaaggcgagcaattgtcgaaactaattaatgttgtacggactattgctgtcatctcgtggtaaatgcgtgttccaggtcgaatactacttgcacacaggcgagcggggccccataaaagtgttgccgatttgttaagttgtcttttcggtttttctactctgttattccttacttccctttttaagaactctttttatccttcatttaggatcttgcacgtttccgcctcatcacttgaattaaaacatgtctctgtcagtaaaccttggcgtttctattgttcttcatagttcaacttttattattacccgccctgcgcgtttacatttttccagcaacagccagcgaaaaattagaaaatctggttgttgacacctcaagaacaagggcaattagcctcagcgtcgaatatagatcatattagaatacctatagctccatcaaaagaaatacacaagatc

SEQ ID NO: 13 - DNA, 705 bp, Saccharomyces cerevisiae
  source 1..705: mol_type = genomic DNA
ttcgtgctttgtgataaagtgtttcacgtcatccgacatgacttcgtagttatggactgaactgtgtggtgaggttccatgatttcttaggtccagcagatacatgtctcttcccaatttcttgttaaggttacggccaatgcttcggttgttgagcttgttaccgaataagccgtgaagtatgataataggtggtcttggcttcccttcatccccagtttttactgcatctctcttgattatgtcatatgaaaggtccagtgggacttgcttttgttgcagcacctttgctaatgaatgaaaggcacatagtgactgcttaaaaatgcaggaacttaaattattccgaatggtattttgtctcacatatattgtcccatactgtgccaagatcccggctttacccagtatcatcattgtaccgttaccaattctcctcgtatatcacggttagtttttaaacctcggggtgacgtttactattggcgtactaatatattcttattttcttttcttttttgttggcagtttcaagcaacacatgtactggataaccaacccccgcacgctcttggaaaaaattgagaaggcatcggacacttgctgatgagtatttcgaaaaattccatgaagatgaggccaagattgtttggaagagattgaaaagaagaagaagaaaaaaagataaaagcaaatcaaaagatc

SEQ ID NO: 14 - DNA, 705 bp, Saccharomyces cerevisiae
  source 1..705: mol_type = genomic DNA
agtcgaacaagaagcaggcaaagtttagagcactgcccctccgcactcaaaaaagaaaaaactaggaggaaaataaaattctcaaccacacaaacacataaacacatacaaatacaaatacaagcttatttacttgacatcgcgcgatcttccactattcagcgccgtccgccctctctcgtgttttttgtttacgcgacaactatgcgaaatccggagcaacgggcaaccgtttggggaaagaccacacccacgcgcgatcgccatggcaacgaggtcgcacacgccccacacccagacctccctgcgagcgggcatgggtacaatgtccccgttgccacagacaccacttcgtagcacagcgcagagcgtagcgtgttgttgctgctgacaaaagaaaatttttcttagcaaagcaaaggaggggaagcacgggcagatagcaccgtaccatacccttggaaactcgaaatgaacgaagcaggaaatgagagaatgagagttttgtaggtatatatagcggtagtgtttgcgcgttaccatcatcttctggatctatctattgttcttttcctcatcactttcccctttttcgctcttcttcttgtcttttatttctttcttttttttaattgttccctcgattggctatctaccaaagaatccaaacttaatacacgtatttatttgtccaattaccagatc

SEQ ID NO: 15 - DNA, 705 bp, Saccharomyces cerevisiae
  source 1..705: mol_type = genomic DNA
tgacccaacatcagatgacccaaggtccacctcttattaaaggacgtttgatccttcgacaccatggctctgttgaacttttatctgagagaggaaaaaaaggaaggaaaaaaaagaagaaacttcctttatttatttgtcttaaccacaacacacaatgcaataagatgcaatataatatcaaagccaatatcttatgttgctgatcctgagaaggaatatatacaatttatgtagtaaaataccttttcttctgcgagttgcaagaaatagaaaagactccgattgcgcatcgccagaataaaatttcacaaccacactttttggctgaactttttattacctgattaaacagagagagaaaaggtagaggtcaaaattttttaagcaaaactaaaaaagatgcaaaatcacgtgctgaaaatctaacataagggttaagattagagttttataggacttgttttgtaatatttcaaatacgagctaaccctactgatttcaattaggtctaatttagggttgagctgcactgaaatttcggaaattttgggttattttaaatgagacagaagaactacagagatacgttcttcagactttaaagcttatctccacaaagaattggtcaagaaatcatcctagaaaaacacgtttgctcactcgatcttaatcacatagagtgctggaacgggaagaaagatc

SEQ ID NO: 16 - DNA, 699 bp, Saccharomyces cerevisiae
  source 1..699: mol_type = genomic DNA
ccttgtgaaattgcaaatatggtgatttgaaacgtttcctagtgcagcaggatcacagataacgtgtaaagggcttagcagttgataatcctctctagttaagacctaaacaaaatgctgtcactaaccgtagtattaaatgacacactttggtgactttcgttaatggggatgtggtagtggccattgccaataaacaaaaagaacagggaaagaagtagaaagtgatataagtttgcttgccacttttcgtttttcacgaaaaaaacaggcgaaaaaaaatgctagacaagtacccggctgaatcacacctcgttaacagtgactttcggtgacagatacccgattgggcacccggctggtaagttatgatagaaagccaacgctgtactattggcttagctatggcaatattttgattatcagctagttttattaacgttataattagtgtaaccagtttttcatctatttcatttatttcatttatttactttaattgcagatccccctaacgcgtttaaagcttttattcactagcttatgtattttttataggaaacgcgacgcgtaacatcgcgcaaatgaaggttttgatgtattataatgaggtattcttccttatatacatcgatgaaaagcgttgacagcatacattggaaagaaataggaaacggacaccggaagaaaaaatagatc

SEQ ID NO: 17 - DNA, 705 bp, Saccharomyces cerevisiae
  source 1..705: mol_type = genomic DNA
gtgttgttatccgatacaaccggatatttttcttttaatgagtctaaaccgtgatagcttcaggttaatacaatcaaaaaaagctcaaatattcttttaatgccgcgttcacagattccaattgaatacaactaggtagttcattatatgaagcctttgctactatttttcactatagtctgccttcaccttaatgcagacatccacatattttaatcactttaaaataaaaaggaagatatattagaagctatgatccaatctgtaagccagattaaaattcacgaactcttctttcatttgaattgaatgctttgagttggggtagattatcgcaaattactcatcacatttattgactacgaacttgctgatgtcctttttttatttatatttttcttcagtgaagcgattttttttttacacagaccaagacggaaaaaagtagctaaggaagaaaacaaaatcatgaaaaaaatgtgaagtgatcatgcacatcgcatcaacttaaacattggcttagagatatatagagttagagtttacggcaacctttaagcaccaataccttttggcatagtctaaagacctggttcttaattttaaacaaatttaactaaagatttccctatcaaagaagtaacgagttgacagattttctcaaaataaatcgatactgcatttctaggcatatccagcgagatc

SEQ ID NO: 18 - DNA, 1356 bp, Saccharomyces cerevisiae
  source 1..1356: mol_type = genomic DNA
atgtcagagttgagagccttcagtgccccagggaaagcgttactagctggtggatatttagttttagatacaaaatatgaagcatttgtagtcggattatcggcaagaatgcatgctgtagcccatccttacggttcattgcaagggtctgataagtttgaagtgcgtgtgaaaagtaaacaatttaaagatggggagtggctgtaccatataagtcctaaaagtggcttcattcctgtttcgataggcggatctaagaaccctttcattgaaaaagttatcgctaacgtatttagctactttaaacctaacatggacgactactgcaatagaaacttgttcgttattgatattttctctgatgatgcctaccattctcaggaggatagcgttaccgaacatcgtggcaacagaagattgagttttcattcgcacagaattgaagaagttcccaaaacagggctgggctcctcggcaggtttagtcacagttttaactacagctttggcctccttttttgtatcggacctggaaaataatgtagacaaatatagagaagttattcataatttagcacaagttgctcattgtcaagctcagggtaaaattggaagcgggtttgatgtagcggcggcagcatatggatctatcagatatagaagattcccacccgcattaatctctaatttgccagatattggaagtgctacttacggcagtaaactggcgcatttggttgatgaagaagactggaatattacgattaaaagtaaccatttaccttcgggattaactttatggatgggcgatattaagaatggttcagaaacagtaaaactggtccagaaggtaaaaaattggtatgattcgcatatgccagaaagcttgaaaatatatacagaactcgatcatgcaaattctagatttatggatggactatctaaactagatcgcttacacgagactcatgacgattacagcgatcagatatttgagtctcttgagaggaatgactgtacctgtcaaaagtatcctgaaatcacagaagttagagatgcagttgccacaattagacgttcctttagaaaaataactaaagaatctggtgccgatatcgaacctcccgtacaaactagcttattggatgattgccagaccttaaaaggagttcttacttgcttaatacctggtgctggtggttatgacgccattgcagtgattactaagcaagatgttgatcttagggctcaaaccgctaatgacaaaagattttctaaggttcaatggctggatgtaactcaggctgactggggtgttaggaaagaaaaagatccggaaacttatcttgataaataa

SEQ ID NO: 19 - DNA, 1197 bp, Saccharomyces cerevisiae
  source 1..1197: mol_type = genomic DNA
atgtctcagaacgtttacattgtatcgactgccagaaccccaattggttcattccagggttctctatcctccaagacagcagtggaattgggtgctgttgctttaaaaggcgccttggctaaggttccagaattggatgcatccaaggattttgacgaaattatttttggtaacgttctttctgccaatttgggccaagctccggccagacaagttgctttggctgccggtttgagtaatcatatcgttgcaagcacagttaacaaggtctgtgcatccgctatgaaggcaatcattttgggtgctcaatccatcaaatgtggtaatgctgatgttgtcgtagctggtggttgtgaatctatgactaacgcaccatactacatgccagcagcccgtgcgggtgccaaatttggccaaactgttcttgttgatggtgtcgaaagagatgggttgaacgatgcgtacgatggtctagccatgggtgtacacgcagaaaagtgtgcccgtgattgggatattactagagaacaacaagacaattttgccatcgaatcctaccaaaaatctcaaaaatctcaaaaggaaggtaaattcgacaatgaaattgtacctgttaccattaagggatttagaggtaagcctgatactcaagtcacgaaggacgaggaacctgctagattacacgttgaaaaattgagatctgcaaggactgttttccaaaaagaaaacggtactgttactgccgctaacgcttctccaatcaacgatggtgctgcagccgtcatcttggtttccgaaaaagttttgaaggaaaagaatttgaagcctttggctattatcaaaggttggggtgaggccgctcatcaaccagctgattttacatgggctccatctcttgcagttccaaaggctttgaaacatgctggcatcgaagacatcaattctgttgattactttgaattcaatgaagccttttcggttgtcggtttggtgaacactaagattttgaagctagacccatctaaggttaatgtatatggtggtgctgttgctctaggtcacccattgggttgttctggtgctagagtggttgttacactgctatccatcttacagcaagaaggaggtaagatcggtgttgccgccatttgtaatggtggtggtggtgcttcctctattgtcattgaaaagatatga

SEQ ID NO: 20 - DNA, 1332 bp, Saccharomyces cerevisiae
  source 1..1332: mol_type = genomic DNA
atgtcattaccgttcttaacttctgcaccgggaaaggttattatttttggtgaacactctgctgtgtacaacaagcctgccgtcgctgctagtgtgtctgcgttgagaacctacctgctaataagcgagtcatctgcaccagatactattgaattggacttcccggacattagctttaatcataagtggtccatcaatgatttcaatgccatcaccgaggatcaagtaaactcccaaaaattggccaaggctcaacaagccaccgatggcttgtctcaggaactcgttagtcttttggatccgttgttagctcaactatccgaatccttccactaccatgcagcgttttgtttcctgtatatgtttgtttgcctatgcccccatgccaagaatattaagttttctttaaagtctactttacccatcggtgctgggttgggctcaagcgcctctatttctgtatcactggccttagctatggcctacttgggggggttaataggatctaatgacttggaaaagctgtcagaaaacgataagcatatagtgaatcaatgggccttcataggtgaaaagtgtattcacggtaccccttcaggaatagataacgctgtggccacttatggtaatgccctgctatttgaaaaagactcacataatggaacaataaacacaaacaattttaagttcttagatgatttcccagccattccaatgatcctaacctatactagaattccaaggtctacaaaagatcttgttgctcgcgttcgtgtgttggtcaccgagaaatttcctgaagttatgaagccaattctagatgccatgggtgaatgtgccctacaaggcttagagatcatgactaagttaagtaaatgtaaaggcaccgatgacgaggctgtagaaactaataatgaactgtatgaacaactattggaattgataagaataaatcatggactgcttgtctcaatcggtgtttctcatcctggattagaacttattaaaaatctgagcgatgatttgagaattggctccacaaaacttaccggtgctggtggcggcggttgctctttgactttgttacgaagagacattactcaagagcaaattgacagcttcaaaaagaaattgcaagatgattttagttacgagacatttgaaacagacttgggtgggactggctgctgtttgttaagcgcaaaaaatttgaataaagatcttaaaatcaaatccctagtattccaattatttgaaaataaaactaccacaaagcaacaaattgacgatctattattgccaggaaacacgaatttaccatggacttcataa

SEQ ID NO: 21 - DNA, 1476 bp, Saccharomyces cerevisiae
  source 1..1476: mol_type = genomic DNA
atgaaactctcaactaaactttgttggtgtggtattaaaggaagacttaggccgcaaaagcaacaacaattacacaatacaaacttgcaaatgactgaactaaaaaaacaaaagaccgctgaacaaaaaaccagacctcaaaatgtcggtattaaaggtatccaaatttacatcccaactcaatgtgtcaaccaatctgagctagagaaatttgatggcgtttctcaaggtaaatacacaattggtctgggccaaaccaacatgtcttttgtcaatgacagagaagatatctactcgatgtccctaactgttttgtctaagttgatcaagagttacaacatcgacaccaacaaaattggtagattagaagtcggtactgaaactctgattgacaagtccaagtctgtcaagtctgtcttgatgcaattgtttggtgaaaacactgacgtcgaaggtattgacacgcttaatgcctgttacggtggtaccaacgcgttgttcaactctttgaactggattgaatctaacgcatgggatggtagagacgccattgtagtttgcggtgatattgccatctacgataagggtgccgcaagaccaaccggtggtgccggtactgttgctatgtggatcggtcctgatgctccaattgtatttgactctgtaagagcttcttacatggaacacgcctacgatttttacaagccagatttcaccagcgaatatccttacgtcgatggtcatttttcattaacttgttacgtcaaggctcttgatcaagtttacaagagttattccaagaaggctatttctaaagggttggttagcgatcccgctggttcggatgctttgaacgttttgaaatatttcgactacaacgttttccatgttccaacctgtaaattggtcacaaaatcatacggtagattactatataacgatttcagagccaatcctcaattgttcccagaagttgacgccgaattagctactcgcgattatgacgaatctttaaccgataagaacattgaaaaaacttttgttaatgttgctaagccattccacaaagagagagttgcccaatctttgattgttccaacaaacacaggtaacatgtacaccgcatctgtttatgccgcctttgcatctctattaaactatgttggatctgacgacttacaaggcaagcgtgttggtttattttcttacggttccggtttagctgcatctctatattcttgcaaaattgttggtgacgtccaacatattatcaaggaattagatattactaacaaattagccaagagaatcaccgaaactccaaaggattacgaagctgccatcgaattgagagaaaatgcccatttgaagaagaacttcaaacctcaaggttccattgagcatttgcaaagtggtgtttactacttgaccaacatcgatgacaaatttagaagatcttacgatgttaaaaaataa

SEQ ID NO: 22 - DNA, 1191 bp, Saccharomyces cerevisiae
  source 1..1191: mol_type = genomic DNA
atgaccgtttacacagcatccgttaccgcacccgtcaacatcgcaacccttaagtattgggggaaaagggacacgaagttgaatctgcccaccaattcgtccatatcagtgactttatcgcaagatgacctcagaacgttgacctctgcggctactgcacctgagtttgaacgcgacactttgtggttaaatggagaaccacacagcatcgacaatgaaagaactcaaaattgtctgcgcgacctacgccaattaagaaaggaaatggaatcgaaggacgcctcattgcccacattatctcaatggaaactccacattgtctccgaaaataactttcctacagcagctggtttagcttcctccgctgctggctttgctgcattggtctctgcaattgctaagttataccaattaccacagtcaacttcagaaatatctagaatagcaagaaaggggtctggttcagcttgtagatcgttgtttggcggatacgtggcctgggaaatgggaaaagctgaagatggtcatgattccatggcagtacaaatcgcagacagctctgactggcctcagatgaaagcttgtgtcctagttgtcagcgatattaaaaaggatgtgagttccactcagggtatgcaattgaccgtggcaacctccgaactatttaaagaaagaattgaacatgtcgtaccaaagagatttgaagtcatgcgtaaagccattgttgaaaaagatttcgccacctttgcaaaggaaacaatgatggattccaactctttccatgccacatgtttggactctttccctccaatattctacatgaatgacacttccaagcgtatcatcagttggtgccacaccattaatcagttttacggagaaacaatcgttgcatacacgtttgatgcaggtccaaatgctgtgttgtactacttagctgaaaatgagtcgaaactctttgcatttatctataaattgtttggctctgttcctggatgggacaagaaatttactactgagcagcttgaggctttcaaccatcaatttgaatcatctaactttactgcacgtgaattggatcttgagttgcaaaaggatgttgccagagtgattttaactcaagtcggttcaggcccacaagaaacaaacgaatctttgattgacgcaaagactggtctaccaaaggaataa

SEQ ID NO: 23 - DNA, 1512 bp, Saccharomyces cerevisiae
  source 1..1512: mol_type = genomic DNA
atgccagttttaaccaataaaacagtcatttctggatcgaaagtcaaaagtttatcatctgcgcaatcgagctcatcaggaccttcatcatctagtgaggaagatgattcccgcgatattgaaagcttggataagaaaatacgtcctttagaagaattagaagcattattaagtagtggaaatacaaaacaattgaagaacaaagaggtcgctgccttggttattcacggtaagttacctttgtacgctttggagaaaaaattaggtgatactacgagagcggttgcggtacgtaggaaggctctttcaattttggcagaagctcctgtattagcatctgatcgtttaccatataaaaattatgactacgaccgcgtatttggcgcttgttgtgaaaatgttataggttacatgcctttgcccgttggtgttataggccccttggttatcgatggtacatcttatcatataccaatggcaactacagagggttgtttggtagcttctgccatgcgtggctgtaaggcaatcaatgctggcggtggtgcaacaactgttttaactaaggatggtatgacaagaggcccagtagtccgtttcccaactttgaaaagatctggtgcctgtaagatatggttagactcagaagagggacaaaacgcaattaaaaaagcttttaactctacatcaagatttgcacgtctgcaacatattcaaacttgtctagcaggagatttactcttcatgagatttagaacaactactggtgacgcaatgggtatgaatatgatttctaaaggtgtcgaatactcattaaagcaaatggtagaagagtatggctgggaagatatggaggttgtctccgtttctggtaactactgtaccgacaaaaaaccagctgccatcaactggatcgaaggtcgtggtaagagtgtcgtcgcagaagctactattcctggtgatgttgtcagaaaagtgttaaaaagtgatgtttccgcattggttgagttgaacattgctaagaatttggttggatctgcaatggctgggtctgttggtggatttaacgcacatgcagctaatttagtgacagctgttttcttggcattaggacaagatcctgcacaaaatgttgaaagttccaactgtataacattgatgaaagaagtggacggtgatttgagaatttccgtatccatgccatccatcgaagtaggtaccatcggtggtggtactgttctagaaccacaaggtgccatgttggacttattaggtgtaagaggcccgcatgctaccgctcctggtaccaacgcacgtcaattagcaagaatagttgcctgtgccgtcttggcaggtgaattatccttatgtgctgccctagcagccggccatttggttcaaagtcatatgacccacaacaggaaacctgctgaaccaacaaaacctaacaatttggacgccactgatataaatcgtttgaaagatgggtccgtcacctgcattaaatcctaa

SEQ ID NO: 24 - DNA, 867 bp, Saccharomyces cerevisiae
  source 1..867: mol_type = genomic DNA
atgactgccgacaacaatagtatgccccatggtgcagtatctagttacgccaaattagtgcaaaaccaaacacctgaagacattttggaagagtttcctgaaattattccattacaacaaagacctaatacccgatctagtgagacgtcaaatgacgaaagcggagaaacatgtttttctggtcatgatgaggagcaaattaagttaatgaatgaaaattgtattgttttggattgggacgataatgctattggtgccggtaccaagaaagtttgtcatttaatggaaaatattgaaaagggtttactacatcgtgcattctccgtctttattttcaatgaacaaggtgaattacttttacaacaaagagccactgaaaaaataactttccctgatctttggactaacacatgctgctctcatccactatgtattgatgacgaattaggtttgaagggtaagctagacgataagattaagggcgctattactgcggcggtgagaaaactagatcatgaattaggtattccagaagatgaaactaagacaaggggtaagtttcactttttaaacagaatccattacatggcaccaagcaatgaaccatggggtgaacatgaaattgattacatcctattttataagatcaacgctaaagaaaacttgactgtcaacccaaacgtcaatgaagttagagacttcaaatgggtttcaccaaatgatttgaaaactatgtttgctgacccaagttacaagtttacgccttggtttaagattatttgcgagaattacttattcaactggtgggagcaattagatgacctttctgaagtggaaaatgacaggcaaattcatagaatgctataa

SEQ ID NO: 25 - protein, 451 aa, Saccharomyces cerevisiae
MSELRAFSAPGKALLAGGYLVLDTKYEAFVVGLSARMHAVAHPYGSLQGSDKFEVRVKSKQFKDGEWLYHISPKSGFIPVSIGGSKNPFIEKVIANVFSYFKPNMDDYCNRNLFVIDIFSDDAYHSQEDSVTEHRGNRRLSFHSHRIEEVPKTGLGSSAGLVTVLTTALASFFVSDLENNVDKYREVIHNLAQVAHCQAQGKIGSGFDVAAAAYGSIRYRRFPPALISNLPDIGSATYGSKLAHLVDEEDWNITIKSNHLPSGLTLWMGDIKNGSETVKLVQKVKNWYDSHMPESLKIYTELDHANSRFMDGLSKLDRLHETHDDYSDQIFESLERNDCTCQKYPEITEVRDAVATIRRSFRKITKESGADIEPPVQTSLLDDCQTLKGVLTCLIPGAGGYDAIAVITKQDVDLRAQTANDKRFSKVQWLDVTQADWGVRKEKDPETYLDK

SEQ ID NO: 26 - protein, 398 aa, Saccharomyces cerevisiae
MSQNVYIVSTARTPIGSFQGSLSSKTAVELGAVALKGALAKVPELDASKDFDEIIFGNVLSANLGQAPARQVALAAGLSNHIVASTVNKVCASAMKAIILGAQSIKCGNADVVVAGGCESMTNAPYYMPAARAGAKFGQTVLVDGVERDGLNDAYDGLAMGVHAEKCARDWDITREQQDNFAIESYQKSQKSQKEGKFDNEIVPVTIKGFRGKPDTQVTKDEEPARLHVEKLRSARTVFQKENGTVTAANASPINDGAAAVILVSEKVLKEKNLKPLAIIKGWGEAAHQPADFTWAPSLAVPKALKHAGIEDINSVDYFEFNEAFSVVGLVNTKILKLDPSKVNVYGGAVALGHPLGCSGARVVVTLLSILQQEGGKIGVAAICNGGGGASSIVIEKI

SEQ ID NO: 27 - protein, 443 aa, Saccharomyces cerevisiae
MSLPFLTSAPGKVIIFGEHSAVYNKPAVAASVSALRTYLLISESSAPDTIELDFPDISFNHKWSINDFNAITEDQVNSQKLAKAQQATDGLSQELVSLLDPLLAQLSESFHYHAAFCFLYMFVCLCPHAKNIKFSLKSTLPIGAGLGSSASISVSLALAMAYLGGLIGSNDLEKLSENDKHIVNQWAFIGEKCIHGTPSGIDNAVATYGNALLFEKDSHNGTINTNNFKFLDDFPAIPMILTYTRIPRSTKDLVARVRVLVTEKFPEVMKPILDAMGECALQGLEIMTKLSKCKGTDDEAVETNNELYEQLLELIRINHGLLVSIGVSHPGLELIKNLSDDLRIGSTKLTGAGGGGCSLTLLRRDITQEQIDSFKKKLQDDFSYETFETDLGGTGCCLLSAKNLNKDLKIKSLVFQLFENKTTTKQQIDDLLLPGNTNLPWTS

SEQ ID NO: 28 - protein, 491 aa, Saccharomyces cerevisiae
MKLSTKLCWCGIKGRLRPQKQQQLHNTNLQMTELKKQKTAEQKTRPQNVGIKGIQIYIPTQCVNQSELEKFDGVSQGKYTIGLGQTNMSFVNDREDIYSMSLTVLSKLIKSYNIDTNKIGRLEVGTETLIDKSKSVKSVLMQLFGENTDVEGIDTLNACYGGTNALFNSLNWIESNAWDGRDAIVVCGDIAIYDKGAARPTGGAGTVAMWIGPDAPIVFDSVRASYMEHAYDFYKPDFTSEYPYVDGHFSLTCYVKALDQVYKSYSKKAISKGLVSDPAGSDALNVLKYFDYNVFHVPTCKLVTKSYGRLLYNDFRANPQLFPEVDAELATRDYDESLTDKNIEKTFVNVAKPFHKERVAQSLIVPTNTGNMYTASVYAAFASLLNYVGSDDLQGKRVGLFSYGSGLAASLYSCKIVGDVQHIIKELDITNKLAKRITETPKDYEAAIELRENAHLKKNFKPQGSIEHLQSGVYYLTNIDDKFRRSYDVKK

SEQ ID NO: 29 - protein, 396 aa, Saccharomyces cerevisiae
MTVYTASVTAPVNIATLKYWGKRDTKLNLPTNSSISVTLSQDDLRTLTSAATAPEFERDTLWLNGEPHSIDNERTQNCLRDLRQLRKEMESKDASLPTLSQWKLHIVSENNFPTAAGLASSAAGFAALVSAIAKLYQLPQSTSEISRIARKGSGSACRSLFGGYVAWEMGKAEDGHDSMAVQIADSSDWPQMKACVLVVSDIKKDVSSTQGMQLTVATSELFKERIEHVVPKRFEVMRKAIVEKDFATFAKETMMDSNSFHATCLDSFPPIFYMNDTSKRIISWCHTINQFYGETIVAYTFDAGPNAVLYYLAENESKLFAFIYKLFGSVPGWDKKFTTEQLEAFNHQFESSNFTARELDLELQKDVARVILTQVGSGPQETNESLIDAKTGLPKE

SEQ ID NO: 30 - protein, 503 aa, Saccharomyces cerevisiae
MPVLTNKTVISGSKVKSLSSAQSSSSGPSSSSEEDDSRDIESLDKKIRPLEELEALLSSGNTKQLKNKEVAALVIHGKLPLYALEKKLGDTTRAVAVRRKALSILAEAPVLASDRLPYKNYDYDRVFGACCENVIGYMPLPVGVIGPLVIDGTSYHIPMATTEGCLVASAMRGCKAINAGGGATTVLTKDGMTRGPVVRFPTLKRSGACKIWLDSEEGQNAIKKAFNSTSRFARLQHIQTCLAGDLLFMRFRTTTGDAMGMNMISKGVEYSLKQMVEEYGWEDMEVVSVSGNYCTDKKPAAINWIEGRGKSVVAEATIPGDVVRKVLKSDVSALVELNIAKNLVGSAMAGSVGGFNAHAANLVTAVFLALGQDPAQNVESSNCITLMKEVDGDLRISVSMPSIEVGTIGGGTVLEPQGAMLDLLGVRGPHATAPGTNARQLARIVACAVLAGELSLCAALAAGHLVQSHMTHNRKPAEPTKPNNLDATDINRLKDGSVTCIKS

SEQ ID NO: 31 - protein, 288 aa, Saccharomyces cerevisiae
MTADNNSMPHGAVSSYAKLVQNQTPEDILEEFPEIIPLQQRPNTRSSETSNDESGETCFSGHDEEQIKLMNENCIVLDWDDNAIGAGTKKVCHLMENIEKGLLHRAFSVFIFNEQGELLLQQRATEKITFPDLWTNTCCSHPLCIDDELGLKGKLDDKIKGAITAAVRKLDHELGIPEDETKTRGKFHFLNRIHYMAPSNEPWGEHEIDYILFYKINAKENLTVNPNVNEVRDFKWVSPNDLKTMFADPSYKFTPWFKIICENYLFNWWEQLDDLSEVENDRQIHRML

SEQ ID NO: 32 - DNA, 48 bp, synthetic construct
  source 1..48: mol_type = other DNA
tttcgtctcgtcggggtctcgtatgtctcagaacgtttacattgtatc

SEQ ID NO: 33 - DNA, 54 bp, synthetic construct
  source 1..54: mol_type = other DNA
tttcgtctctggtcggtctccggattcatatcttttcaatgacaatagaggaag

SEQ ID NO: 34 - DNA, 43 bp, synthetic construct
  source 1..43: mol_type = other DNA
tttcgtctcgtcggggtctcgtatgtcagagttgagagccttc

SEQ ID NO: 35 - DNA, 52 bp, synthetic construct
  source 1..52: mol_type = other DNA
tttcgtctctggtcggtctccggatttatttatcaagataagtttccggatc

SEQ ID NO: 36 - DNA, 47 bp, synthetic construct
  source 1..47: mol_type = other DNA
tttcgtctcgtcggggtctcgtatgtcattaccgttcttaacttctg

SEQ ID NO: 37 - DNA, 50 bp, synthetic construct
  source 1..50: mol_type = other DNA
tttcgtctctggtcggtctccggatttatgaagtccatggtaaattcgtg

SEQ ID NO: 38 - DNA, 47 bp, synthetic construct
  source 1..47: mol_type = other DNA
tttcgtctcgtcggggtctcgtatgccagttttaaccaataaaacag

SEQ ID NO: 39 - DNA, 48 bp, synthetic construct
  source 1..48: mol_type = other DNA
tttcgtctctggtcggtctccggatttaggatttaatgcaggtgacgg

SEQ ID NO: 40 - DNA, 48 bp, synthetic construct
  source 1..48: mol_type = other DNA
tttcgtctcgtcggggtctcgtatgaaactctcaactaaactttgttg

SEQ ID NO: 41 - DNA, 27 bp, synthetic construct
  source 1..27: mol_type = other DNA
tttcgtctcatggcgtccctaccatcc

SEQ ID NO: 42 - DNA, 29 bp, synthetic construct
  source 1..29: mol_type = other DNA
tttcgtctccgccattgtagtttgcggtg

SEQ ID NO: 43 - DNA, 61 bp, synthetic construct
  source 1..61: mol_type = other DNA
tttcgtctctggtcggtctccggatttattttttaacatcgtaagatcttctaaatttgtc

SEQ ID NO: 44 - DNA, 43 bp, synthetic construct
  source 1..43: mol_type = other DNA
tttcgtctcgtcggggtctcgtatgaccgtttacacagcatcc

SEQ ID NO: 45 - DNA, 33 bp, synthetic construct
  source 1..33: mol_type = other DNA
tttcgtctcttgcagagaccaatgcagcaaagc

SEQ ID NO: 46 - DNA, 36 bp, synthetic construct
  source 1..36: mol_type = other DNA
ttcgtctcctgcaattgctaagttataccaattacc

SEQ ID NO: 47 - DNA, 53 bp, synthetic construct
  source 1..53: mol_type = other DNA
tttcgtctctggtctcggtctccggatttattcctttggtagaccagtctttg

SEQ ID NO: 48 - DNA, 42 bp, synthetic construct
  source 1..42: mol_type = other DNA
tttcgtctcgtcggggtctcgtatgactgccgacaacaatag

SEQ ID NO: 49 - DNA, 32 bp, synthetic construct
  source 1..32: mol_type = other DNA
tttcgtctctcatttgaagtctcactagatcg

SEQ ID NO: 50 - DNA, 29 bp, synthetic construct
  source 1..29: mol_type = other DNA
tttcgtctcaaatgacgaaagcggagaaa

SEQ ID NO: 51 - DNA, 52 bp, synthetic construct
  source 1..52: mol_type = other DNA
tttcgtctctggtcggtctccggatttatagcattctatgaatttgcctgtc

SEQ ID NO: 52 - DNA, 59 bp, synthetic construct
  source 1..59: mol_type = other DNA
tttcgtctctggtctccggattcataacttagatatcttttcaatgacaatagaggaag

SEQ ID NO: 53 - DNA, 64 bp, synthetic construct
  source 1..64: mol_type = other DNA
tttcgtctcgggtctccggatttataacttagattttttaacatcgtaagatcttctaaatttg

SEQ ID NO: 54 - DNA, 59 bp, synthetic construct
  source 1..59: mol_type = other DNA
tttcgtctctggtcggtctccggatttataacttagatgaagtccatggtaaattcgtg

SEQ ID NO: 55 - DNA, 57 bp, synthetic construct
  source 1..57: mol_type = other DNA
tttcgtctctggtctccggatttataacttagatttatcaagataagtttccggatc

SEQ ID NO: 56 - DNA, 56 bp, synthetic construct
  source 1..56: mol_type = other DNA
atgcgtctctggtctccggatctataacttagattcctttggtagaccagtctttg

SEQ ID NO: 57 - DNA, 57 bp, synthetic construct
  source 1..57: mol_type = other DNA
tttcgtctctggtcggtctccggatttataacttagaggatttaatgcaggtgacgg

SEQ ID NO: 58 - DNA, 57 bp, synthetic construct
  source 1..57: mol_type = other DNA
tttcgtctctggtctccggatttataacttagatagcattctatgaatttgcctgtc

SEQ ID NO: 59 - DNA, 44 bp, synthetic construct
  source 1..44: mol_type = other DNA
tttcgtctcgtcggggtctcgtatggaagagagttcatcaaagc

SEQ ID NO: 60 - DNA, 45 bp, synthetic construct
  source 1..45: mol_type = other DNA
tgacgtctcgccatgccagaaccttgtgtaaaaaacagggcatcg

SEQ ID NO: 61 - DNA, 36 bp, synthetic construct
  source 1..36: mol_type = other DNA
tttcgtctcgatggcttcagaaaaagaaattaggag

SEQ ID NO: 62 - DNA, 53 bp, synthetic construct
  source 1..53: mol_type = other DNA
tttcgtctcgggtcggtctcgggatctatttgcttctcttgtaaactttgttc

SEQ ID NO: 63 - DNA, 58 bp, synthetic construct
  source 1..58: mol_type = other DNA
tttcgtctctggtctctggatttataacttagatttgcttctcttgtaaactttgttc

SEQ ID NO: 64 - DNA, 41 bp, synthetic construct
  source 1..41: mol_type = other DNA
tttcgtctcgtcggggtctcgatccgctctaaccgaaaagg

SEQ ID NO: 65 - DNA, 41 bp, synthetic construct; identical to SEQ ID NO: 64
  source 1..41: mol_type = other DNA

SEQ ID NO: 66 - DNA, 36 bp, synthetic construct
  source 1..36: mol_type = other DNA
tttcgtctcgtcggtctcacaatcggccggtctggc

SEQ ID NO: 67 - DNA, 46 bp, synthetic construct
  source 1..46: mol_type = other DNA
tttcgtctcgggtctcaagggtaagaacctacacacaaaagacaca

SEQ ID NO: 68 - DNA, 52 bp, synthetic construct
  source 1..52: mol_type = other DNA
tttcgtctcgtcggtctcagagtcttctaactatatggtctccagatcttta

SEQ ID NO: 69 - DNA, 40 bp, synthetic construct
  source 1..40: mol_type = other DNA
tttcgtctcgggtctcatcggatgcgtaggggtagttgtg

SEQ ID NO: 70 - DNA, 52 bp, synthetic construct
  source 1..52: mol_type = other DNA
tttcgtctcgtcggtctcacaataaaaattcttactttttttttggatggac

SEQ ID NO: 71 - DNA, 46 bp, synthetic construct
  source 1..46: mol_type = other DNA
tttcgtctcgggtctcaagggaatagatcaaaaatcatcgcttcgc

SEQ ID NO: 72 - DNA, 39 bp, synthetic construct
  source 1..39: mol_type = other DNA
tttcgtctcgtcggtctcagagtgctgcctctgtttgcg

SEQ ID NO: 73 - DNA, 48 bp, synthetic construct
  source 1..48: mol_type = other DNA
tttcgtctcgggtctcatcggaatctcactggagatgttgttaagtag

SEQ ID NO: 74 - DNA, 41 bp, synthetic construct
  source 1..41: mol_type = other DNA
tttcgtctcgtcggtctcacaatggattgcgcttgcctttg

SEQ ID NO: 75 - DNA, 49 bp, synthetic construct
  source 1..49: mol_type = other DNA
tttcgtctcgggtctcaaggggaagttaatacctttaggttggttttcc

SEQ ID NO: 76 - DNA, 40 bp, synthetic construct
  source 1..40: mol_type = other DNA
tttcgtctcgtcggtctcagagttgctgaacgtggggttc

SEQ ID NO: 77 - DNA, 45 bp, synthetic construct
  source 1..45: mol_type = other DNA
tttcgtctcgggtctcatcggcaagtttcaaatctcccttggtac

SEQ ID NO: 78 - DNA, 55 bp, synthetic construct
  source 1..55: mol_type = other DNA
tttcgtctcgtcggtctcatacaaacgacattactatatatataatataggaagc

SEQ ID NO: 79 - DNA, 40 bp, synthetic construct
  source 1..40: mol_type = other DNA
tttcgtctcgggtctcgactccgcatctgtgcggtatttc

SEQ ID NO: 80 - DNA, 46 bp, synthetic construct
  source 1..46: mol_type = other DNA
tttcgtctcgtcggggtctcgtatggcttcagaaaaagaaattagg

SEQ ID NO: 81 - DNA, 45 bp, synthetic construct
  source 1..45: mol_type = other DNA
tttcgtctcgtcggggtctcgtatgggaaagctattacaattggc

SEQ ID NO: 82 - DNA, 45 bp, synthetic construct
  source 1..45: mol_type = other DNA
tttcgtctcgggtcggtctccggattcacgctctgtgtaaagtgt

SEQ ID NO: 83 - DNA, 51 bp, synthetic construct
  source 1..51: mol_type = other DNA
tttcgtctcgccatagaaccaccacctttgcttctcttgtaaactttgttc

SEQ ID NO: 84 - DNA, 29 bp, synthetic construct
  source 1..29: mol_type = other DNA
tttcgtctcgatggagcgtcagtcaatgg

SEQ ID NO: 85 - DNA, 33 bp, synthetic construct
  source 1..33: mol_type = other DNA
tttcgtctcgatgggaaagctattacaattggc

SEQ ID NO: 86 - DNA, 57 bp, synthetic construct
  source 1..57: mol_type = other DNA
tttcgtctcgccatagaaccaccacccgctctgtgtaaagtgtatatataataaaac

SEQ ID NO: 87 - DNA, 60 bp, synthetic construct
  source 1..60: mol_type = other DNA
cgggtggcgaatgggactttattcgtctattaagatcctggttttagagctagaaatagc

SEQ ID NO: 88 - DNA, 60 bp, synthetic construct
  source 1..60: mol_type = other DNA
gctatttctagctctaaaaccaggatcttaatagacgaataaagtcccattcgccacccg

SEQ ID NO: 89 - DNA, 60 bp, synthetic construct
  source 1..60: mol_type = other DNA
cgggtggcgaatgggactttatatcaaaatcaatagctaagttttagagctagaaatagc

SEQ ID NO: 90 - DNA, 60 bp, synthetic construct
  source 1..60: mol_type = other DNA
gctatttctagctctaaaacttagctattgattttgatataaagtcccattcgccacccg

SEQ ID NO: 91 - DNA, 60 bp, synthetic construct
  source 1..60: mol_type = other DNA
cgggtggcgaatgggacttttcgttcgggcgagagtgcgcgttttagagctagaaatagc

SEQ ID NO: 92 - DNA, 60 bp, synthetic construct
  source 1..60: mol_type = other DNA
gctatttctagctctaaaacgcgcactctcgcccgaacgaaaagtcccattcgccacccg

SEQ ID NO: 93 - DNA, 19 bp, synthetic construct
  source 1..19: mol_type = other DNA
cacacactgcgttctcttg

SEQ ID NO: 94 - DNA, 23 bp, synthetic construct
  source 1..23: mol_type = other DNA
cagttcagtctagatgcgaattc

SEQ ID NO: 95 - DNA, 20 bp, synthetic construct
  source 1..20: mol_type = other DNA
gctaaggtagagggtgaacg

SEQ ID NO: 96 - DNA, 25 bp, synthetic construct
  source 1..25: mol_type = other DNA
ggtttggtatatgaggaatgtgatg

SEQ ID NO: 97 - DNA, 31 bp, synthetic construct
  source 1..31: mol_type = other DNA
gtaactgagctgtcatttatattgaattttc

SEQ ID NO: 98 - DNA, 16 bp, synthetic construct
  source 1..16: mol_type = other DNA
gctgtcgccgaagaag

SEQ ID NO: 99 - DNA, 26 bp, synthetic construct
  source 1..26: mol_type = other DNA
ccctctgatatagctttaagacttga

SEQ ID NO: 100 - DNA, 28 bp, synthetic construct
  source 1..28: mol_type = other DNA
ctacctgactagattttcattttgtttc

SEQ ID NO: 101 - DNA, 25 bp, synthetic construct
  source 1..25: mol_type = other DNA
cgcttagattaaatggcgttattgg

SEQ ID NO: 102 - DNA, 20 bp, synthetic construct
  source 1..20: mol_type = other DNA
gaagtactcgccgatagtgg

SEQ ID NO: 103 - DNA, 26 bp, synthetic construct
  source 1..26: mol_type = other DNA
gtaaaggaccagatttgaaatttctg

SEQ ID NO: 104 - DNA, 21 bp, synthetic construct
  source 1..21: mol_type = other DNA
cactgctatccatcttacagc

SEQ ID NO: 105 - DNA, 21 bp, synthetic construct
  source 1..21: mol_type = other DNA
caaccgctctcgtagtatcac

SEQ ID NO: 106 - DNA, 25 bp, synthetic construct
  source 1..25: mol_type = other DNA
gataaataaatcctaactcgaggcc

SEQ ID NO: 107 - DNA, 25 bp, synthetic construct
  source 1..25: mol_type = other DNA
gcactctcgagttattatagcattc

SEQ ID NO: 108 - DNA, 22 bp, synthetic construct
  source 1..22: mol_type = other DNA
gcaaagtggtgtttactacttg

SEQ ID NO: 109 - DNA, 21 bp, synthetic construct
  source 1..21: mol_type = other DNA
catagctaaggccagtgatac

SEQ ID NO: 110 - DNA, 21 bp, synthetic construct
  source 1..21: mol_type = other DNA
cacgaatttaccatggacttc

SEQ ID NO: 111 - DNA, 19 bp, synthetic construct
  source 1..19: mol_type = other DNA
gtctgcgatttgtactgcc

SEQ ID NO: 112 - DNA, 21 bp, synthetic construct
  source 1..21: mol_type = other DNA
gatacttggaatcctggctgg

SEQ ID NO: 113 - DNA, 25 bp, synthetic construct
  source 1..25: mol_type = other DNA
gctaatgtcttcttctgatggtctg

SEQ ID NO: 114 - DNA, 21 bp, synthetic construct
  source 1..21: mol_type = other DNA
gtctgtgcatccgctatgaag

SEQ ID NO: 115 - DNA, 21 bp, synthetic construct
  source 1..21: mol_type = other DNA
ctgctggcatgtagtatggtg

SEQ ID NO: 116 - DNA, 22 bp, synthetic construct
  source 1..22: mol_type = other DNA
gatggtagagacgccattgtag

SEQ ID NO: 117 - DNA, 20 bp, synthetic construct
  source 1..20: mol_type = other DNA
gcgtgttccatgtaagaagc

SEQ ID NO: 118 - DNA, 18 bp, synthetic construct
  source 1..18: mol_type = other DNA
aagcagacccgtttgacg

SEQ ID NO: 119 - DNA, 19 bp, synthetic construct
  source 1..19: mol_type = other DNA
tgacccggtcttcctcatg

SEQ ID NO: 120 - DNA, 20 bp, synthetic construct
  source 1..20: mol_type = other DNA
ccgtatccatgccatccatc

SEQ ID NO: 121 - DNA, 20 bp, synthetic construct
  source 1..20: mol_type = other DNA
gaatagttgcctgtgccgtc

SEQ ID NO: 122 - DNA, 19 bp, synthetic construct
  source 1..19: mol_type = other DNA
gccatcaccgaggatcaag

SEQ ID NO: 123 - DNA, 19 bp, synthetic construct
  source 1..19: mol_type = other DNA
gctgcatggtagtggaagg

SEQ ID NO: 124 - DNA, 22 bp, synthetic construct
  source 1..22: mol_type = other DNA
gatgatgcctaccattctcagg

SEQ ID NO: 125 - DNA, 20 bp, synthetic construct
  source 1..20: mol_type = other DNA
ctgtgactaaacctgccgag

SEQ ID NO: 126 - DNA, 24 bp, synthetic construct
  source 1..24: mol_type = other DNA
ctgaagatggtcatgattccatgg

SEQ ID NO: 127 - DNA, 20 bp, synthetic construct
  source 1..20: mol_type = other DNA
tgccacggtcaattgcatac

SEQ ID NO: 128 - DNA, 21 bp, synthetic construct
  source 1..21: mol_type = other DNA
ctacatcgtgcattctccgtc

SEQ ID NO: 129 - DNA, 26 bp, synthetic construct
  source 1..26: mol_type = other DNA
cttatcgtctagcttacccttcaaac

SEQ ID NO: 130 - DNA, 825 bp, Saccharomyces cerevisiae
  source 1..825: mol_type = genomic DNA
atgatctctgtcatggcggatgagaaacataaggagtattttaagctatactactttcagtacatgataattggtctatgtacgatattattcctctattcggagatatccctggtacctaggggccaaaacatcgaatttagtcttgatgaccccagtatatcaaaacgttatgtacctaacgaactcgtgggcccactagaatgtttgattttgagtgttggactgagtaacatggtcgtcttctggacctgcatgtttgacaaggacttactgaagaagaatagagtaaagagactaagagagaggccggacggaatctcgaacgattttcacttcatgcatactagcattctatgtctgatgctgattataagcataaatgctgccctaacaggcgccttaaagttgattataggaaacttgaggcctgactttgttgatagatgtatacctgacctccaaaagatgagtgattcagattctttggtttttggcttggacatttgcaagcagactaacaaatggattctatacgaaggcttaaaaagcactccaagcggacattcaagtttcatagtcagtaccatgggctttacatatctttggcaaagggttttcaccacacgcaatacaagaagttgcatttggtgccctttattagctctagtagtaatggtttcaagggttatcgatcacagacatcattggtacgatgttgtctctggagctgttctagcatttttagtcatttattgttgctggaaatggacatttacaaacttggcgaaaagagacatacttccttcaccggttagtgtttag

SEQ ID NO: 131 - DNA, 870 bp, Saccharomyces cerevisiae
  source 1..870: mol_type = genomic DNA
atgaacagagtttcgtttattaaaacgcctttcaacataggggcgaaatggagattagaagatgtctttttgctcattatcatgatacttcttaactacccagtgtattaccaacaaccgttcgaacgtcagttttacattaacgatctcactatatcgcatccttatgcgacaactgaacgtgtaaataacaacatgttgtttgtttatagttttgtcgtgccatctttaaccatattgataattggttccattttggccgatagaagacatttgatttttattttgtacacatctctccttggtttatcactcgcttggttcagtacgagtttctttacaaacttcatcaagaattggattggaagactaagaccagattttctagatcgttgccaacctgttgaaggcttgccattggacactttatttactgcaaaagatgtgtgtacgactaagaatcacgaacgtctgttggatgggtttaggacaactccgtcaggtcattcaagtgaaagctttgcaggactgggttatttgtacttctggctatgtgggcaacttttgactgaatcaccgttgatgcctttatggagaaaaatggtggcctttctaccactgttaggagctgcactaattgctctatccagaactcaagattacagacatcatttcgtcgatgtaattttagggtctatgttgggttatataatggcacactttttctacagaagaatcttcccacccattgatgatcctcttccgttcaaaccattgatggacgattcagatgtcaccctggaggaagcagtcacccatcagaggatcccggatgaggaattacatcctttgtccgatgaaggtatgtaa

SEQ ID NO: 132 - DNA, 1761 bp, Saccharomyces cerevisiae
  source 1..1761: mol_type = genomic DNA
atgctttctgaaaacacgactattctgatggctaacggtgaaattaaagacatcgcaaacgtcacggctaactcttacgttatgtgcgcagatggctccgctgcccgcgtcataaatgtcacacagggctatcagaaaatctataatatacagcaaaaaaccaaacacagagcttttgaaggtgaacctggtaggttagatcccaggcgtagaacagtttatcagcgtcttgcattacaatgtactgcaggtcataaattgtcagtcagggtccctaccaaaccactgttggaaaaaagtggtagaaatgccaccaaatataaagtgagatggagaaatctgcagcaatgtcagacgcttgatggtaggataataataattccaaaaaaccatcataagacattcccaatgacagttgaaggtgagtttgccgcaaaacgcttcatagaagaaatggagcgctctaaaggagaatatttcaactttgacattgaagttagagatttggattatcttgatgctcaattgagaatttctagctgcataagatttggtccagtactcgcaggaaatggtgttttatctaaatttctcactggacgtagtgaccttgtaactcctgctgtaaaaagtatggcttggatgcttggtctgtggttaggtgacagtacaacaaaagagccagaaatctcagtagatagcttggatcctaagctaatggagagtttaagagaaaatgcgaaaatctggggtctctaccttacggtttgtgacgatcacgttccgctacgtgccaaacatgtaaggcttcattatggagatggtccagatgaaaacaggaagacaaggaatttgaggaaaaataatccattctggaaagctgtcacaattttaaagtttaaaagggatcttgatggagagaagcaaatccctgaatttatgtacggcgagcatatagaagttcgtgaagcattcttagccggcttgatcgactcagatgggtacgttgtgaaaaagggcgaaggccctgaatcttataaaatagcaattcaaactgtttattcatccattatggacggaattgtccatatttcaagatctcttggtatgtcagctactgtgacgaccaggtcagctagggaggaaatcattgaaggaagaaaagtccaatgtcaatttacatacgactgtaatgttgctgggggaacaacttcacagaatgttttgtcatattgtcgaagtggtcacaaaacaagagaagttccgccaattataaaaagggaacccgtatatttcagcttcacggatgatttccagggtgagagtactgtatatgggcttacgatagaaggccataaaaatttcttgcttggcaacaaaatagaagtgaaatcatgtcgaggctgctgtgtgggagaacagcttaaaatatcacaaaaaaagaatctaaaacactgtgttgcttgtcccagaaagggaatcaagtatttttataaagattggagtggtaaaaatcgagtatgtgctagatgctatggaagatacaaattcagcggtcatcactgtataaattgcaagtatgtaccagaagcacgtgaagtgaaaaaggcaaaagacaaaggcgaaaaattgggcattacgcccgaaggtttgccagttaaaggaccagagtgtataaaatgtggcggaatcttacagtttgatgctgtccgcgggcctcataagagttgtggtaacaacgcaggtgcgcgcatctgctaa

SEQ ID NO: 133 - DNA, 1491 bp, Saccharomyces cerevisiae
  source 1..1491: mol_type = genomic DNA
atgtctgctgttaacgttgcacctgaattgattaatgccgacaacacaattacctacgatgcgattgtcatcggtgctggtgttatcggtccatgtgttgctactggtctagcaagaaagggtaagaaagttcttatcgtagaacgtgactgggctatgcctgatagaattgttggtgaattgatgcaaccaggtggtgttagagcattgagaagtctgggtatgattcaatctatcaacaacatcgaagcatatcctgttaccggttataccgtctttttcaacggcgaacaagttgatattccatacccttacaaggccgatatccctaaagttgaaaaattgaaggacttggtcaaagatggtaatgacaaggtcttggaagacagcactattcacatcaaggattacgaagatgatgaaagagaaaggggtgttgcttttgttcatggtagattcttgaacaacttgagaaacattactgctcaagagccaaatgttactagagtgcaaggtaactgtattgagatattgaaggatgaaaagaatgaggttgttggtgccaaggttgacattgatggccgtggcaaggtggaattcaaagcccacttgacatttatctgtgacggtatcttttcacgtttcagaaaggaattgcacccagaccatgttccaactgtcggttcttcgtttgtcggtatgtctttgttcaatgctaagaatcctgctcctatgcacggtcacgttattcttggtagtgatcatatgccaatcttggtttaccaaatcagtccagaagaaacaagaatcctttgtgcttacaactctccaaaggtcccagctgatatcaagagttggatgattaaggatgtccaacctttcattccaaagagtctacgtccttcatttgatgaagccgtcagccaaggtaaatttagagctatgccaaactcctacttgccagctagacaaaacgacgtcactggtatgtgtgttatcggtgacgctctaaatatgagacatccattgactggtggtggtatgactgtcggtttgcatgatgttgtcttgttgattaagaaaataggtgacctagacttcagcgaccgtgaaaaggttttggatgaattactagactaccatttcgaaagaaagagttacgattccgttattaacgttttgtcagtggctttgtattctttgttcgctgctgacagcgataacttgaaggcattacaaaaaggttgtttcaaatatttccaaagaggtggcgattgtgtcaacaaacccgttgaatttctgtctggtgtcttgccaaagcctttgcaattgaccagggttttcttcgctgtcgctttttacaccatttacttgaacatggaagaacgtggtttcttgggattaccaatggctttattggaaggtattatgattttgatcacagctattagagtattcaccccatttttgtttggtgagttgattggttaa

SEQ ID NO: 134 - DNA, 987 bp, Saccharomyces cerevisiae
  source 1..987: mol_type = genomic DNA
atgttaactctagagtctgcattaactggcgctgtggcttcggcaatggccaatattgcagtttatccgctggatttatcgaagacgatcattcagtcacaagtatctccttcttcaagtgaggatagtaacgaaggtaaagttttgcccaataggagatataagaatgttgtagattgcatgataaacatattcaaagaaaagggtattttgggtctgtatcaaggtatgacagtcactacggtggccacatttgtccagaattttgtttatttcttttggtacacatttatcagaaagtcctacatgaaacataagctgttaggactgcaatcactgaaaaaccgcgatggtcctatcacaccttctacgattgaagaattggtacttggggtagcagctgccagtatatcgcaactttttactagtcccatggctgtggtagctacaagacaacaaacagtccattctgcagagtctgccaaatttaccaacgttattaaggacatttaccgtgaaaataatggggacataactgctttttggaaaggtttaagaacaggtttagcattgacgataaatccttccatcacatatgcctcttttcaaagacttaaagaagtttttttccatgaccattccaacgatgctggcagtttgtcagcagtgcaaaatttcattttgggtgtcctttccaagatgatttcgactctagttacgcaacccttgattgtcgctaaagcaatgcttcaaagcgctggctctaaattcactactttccaagaagcgctactatacttgtacaaaaatgaagggttaaaatctctttggaagggagttcttcctcaattgacaaagggtgtcattgtgcaaggtctgttgtttgctttcagaggagaattgacaaaatctttaaagaggctaatattcttgtactcttcttttttcctaaagcacaacggacaacgcaagctggcttccacttga

SEQ ID NO: 135 - DNA, 1239 bp, Saccharomyces cerevisiae
  source 1..1239: mol_type = genomic DNA
atgacaaagattaaggtagctaaccccattgtggaaatggacggcgatgagcaaacaagaataatctggcatttaatcagggacaagttagtcttgccctatcttgacgttgatttgaagtactacgatctttccgtggagtatcgtgaccagactaatgatcaagtaactgtggattctgccaccgcgactttaaagtatggagtagctgtcaaatgcgcgactattacacccgatgaggcaagggtcgaggaatttcatttgaaaaagatgtggaaatctccaaatggtactattagaaacattttgggtggtacagtgttcagagaacctattattatccctagaattccaaggctagttcctcaatgggagaagcccatcatcattgggagacacgcattcggcgatcagtacaaagctaccgatgtaatagtccctgaagaaggcgagttgaggcttgtttataaatccaagagcggaactcatgatgtagatctgaaggtatttgactacccagaacatggtggggttgccatgatgatgtacaacactacagattcgatcgaagggtttgcgaaggcctcctttgaattggccattgaaaggaagttaccattatattccactactaagaatactattttgaagaagtatgatggtaaattcaaagatgttttcgaagccatgtatgctagaagttataaagagaagtttgaatcccttggcatctggtacgagcaccgtttaattgatgatatggtggcccaaatgttgaaatctaaaggtggatacataattgccatgaaaaattacgacggtgacgtagaatcagatattgttgcacaaggatttggctccttggggttaatgacatctgtgttgattaccccggacggtaaaacctttgaaagcgaagccgcccacggtacagtaacaagacattttagacagcatcagcaaggaaaggagacgtcaacaaattccattgcatcaattttcgcgtggactagaggtattattcaaaggggtaaacttgataatactccagatgtagttaagttcggccaaatattggaaagcgctacggtaaatacagtgcaagaagatggaatcatgactaaagatttggcgctcattctcggtaagtctgaaagatccgcttatgtcactaccgaggagttcattgacgcggtggaatctagattgaaaaaagagttcgaggcagctgcattgtaa

SEQ ID NO: 136 - DNA, 1263 bp, Saccharomyces cerevisiae
  source 1..1263: mol_type = genomic DNA
atgagtaaaattaaagttgttcatcccatcgtggaaatggacggtgatgagcagacaagagttatttggaaacttatcaaagaaaaattgatattgccatatttagatgtggatttaaaatactatgacctttcaatccaagagcgtgataggactaatgatcaagtaacaaaggattcttcttatgctaccctaaaatatggggttgctgtcaaatgtgccactataacacccgatgaggcaagaatgaaagaatttaaccttaaagaaatgtggaaatctccaaatggaacaatcagaaacatcctaggtggaactgtatttagagaacccatcattattccaaaaatacctcgtctagtccctcactgggagaaacctataattataggccgtcatgcttttggtgaccaatatagggctactgacatcaagattaaaaaagcaggcaaactaaggttacagtttagctcagatgacggtaaagaaaacatcgatttaaaggtttatgaatttcctaaaagtggtgggatcgcaatggcaatgtttaatacaaatgattccattaaagggttcgcaaaggcatccttcgaattagctctcaaaagaaaactaccgttattctttacaaccaaaaacactattctgaaaaattatgataatcagttcaaacaaattttcgataatttgttcgataaagaatataaggaaaagtttcaggctttaaaaataacgtacgagcatcgtttgattgatgatatggtagcacagatgctaaaatcaaagggcgggtttataatcgccatgaagaattatgatggcgatgtccagtctgacattgtggcacaaggatttgggtctcttggtttaatgacgtccatattgattacacctgatggtaaaacgtttgaaagcgaggctgcccatggtacggtgaccagacattttagaaaacatcaaagaggcgaagaaacatcaacaaattcaatagcctcaatatttgcctggacaagggcaattatacaaagaggaaaattagacaatacagatgatgttataaaatttggaaacttactagaaaaggctactttggacacagttcaagtgggcggaaaaatgaccaaggatttagcattgatgcttggaaagactaatagatcatcatatgtaaccacagaagagtttattgatgaagttgccaagaggcttcaaaacatgatgctcagctccaatgaagacaagaaaggtatgtgcaaactataa

SEQ ID NO: 137 - DNA, 1383 bp, Saccharomyces cerevisiae
  source 1..1383: mol_type = genomic DNA
atgacagttccttatctaaattcaaacagaaatgttgcatcatatttacaatcaaattcaagccaagaaaagactctaaaagagagatttagcgaaatctaccccatccatgctcaagatgtaaggcaattcgttaaagagcatggcaaaactaaaattagcgatgttctattagaacaggtatatggtggtatgagaggtattccagggagcgtatgggaaggttccgttttggacccagaagacggtattcgtttcagaggtcgtacgatcgccgacattcaaaaggacctgcccaaggcaaaaggaagctcacaaccactaccagaagctctcttttggttattgctaactggcgaggttccaactcaagcgcaagttgaaaacttatcagctgatctaatgtcaagatcggaactacctagtcatgtcgttcaacttttggataatttaccaaaggacttacacccaatggctcaattctctattgctgtaactgccttggaaagcgagtcaaagtttgctaaggcttatgctcaaggaatttccaagcaagattattggagttatacttttgaagattcactagacttgctgggtaaattgccagttattgcagctaaaatttatcgtaatgtattcaaagatggcaaaatgggtgaagtggacccaaatgccgattatgctaaaaatctggtcaacttgattggttctaaggatgaagatttcgtggacttgatgagactttatttaaccattcattcggatcacgaaggtggtaatgtatctgcacatacatcccatcttgtgggctcagcactatcatcaccttatctgtcccttgcatcaggtttgaacgggttggctggcccacttcatgggcgtgctaatcaagaagtactagaatggttatttgcacttaaagaagaggtaaatgatgactactctaaagatacgatcgaaaaatatttatgggatactctaaactcaggaagagtcattcccggttatggtcatgctgtgctaaggaaaactgatcctcgttatatggctcagcgtaagtttgccatggaccattttccagattatgaattattcaagttagtttcatcaatatacgaggtagcacctggcgtattgactgaacatggtaaaaccaaaaatccatggccaaatgtagatgctcactctggtgtcttattacaatattatggactaaaagaatcttctttctataccgttttatttggcgtttcaagggcatttggtattcttgctcaattgatcactgatagggccatcggtgcttccattgaaaggccaaagtcctattctactgagaaatacaaggaattggtcaaaaacattgaaagcaaactatag

SEQ ID NO: 138 - DNA, 1494 bp, Saccharomyces cerevisiae
  source 1..1494: mol_type = genomic DNA
atgtcagcgaaatccattcacgaggccgacggcaaggccctgctcgcacactttctgtccaaggcgcccgtgtgggccgagcagcagcccatcaacacgtttgaaatgggcacacccaagctggcgtctctgacgttcgaggacggcgtggcccccgagcagatcttcgccgccgctgaaaagacctacccctggctgctggagtccggcgccaagtttgtggccaagcccgaccagctcatcaagcgacgaggcaaggccggcctgctggtactcaacaagtcgtgggaggagtgcaagccctggatcgccgagcgggccgccaagcccatcaacgtggagggcattgacggagtgctgcgaacgttcctggtcgagccctttgtgccccacgaccagaagcacgagtactacatcaacatccactccgtgcgagagggcgactggatcctcttctaccacgagggaggagtcgacgtcggcgacgtggacgccaaggccgccaagatcctcatccccgttgacattgagaacgagtacccctccaacgccacgctcaccaaggagctgctggcacacgtgcccgaggaccagcaccagaccctgctcgacttcatcaaccggctctacgccgtctacgtcgatctgcagtttacgtatctggagatcaaccccctggtcgtgatccccaccgcccagggcgtcgaggtccactacctggatcttgccggcaagctcgaccagaccgcagagtttgagtgcggccccaagtgggctgctgcgcggtcccccgccgctctgggccaggtcgtcaccattgacgccggctccaccaaggtgtccatcgacgccggccccgccatggtcttccccgctcctttcggtcgagagctgtccaaggaggaggcgtacattgcggagctcgattccaagaccggagcttctctgaagctgactgttctcaatgccaagggccgaatctggacccttgtggctggtggaggagcctccgtcgtctacgccgacgccattgcgtctgccggctttgctgacgagctcgccaactacggcgagtactctggcgctcccaacgagacccagacctacgagtacgccaaaaccgtactggatctcatgacccggggcgacgctcaccccgagggcaaggtactgttcattggcggaggaatcgccaacttcacccaggttggatccaccttcaagggcatcatccgggccttccgggactaccagtcttctctgcacaaccacaaggtgaagatttacgtgcgacgaggcggtcccaactggcaggagggtctgcggttgatcaagtcggctggcgacgagctgaatctgcccatggagatttacggccccgacatgcacgtgtcgggtattgttcctttggctctgcttggaaagcggcccaagaatgtcaagccttttggcaccggaccttctactgaggcttccactcctctcggagtttaa

SEQ ID NO: 139 - DNA, 1953 bp, Saccharomyces cerevisiae
  source 1..1953: mol_type = genomic DNA
atgtctgccaacgagaacatctcccgattcgacgcccctgtgggcaaggagcaccccgcctacgagctcttccataaccacacacgatctttcgtctatggtctccagcctcgagcctgccagggtatgctggacttcgacttcatctgtaagcgagagaacccctccgtggccggtgtcatctatcccttcggcggccagttcgtcaccaagatgtactggggcaccaaggagactcttctccctgtctaccagcaggtcgagaaggccgctgccaagcaccccgaggtcgatgtcgtggtcaactttgcctcctctcgatccgtctactcctctaccatggagctgctcgagtacccccagttccgaaccatcgccattattgccgagggtgtccccgagcgacgagcccgagagatcctccacaaggcccagaagaagggtgtgaccatcattggtcccgctaccgtcggaggtatcaagcccggttgcttcaaggttggaaacaccggaggtatgatggacaacattgtcgcctccaagctctaccgacccggctccgttgcctacgtctccaagtccggaggaatgtccaacgagctgaacaacattatctctcacaccaccgacggtgtctacgagggtattgctattggtggtgaccgataccctggtactaccttcattgaccatatcctgcgatacgaggccgaccccaagtgtaagatcatcgtcctccttggtgaggttggtggtgttgaggagtaccgagtcatcgaggctgttaagaacggccagatcaagaagcccatcgtcgcttgggccattggtacttgtgcctccatgttcaagactgaggttcagttcggccacgccggctccatggccaactccgacctggagactgccaaggctaagaacgccgccatgaagtctgctggcttctacgtccccgataccttcgaggacatgcccgaggtccttgccgagctctacgagaagatggtcgccaagggcgagctgtctcgaatctctgagcctgaggtccccaagatccccattgactactcttgggcccaggagcttggtcttatccgaaagcccgctgctttcatctccactatttccgatgaccgaggccaggagcttctgtacgctggcatgcccatttccgaggttttcaaggaggacattggtatcggcggtgtcatgtctctgctgtggttccgacgacgactccccgactacgcctccaagtttcttgagatggttctcatgcttactgctgaccacggtcccgccgtatccggtgccatgaacaccattatcaccacccgagctggtaaggatctcatttcttccctggttgctggtctcctgaccattggtacccgattcggaggtgctcttgacggtgctgccaccgagttcaccactgcctacgacaagggtctgtccccccgacagttcgttgataccatgcgaaagcagaacaagctgattcctggtattggccatcgagtcaagtctcgaaacaaccccgatttccgagtcgagcttgtcaaggactttgttaagaagaacttcccctccacccagctgctcgactacgcccttgctgtcgaggaggtcaccacctccaagaaggacaacctgattctgaacgttgacggtgctattgctgtttcttttgtcgatctcatgcgatcttgcggtgcctttactgtggaggagactgaggactacctcaagaacggtgttctcaacggtctgttcgttctcggtcgatccattggtctcattgcccaccatctcgatcagaagcgactcaagaccggtctgtaccgacatccttgggacgatatcacctacctggttggccaggaggctatccagaagaagcgagtcgagatcagcgccggcgacgtttccaaggccaagactcgatcatag

SEQ ID NO: 140 - DNA, 1335 bp, Saccharomyces cerevisiae
  source 1..1335: mol_type = genomic DNA
atgccatctcatttcgatactgttcaactacacgccggccaagagaaccctggtgacaatgctcacagatccagagctgtaccaatttacgccaccacttcttatgttttcgaaaactctaagcatggttcgcaattgtttggtctagaagttccaggttacgtctattcccgtttccaaaacccaaccagtaatgttttggaagaaagaattgctgctttagaaggtggtgctgctgctttggctgtttcctccggtcaagccgctcaaacccttgccatccaaggtttggcacacactggtgacaacatcgtttccacttcttacttatacggtggtacttataaccagttcaaaatctcgttcaaaagatttggtatcgaggctagatttgttgaaggtgacaatccagaagaattcgaaaaggtctttgatgaaagaaccaaggctgtttatttggaaaccattggtaatccaaagtacaatgttccggattttgaaaaaattgttgcaattgctcacaaacacggtattccagttgtcgttgacaacacatttggtgccggtggttacttctgtcagccaattaaatacggtgctgatattgtaacacattctgctaccaaatggattggtggtcatggtactactatcggtggtattattgttgactctggtaagttcccatggaaggactacccagaaaagttccctcaattctctcaacctgccgaaggatatcacggtactatctacaatgaagcctacggtaacttggcatacatcgttcatgttagaactgaactattaagagatttgggtccattgatgaacccatttgcctctttcttgctactacaaggtgttgaaacattatctttgagagctgaaagacacggtgaaaatgcattgaagttagccaaatggttagaacaatccccatacgtatcttgggtttcataccctggtttagcatctcattctcatcatgaaaatgctaagaagtatctatctaacggtttcggtggtgtcttatctttcggtgtaaaagacttaccaaatgccgacaaggaaactgacccattcaaactttctggtgctcaagttgttgacaatttaaagcttgcctctaacttggccaatgttggtgatgccaagaccttagtcattgctccatacttcactacccacaaacaattaaatgacaaagaaaagttggcatctggtgttaccaaggacttaattcgtgtctctgttggtatcgaatttattgatgacattattgcagacttccagcaatcttttgaaactgttttcgctggccaaaaaccatga

SEQ ID NO: 141 - DNA, 753 bp, Saccharomyces cerevisiae
  source 1..753: mol_type = genomic DNA
atgcctttgaccacaaaacctttatctttgaaaatcaacgccgctctattcgatgttgacggtaccatcatcatctctcaaccagccattgctgctttctggagagatttcggtaaagacaagccttacttcgatgccgaacacgttattcacatctctcacggttggagaacttacgatgccattgccaagttcgctccagactttgctgatgaagaatacgttaacaagctagaaggtgaaatcccagaaaagtacggtgaacactccatcgaagttccaggtgctgtcaagttgtgtaatgctttgaacgccttgccaaaggaaaaatgggctgtcgccacctctggtacccgtgacatggccaagaaatggttcgacattttgaagatcaagagaccagaatacttcatcaccgccaatgatgtcaagcaaggtaagcctcacccagaaccatacttaaagggtagaaacggtttgggtttcccaattaatgaacaagacccatccaaatctaaggttgttgtctttgaagacgcaccagctggtattgctgctggtaaggctgctggctgtaaaatcgttggtattgctaccactttcgatttggacttcttgaaggaaaagggttgtgacatcattgtcaagaaccacgaatctatcagagtcggtgaatacaacgctgaaaccgatgaagtcgaattgatctttgatgactacttatacgctaaggatgacttgttgaaatggtaa

SEQ ID NO: 142 - DNA, 1302 bp, Saccharomyces cerevisiae
  source 1..1302: mol_type = genomic DNA
atgactggtaagaccggtcatattgatggtttgaactccagaatcgaaaagatgagagatttggatccagctcaaagattggttagagttgctgaagctgctggtttggaaccagaagctatttctgctttggctggtaatggtgctttgccattgtctttggctaatggtatgatcgaaaacgtcatcggtaagttcgaattgccattgggtgttgctactaatttcactgttaacggtagagactacttgattccaatggctgttgaagaaccatctgttgttgctgctgcttcttatatggctagaattgctagagaaaacggtggttttactgctcatggtactgctccattgatgagagcacaaattcaagttgttggtttgggtgatccagaaggtgctagacaaagattattggctcataaggctgcttttatggaagctgcagatgctgttgatccagttttggttggtttaggtggtggttgtagagatatcgaagttcacgtttttagagatactccagttggtgccatggttgtcttgcatttgatagttgatgttagagatgctatgggtgctaacactgttaataccatggctgaaagattggctccagaagttgaaagaattgctggtggtactgttagattgaggatcttgtctaatttggccgatttgagattagttagagccagagttgaattggctcctgaaactttgactactcaaggttatgatggtgctgatgttgctagaggtatggttgaagcttgtgctttagctatcgttgatccatatagagctgctactcataacaagggtattatgaacggtatcgatccagttgttgttgccactggtaatgattggagagctattgaagctggtgcacatgcttatgctgctagaactggtcattatacttcattgaccagatgggaattagccaacgatggtagattggttggtactattgaattgcctttggccttgggtttagtaggtggtgctacaaaaactcatccaactgctagagctgcattggctttgatgcaagttgaaactgctactgaattggcacaagttactgctgctgtaggtttggctcaaaacatggctgctattagagctttggctactgaaggtattcaaaggggtcacatgactttacatgctagaaacattgctattatggctggtgctactggtgcagatattgatagagttactagagttattgtcgaagccggtgatgtttctgttgcaagagctaaacaagttttggagaacacctaa

SEQ ID NO: 143 - DNA, 1335 bp, Saccharomyces cerevisiae
  source 1..1335: mol_type = genomic DNA
atgggaaagctattacaattggcattgcatccggtcgagatgaaggcagctttgaagctgaagttttgcagaacaccgctattctccatctatgatcagtccacgtctccatatctcttgcactgtttcgaactgttgaacttgacctccagatcgtttgctgctgtgatcagagagctgcatccagaattgagaaactgtgttactctcttttatttgattttaagggctttggataccatcgaagacgatatgtccatcgaacacgatttgaaaattgacttgttgcgtcacttccacgagaaattgttgttaactaaatggagtttcgacggaaatgcccccgatgtgaaggacagagccgttttgacagatttcgaatcgattcttattgaattccacaaattgaaaccagaatatcaagaagtcatcaaggagatcaccgagaaaatgggtaatggtatggccgactacatcttagatgaaaattacaacttgaatgggttgcaaaccgtccacgactacgacgtgtactgtcactacgtagctggtttggtcggtgatggtttgacccgtttgattgtcattgccaagtttgccaacgaatctttgtattctaatgagcaattgtatgaaagcatgggtcttttcctacaaaaaaccaacatcatcagagattacaatgaagatttggtcgatggtagatccttctggcccaaggaaatctggtcacaatacgctcctcagttgaaggacttcatgaaacctgaaaacgaacaactggggttggactgtataaaccacctcgtcttaaacgcattgagtcatgttatcgatgtgttgacttatttggccggtatccacgagcaatccactttccaattttgtgccattccccaagttatggccattgcaaccttggctttggtattcaacaaccgtgaagtgctacatggcaatgtaaagattcgtaagggtactacctgctatttaattttgaaatcaaggactttgcgtggctgtgtcgagatttttgactattacttacgtgatatcaaatctaaattggctgtgcaagatccaaatttcttaaaattgaacattcaaatctccaagatcgaacagtttatggaagaaatgtaccaggataaattacctcctaacgtgaagccaaatgaaactccaattttcttgaaagttaaagaaagatccagatacgatgatgaattggttccaacccaacaagaagaagagtacaagttcaatatggttttatctatcatcttgtccgttcttcttgggttttattatatatacactttacacagagcgtga

SEQ ID NO: 144 - DNA, 1176 bp, Saccharomyces cerevisiae
  source 1..1176: mol_type = genomic DNA
atgtctgctgctgctgatagattaaacttaacttccggccacttgaatgctggtagaaagagaagttcctcttctgtttctttgaaggctgccgaaaagcctttcaaggttactgtgattggatctggtaactggggtactactattgccaaggtggttgccgaaaattgtaagggatacccagaagttttcgctccaatagtacaaatgtgggtgttcgaagaagagatcaatggtgaaaaattgactgaaatcataaatactagacatcaaaacgtgaaatacttgcctggcatcactctacccgacaatttggttgctaatccagacttgattgattcagtcaaggatgtcgacatcatcgttttcaacattccacatcaatttttgccccgtatctgtagccaattgaaaggtcatgttgattcacacgtcagagctatctcctgtctaaagggttttgaagttggtgctaaaggtgtccaattgctatcctcttacatcactgaggaactaggtattcaatgtggtgctctatctggtgctaacattgccaccgaagtcgctcaagaacactggtctgaaacaacagttgcttaccacattccaaaggatttcagaggcgagggcaaggacgtcgaccataaggttctaaaggccttgttccacagaccttacttccacgttagtgtcatcgaagatgttgctggtatctccatctgtggtgctttgaagaacgttgttgccttaggttgtggtttcgtcgaaggtctaggctggggtaacaacgcttctgctgccatccaaagagtcggtttgggtgagatcatcagattcggtcaaatgtttttcccagaatctagagaagaaacatactaccaagagtctgctggtgttgctgatttgatcaccacctgcgctggtggtagaaacgtcaaggttgctaggctaatggctacttctggtaaggacgcctgggaatgtgaaaaggagttgttgaatggccaatccgctcaaggtttaattacctgcaaagaagttcacgaatggttggaaacatgtggctctgtcgaagacttcccattatttgaagccgtataccaaatcgtttacaacaactacccaatgaagaacctgccggacatgattgaagaattagatctacatgaagattag

SEQ ID NO: 145 - DNA, 1323 bp, Saccharomyces cerevisiae
  source 1..1323: mol_type = genomic DNA
atgcttgctgtcagaagattaacaagatacacattccttaagcgaacgcatccggtgttatatactcgtcgtgcatataaaattttgccttcaagatctactttcctaagaagatcattattacaaacacaactgcactcaaagatgactgctcatactaatatcaaacagcacaaacactgtcatgaggaccatcctatcagaagatcggactctgccgtgtcaattgtacatttgaaacgtgcgcccttcaaggttacagtgattggttctggtaactgggggaccaccatcgccaaagtcattgcggaaaacacagaattgcattcccatatcttcgagccagaggtgagaatgtgggtttttgatgaaaagatcggcgacgaaaatctgacggatatcataaatacaagacaccagaacgttaaatatctacccaatattgacctgccccataatctagtggccgatcctgatcttttacactccatcaagggtgctgacatccttgttttcaacatccctcatcaatttttaccaaacatagtcaaacaattgcaaggccacgtggcccctcatgtaagggccatctcgtgtctaaaagggttcgagttgggctccaagggtgtgcaattgctatcctcctatgttactgatgagttaggaatccaatgtggcgcactatctggtgcaaacttggcaccggaagtggccaaggagcattggtccgaaaccaccgtggcttaccaactaccaaaggattatcaaggtgatggcaaggatgtagatcataagattttgaaattgctgttccacagaccttacttccacgtcaatgtcatcgatgatgttgctggtatatccattgccggtgccttgaagaacgtcgtggcacttgcatgtggtttcgtagaaggtatgggatggggtaacaatgcctccgcagccattcaaaggctgggtttaggtgaaattatcaagttcggtagaatgtttttcccagaatccaaagtcgagacctactatcaagaatccgctggtgttgcagatctgatcaccacctgctcaggcggtagaaacgtcaaggttgccacatacatggccaagaccggtaagtcagccttggaagcagaaaaggaattgcttaacggtcaatccgcccaagggataatcacatgcagagaagttcacgagtggctacaaacatgtgagttgacccaagaattcccattattcgaggcagtctaccagatagtctacaacaacgtccgcatggaagacctaccggagatgattgaagagctagacatcgatgacgaatag